(19) United States
(12) Patent Application Publication
(10) Pub. No.: US 2006/0252716 A1
(43) Pub. Date: Nov. 9, 2006

(54) Method for reversing the immunosuppressive effects of the melanoma inhibitory activity "mia"

(76) Inventors: Karl-Hermann Schlingensiepen, Donaustauf (DE); Reimar Schlingensiepen, Regensburg (DE); Rainer Apfel, Sinzig (DE); Wolfgang Brysch, Göttingen (DE); Piotr Jachimczak, Würzburg (DE); Ulrich Bogdahn, Regensburg (DE)

Correspondence Address: JACOBSON HOLMAN PLLC, 400 SEVENTH STREET N.W., SUITE 600, WASHINGTON, DC, 20004, US

(21) Appl. No.: 10/220,265

(22) PCT Filed: Mar. 10, 2001
(86) PCT No.: PCT/EP01/02695

§ 371 Date: Dec. 4, 2004

(30) Foreign Application Priority Data
Mar. 11, 2000 (EP) 00105189.5

(51) Int. Cl. A61K 48/00

(52) U.S. Cl. 514/44

(57) ABSTRACT

A method for stimulating immune cells and/or the immune system, and/or reducing invasion and/or metastasis of tumor cells by inhibiting expression and/or functional activity of “Melanoma Inhibitory Activity” MIA.

The polypeptide “Melanoma Inhibitory Activity”, MIA, was discovered in 1989 as a factor that inhibits growth of melanoma tumor cells. The antiproliferative action of MIA was also demonstrated in other tumor cells and Peripheral Blood Mononuclear Cells. Thus, CANCER RES. 49; 5358-63, Bogdahn et al, (1989) demonstrated a very strong tumor cell growth-inhibiting effect of a factor called melanoma inhibitory activity (MIA). Three active fraction pools, named MIA-I, MIA-II and MIA-III were identified. Tumor stem cell colony formation was reduced by an astonishing 99.890/0 e.g. by MIA-II.

CANCER RES 50; 6981-86, Weilbach et al. (1990) further demonstrated that MIA inhibits cell proliferation by prolonging of the S-Phase and arrest of the cells in the G2 compartment.

PROC.AACR 40; 79, Jachimczak et al. (1999) further extended these observations to Peripheral Blood Mononuclear Cells (PBMCs), although to only a slight degree: “IL-2-stimulated PBMC proliferation has been only slightly inhibited by addition of MIA”.

CANCER RES. 54; 5695-5701, Blesch et al. (1994) identified MIA as a 131-amino acid precursor, processed into a mature 107-amino acid protein. This publication confirmed that MIA acts as a potent tumor cell growth inhibitor for malignant melanoma cell and further extended this observation to other neuroectodermal tumors and concluded that “ . . . MIA . . . might be attractive as a future antitumor therapeutical substance.”

Controversial data were obtained regarding correlation of MIA expression with melanoma progression. CANCER RES. 57; 3149-53, Bosserhoff et al. (1997) and ANTICANCER RES. 19; 2691-3, Bosserhoff et al. (1997) found enhanced MIA levels in 13-23% of stage I and II melanomas, but in 100% of stage III or stage IV disease.

In contrast, CANCER RES. 55; 6237-43, van Groningen et al. (1995) found MIA mRNA expression in non metastasising cell lines and an inverse correlation of MIA mRNA expression with pigmentation in melanoma metastasis lesions, but notably expression was found to be absent in highly metastasising cell lines. Furthermore, CLIN. CANCER RES. 5; 1099-105, Muhlbauer et al. (1997) concluded that “ . . . MIA amplification seems to be of little value as a surrogate marker for clinical staging or the detection of metastatic disease.”

Surprisingly while the above literature suggested that MIA had the potential as a therapeutic agent to treat melanoma patients it was now found that in contrast MIA is a potent immunosuppressive factor and that agents inhibiting expression and/or function of MIA have therapeutic potential for treatment of neoplasms and immunosuppression.

The present invention therefore pertains to a method for stimulating immune cells and/or the immune system, and/or reducing invasion and/or metastasis of tumor cells by inhibiting expression and/or functional activity of “Melanoma Inhibitory Activity” MIA.

According to the invention the stimulation of the immune system is preferably achieved by inhibiting expression and/or functional activity of “Melanoma Inhibitory Activity” MIA in combination with enhancing expression in target cells and/or target pathogens of the molecules listed under a) to m);

alternatively by vaccination with DNA and/or RNA coding for all or part of the molecules listed under a) to m) and/or polypeptides contained in the molecules listed under a) to m);

by transfection of an organism and/or transfecting the target cells and/or target pathogens with genes coding for the molecules listed under a) to m);

by applying to an organism and/or to the target cells and/or target pathogens the molecules listed under a) to m);

and/or by enhancing the synthesis and/or function of molecules stimulating and/or enhancing and/or upregulating and/or positively regulating the immune response with molecules including the molecules listed under a) to m), wherein

a) represents molecules selected from the group comprising chemokines, including lymphotactin and/or immune cell attracting factors;

b) represents elements selected from the group comprising viruses and/or parts of viruses, including adeno viruses, papilloma viruses, Epstein-barr-Viruses, viruses that are non-pathogenic including Newcastle-Disease virus, Cow-pox-virus;

c) represents molecules selected from the group comprising autologous and/or heterologous MHC-molecules;

d) represents molecules selected from the group comprising molecules involved in antigen processing;

e) represents molecules selected from the group comprising molecules involved in antigen presentation;

f) represents molecules selected from the group comprising molecules involved in mediating immune-cell effects;

g) represents molecules selected from the group comprising molecules involved in mediating immune cell cytotoxic effects;

h) represents moledules selected from the group comprising molecules involved in antigen transportation;

i) represents molecules selected from the group, comprising co-stimulatory molecules;

j) represents molecules selected from the group comprising peptides enhancing recognition by immune cell and/or cytotoxic effects of immune cells;

k) represents molecules selected from the group comprising peptides containing one or more amino acids differing between a protein in the target cell from the other cells within an organism including, but not limited to antigens, specific for melanoma cells and/or melanocytes and/or breast cells and/or breast cancer cells;

according to the invention the inhibition of the syntheses and/or function of MIA is achieved by using molecule of group 1) wherein

l) represents molecules selected from the group comprising the peptides according to j) being

peptides containing one or more mutations and/or amino acid substitutions of the ras protein amino, the p53 protein, the EGF receptor protein, fusion peptides and/or fusion proteins, the retinoblastoma protein, peptides containing one or more mutations and/or amino acid substitutions and/or amino acid substitutions caused by gene rearrangements and/or gene translocations, peptides containing one or more mutations and/or amino acid substitutions of proteins coded by oncogenes and/or protooncogenes, proteins coded by anti-oncogenes and/or tumor suppressor genes;

peptides derived from proteins differing in the target cell by one or amino acids from the proteins expressed by other cells in tile same organism,

peptides derived from viral antigens and/or coded by viral nucleic acids,

peptides derived from proteins over expressed in the target cell compared to a normal cell

and combinations thereof

m) tumor cell extracts and/or tumor cell lysates and/or adjuvants.

In a preferred embodiment of the invention the inhibition of the expression and/or functional activity of MIA is achieved by using at least one nucleic acid molecule, peptide, protein or low molecular weight substance. Preferably, the nucleic acid molecule is an oligo- or polynucleotide molecule, in particular an antisense molecule and/or ribozyme.

Methods for preparing effective antisense oligonucleotides are known to those skilled in the art. A preferred method is disclosed in WO 99/63975, incorporated by reference.

The inhibition of the synthesis and/or function of MIA is preferably achieved by using at molecules comprising the following antisense sequences:

| | | |
|---|---|---|
| MIA-2841-W | GTC AGG AAT CGG GAG | (Seq. ID No 1) |
| MIA-1278-W | CTT GGA GAA GAG ATA C | (Seq. ID No 2) |
| MIA-2842-W | TGC CTC CCC AGA AG | (Seq. ID No 3) |

as well as the following sequences (Seq. ID No 10-39):

AGCCATGGAGATAG
CAGCCATGGAGATAG
ACAGCCATGGAGATAG
CACAGCCATGGAGATAG
CCACAGCCATGGAGAT
GCCATGGAGATAGG
AGCCATGGAGATAGG
CAGCCATGGAGATAGG
ACAGCCATGGAGATAGG
CATGGAGATAGGGT
CATGGAGATAGGGTG
CATGGAGATAGGGTGG
ATGGAGATAGGGTG
ATGGAGATAGGGTGG
ATGGAGATAGGGTGGC
ATGGAGATAGGGTGGCT
GGAGATAGGGTGGC
GGAGATAGGGTGGCT
GAAATAGCCCAGGC
GAAATAGCCCAGGCG
GAAATAGCCCAGGCGAG
GGAAATAGCCCAGG
GGAAATAGCCCAGGC
GTCTTCACATCGAC
GTCTTCACATCGACT
GTCTTCACATCGACTT
GTCTTCACATCGACTTT
GTCTTCACATCGACTTTG
GTCTTCACATCGACTTTG
CCATTTGTCTGTCTTCAC

or parts of the sequences having at least 8 nucleotides.

Methods for synthesizing further antisense oligonucleotides are for example disclosed in WO 98/33904 of the same applicant.

Preferably, the antisense and/or ribozyme molecule is derived by synthesising a sequence wholly or partially complementary to MIA mRNA and testing for inhibitory activity of MIA.

According to the invention the antisense and/or ribozyme molecule is for example integrated into a DNA delivery system, comprising viral and/or non-viral vectors together with lipids selected from the group of anionic lipids, cationic lipids, non-cationic lipids and mixtures thereof.

In a preferred embodiment of the invention the nucleic acid molecules contain flanking sequences and/or vector sequences and/or sequences enhancing the expression and/or transfection of the nucleic acid molecules. In a further preferred embodiment of the invention the nucleic acid molecules are part of one or more vectors and/or viral sequences and/or viral vectors.

According to the invention it is preferred that the antisense and/or ribozyme molecule is modified at one or more of the sugar moieties, the bases and/or the internucleotide linkages as well as the phosphate moieties. For example, the modification of the oligonuclecotides, ribozymes and/or nucleic acids comprises modifications such as phosphorothioate (S-ODN) internucleotide linkages, methylphosphonate internucleotide linkages, phosphoramidate linkages, peptide linkages, 2′-O-alkyl modifications of the sugar, in particular methyl, ethyl, propyl, butyl and the like, 2′-methoxyethoxy modifications of the sugar and/or modifications of the bases. The various modifications may be combined in an oligo- or polynucleotid.

In a further preferred embodiment of the invention the oligonucleotides, ribozymes and/or nucleic acids are coupled to or mixed with folic acid, hormones, steroid hormones such as oestrogene, progesterone, corticosteroids, mineral corticoids, peptides, proteoglycans, glycolipids, phospholipids and derivatives therof.

Inhibition of the expression and/or functional activity of MIA can also be achieved using peptides or proteins.

The peptide and/or protein that can be used in the method of the invention can be obtained by screening an expression library and testing the expression products for inhibiting expression and/or functional activity of MIA.

Alternatively, a synthetic peptide and/or protein can be obtained by screening randomly synthesised peptides and/or polypeptides for inhibiting expression and/or functional activity of MIA.

Suitable peptides binding to MIA are for example the following peptides (SEQ ID No. 40-63):

VPHIPPN
MPPTQVS
QMHPWPP
QPPFWQF
TPPQGLA
IPPYNTL
AVRPAPL
GAKPHPQ
QQLSPLP
GPPPSPV
LPLTPLP
QLNVNHQARADQ
TSASTRPELHYP
TFLPHQMHPWPP
VPHIPPNSMALT
RLTLLVLIMPAP
RKLPPRPRR
VLASQIATTPSP
TPLTKLPSVNHP
PPNSFSSAGGQRT
EQDSRQGQELTKKGL
ETTIVITWTPAPR
TSLLISWDAPAVT
NSLLVSWQPPRAR

and proteins or peptides comprising the forgoing peptides and analogs or derivatives of these peptides.

EMBO J. 20; 340-349, Stoll et al. (2001) disclose the three-dimensional structure of human MIA, thus allowing the computational construction and synthesis of specific peptides binding to MIA.

In a further embodiment of the invention, the inhibition of expression and/or functional activity of MIA as well as of the expression of the MIA gene and/or MIA mRNA is achieved by using an inhibitor of low molecular weight which can for example be obtained by combinatorial chemistry and testing the products for inhibiting expression and/or functional activity of MIA [Fernandes, P. B. in Curr. Opin. Chem. Biol. 1998; 2 (5): 597-803 Technological advances in high-throughput screening].

Low molecular weight molecules (small molecules) as used herein are molecules having up to 100 carbon atoms in combination with further atoms such as N, S, O, P and the like.

Suitable small molecules can also be identified using computational methods. Methods for computational construction are for example disclosed in Murcko, M. A. , Caron, P. R., Charifson, P. S. (1999), Structure-based drug design, Annual Reports in Medicinal Chemistry, vol. 34, Academic Press, San Diego, 1999.

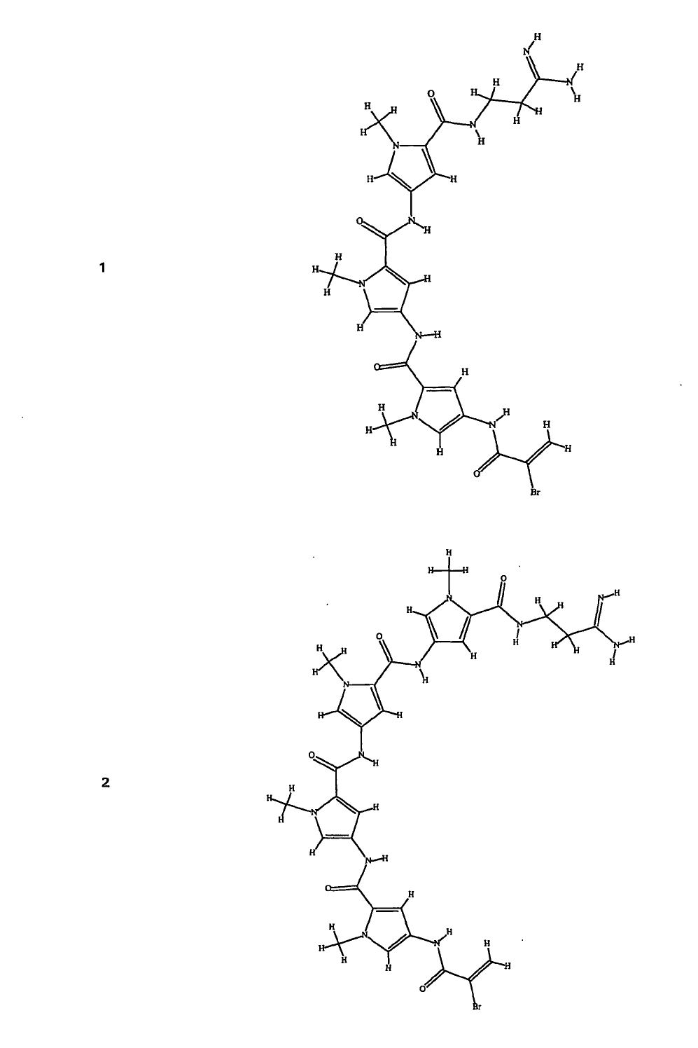

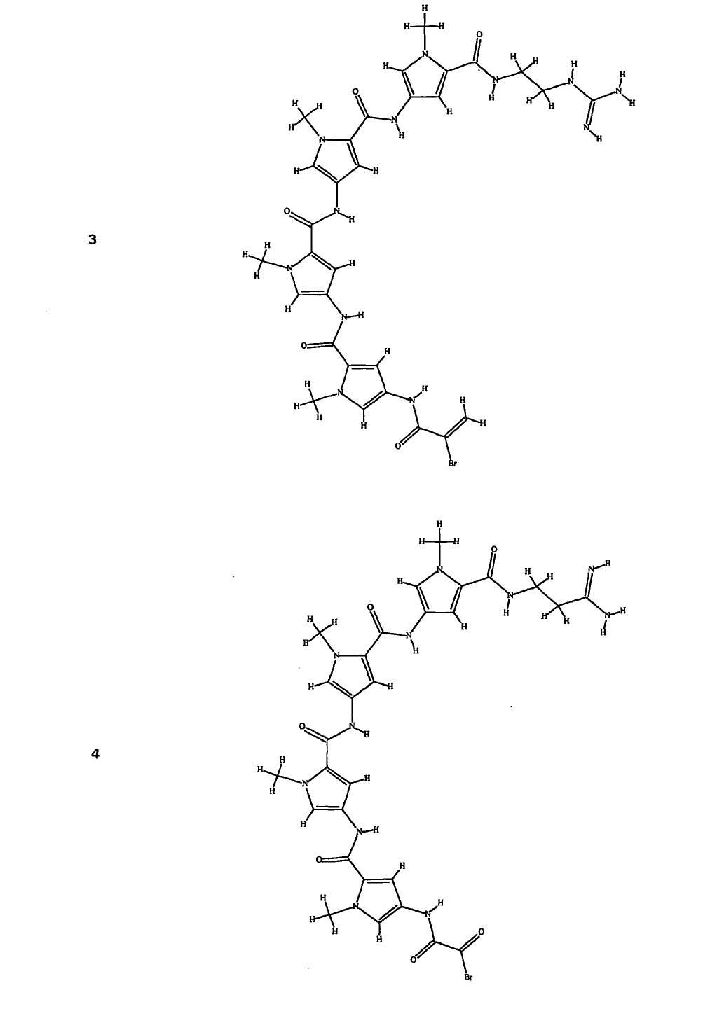

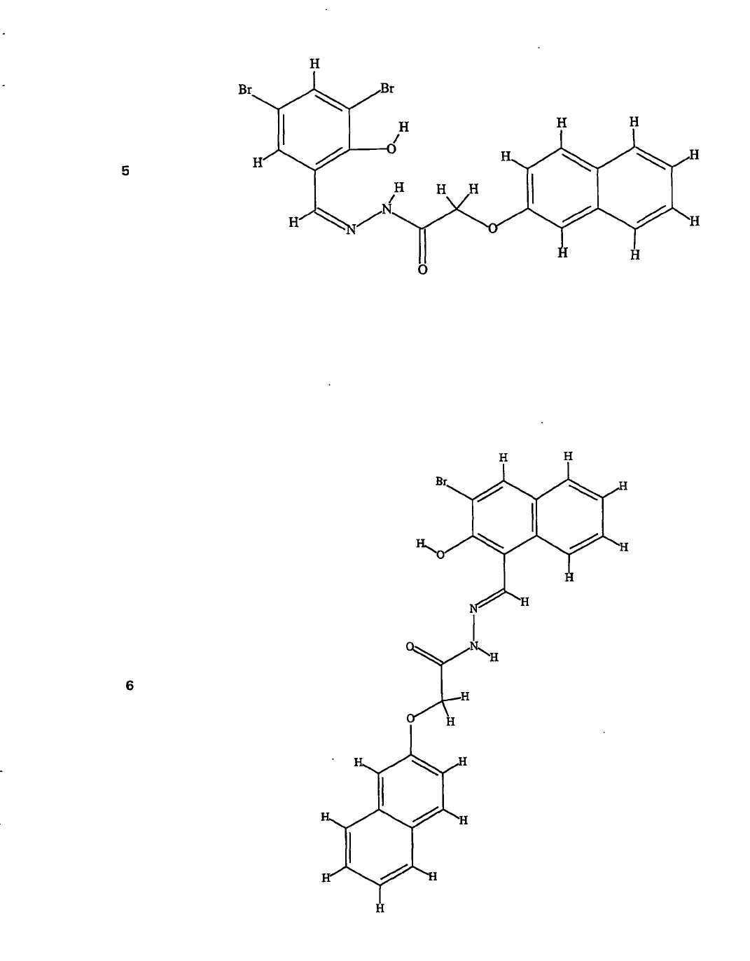

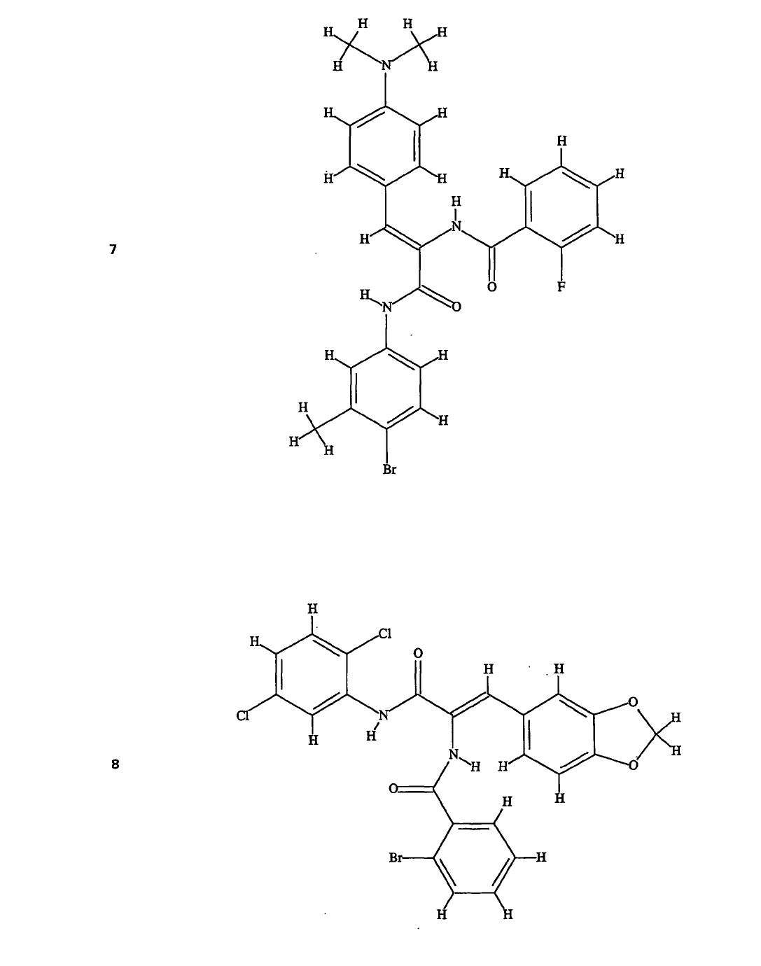

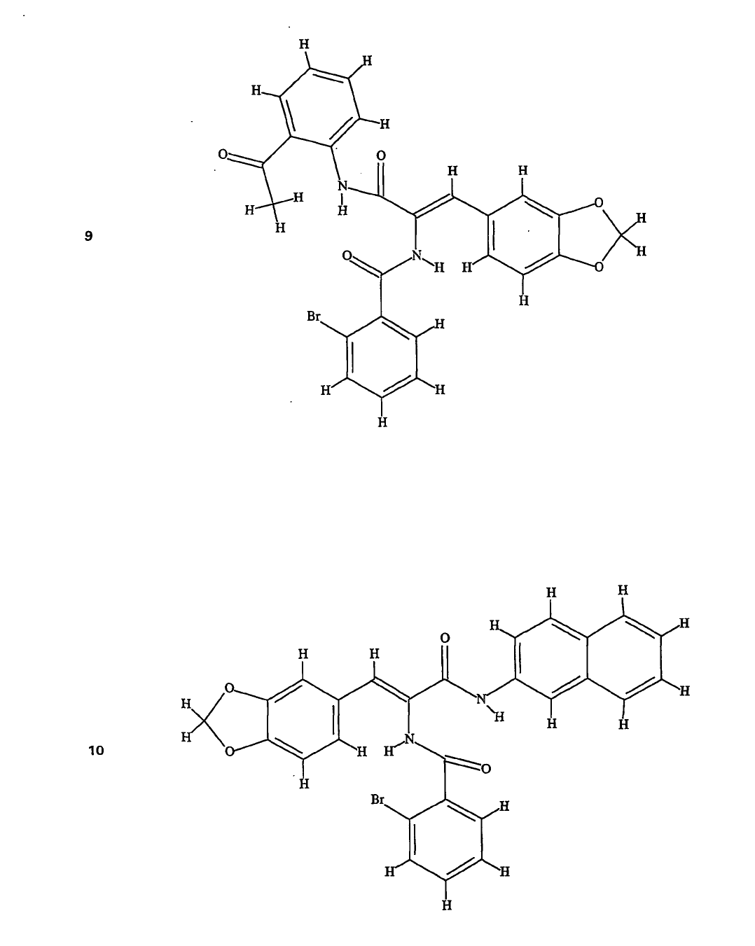

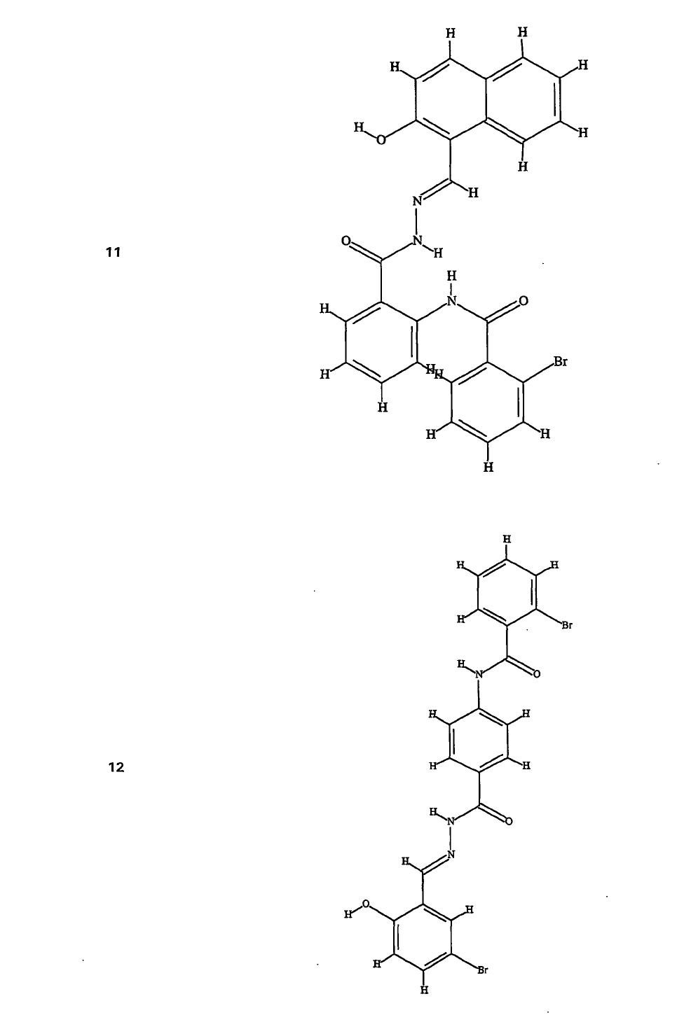

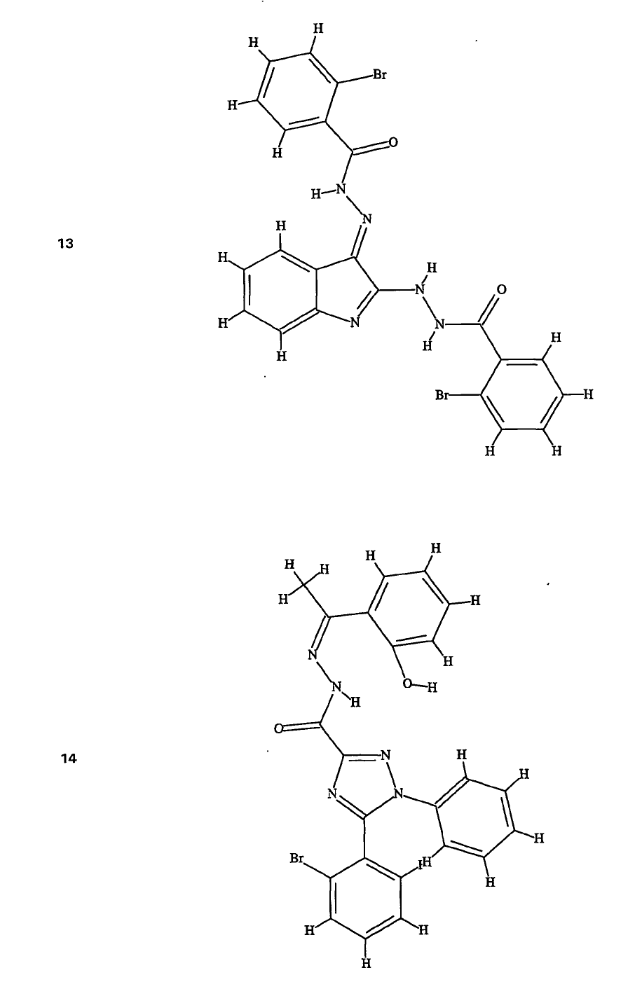

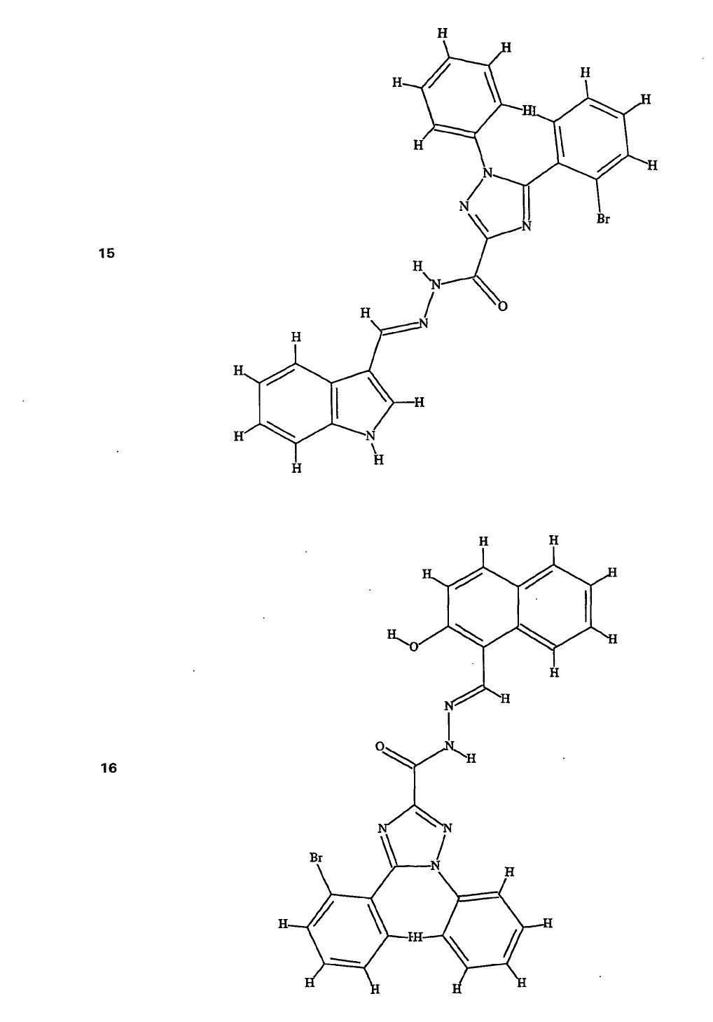

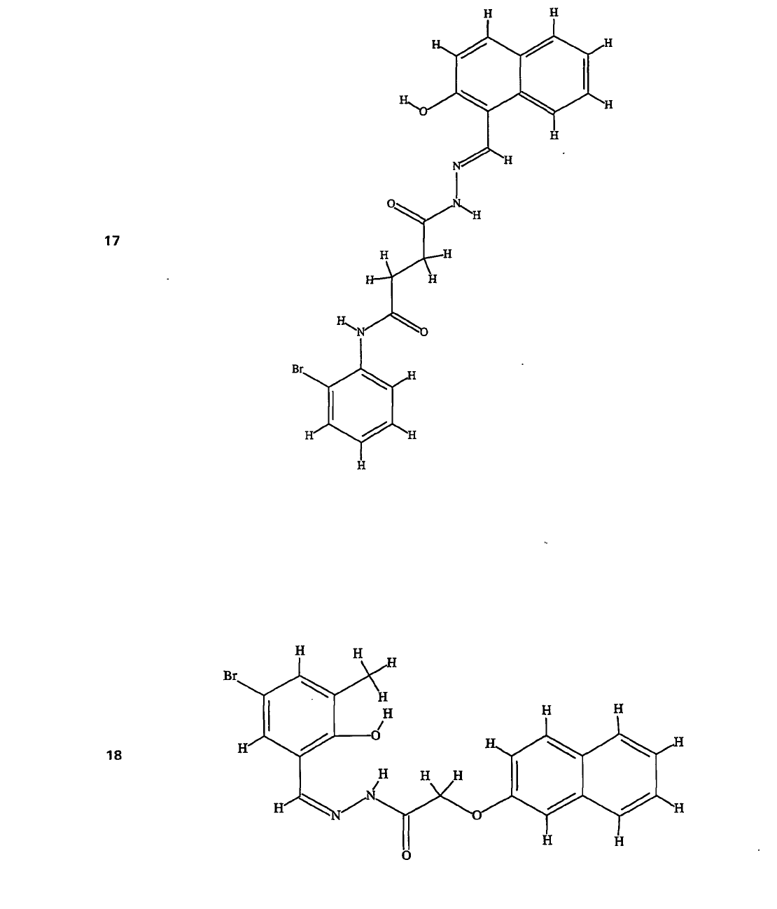

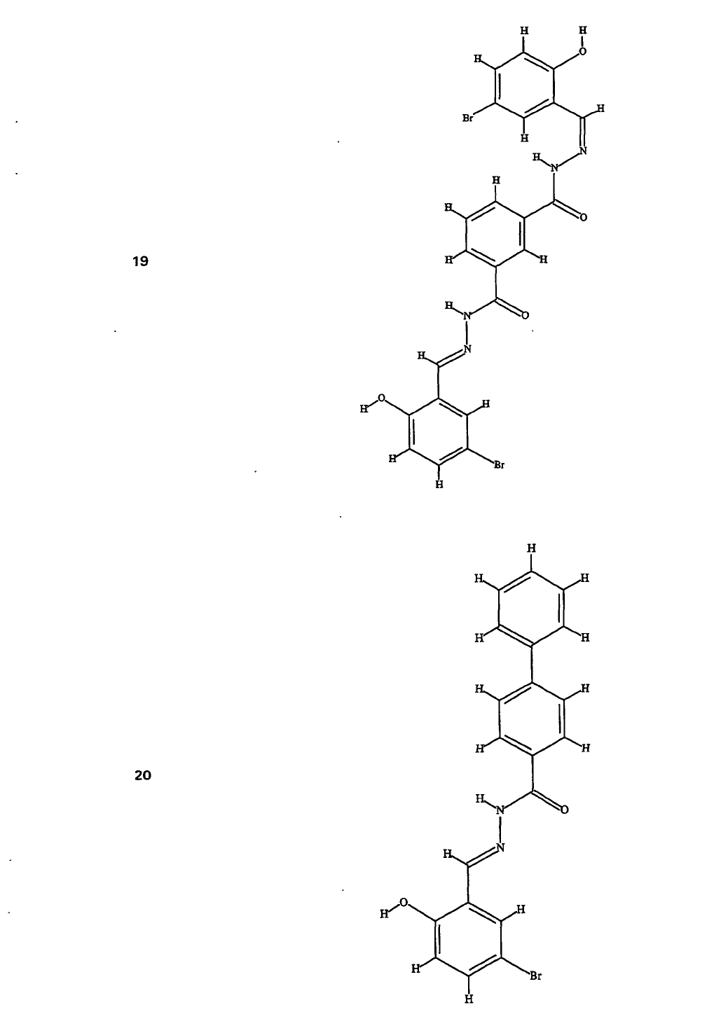

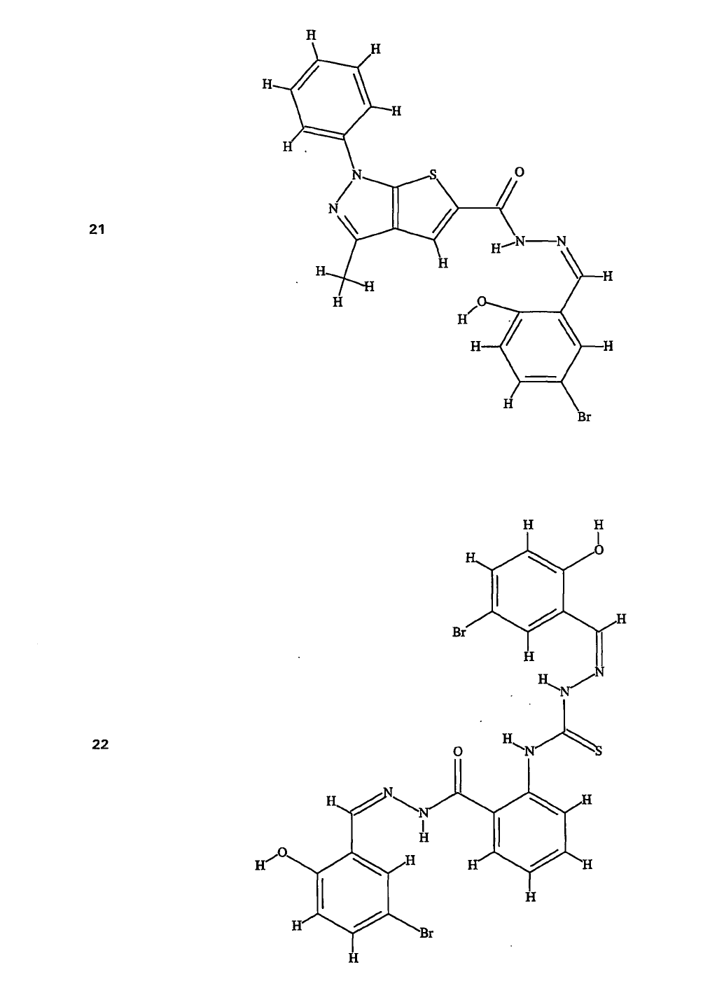

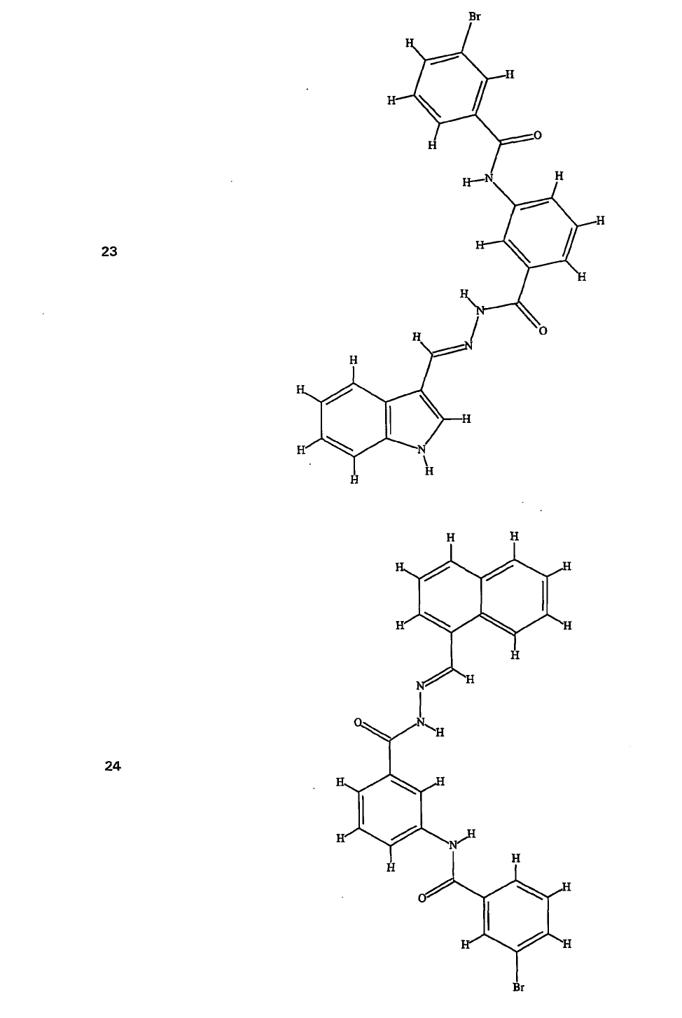

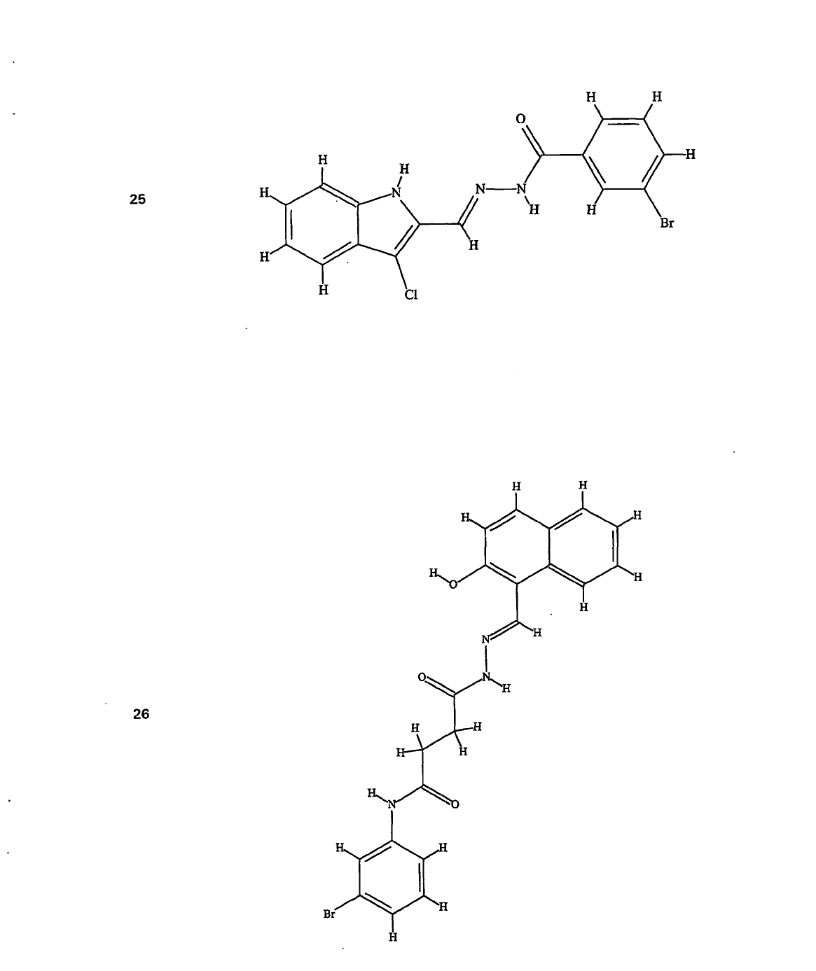

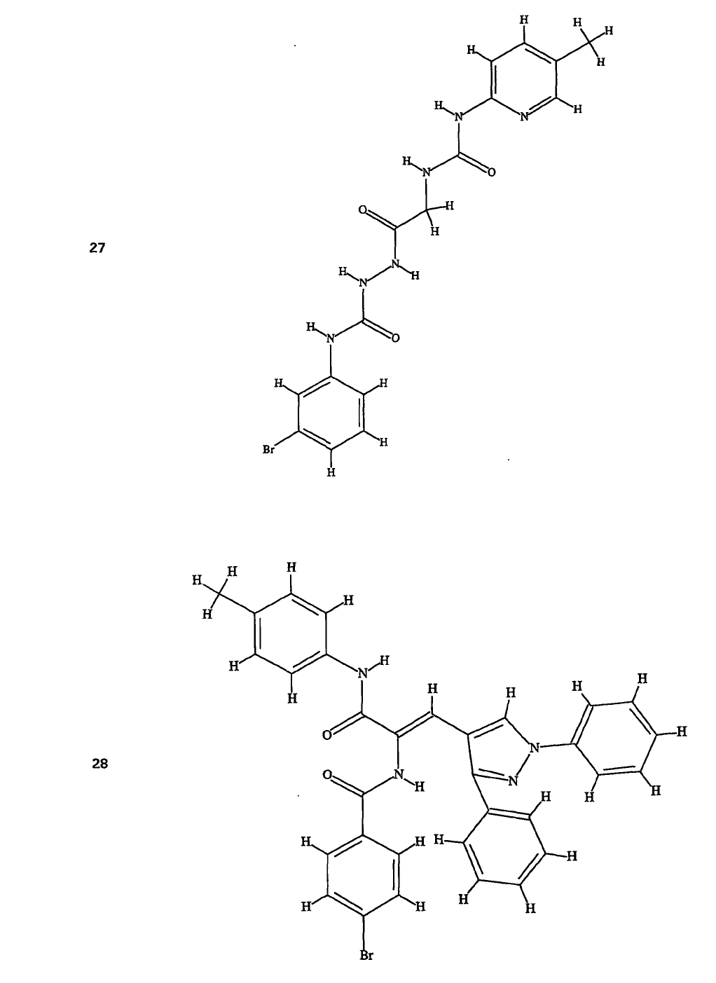

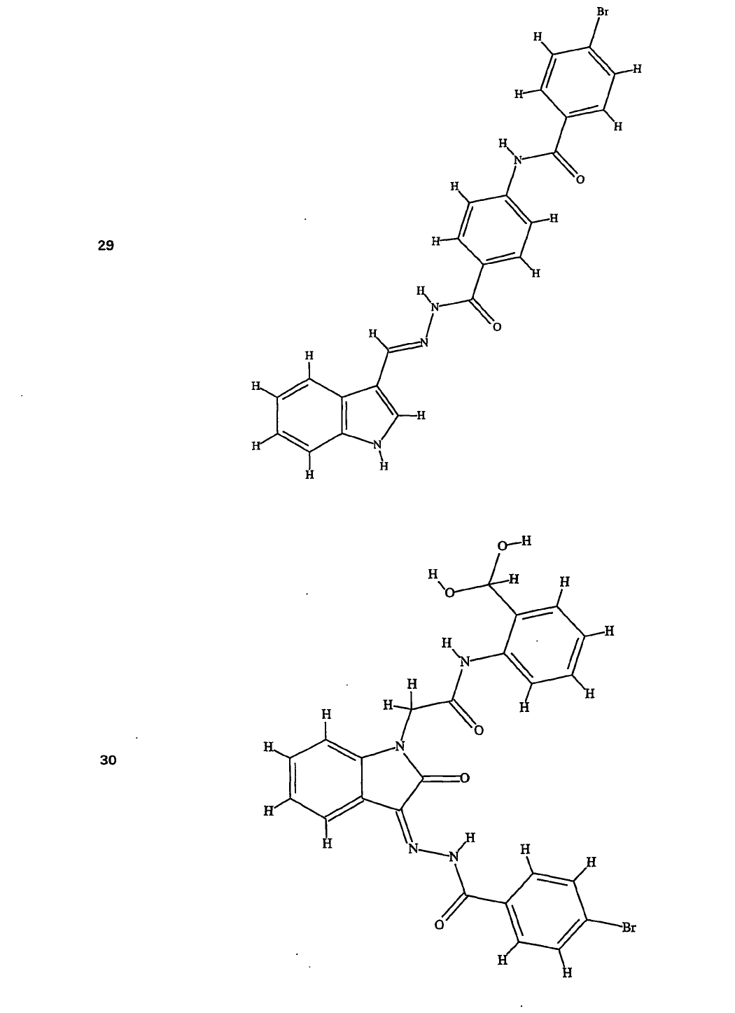

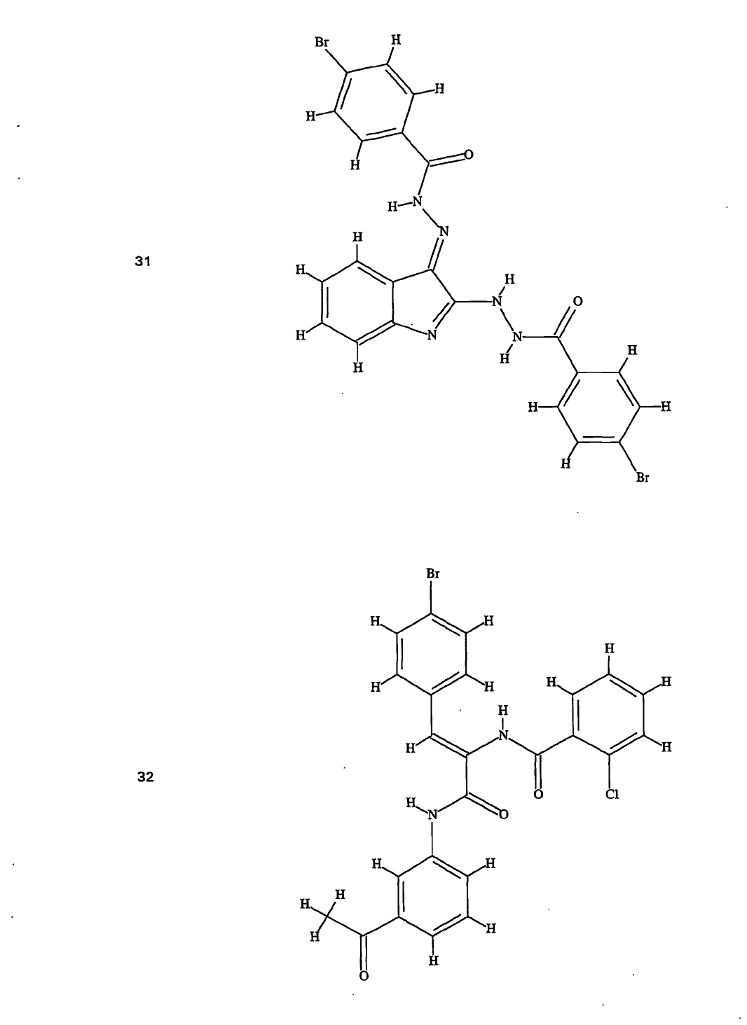

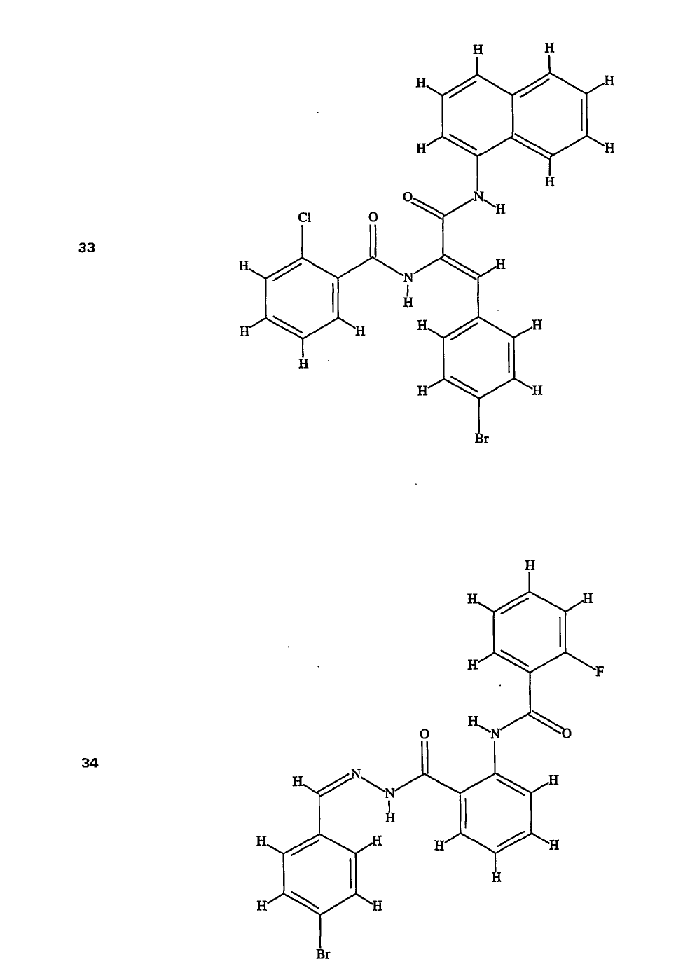

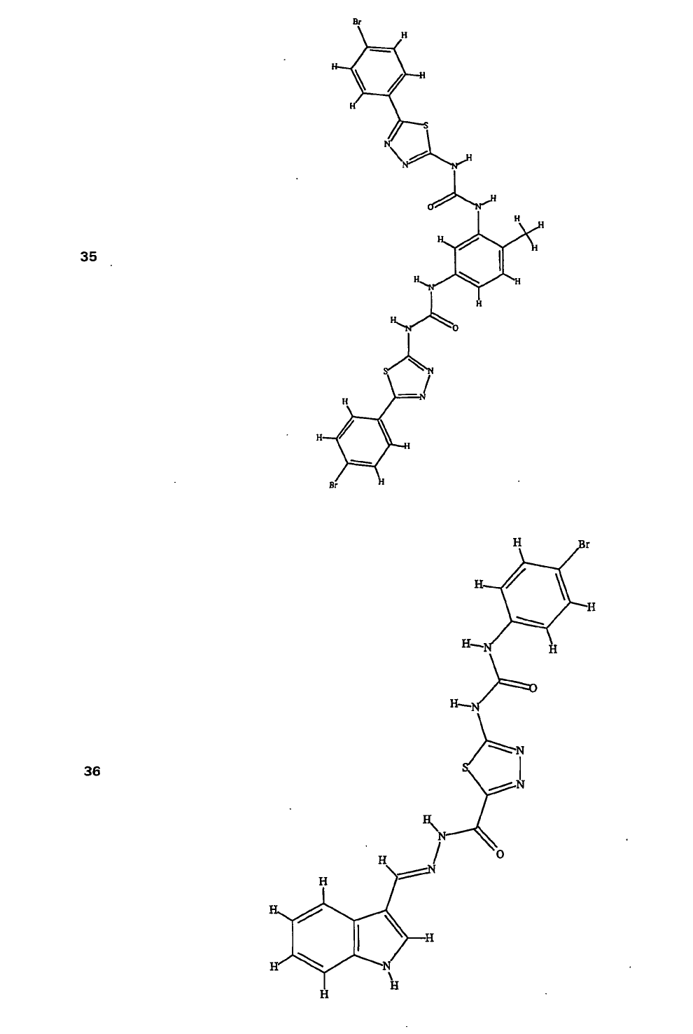

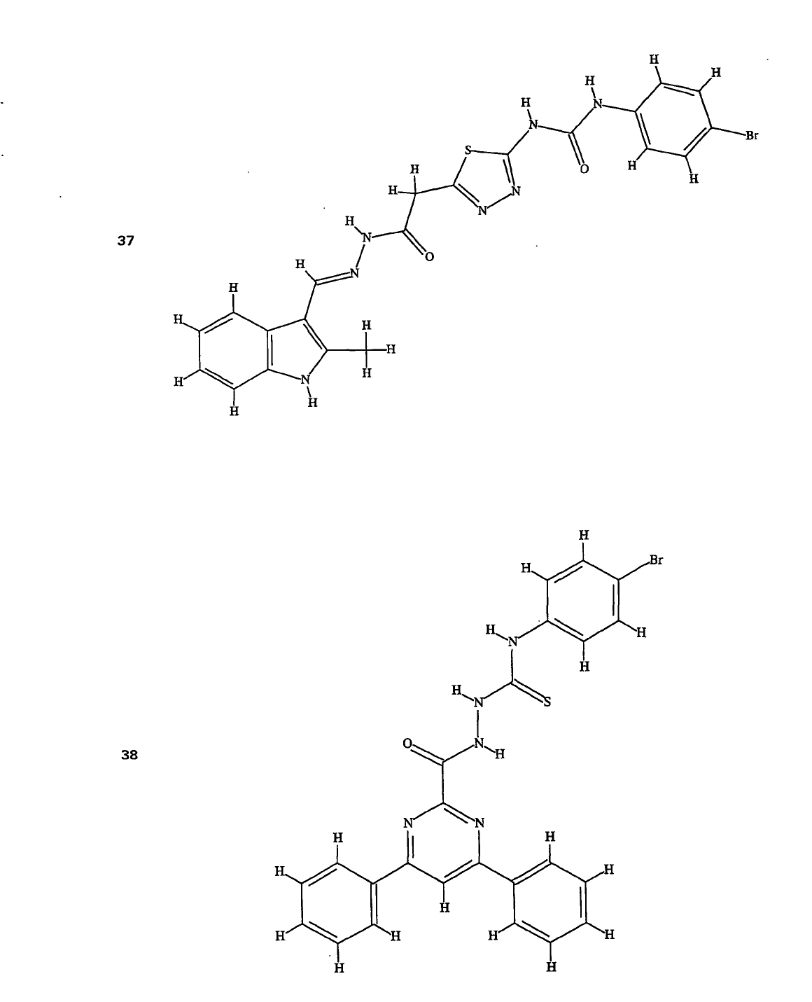

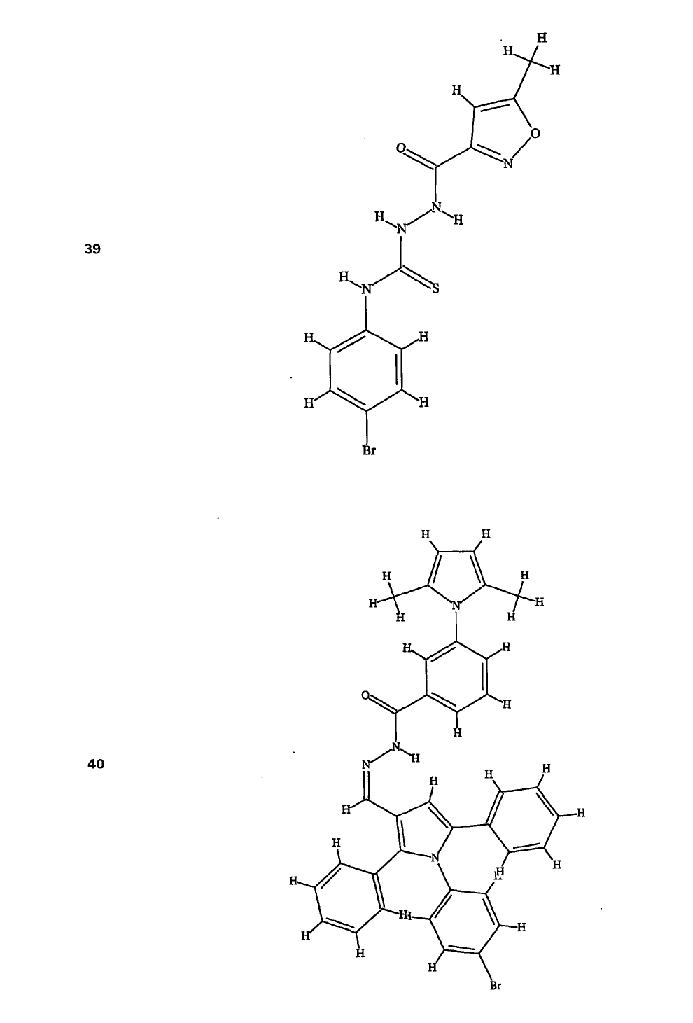

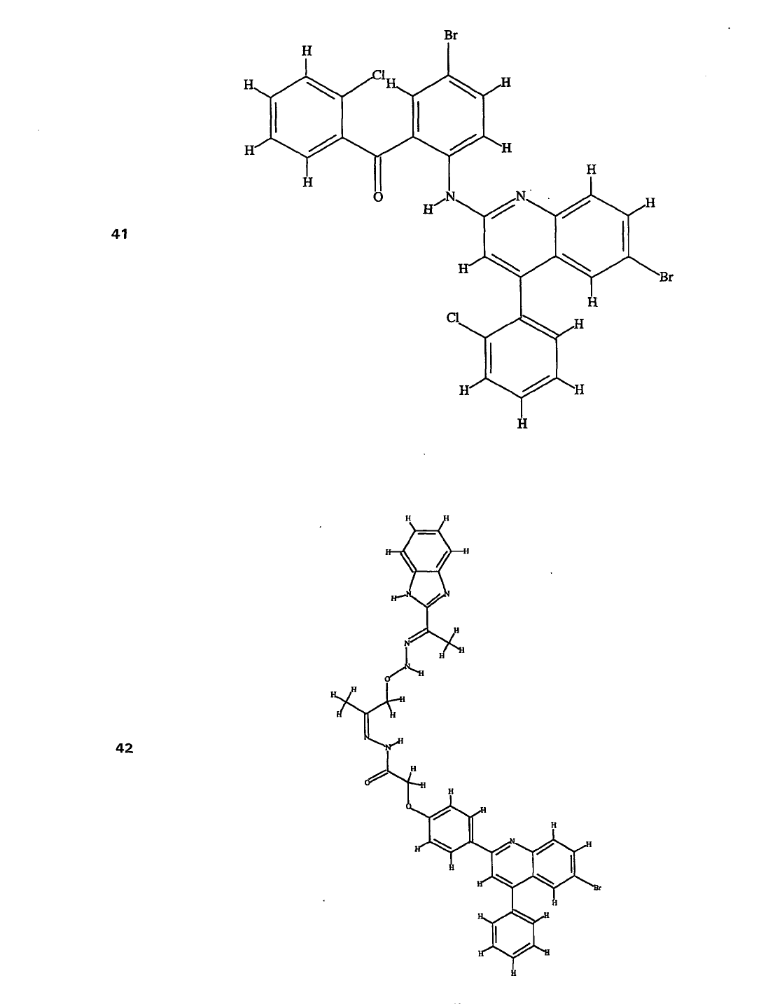

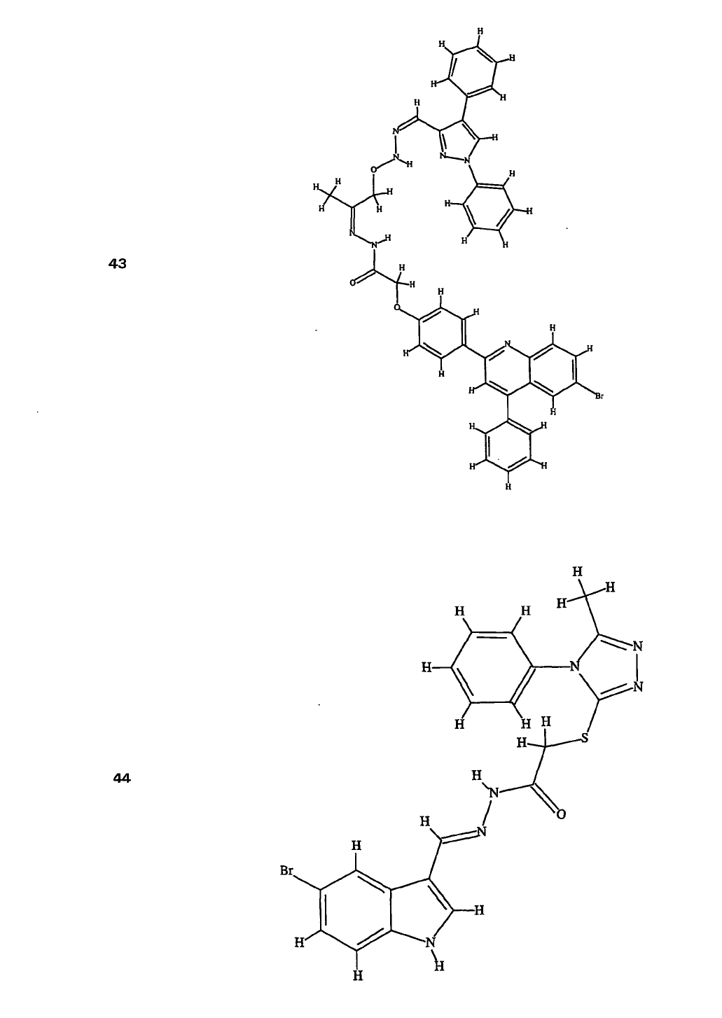

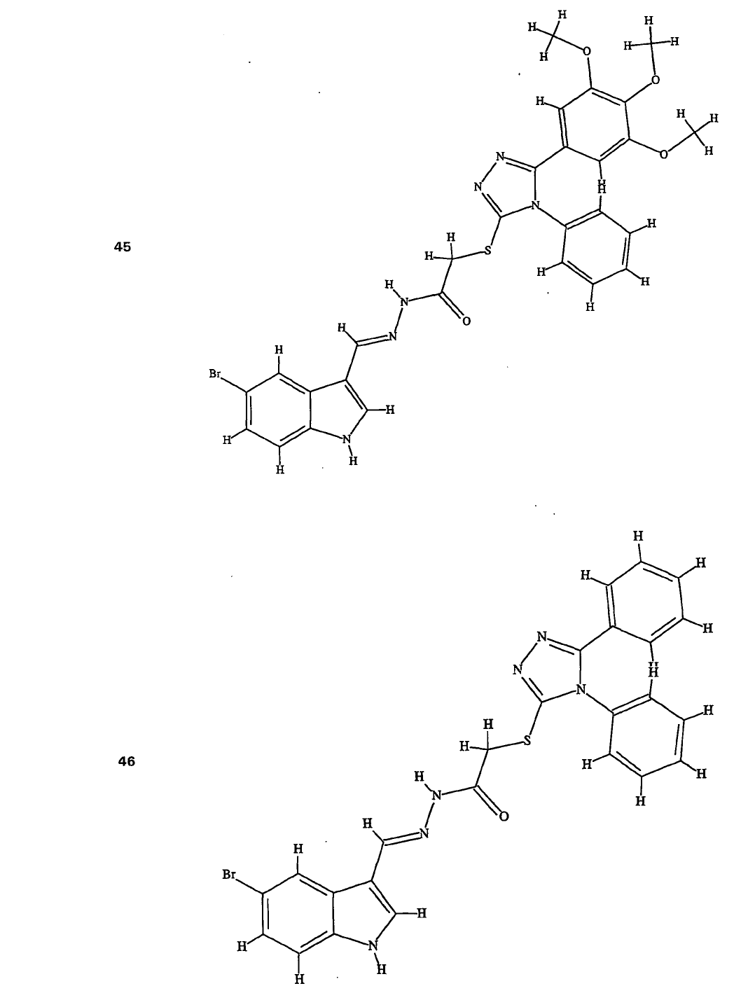

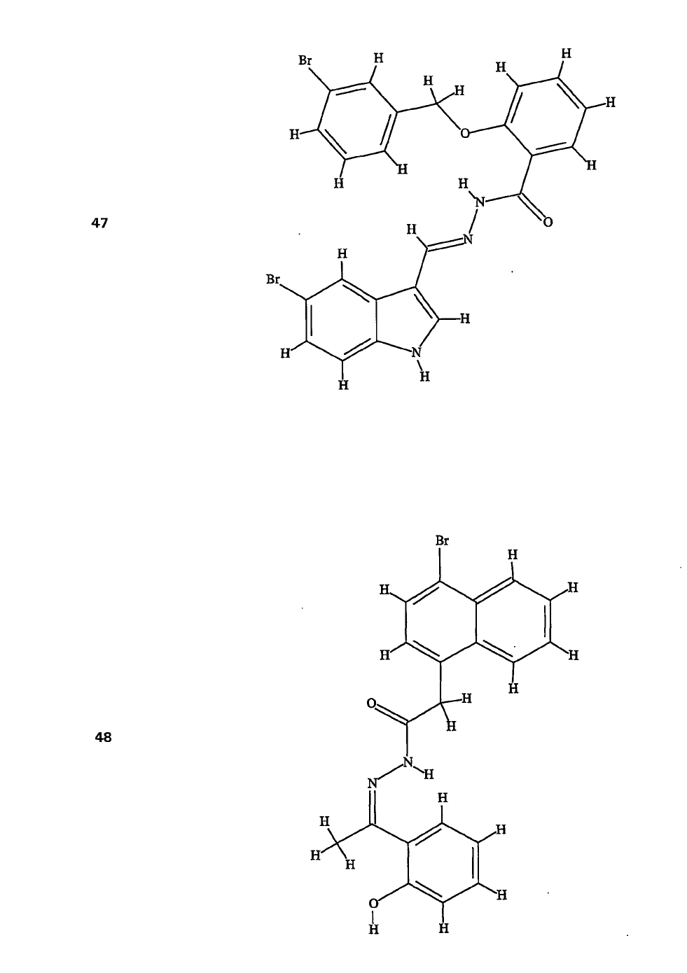

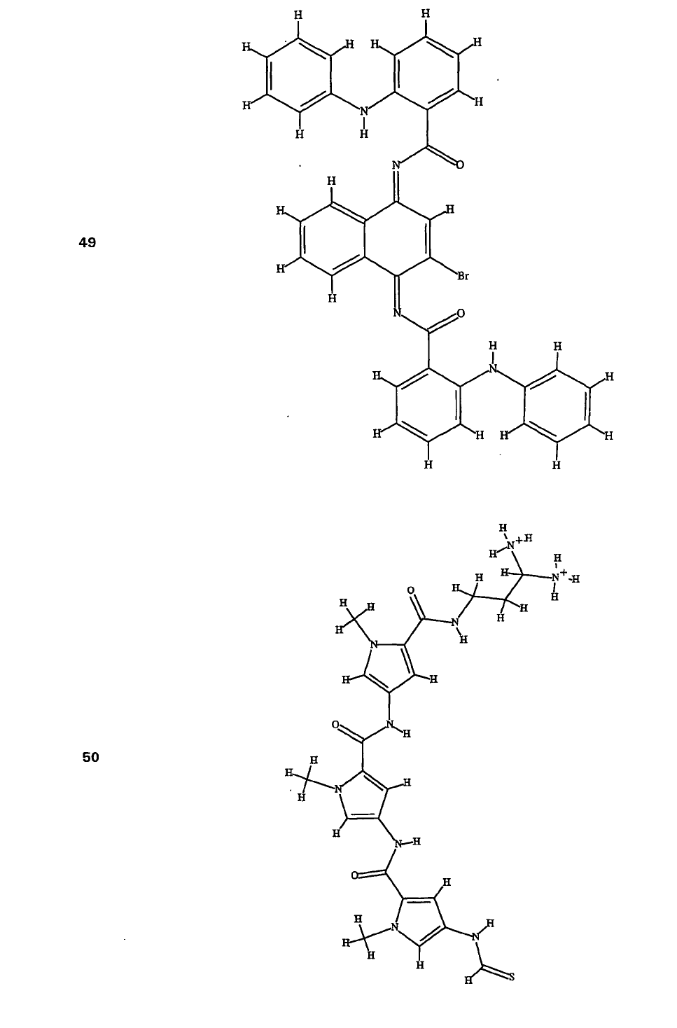

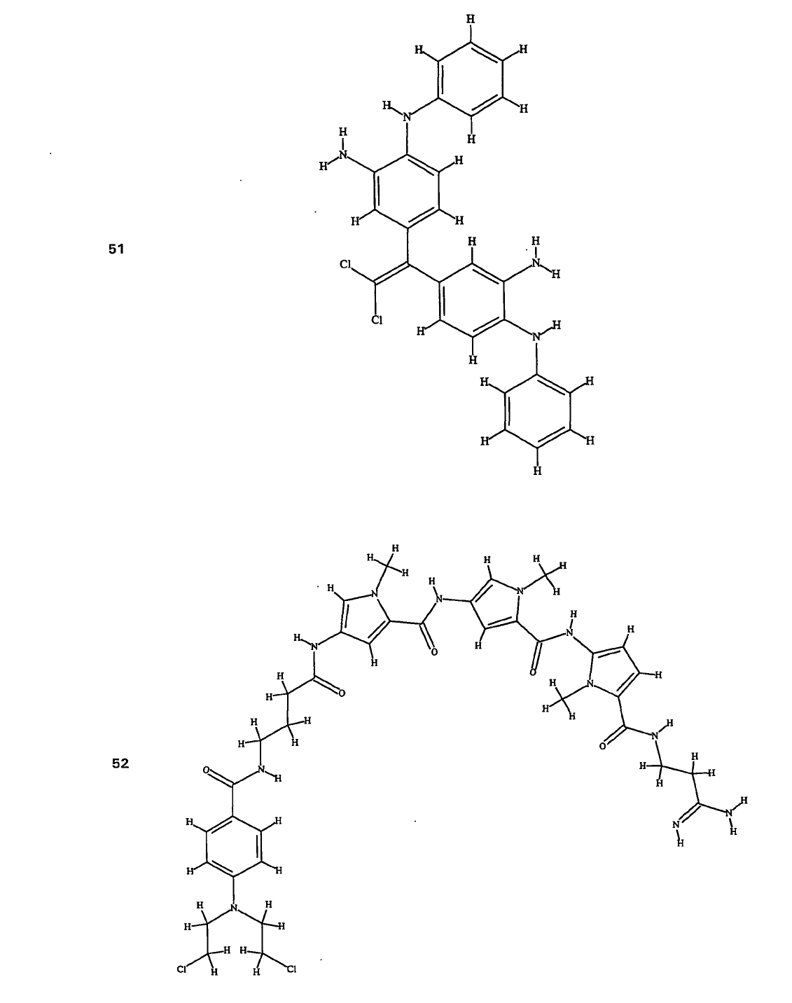

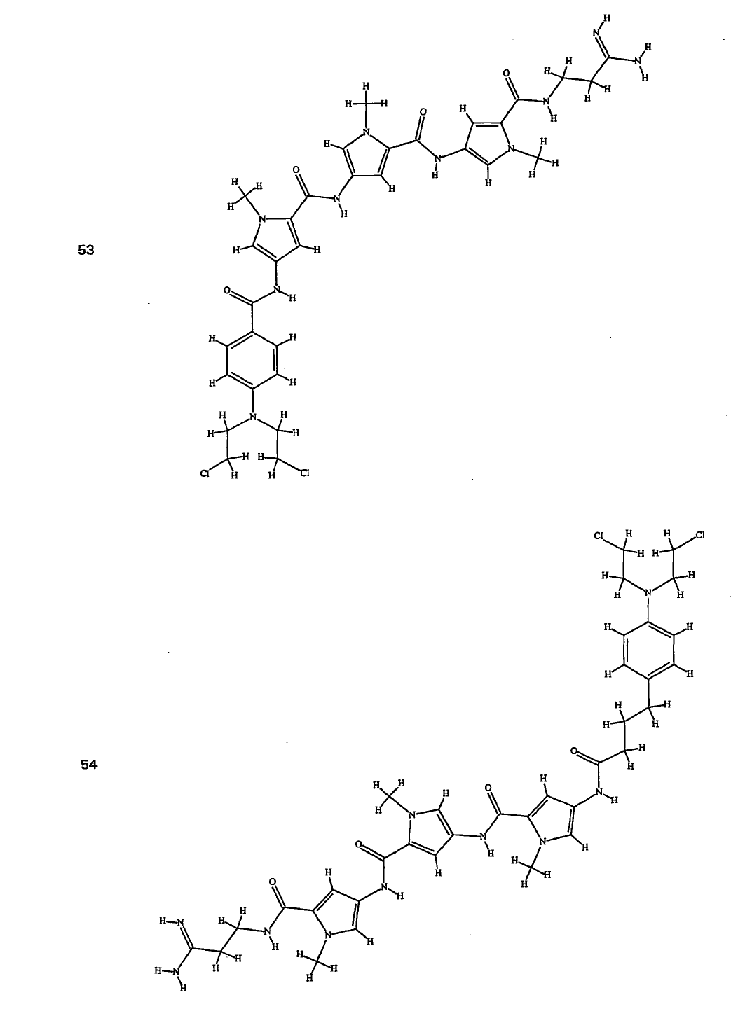

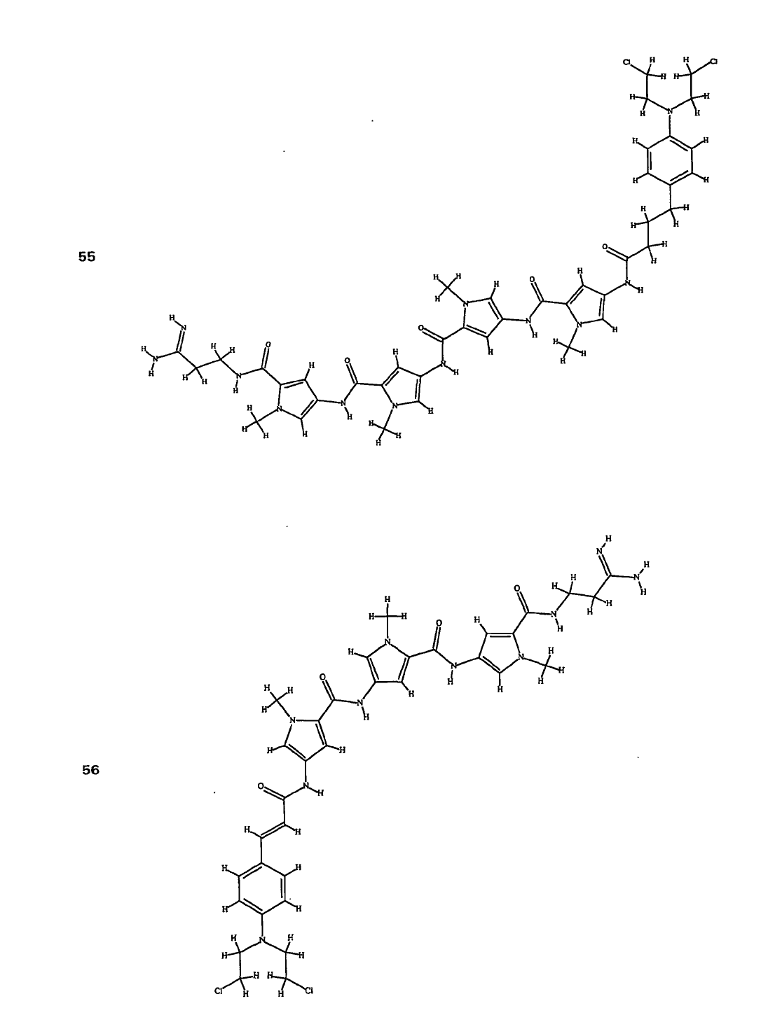

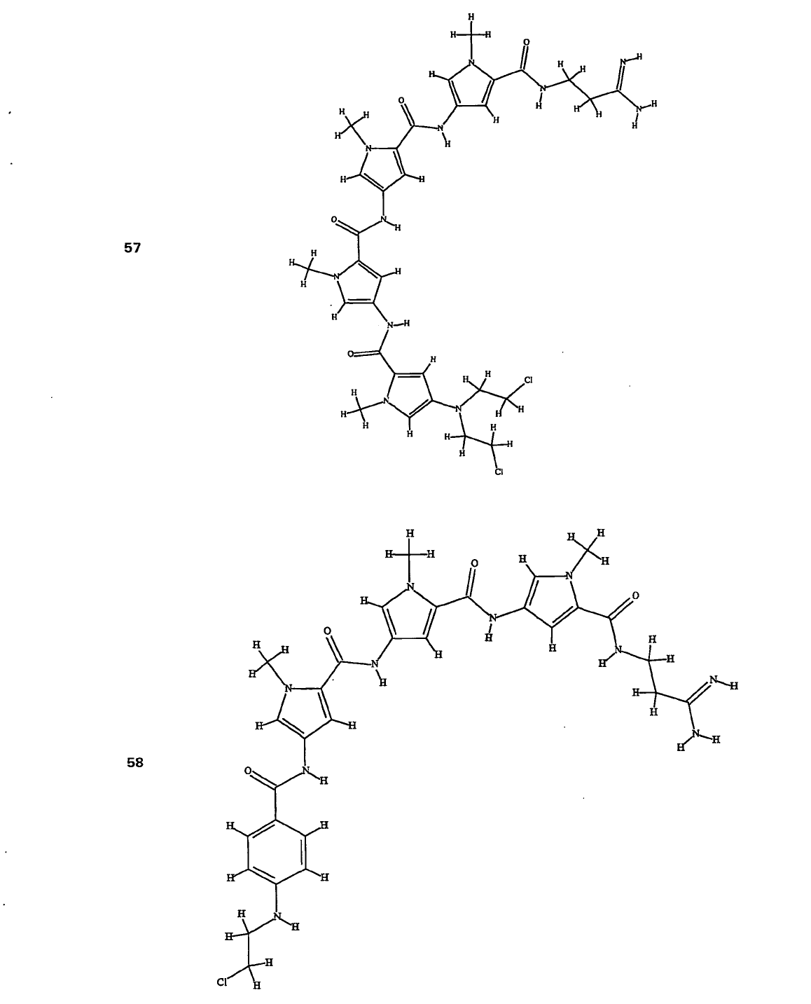

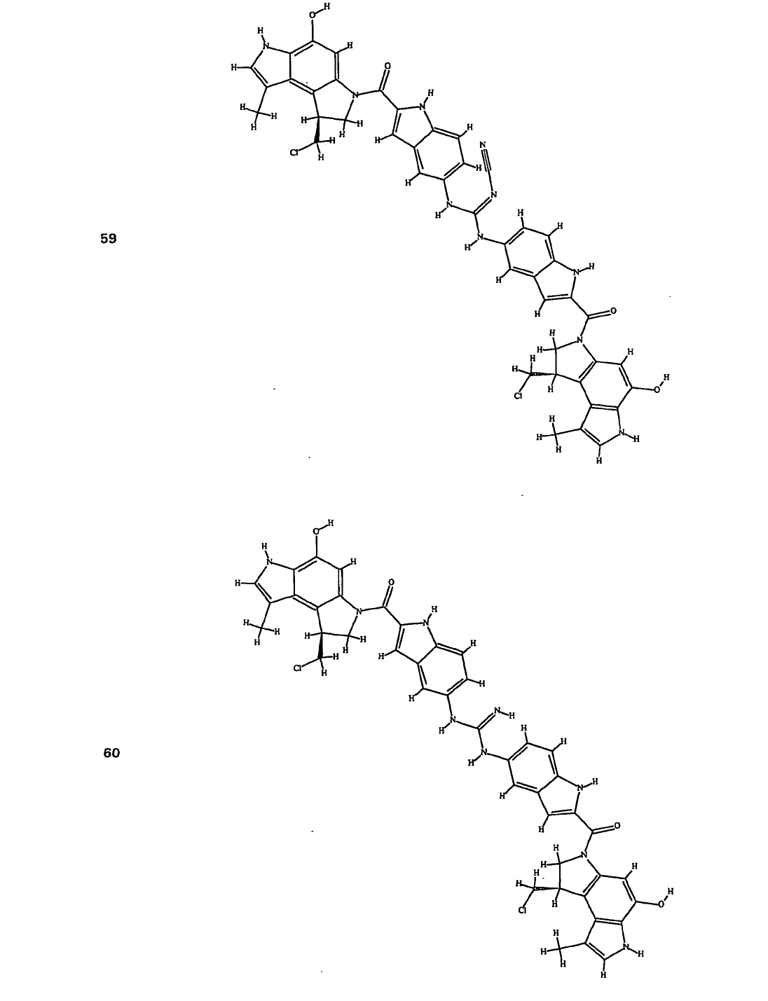

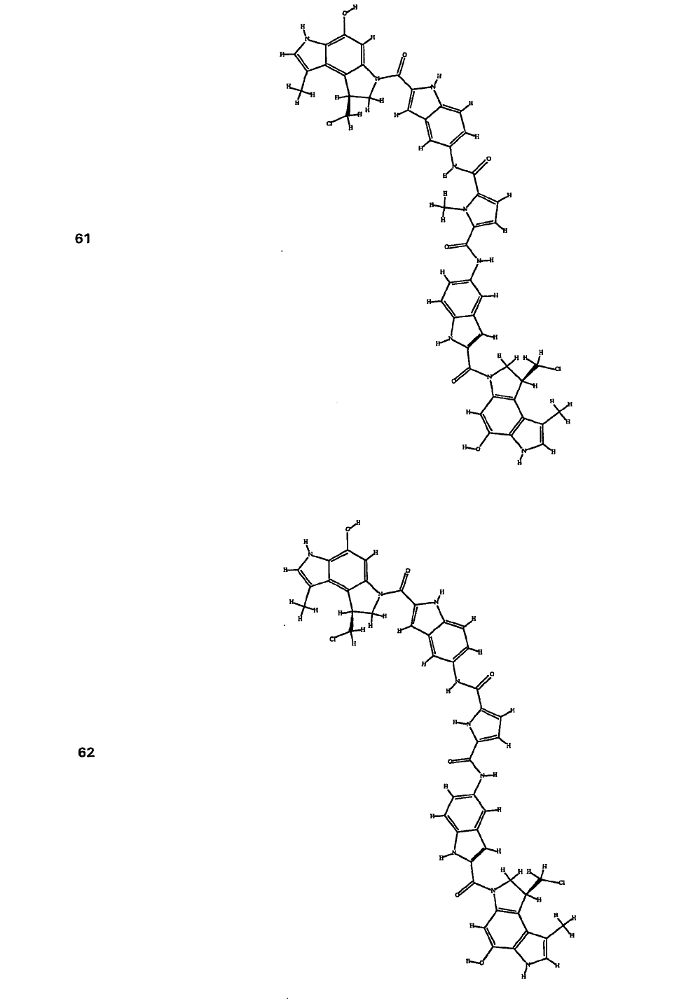

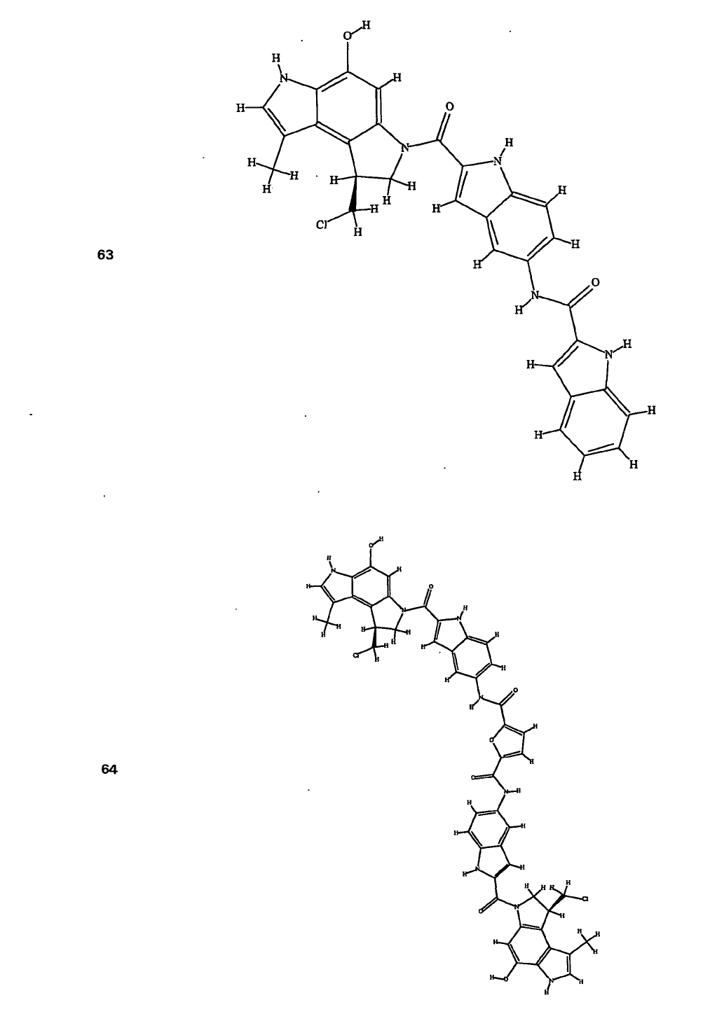

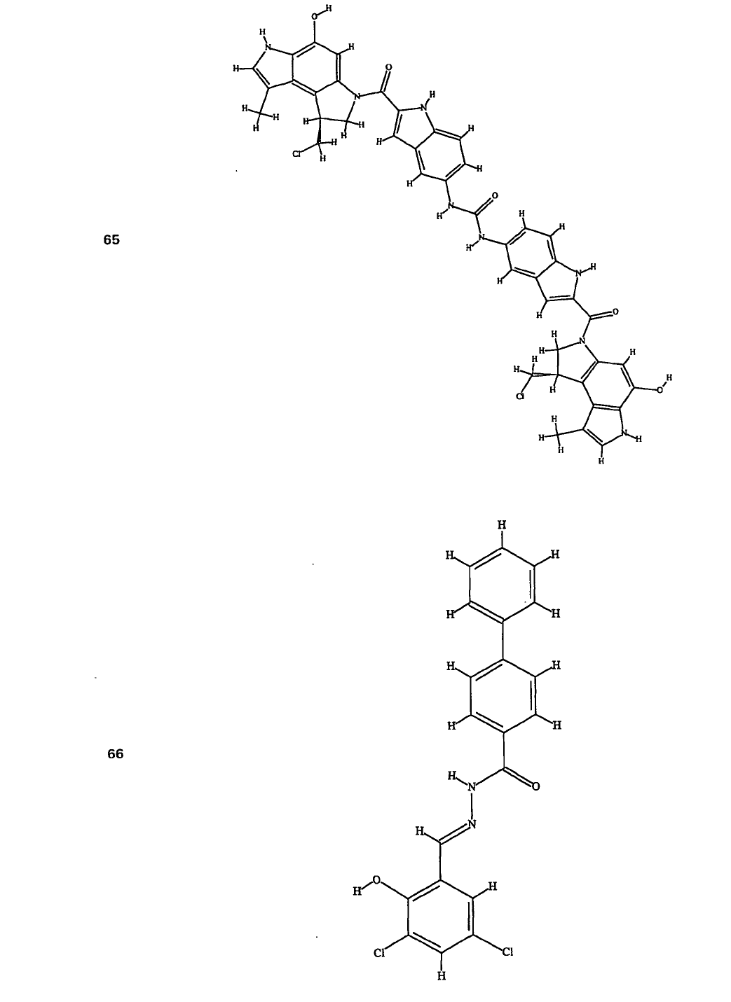

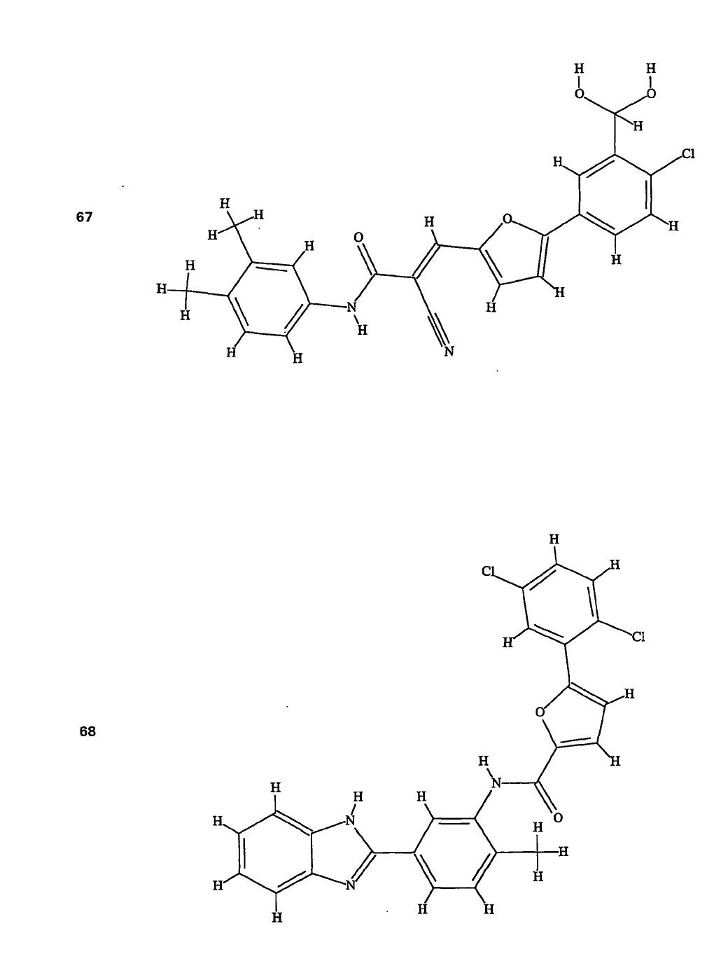

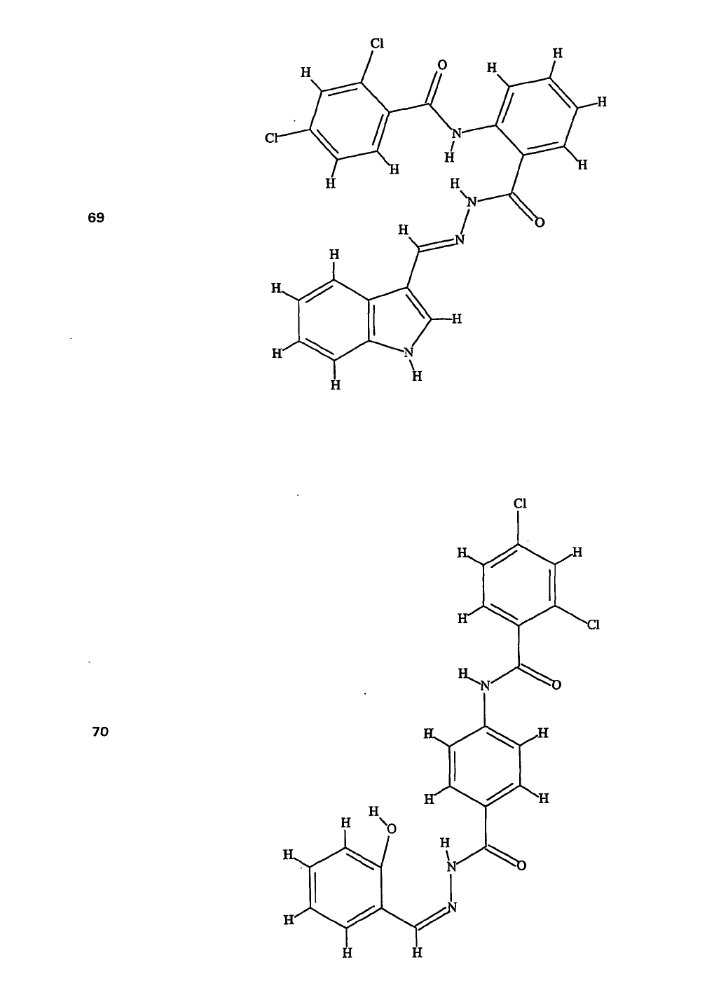

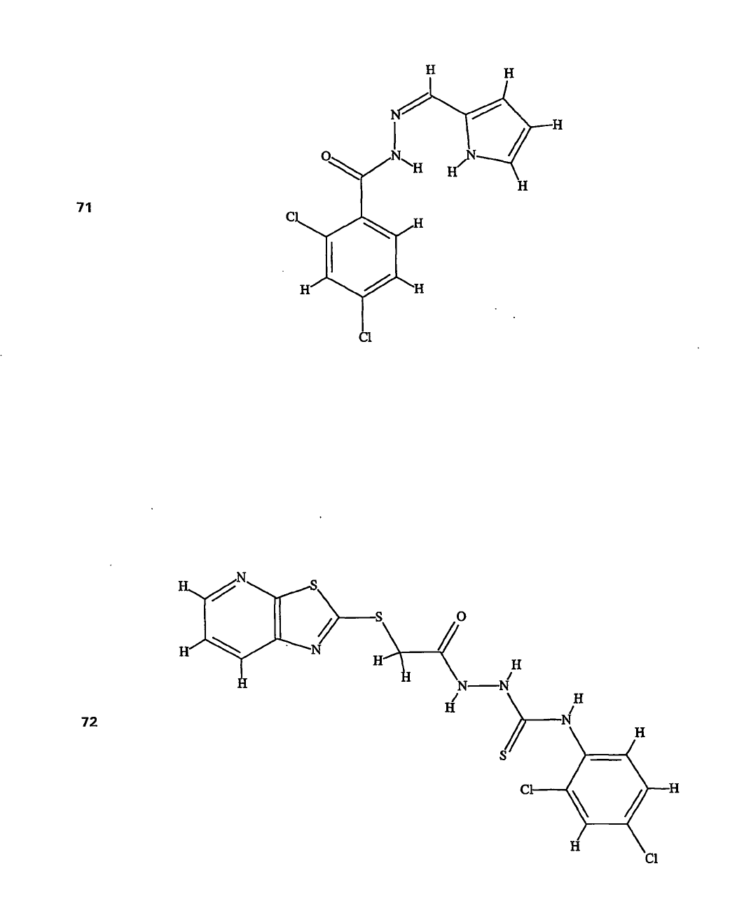

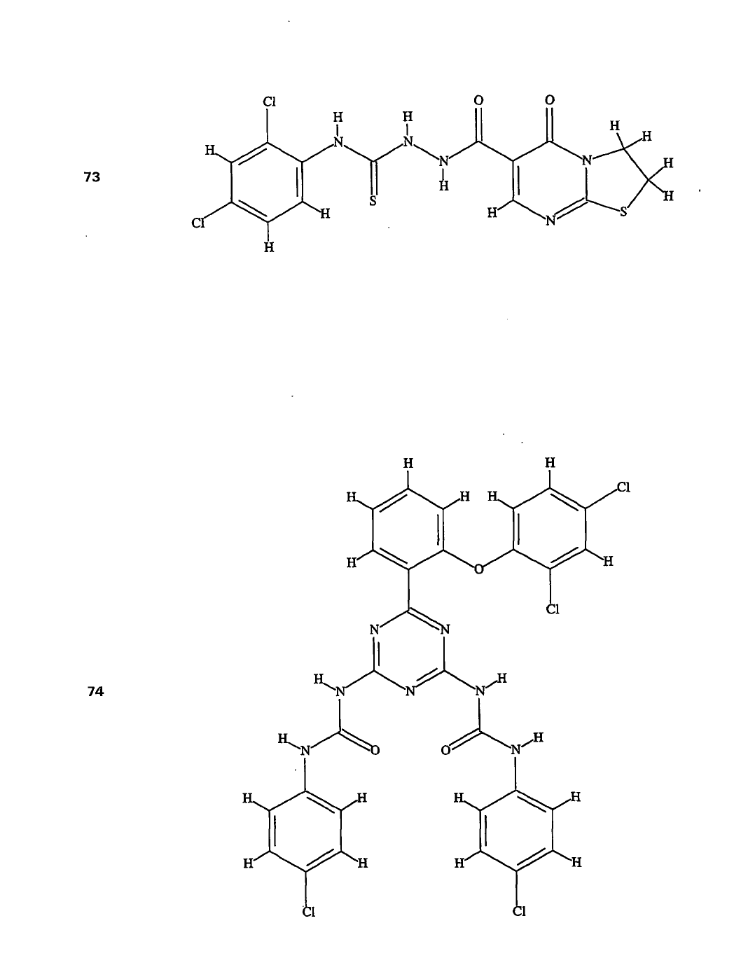

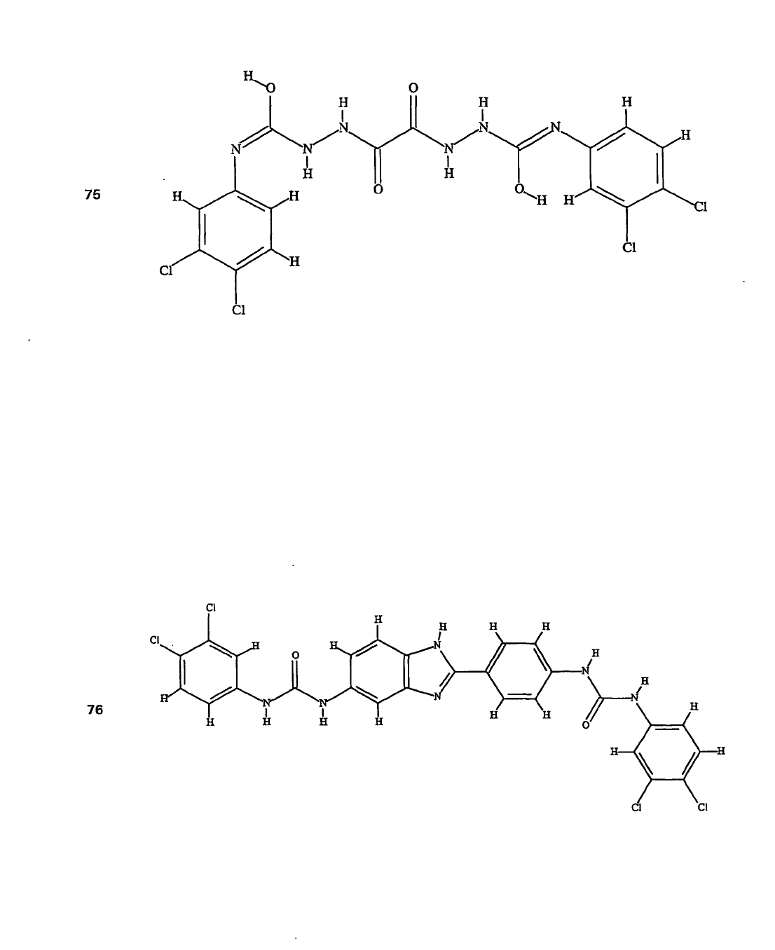

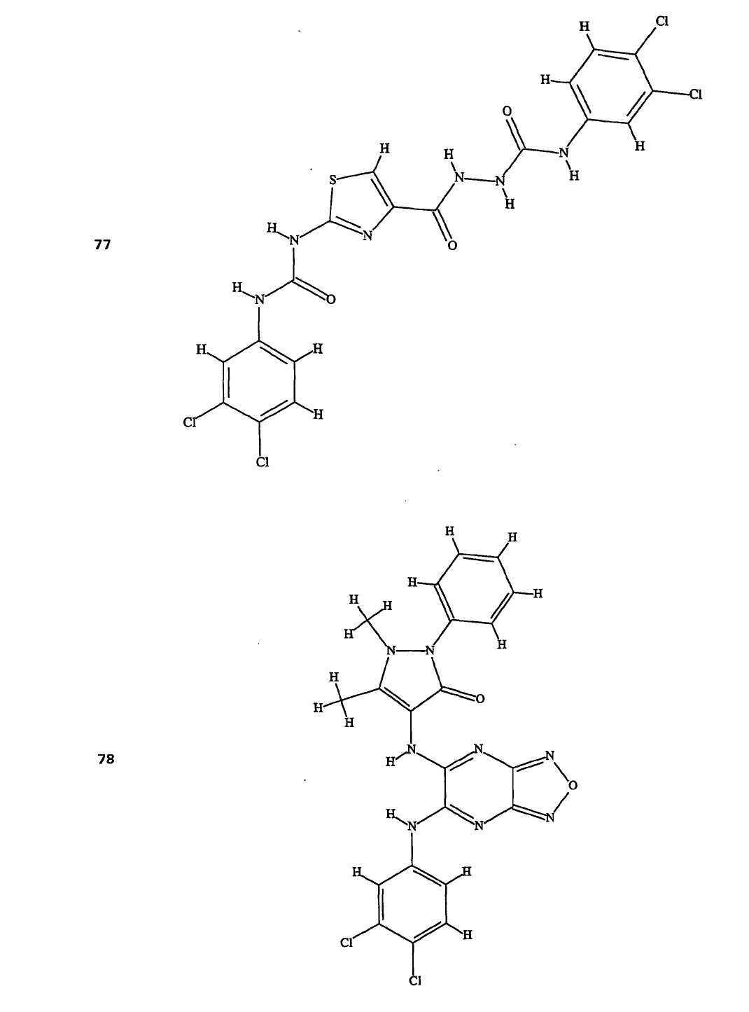

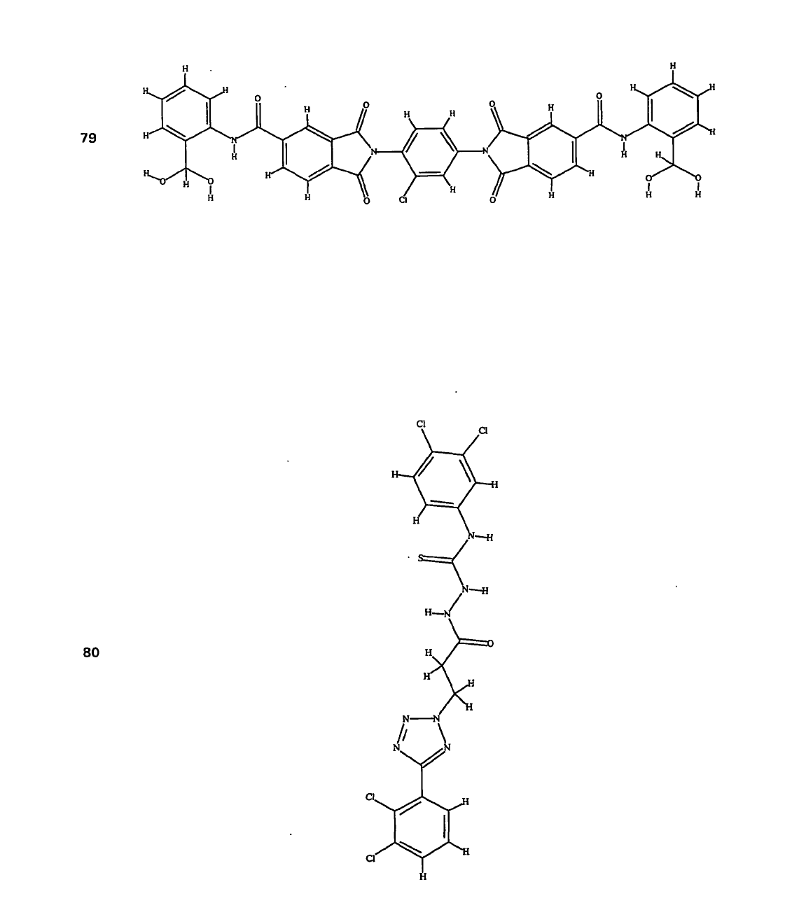

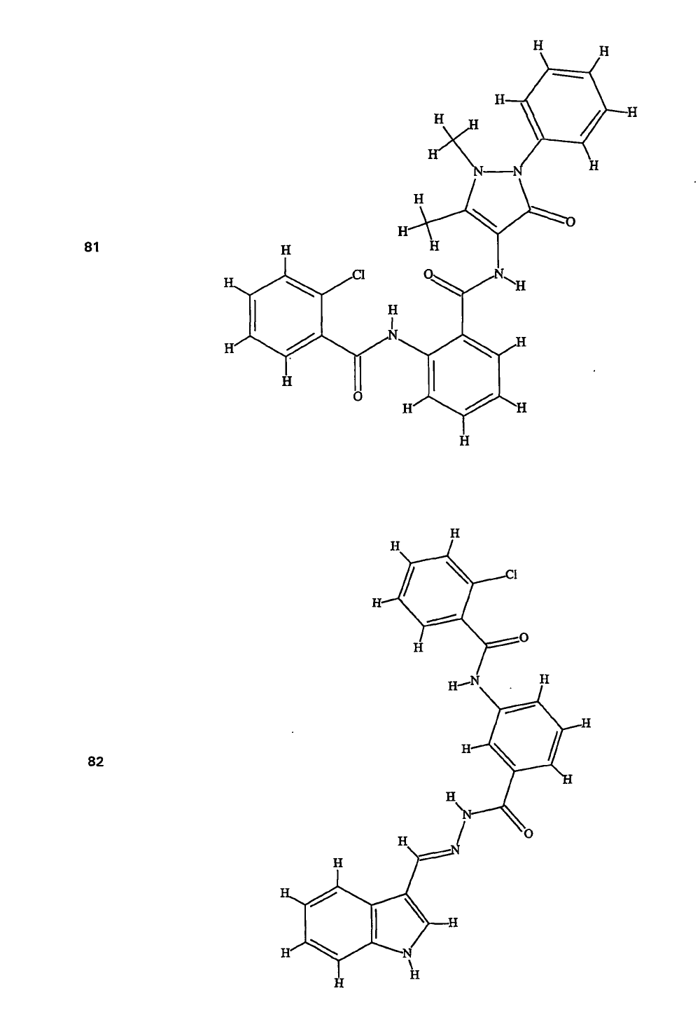

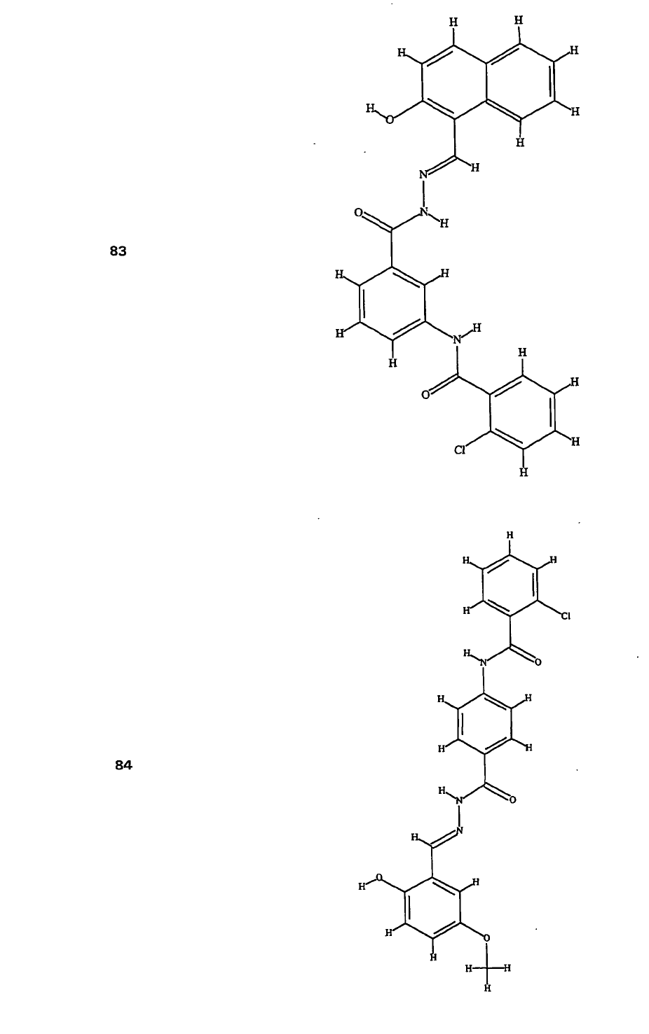

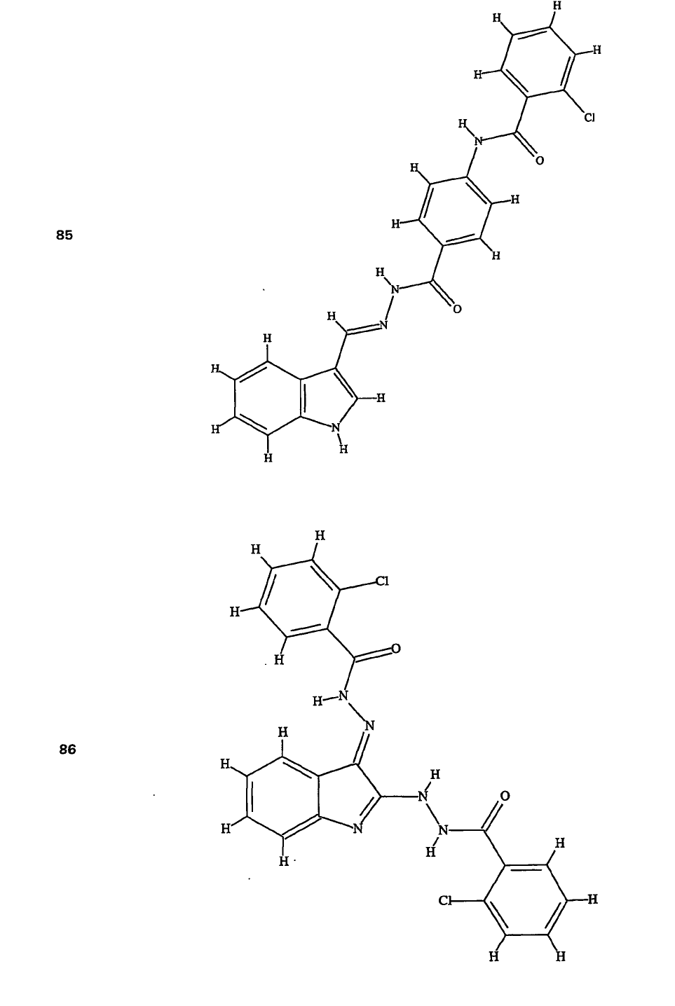

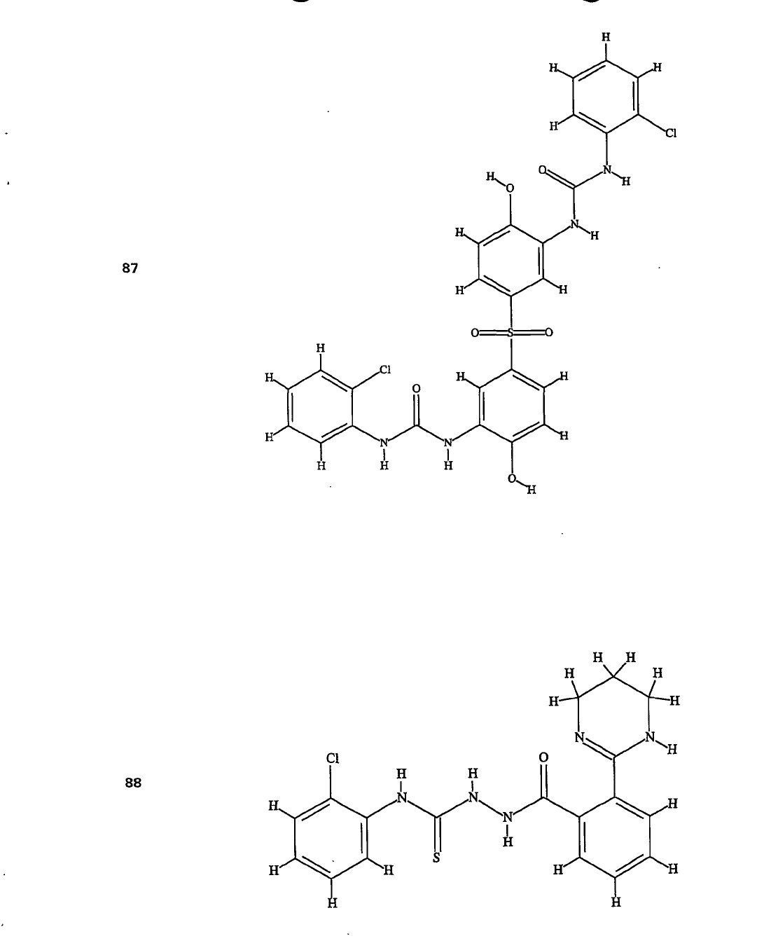

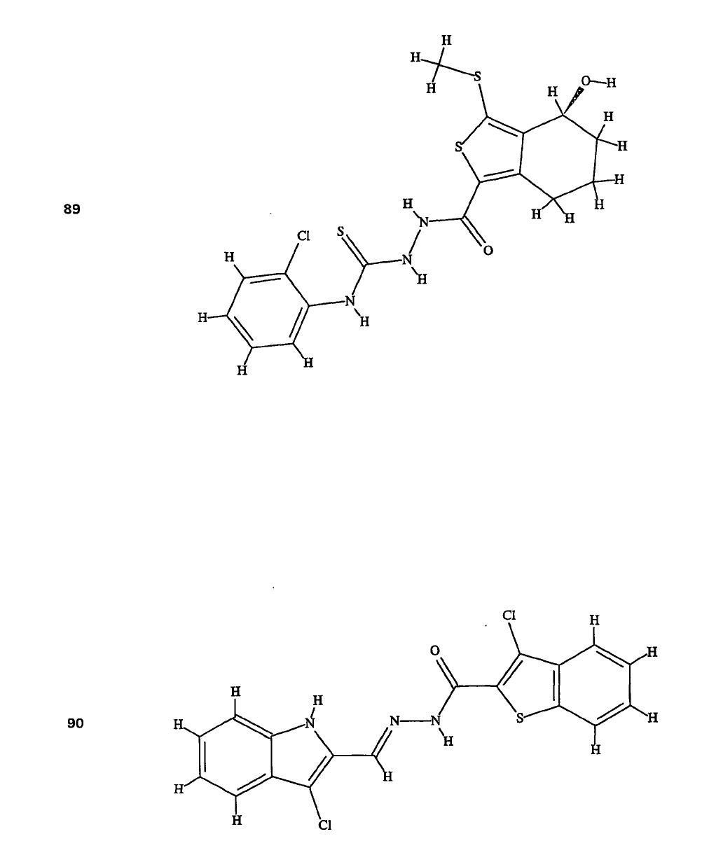

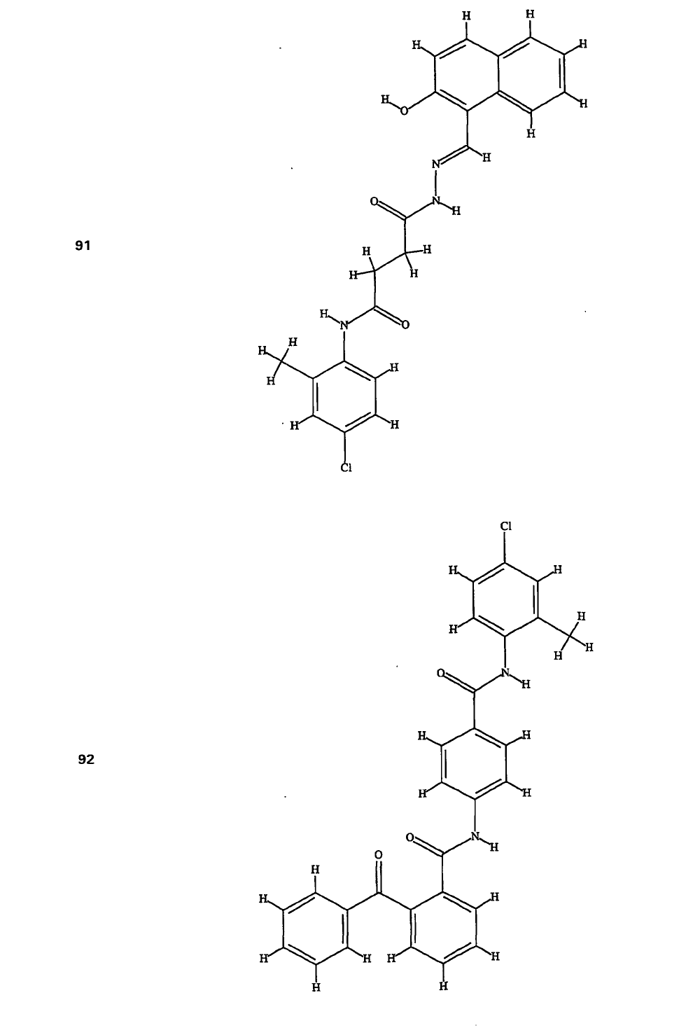

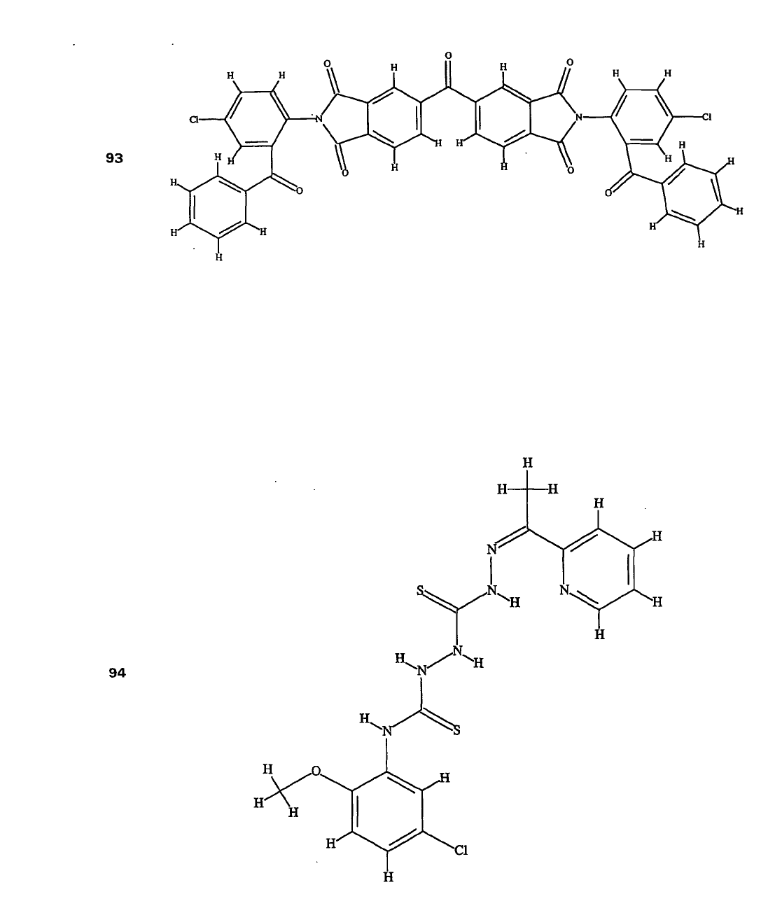

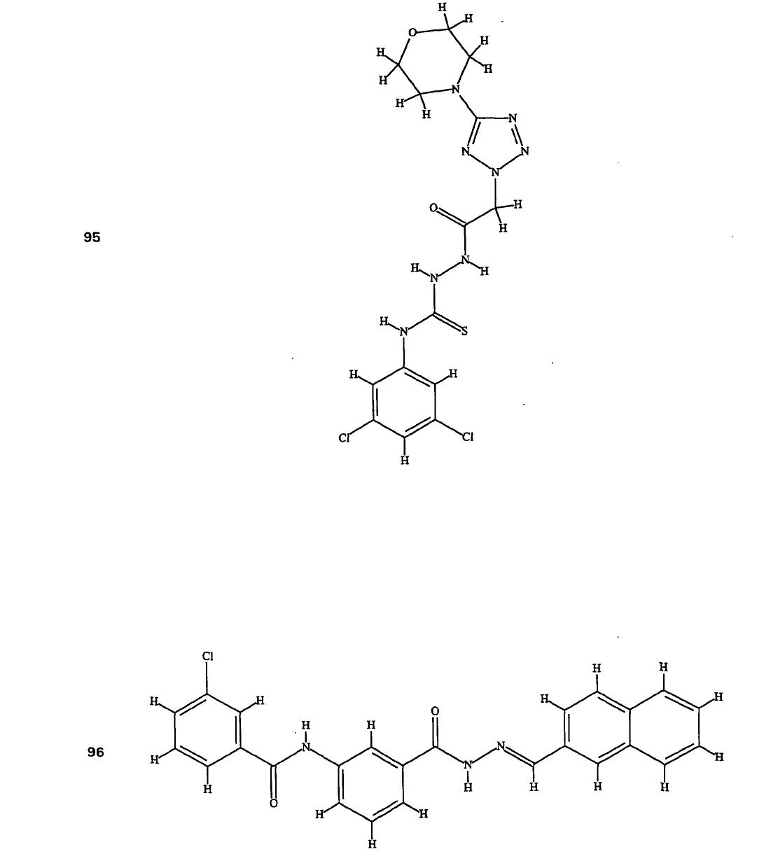

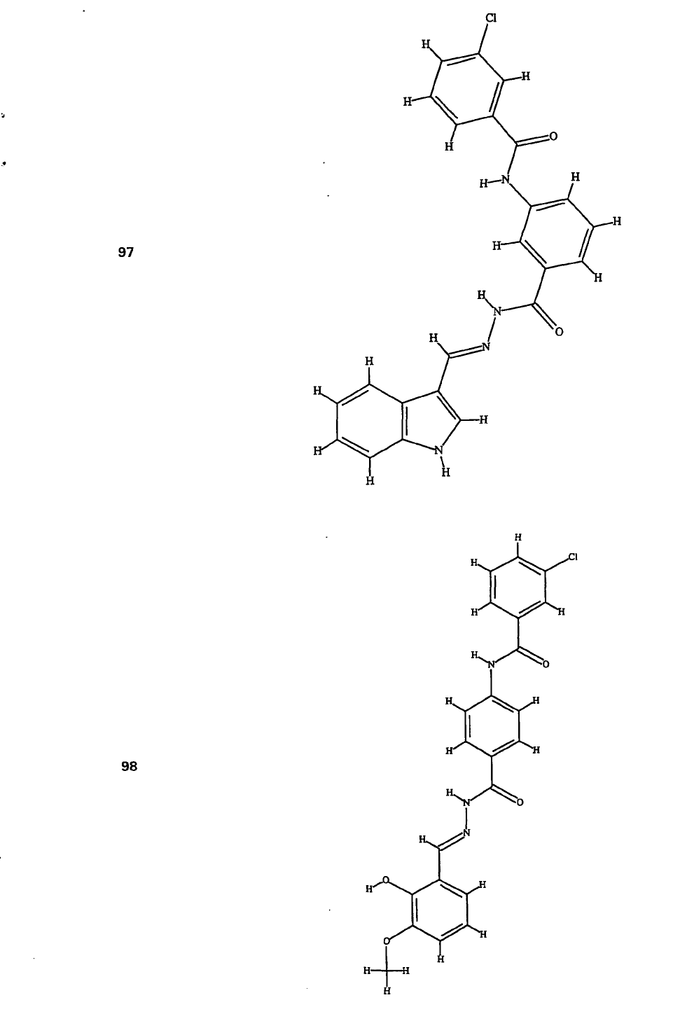

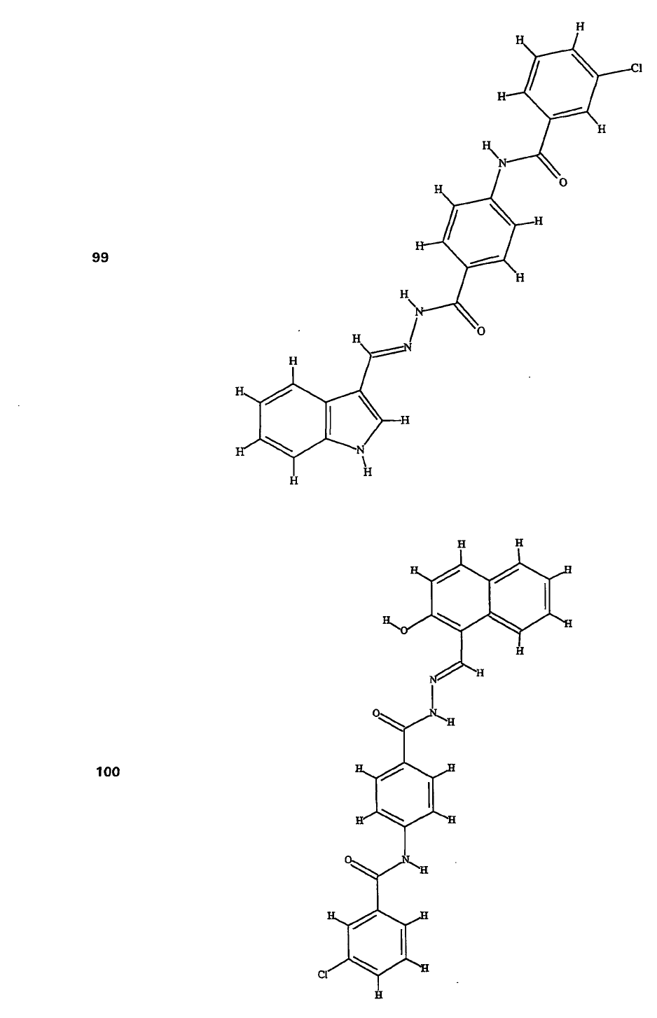

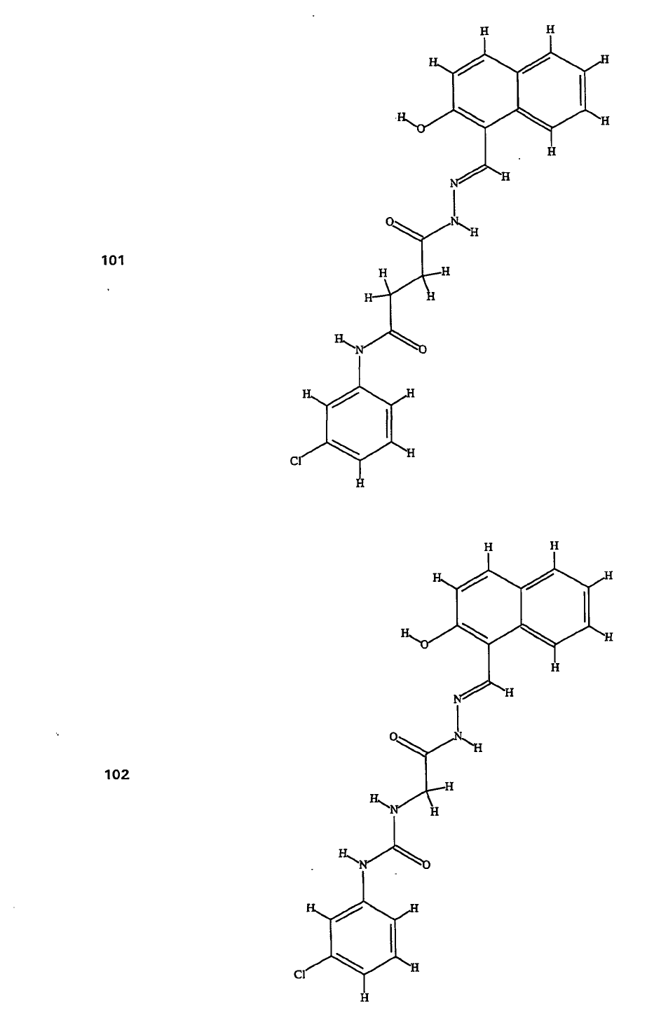

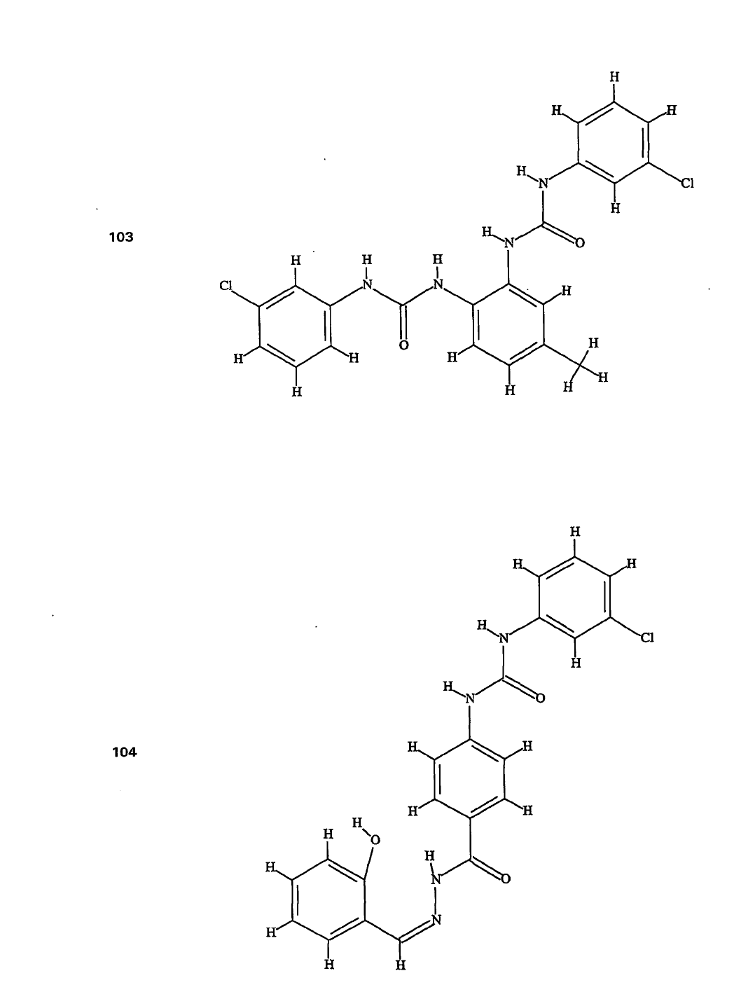

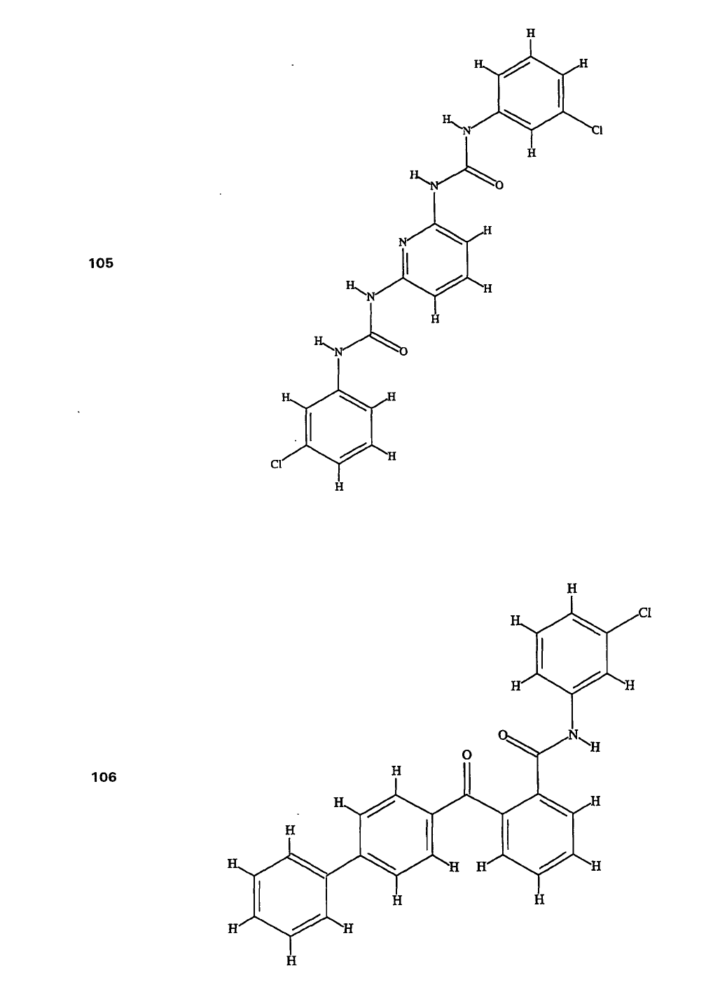

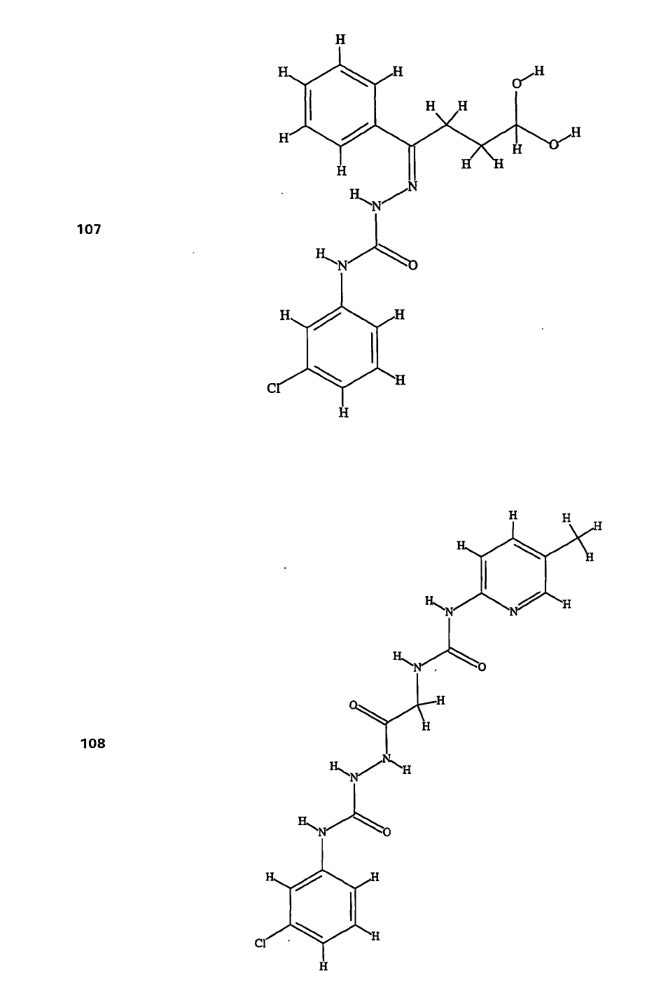

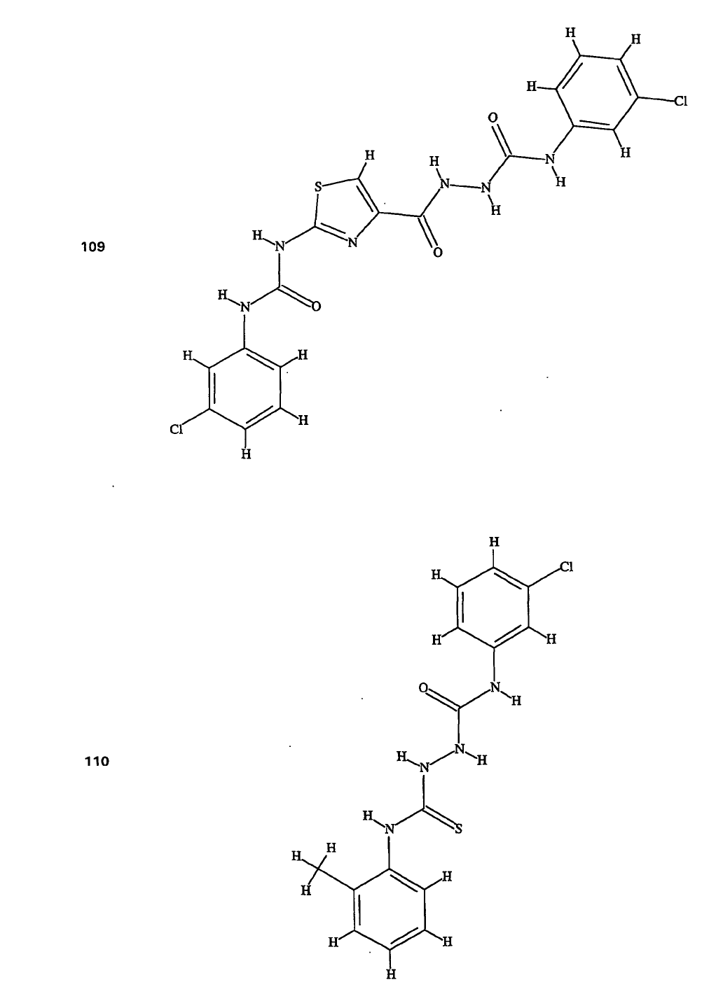

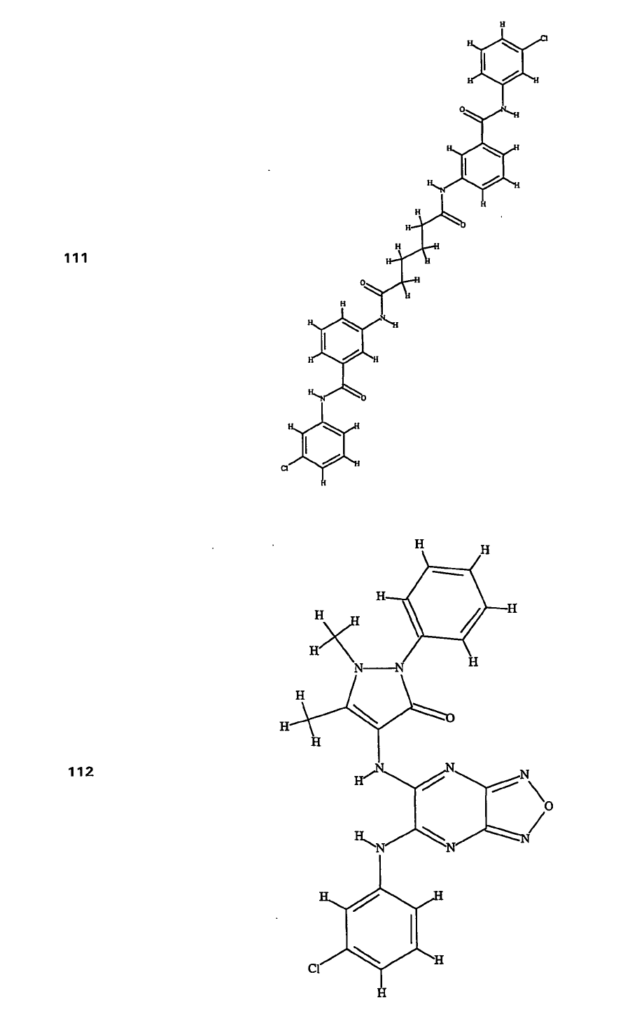

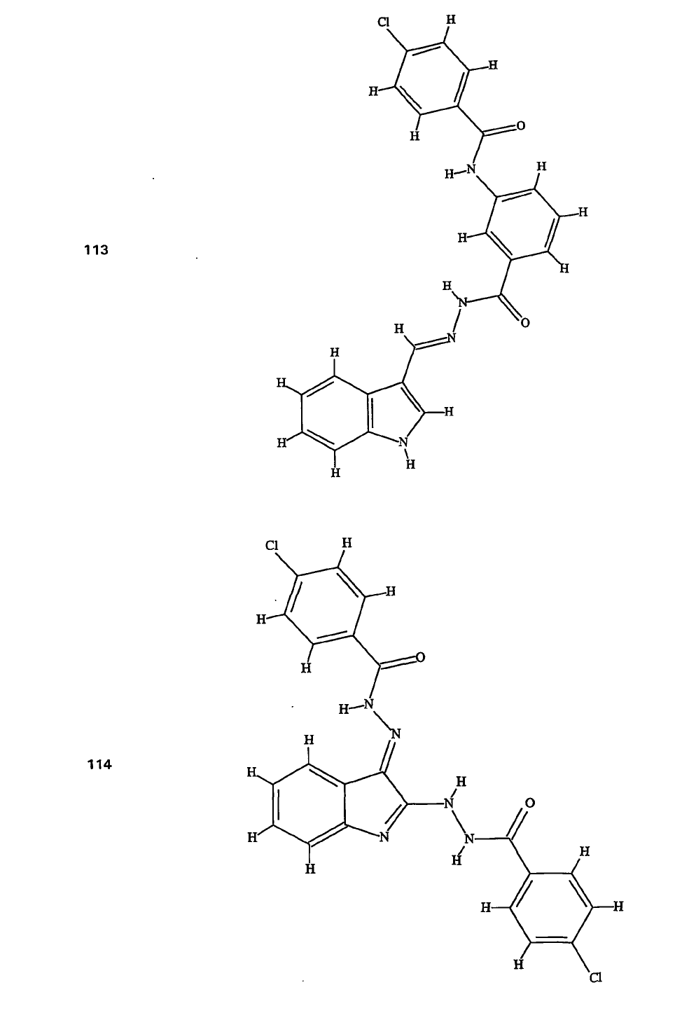

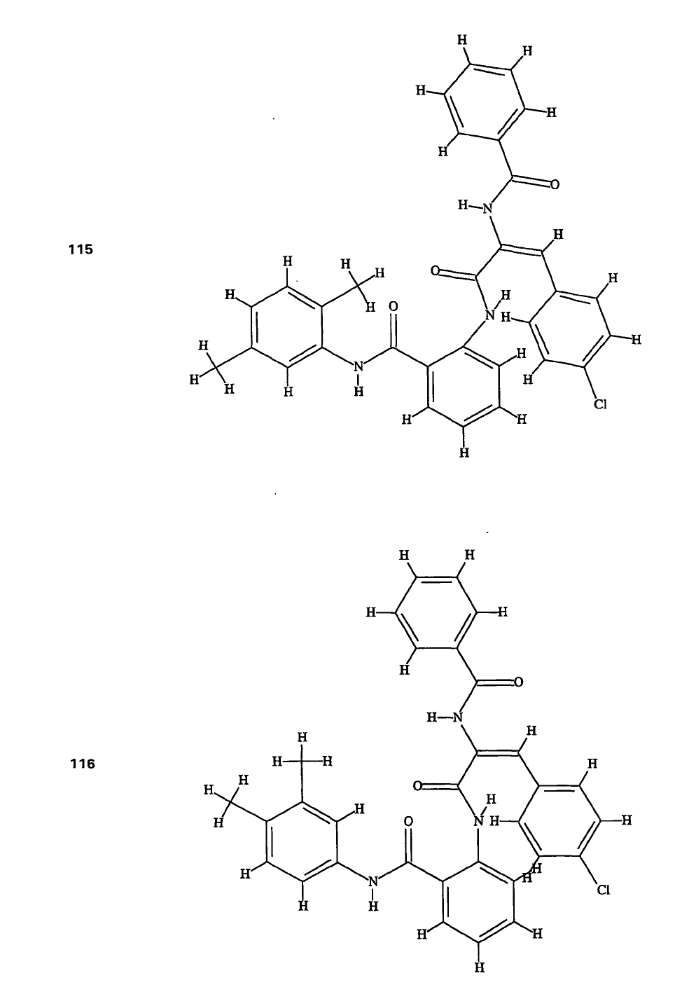

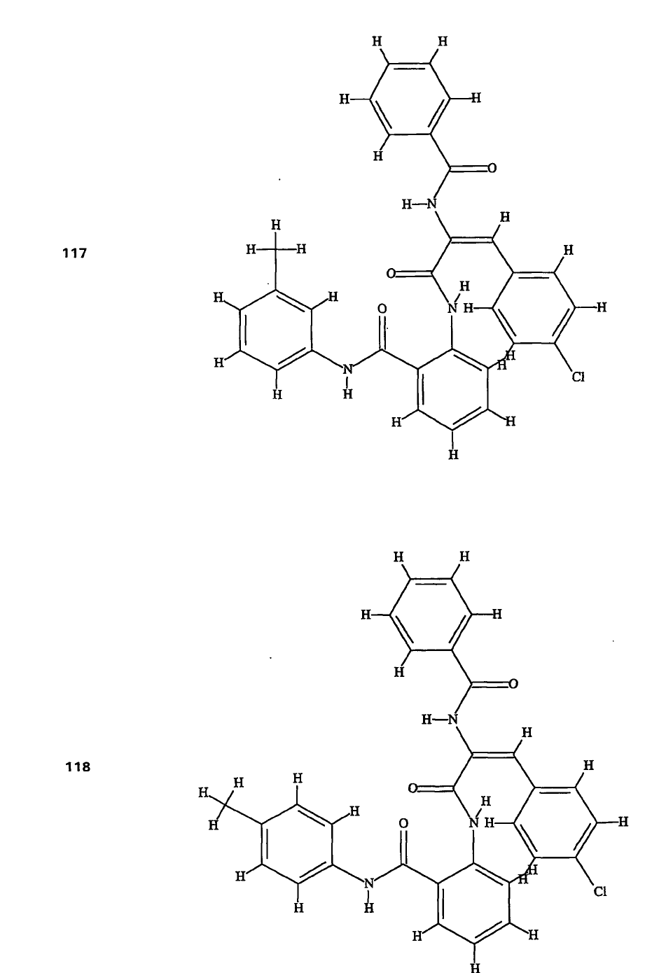

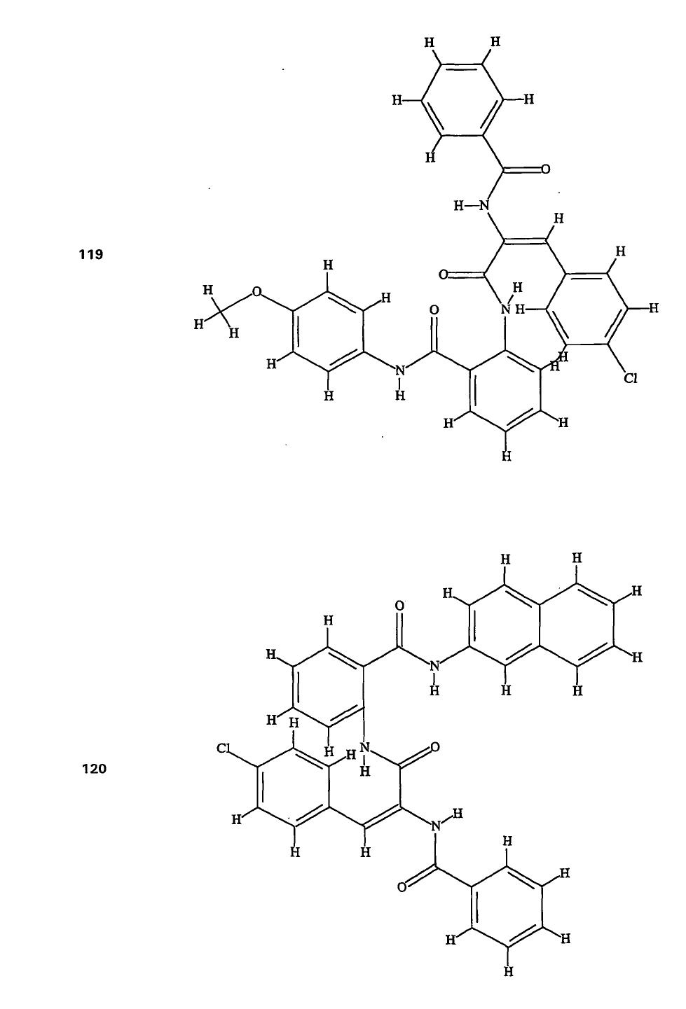

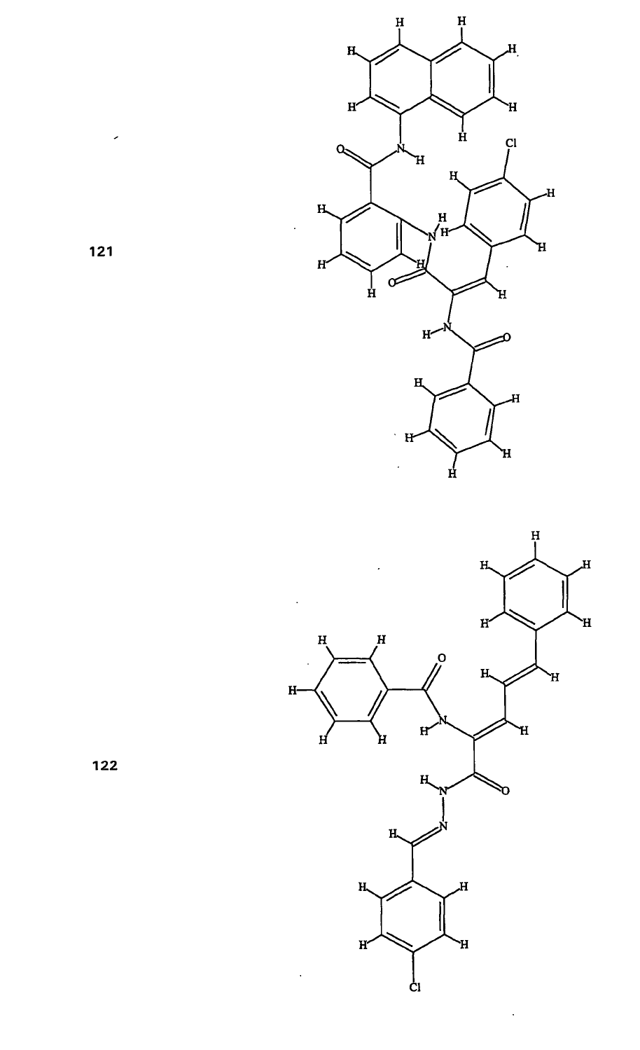

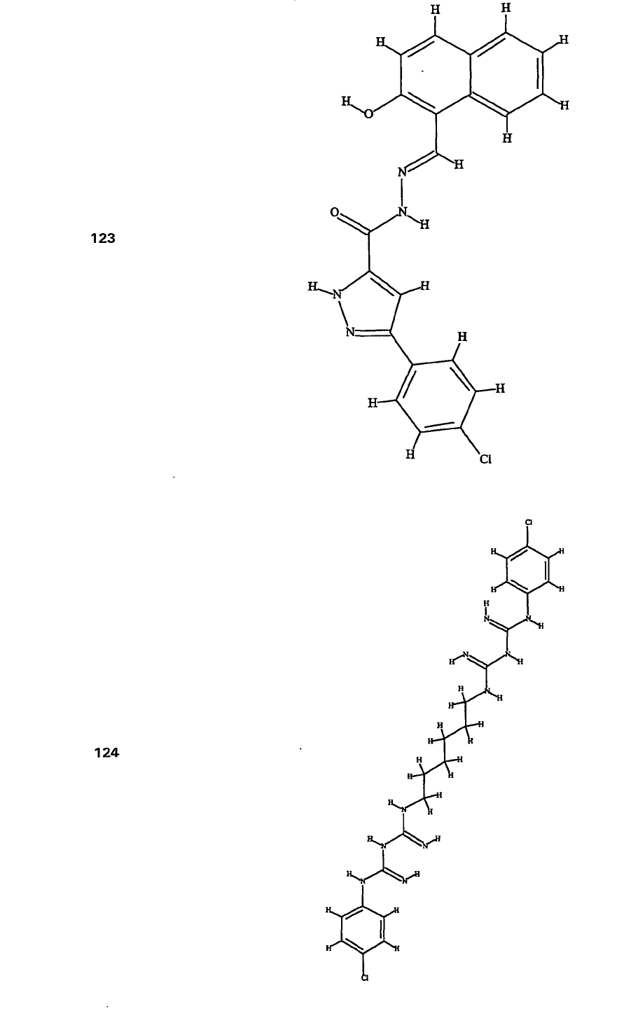

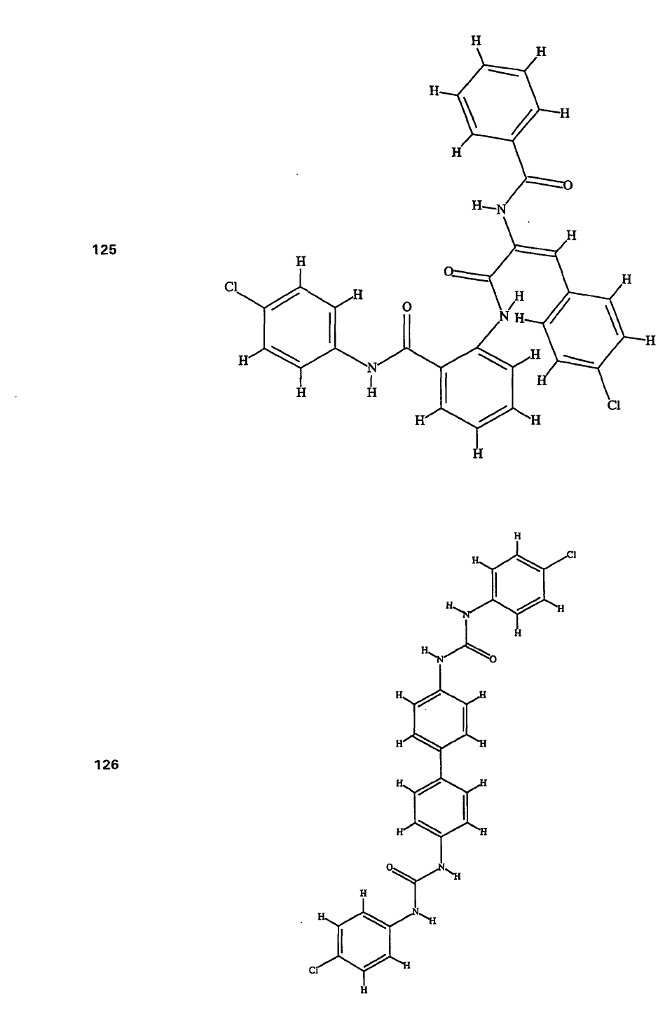

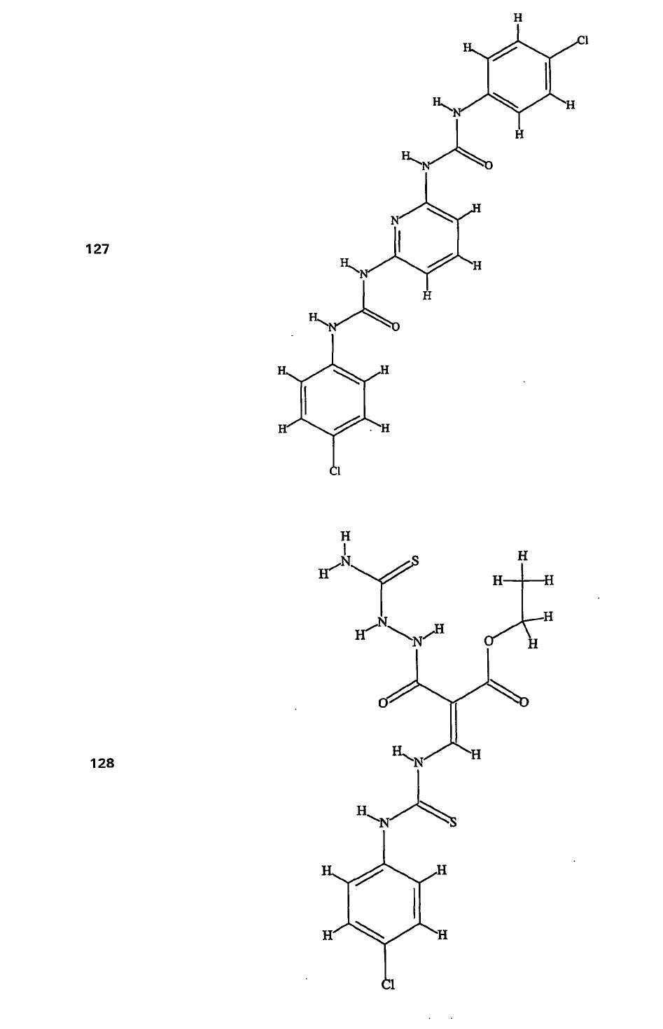

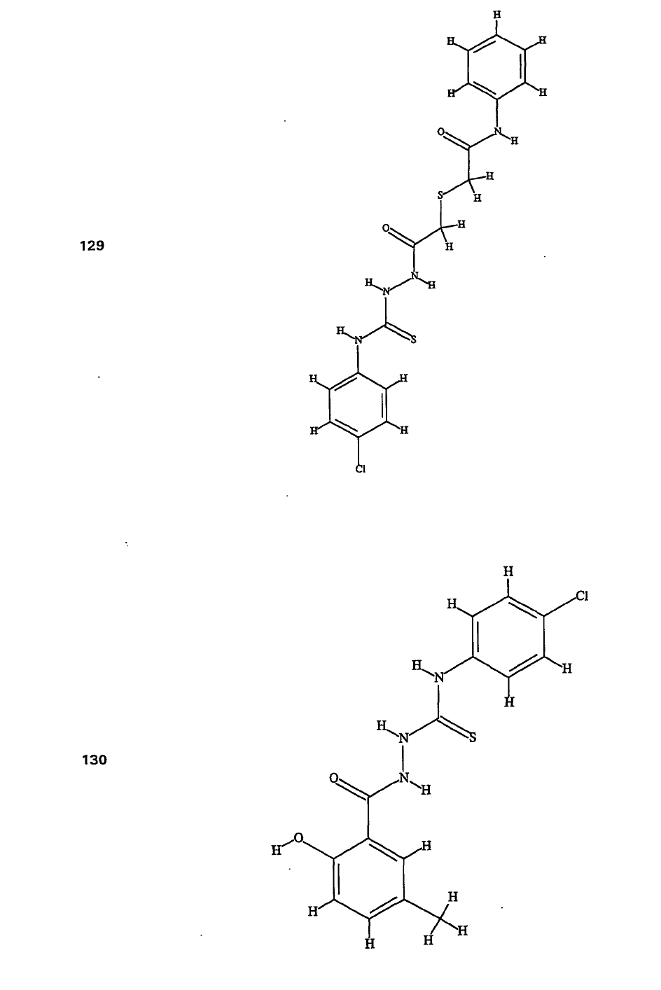

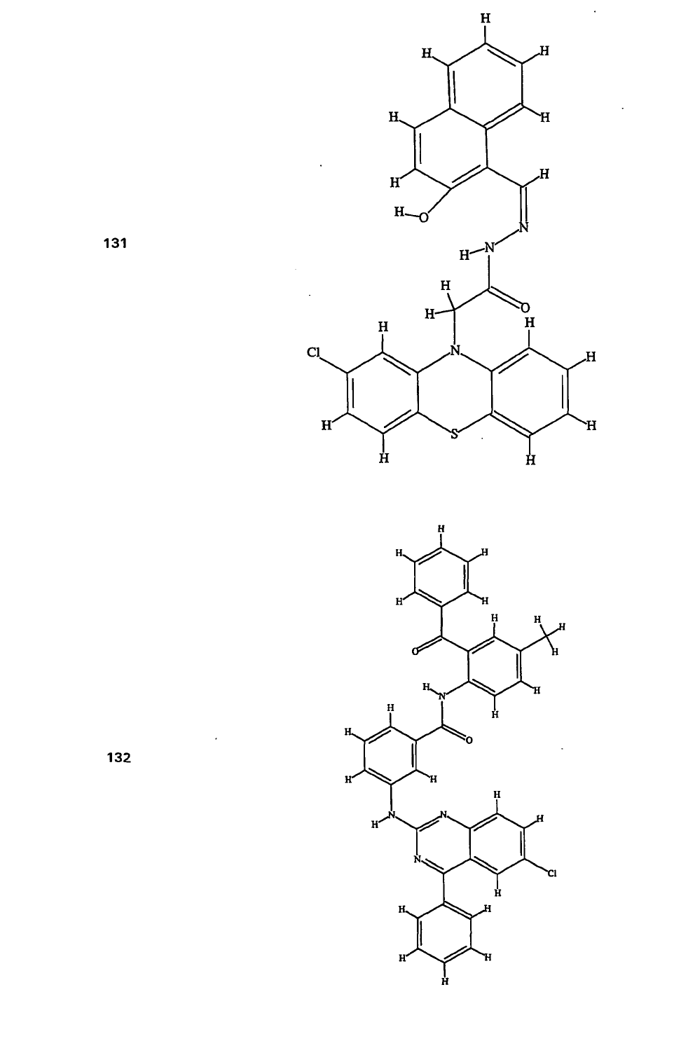

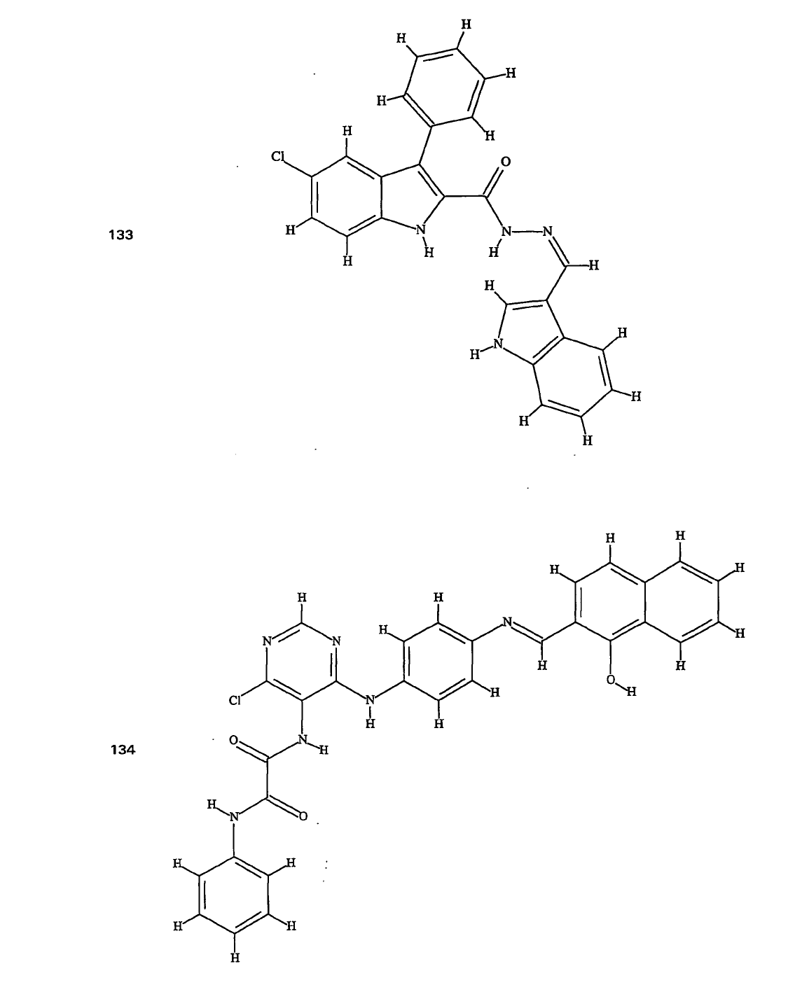

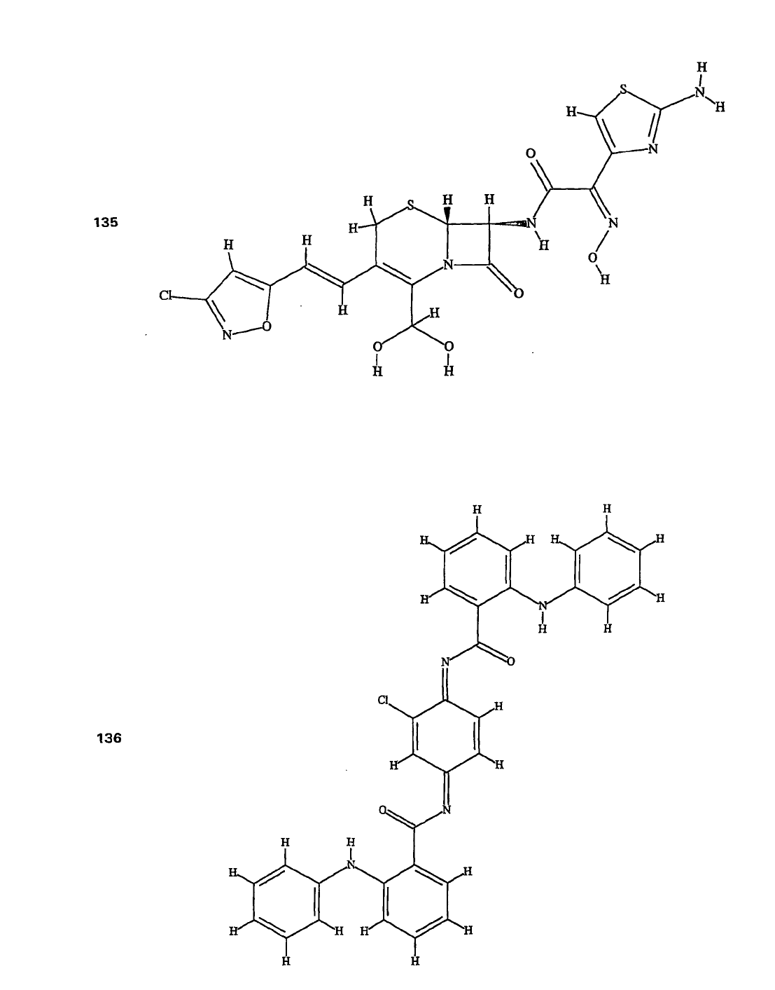

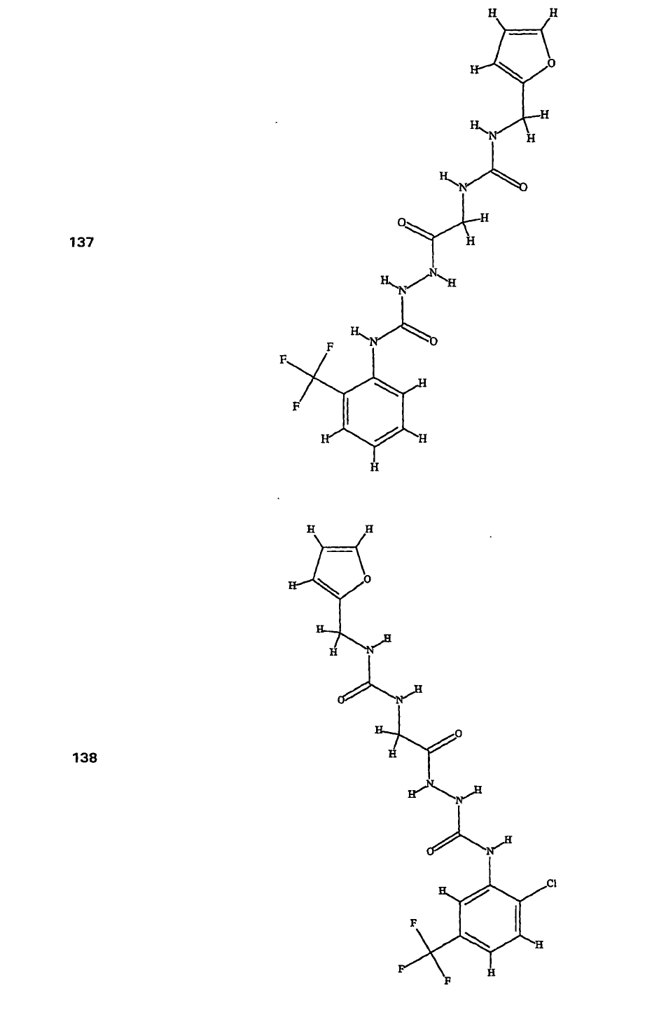

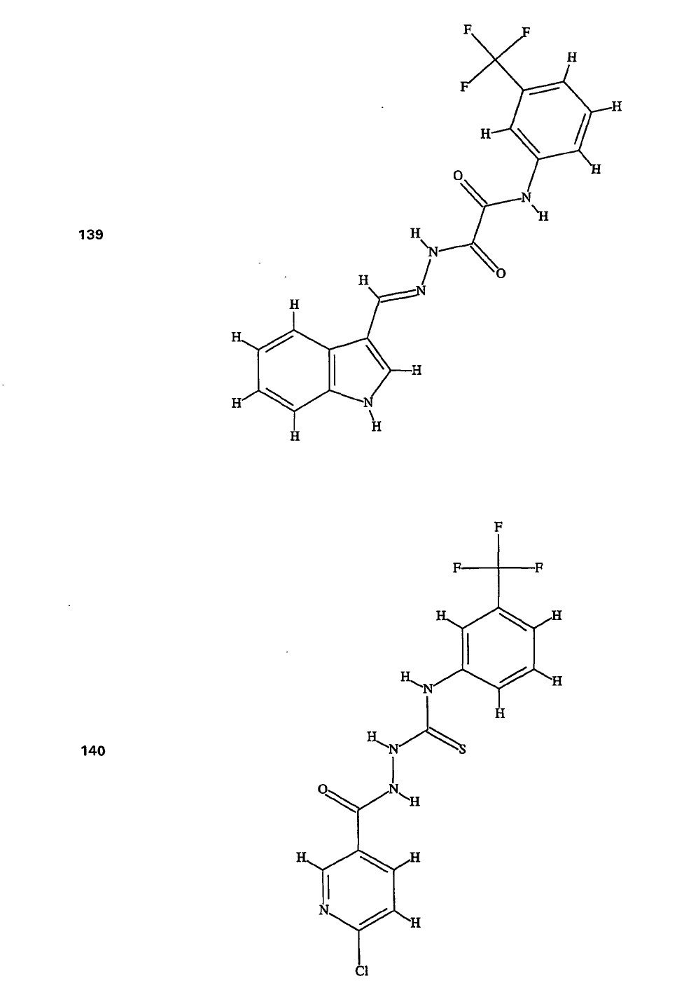

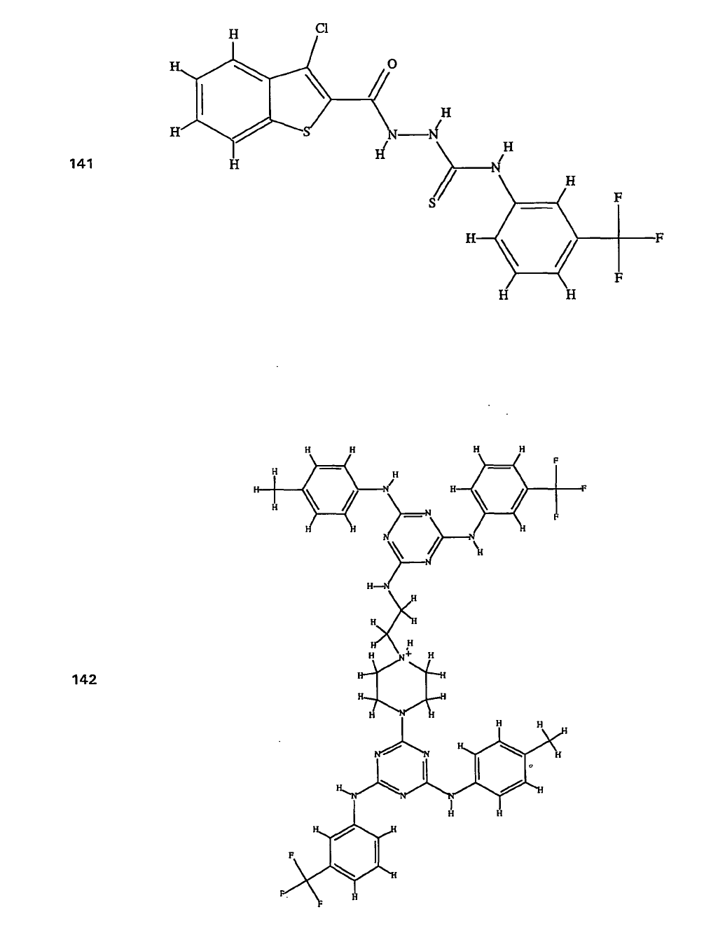

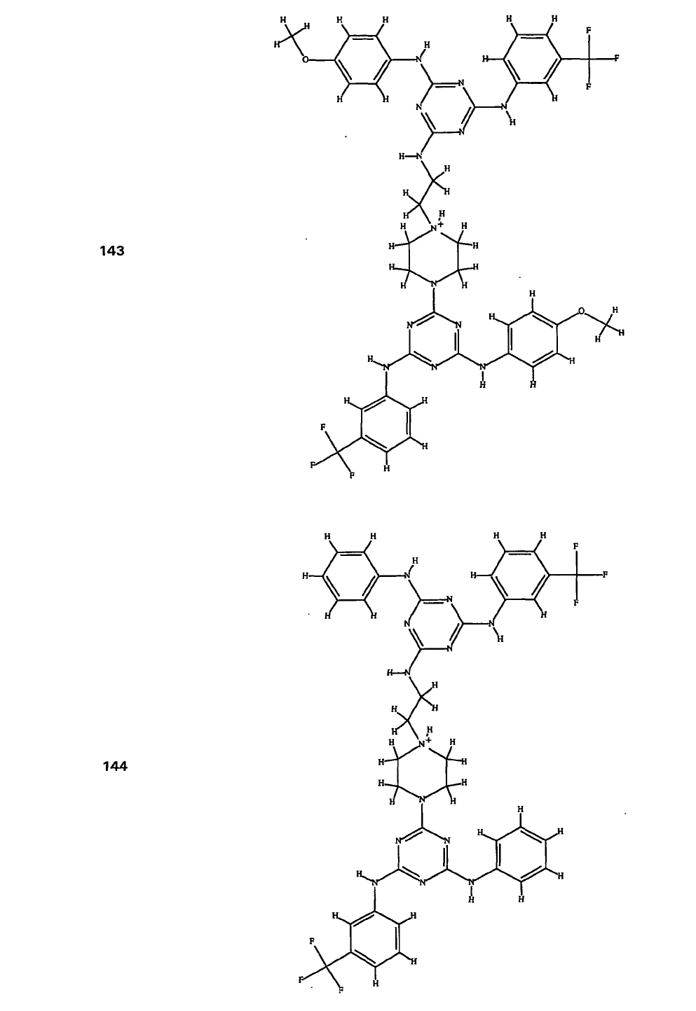

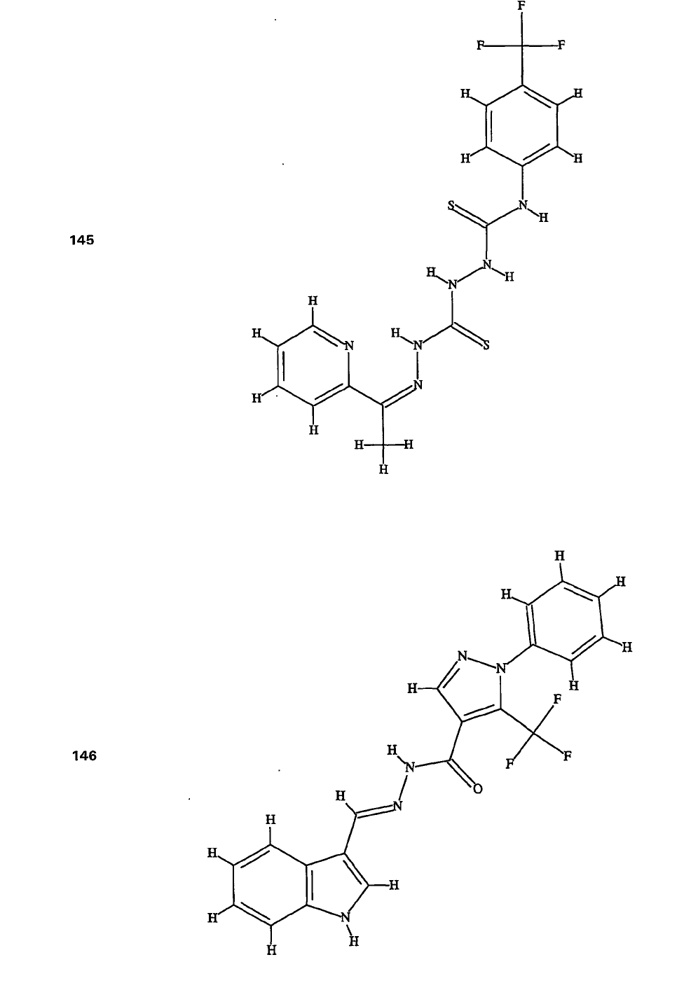

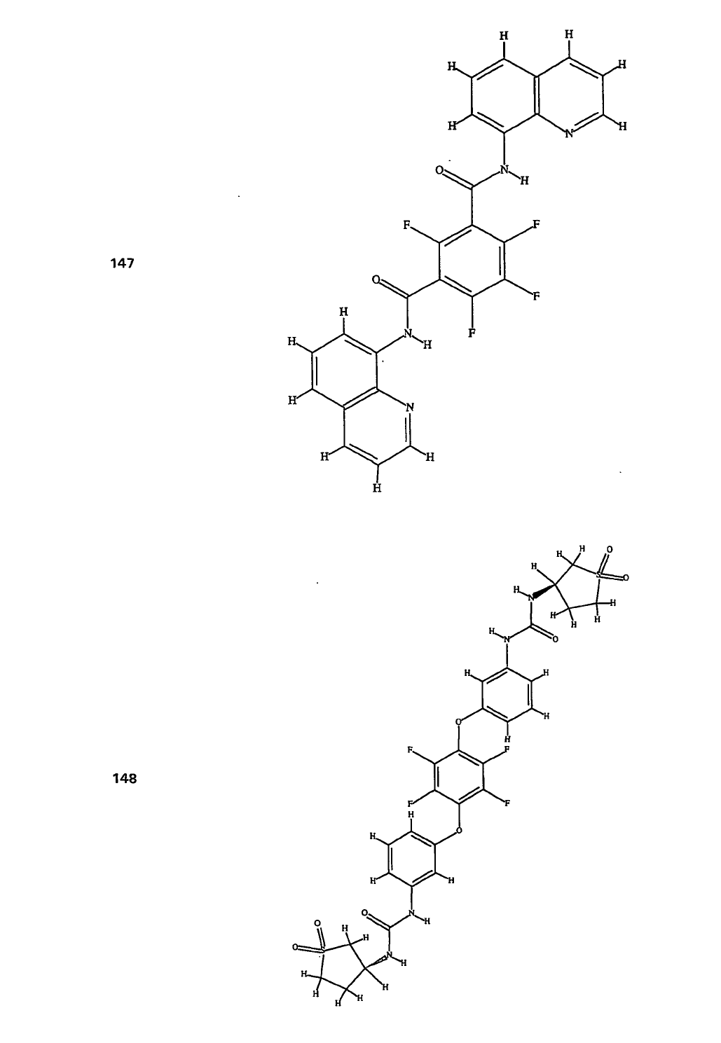

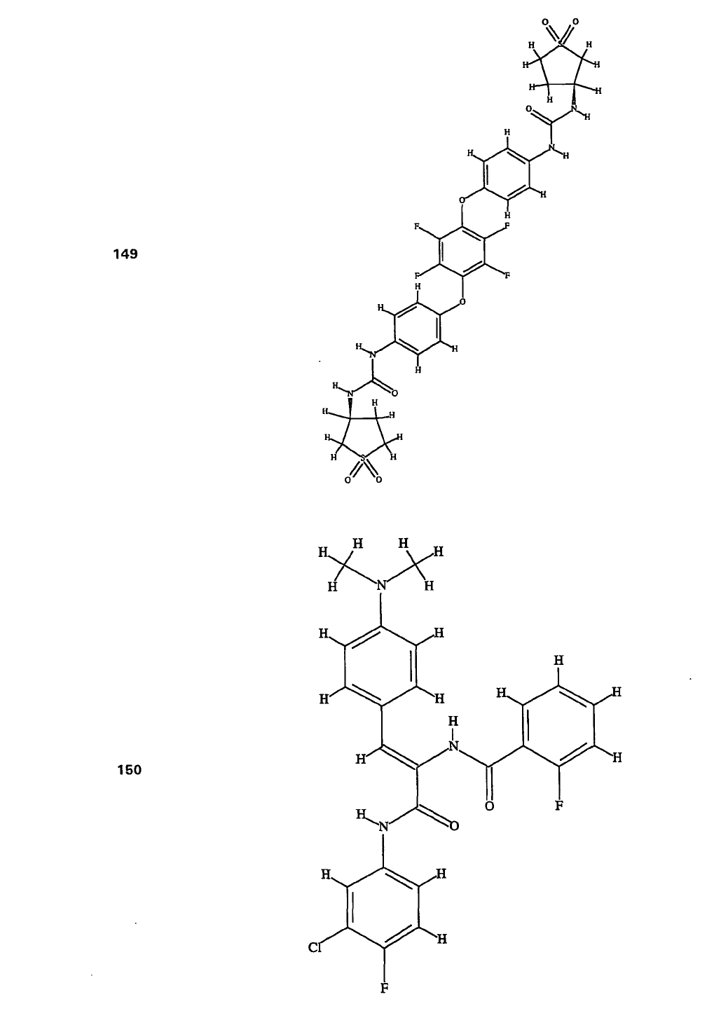

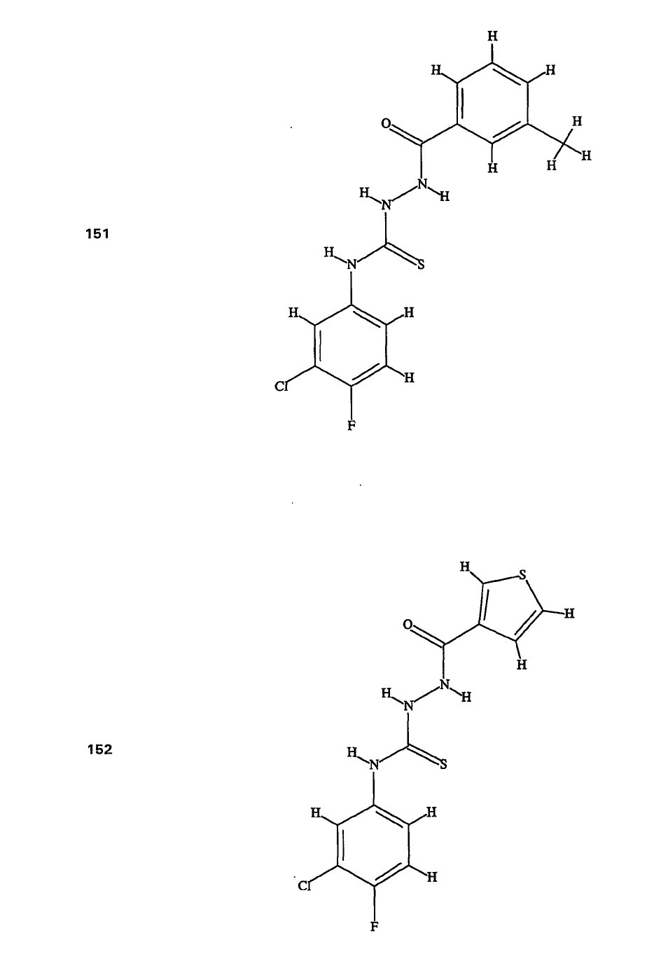

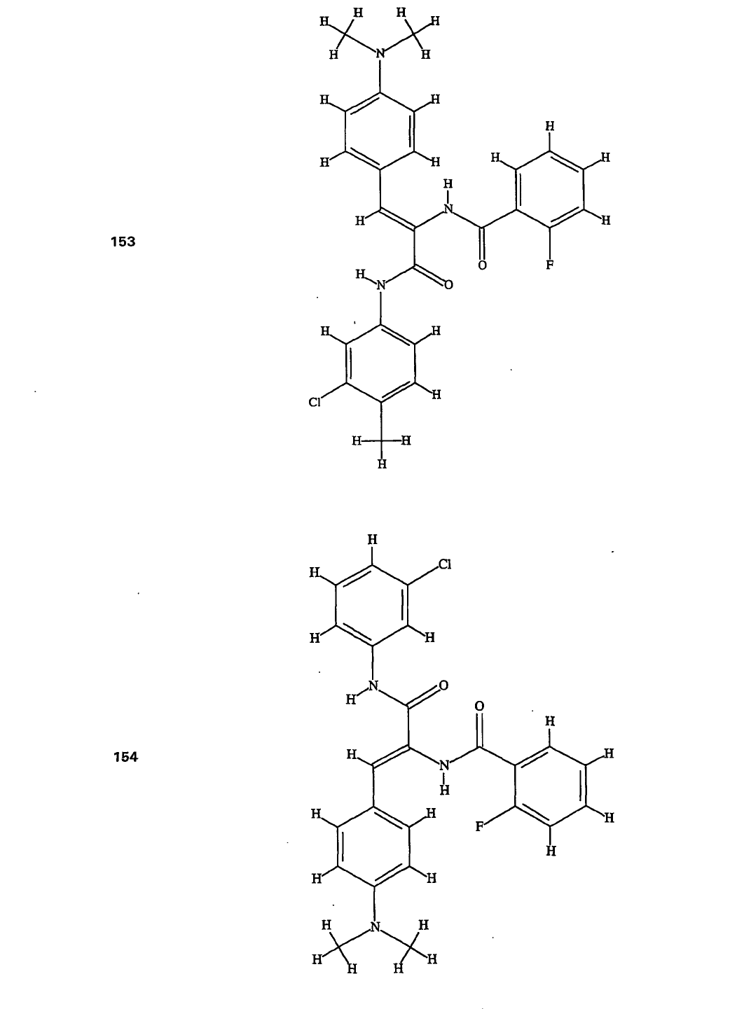

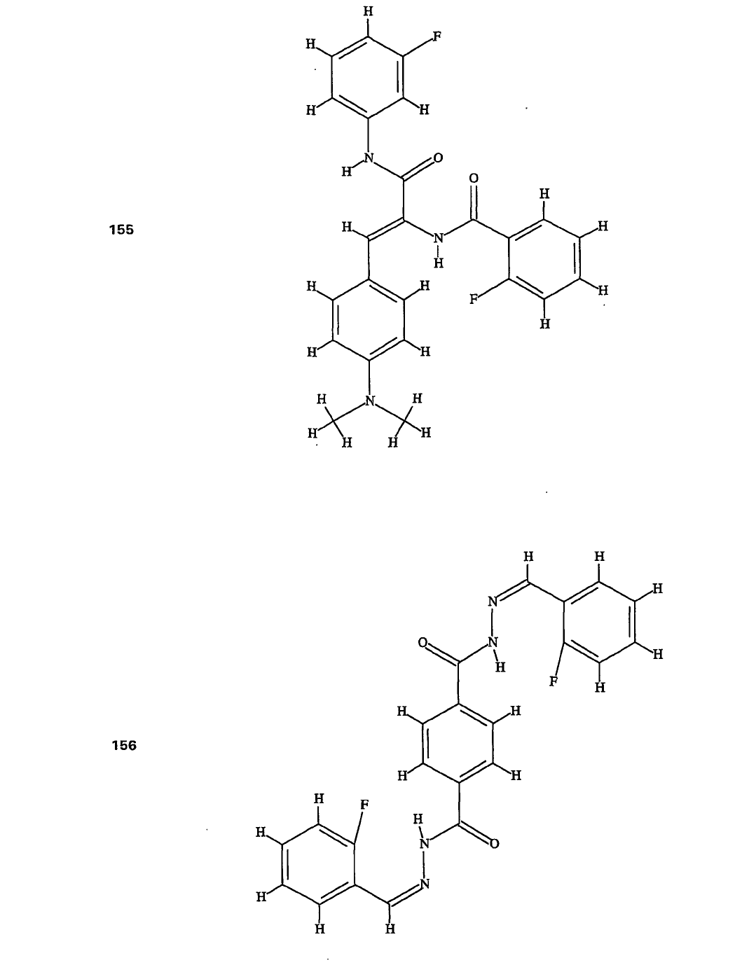

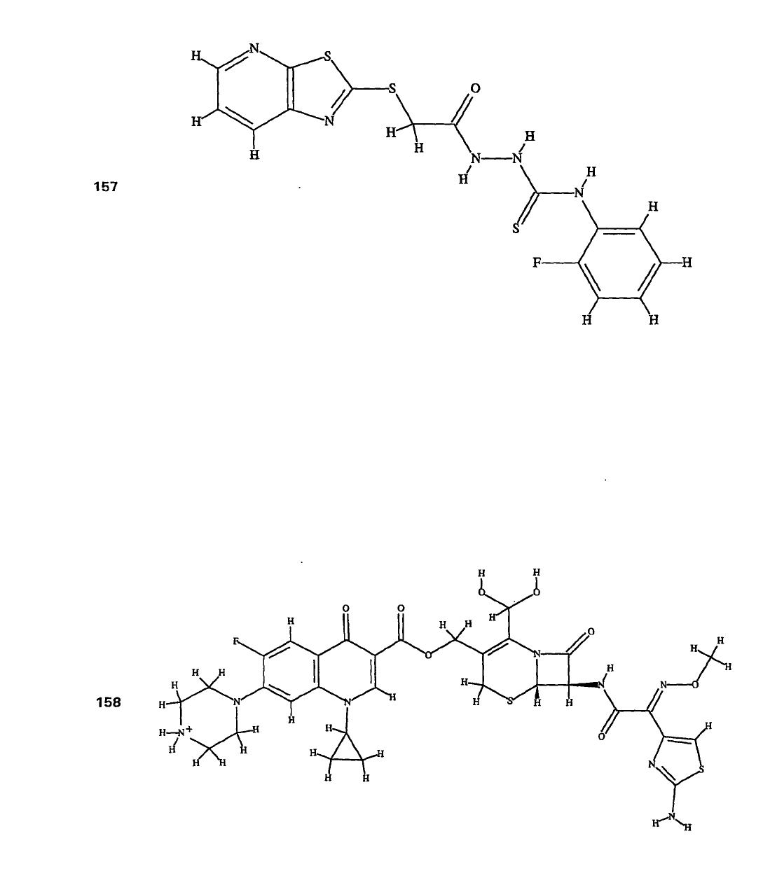

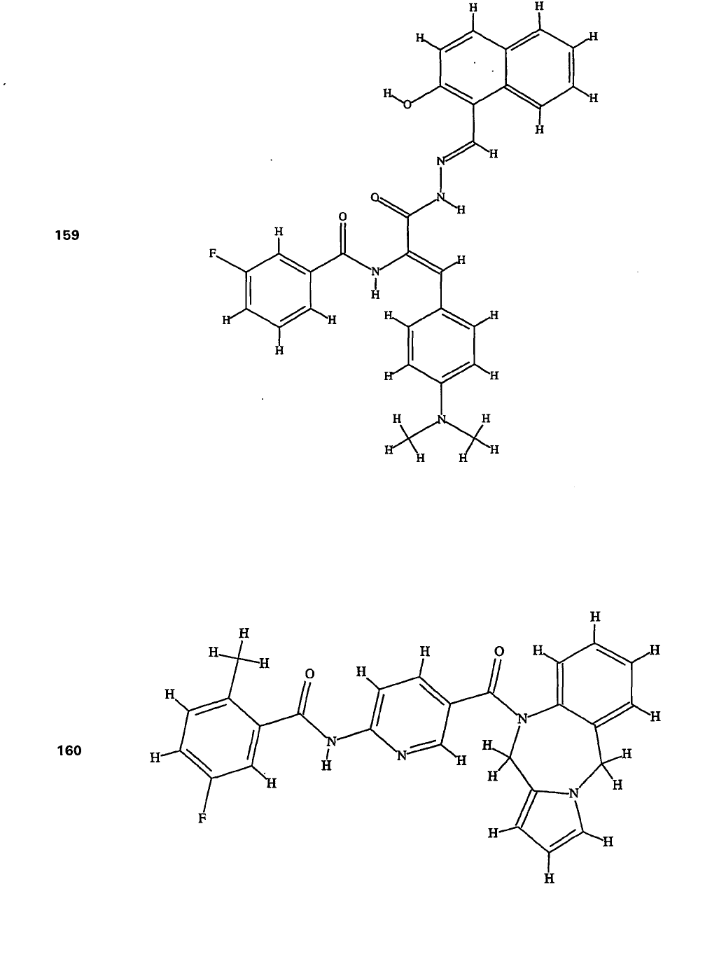

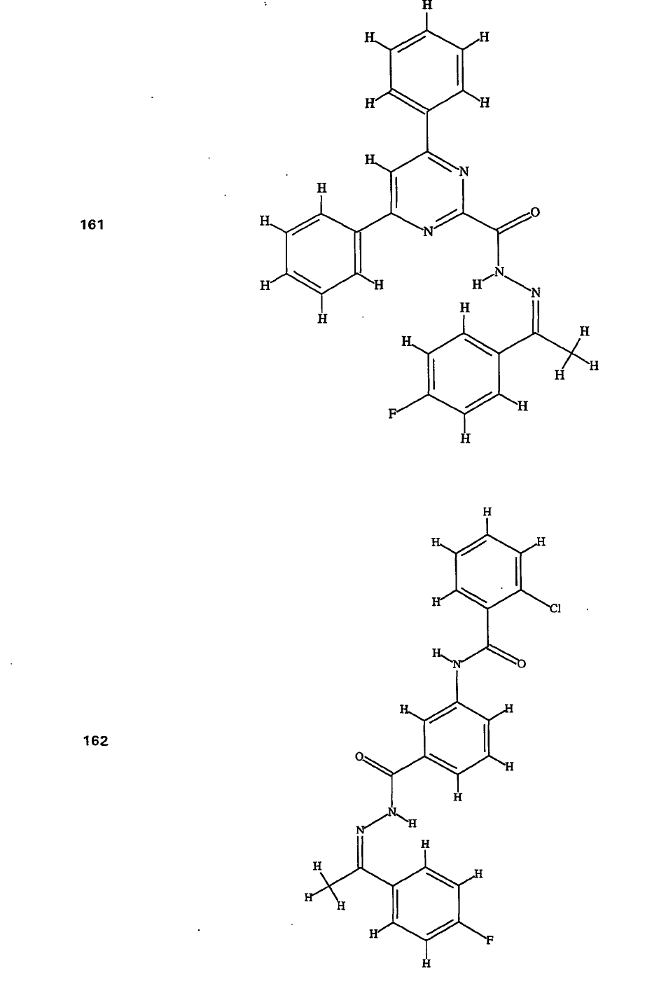

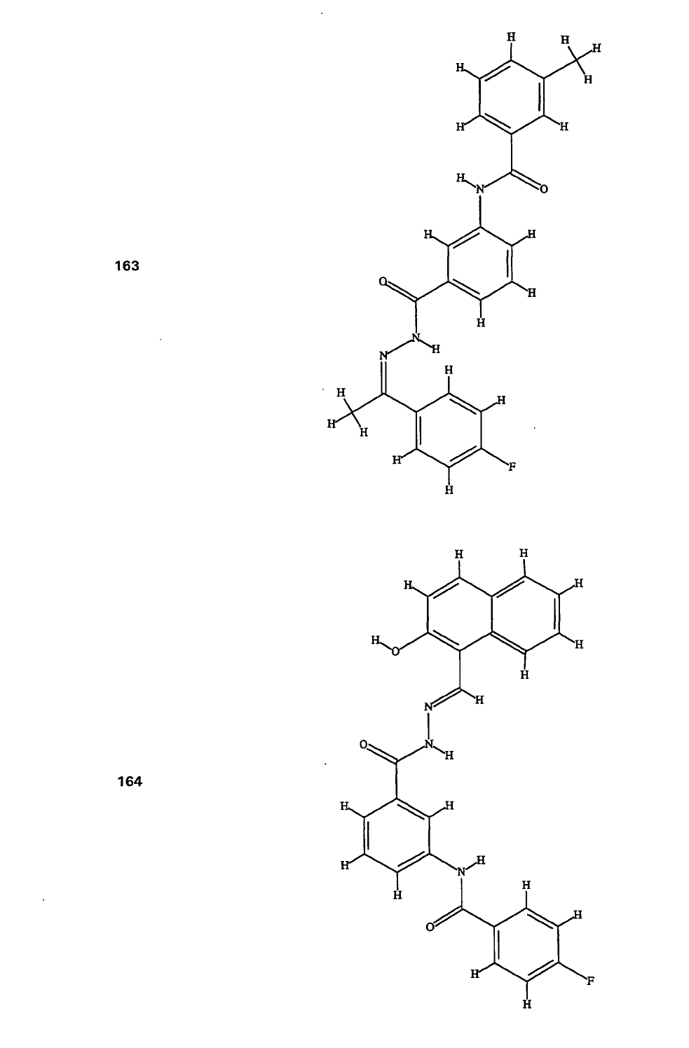

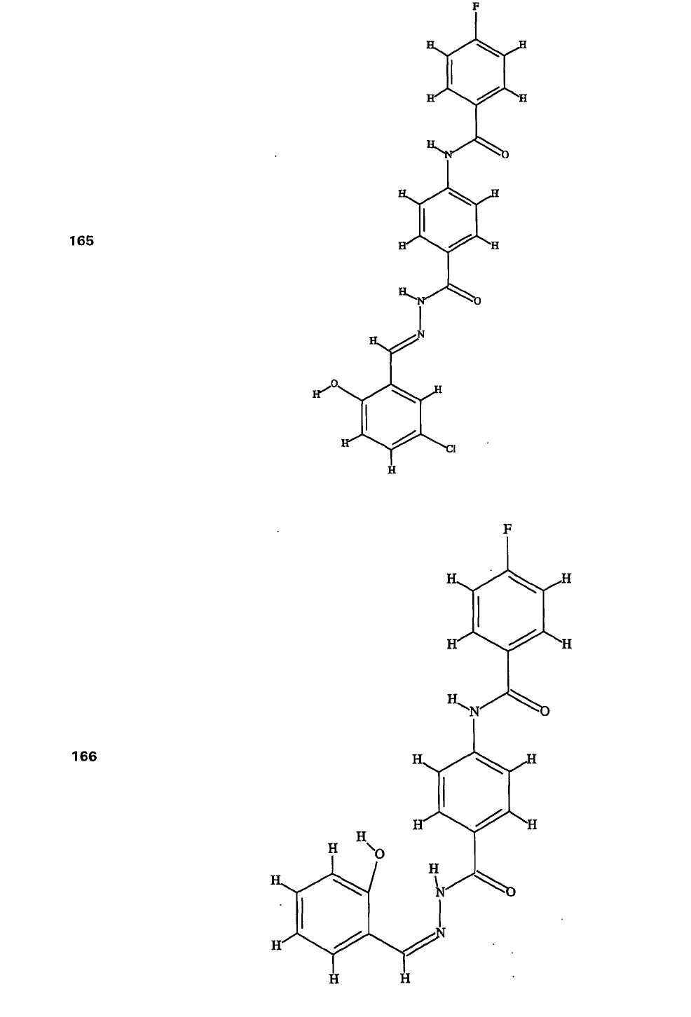

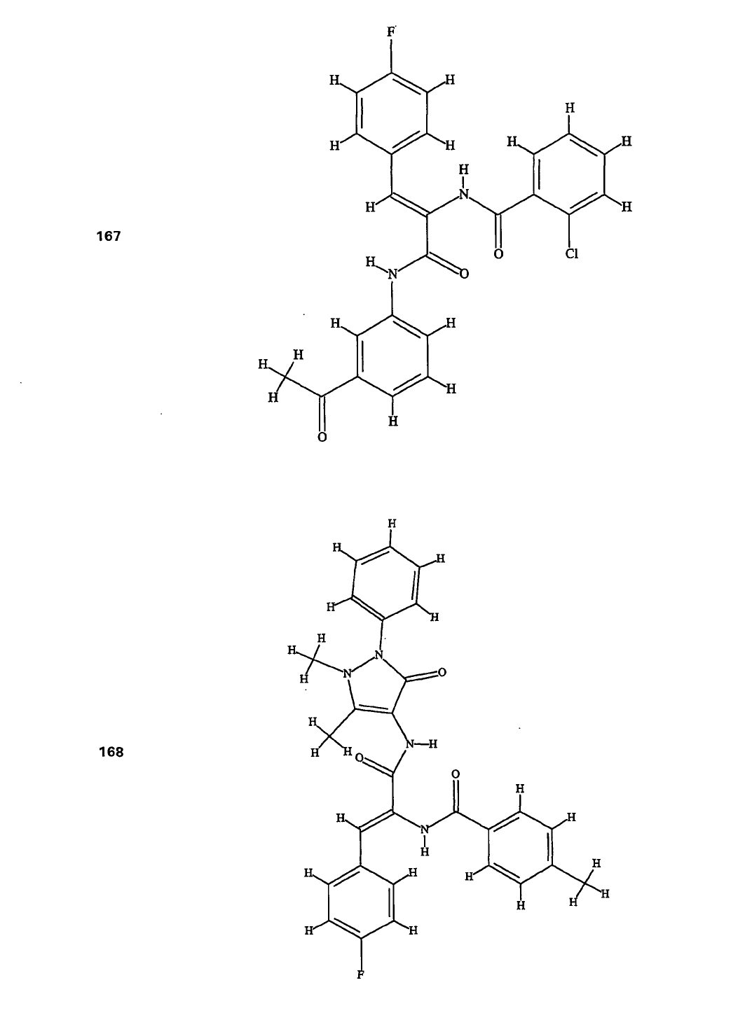

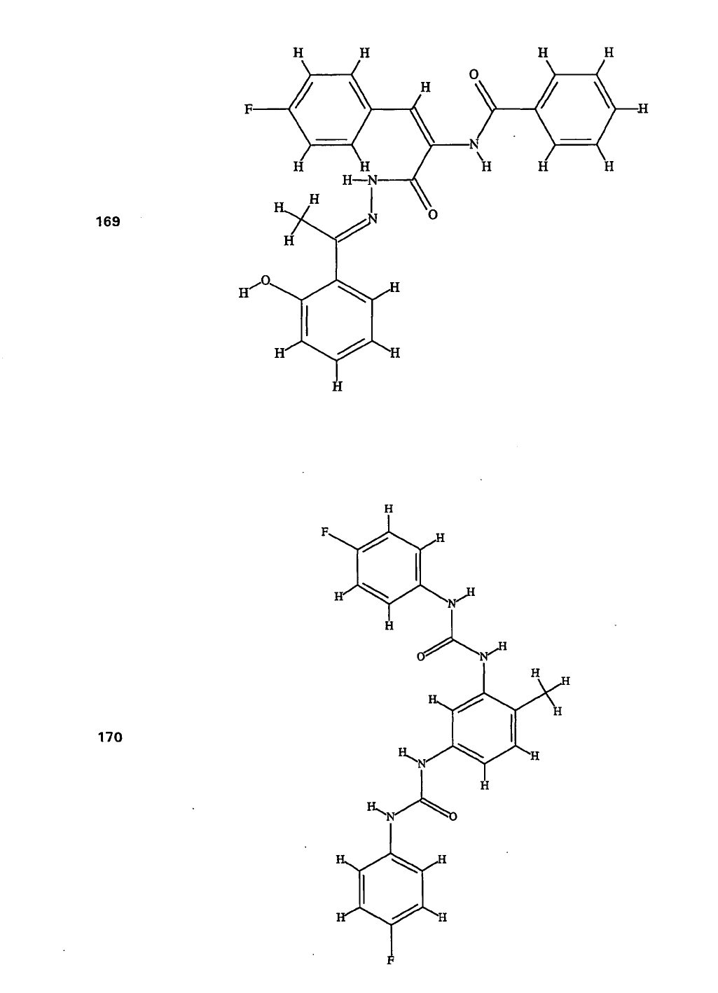

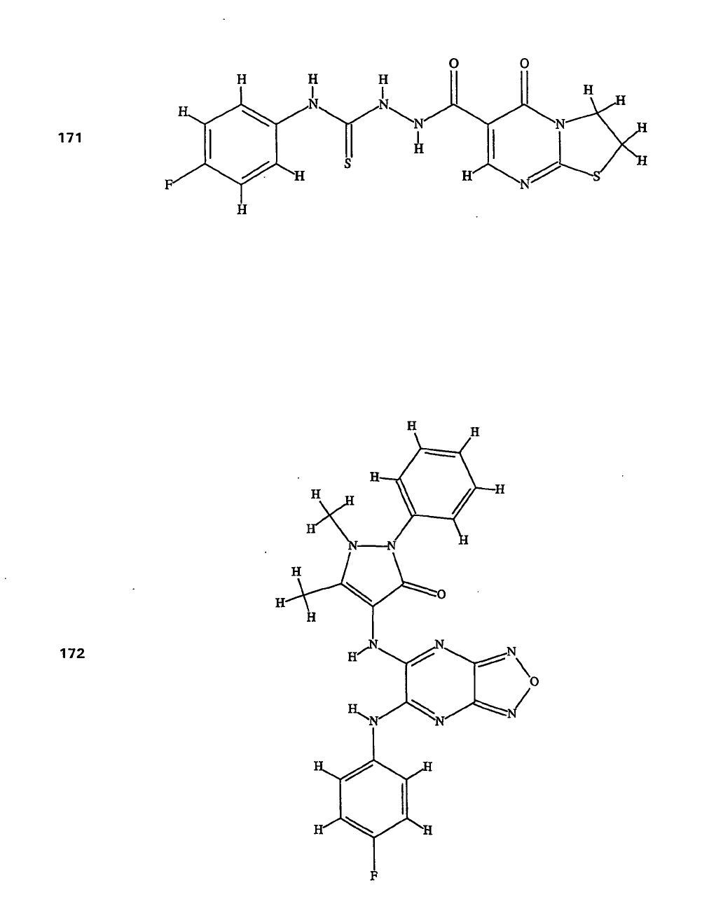

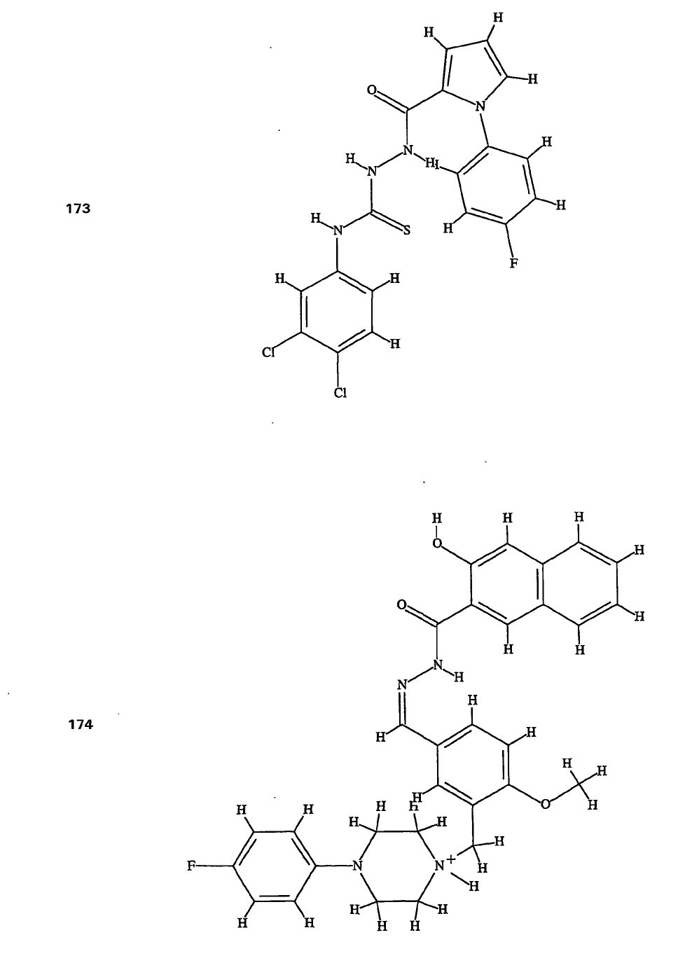

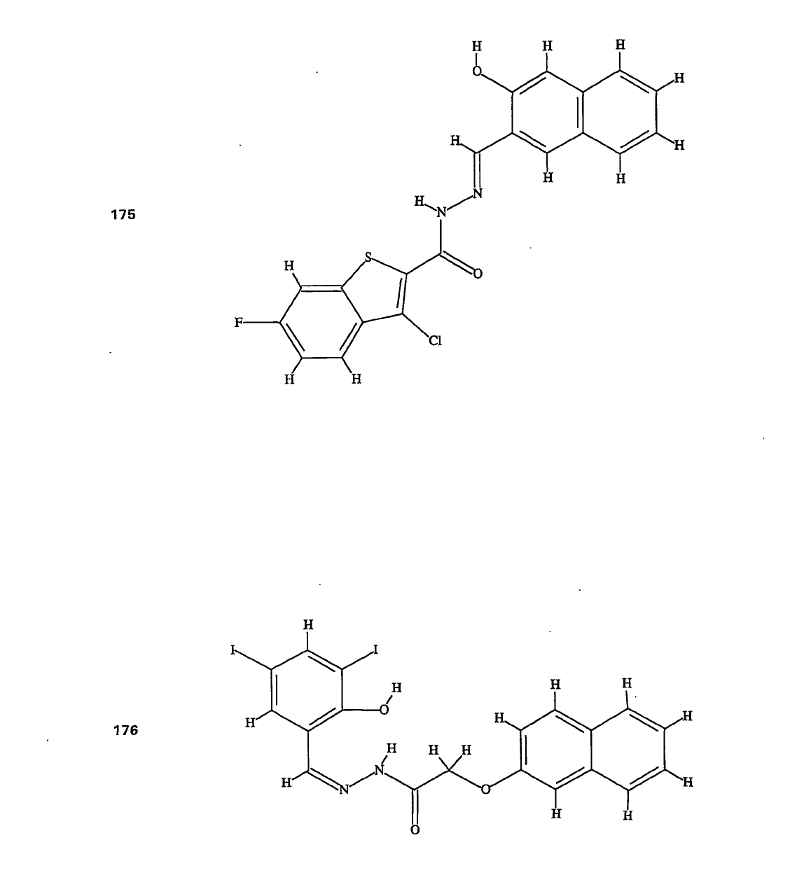

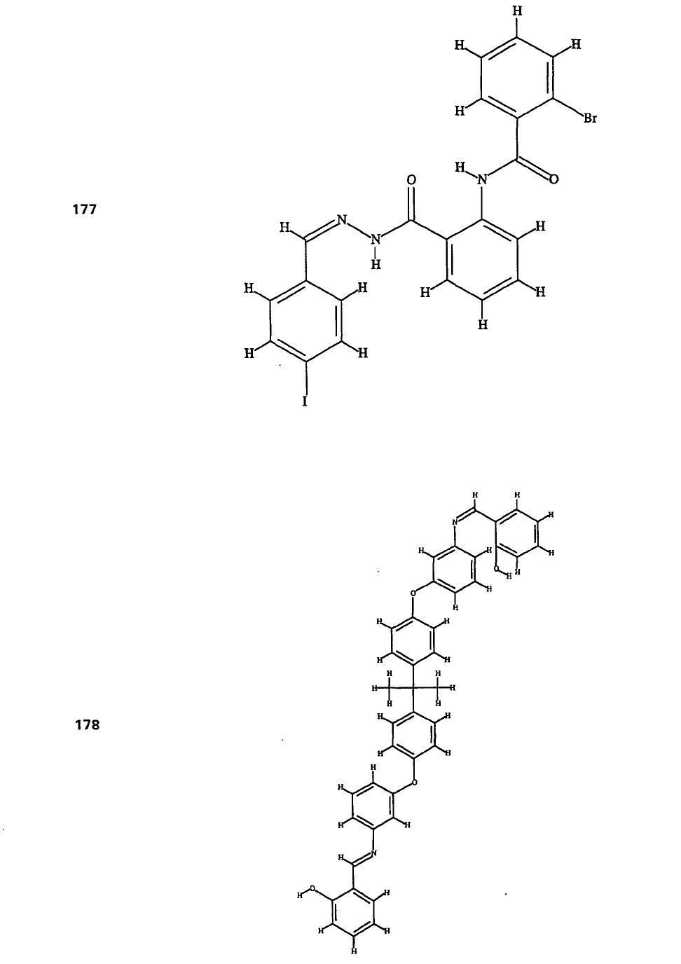

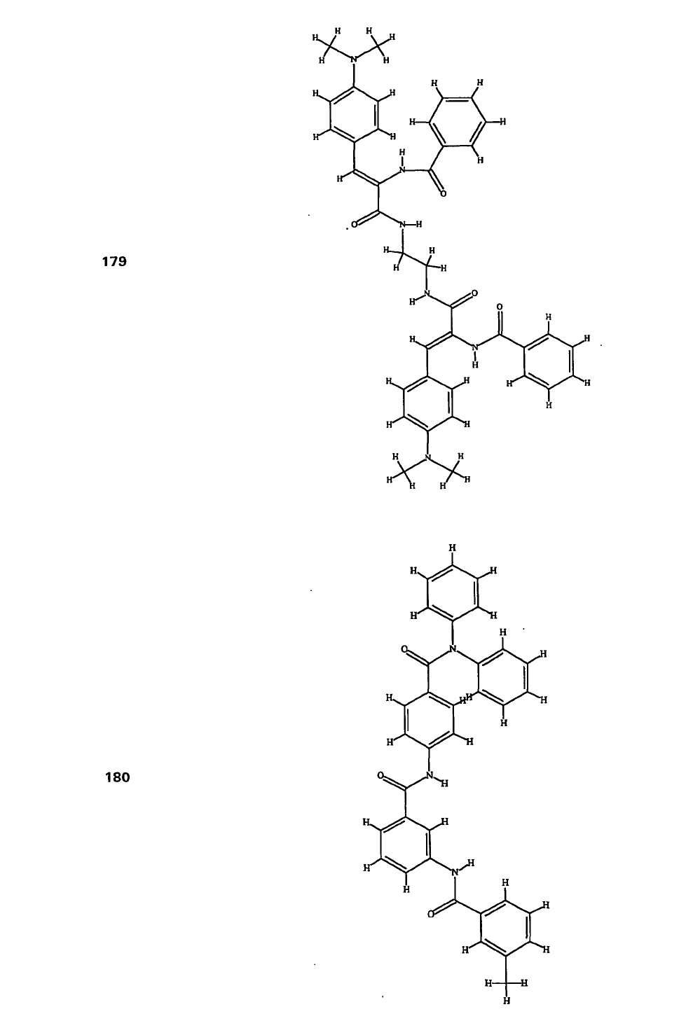

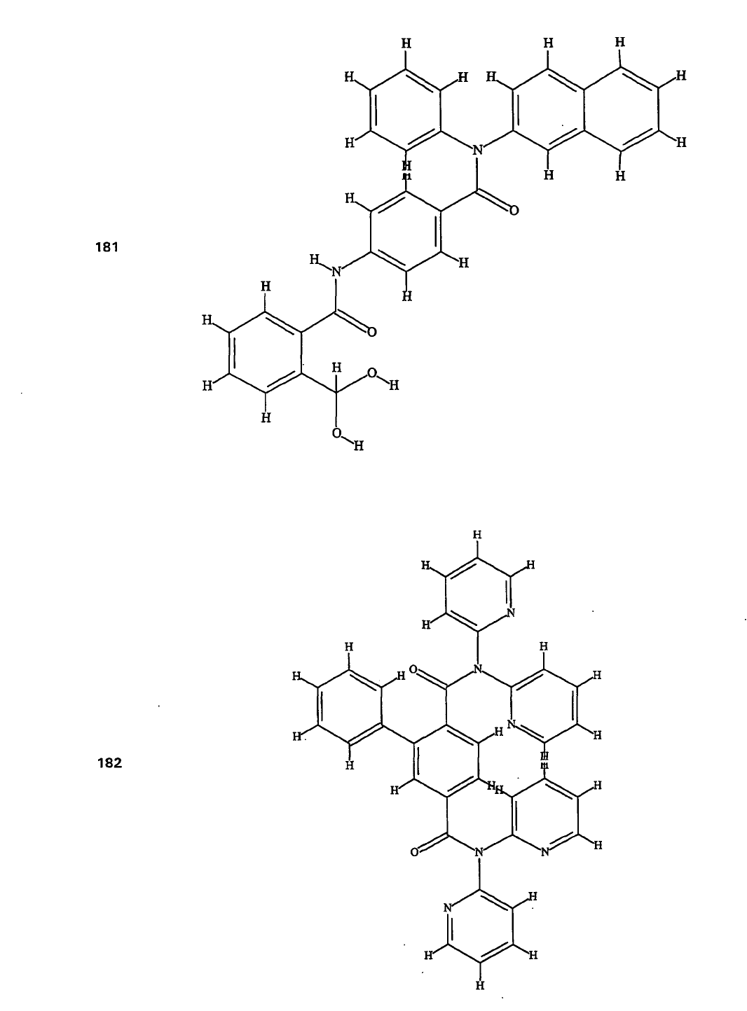

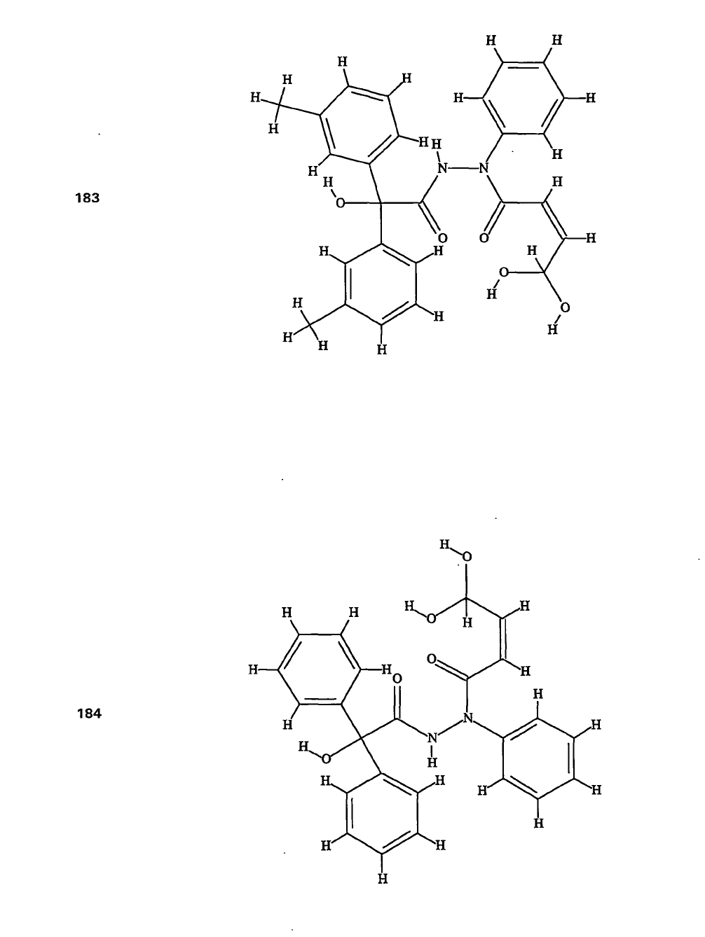

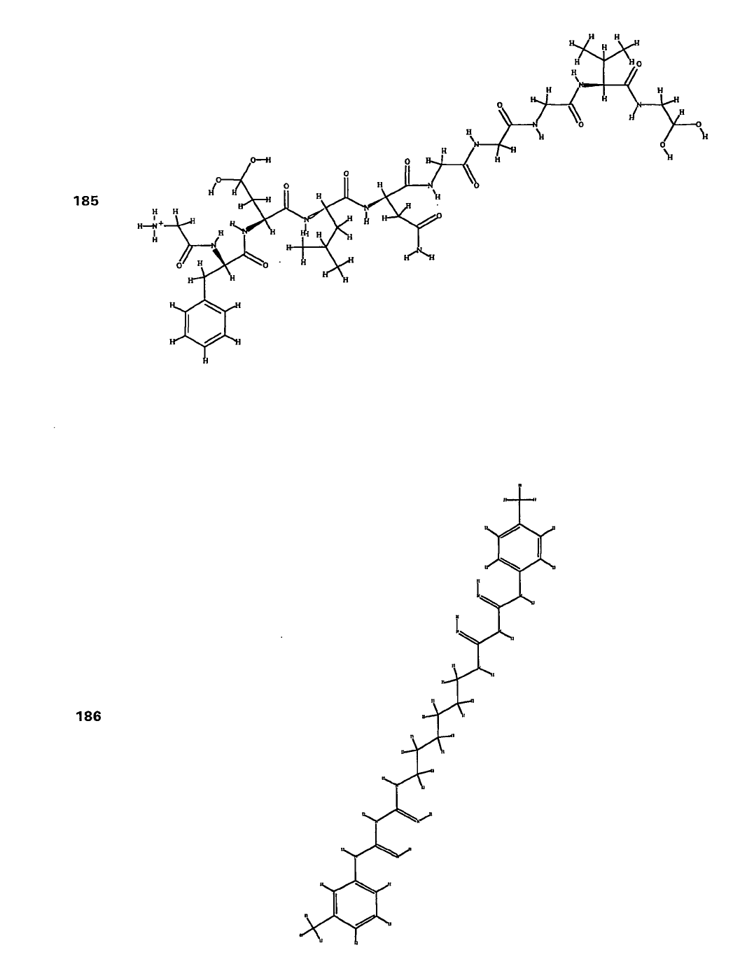

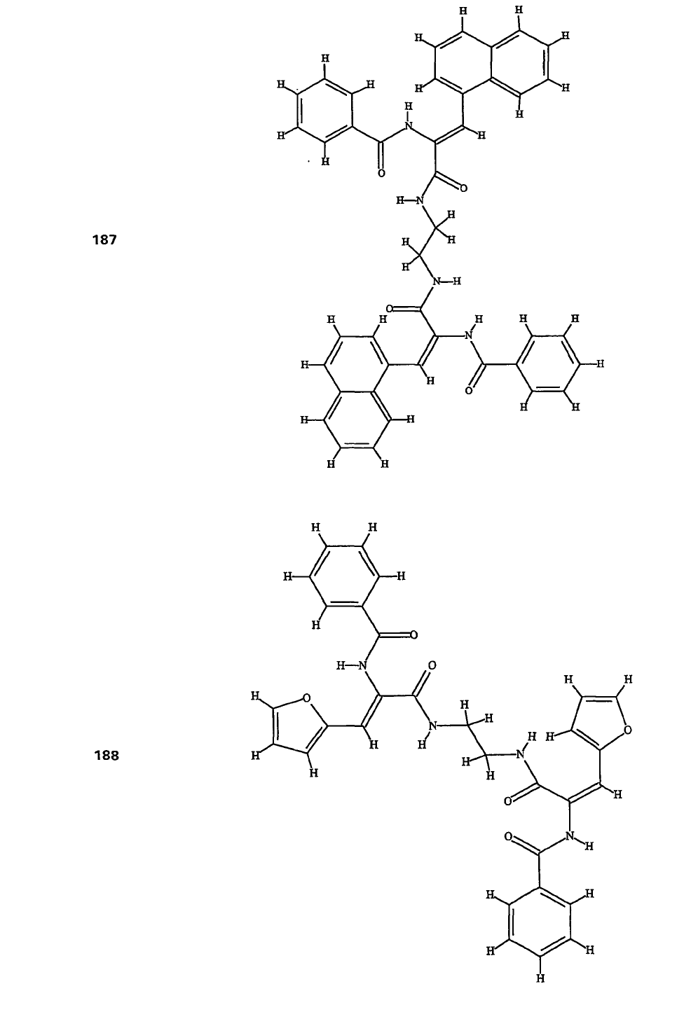

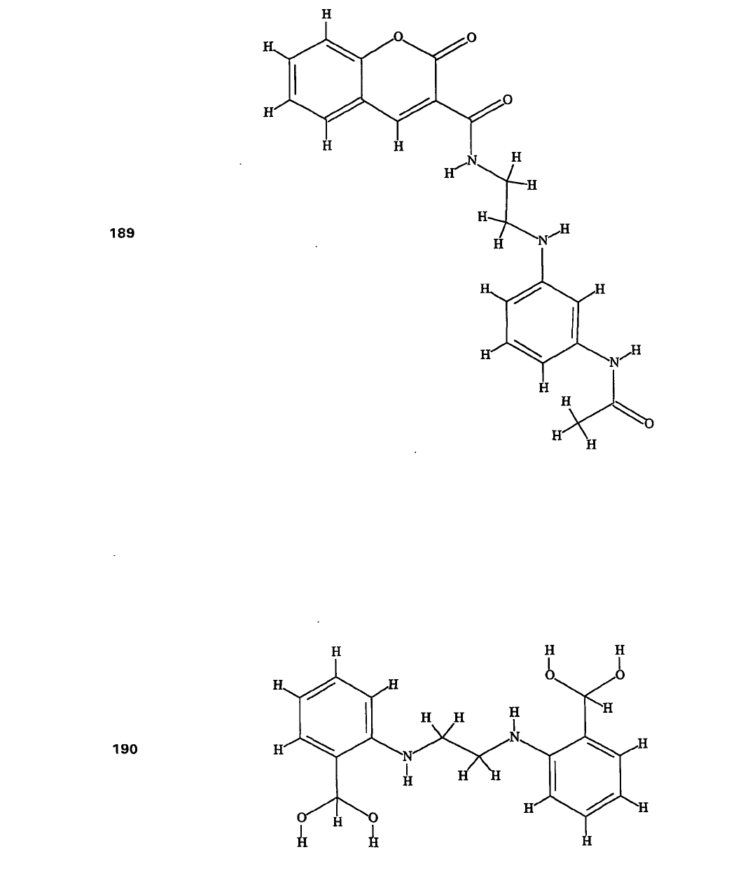

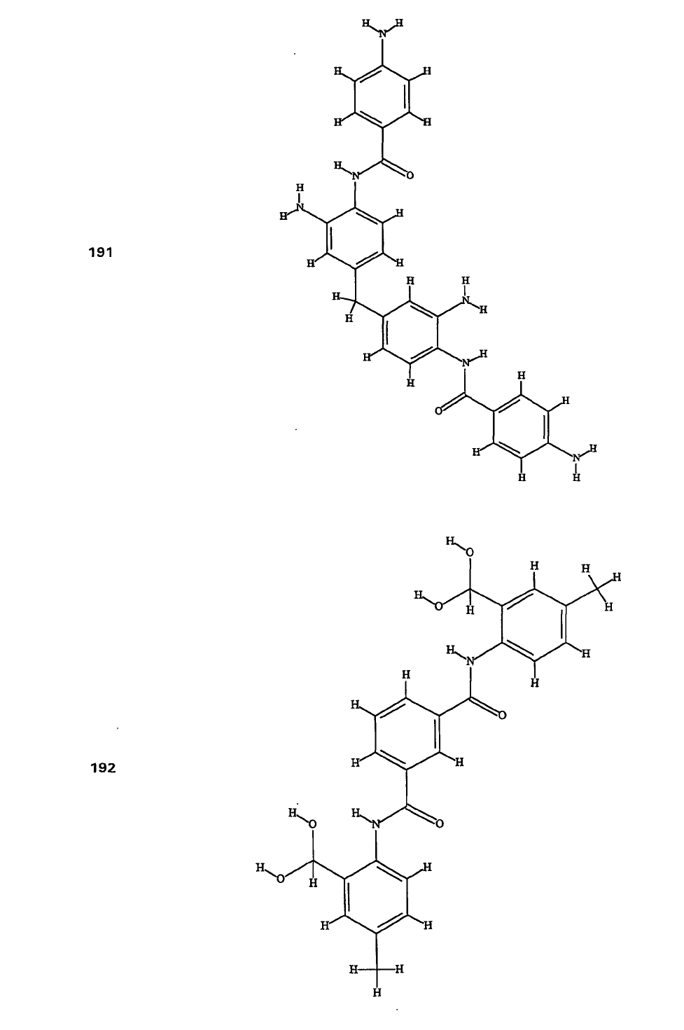

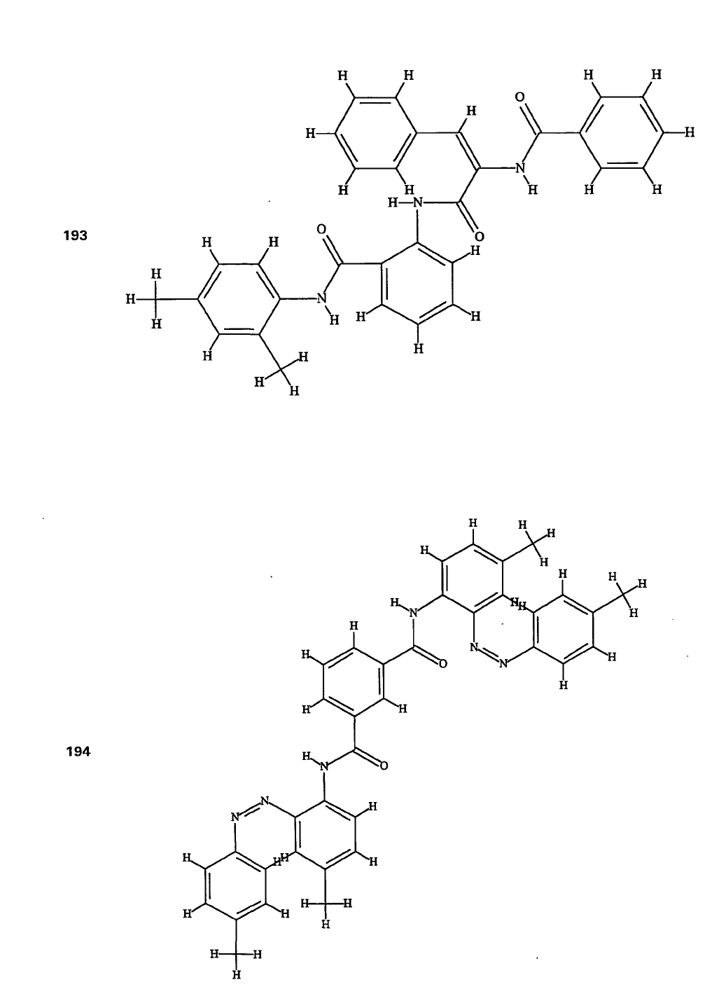

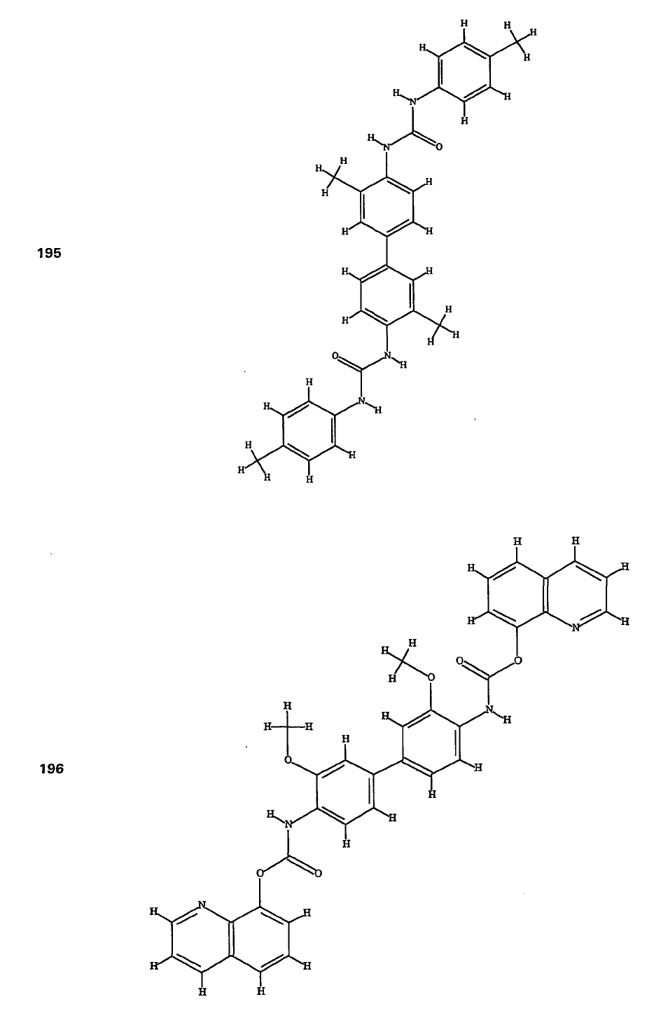

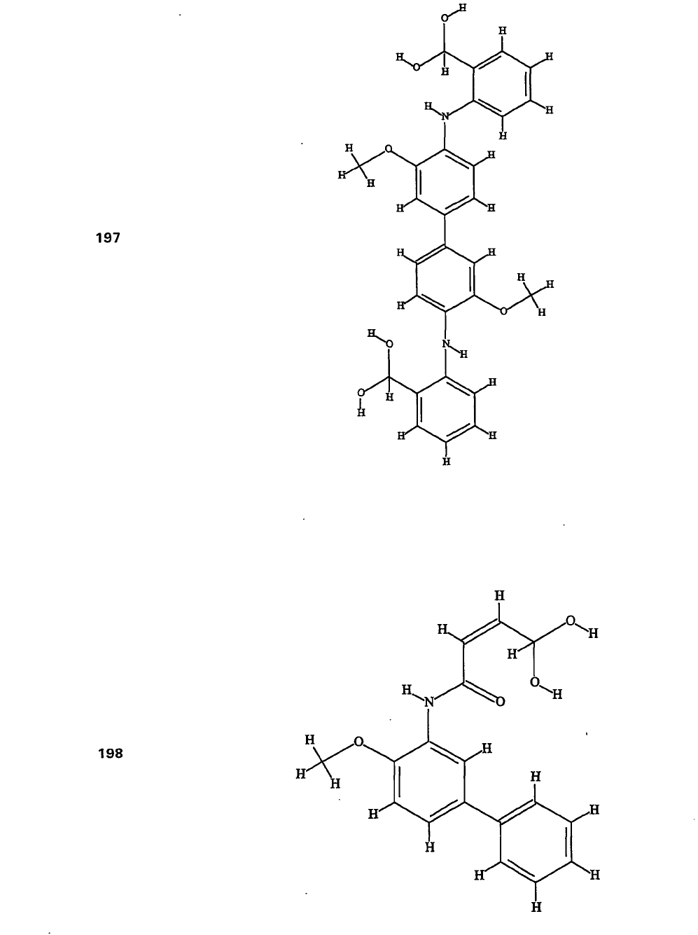

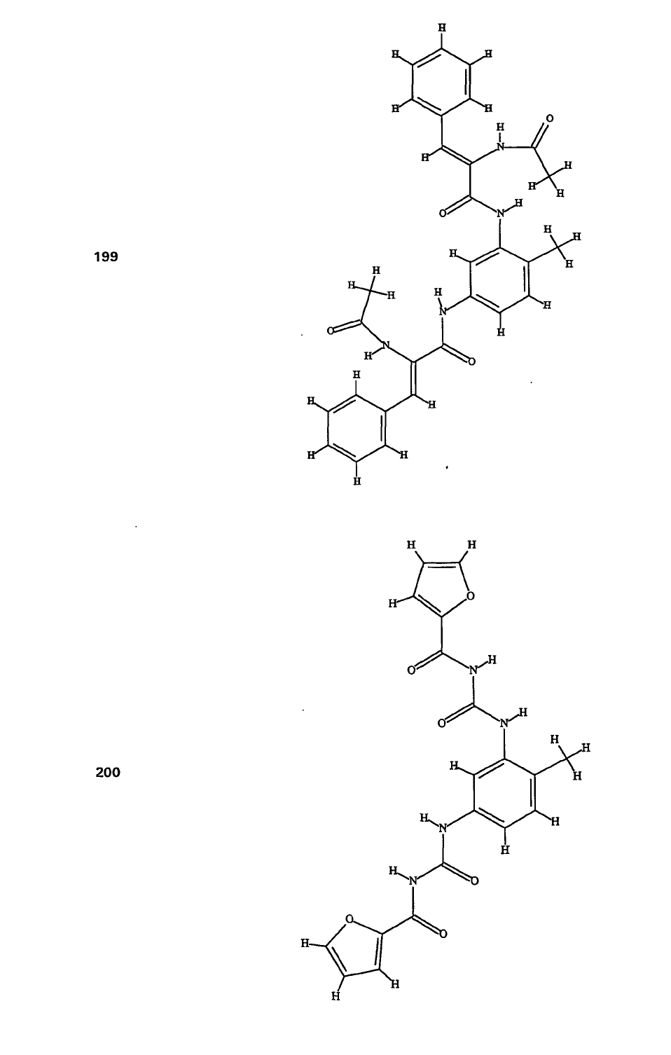

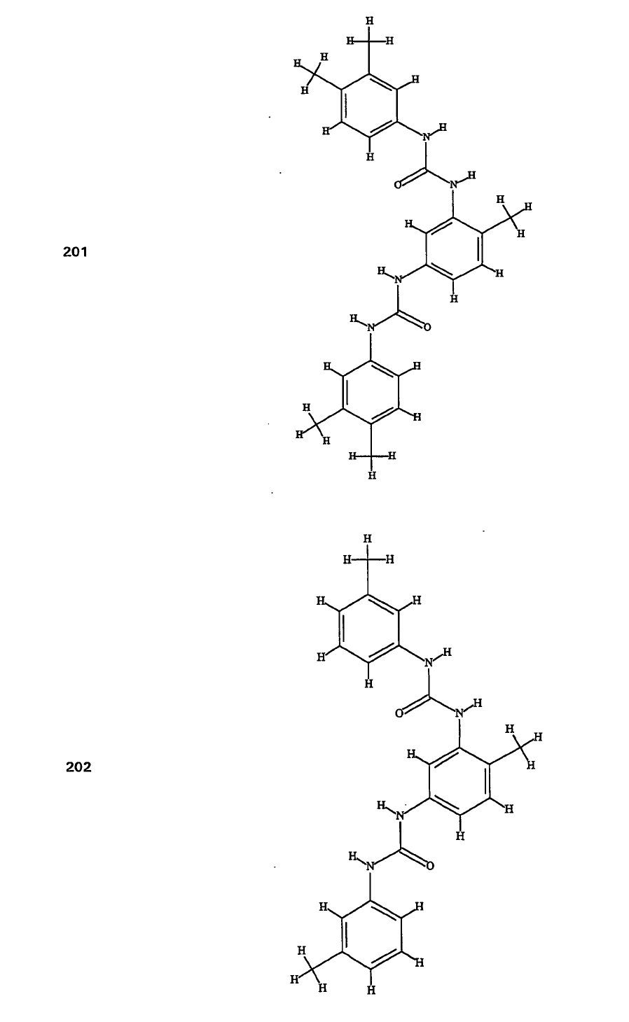

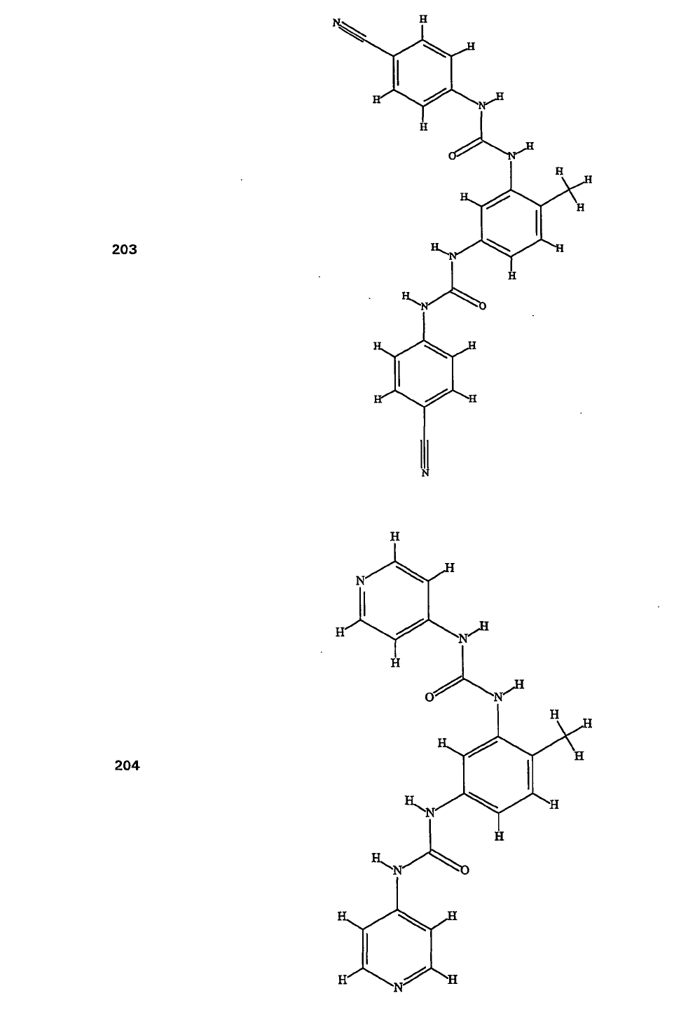

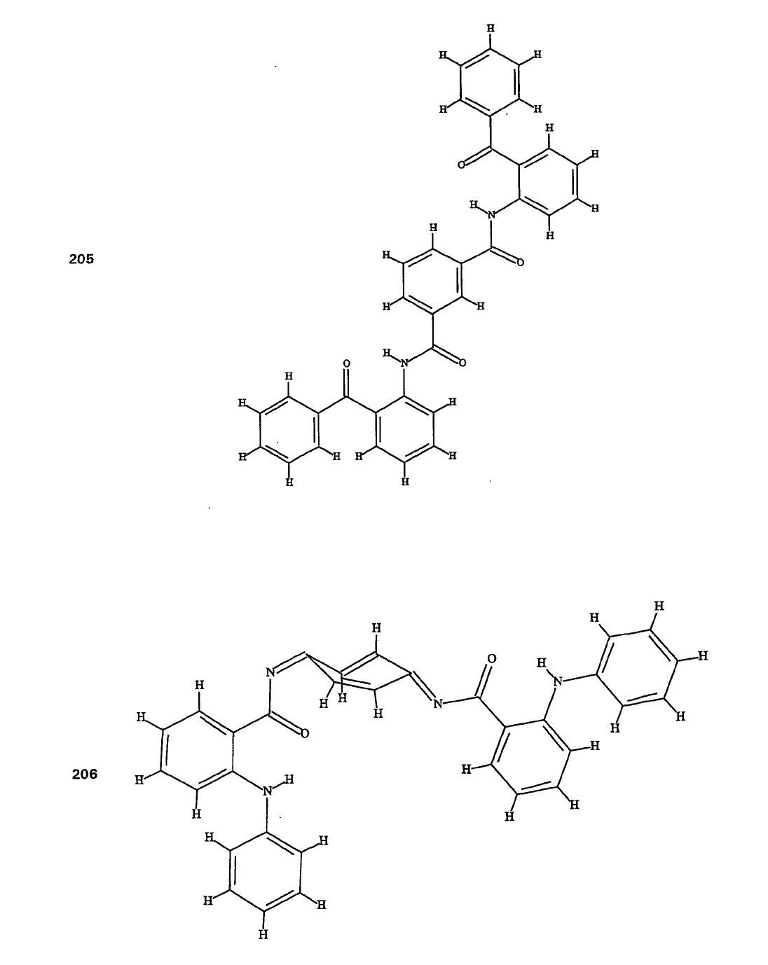

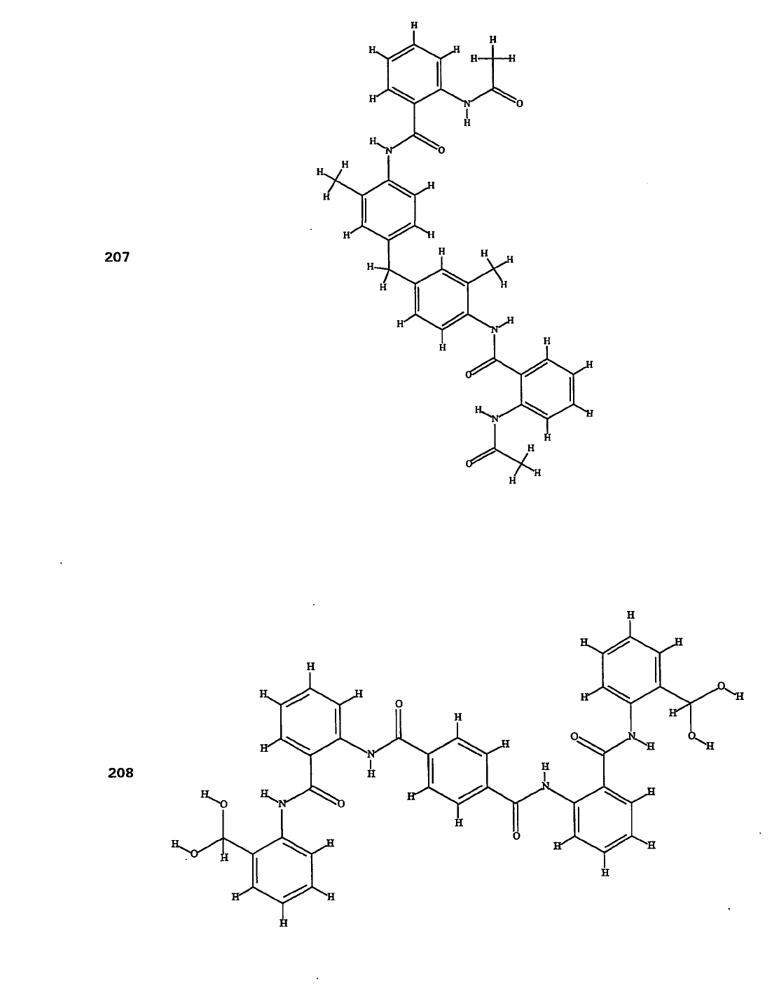

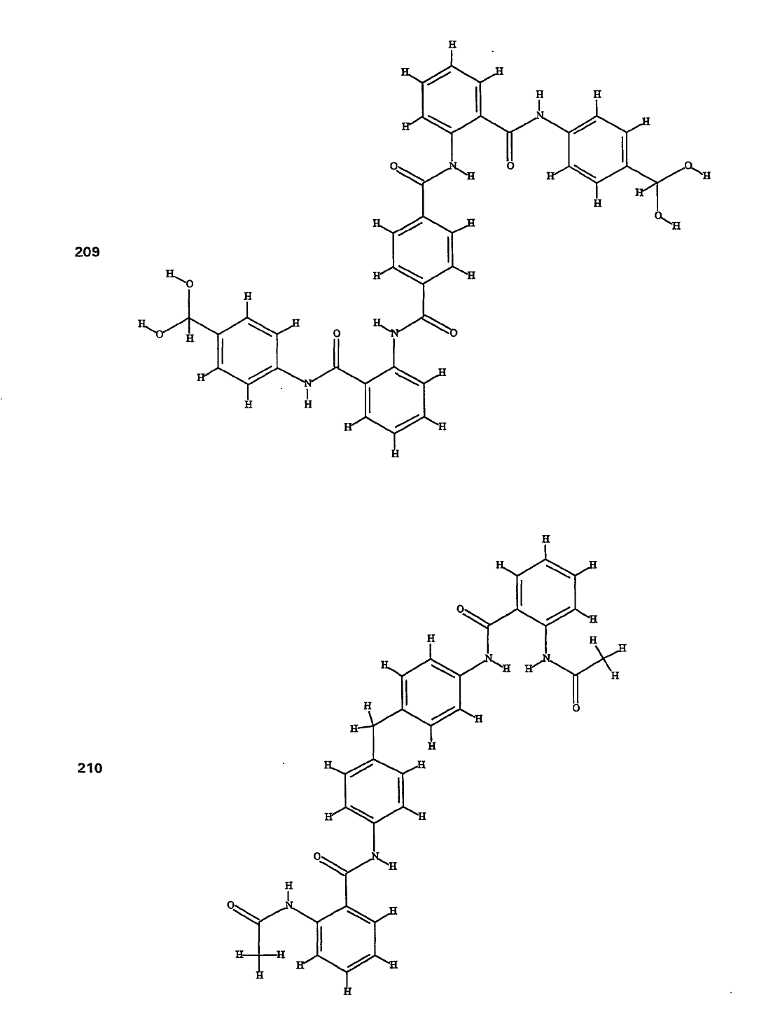

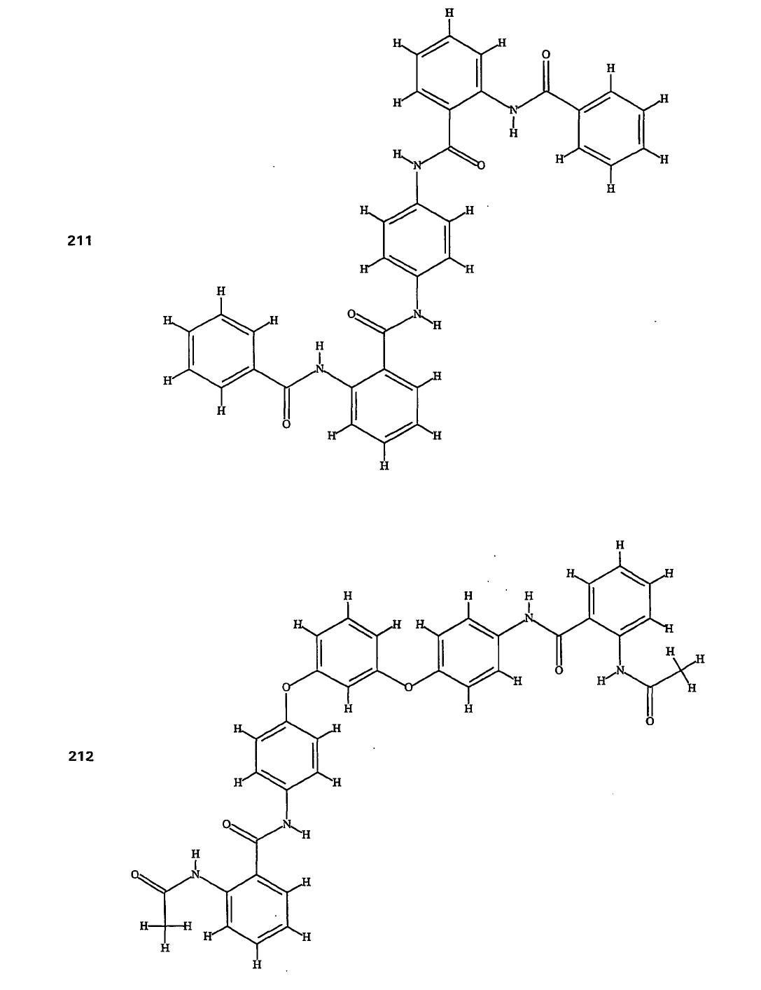

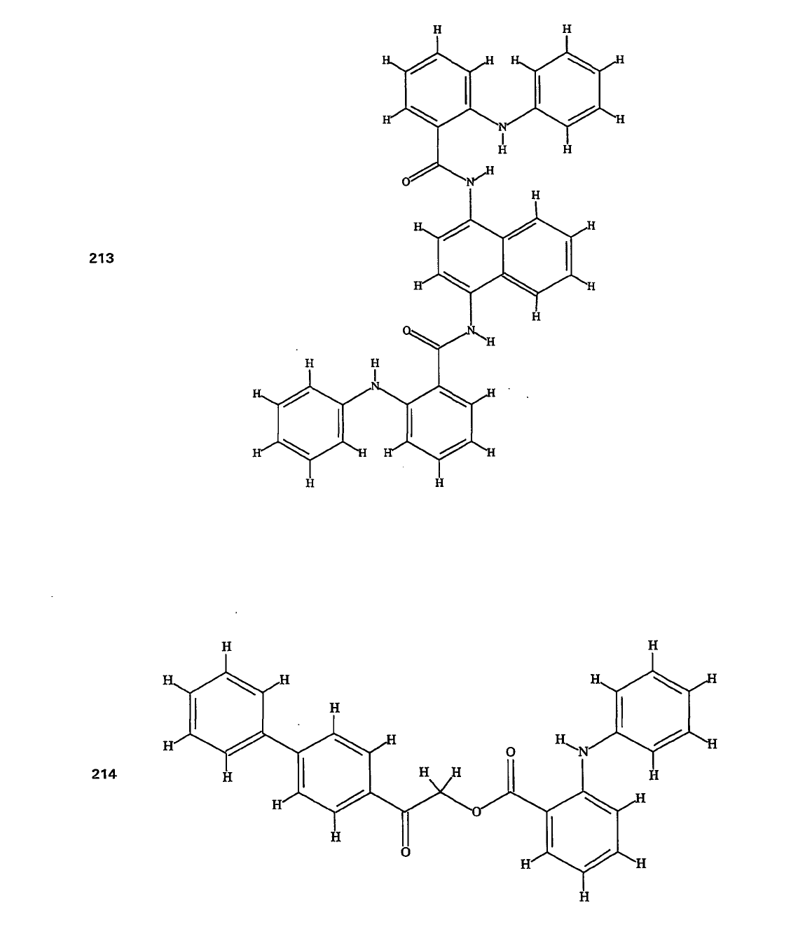

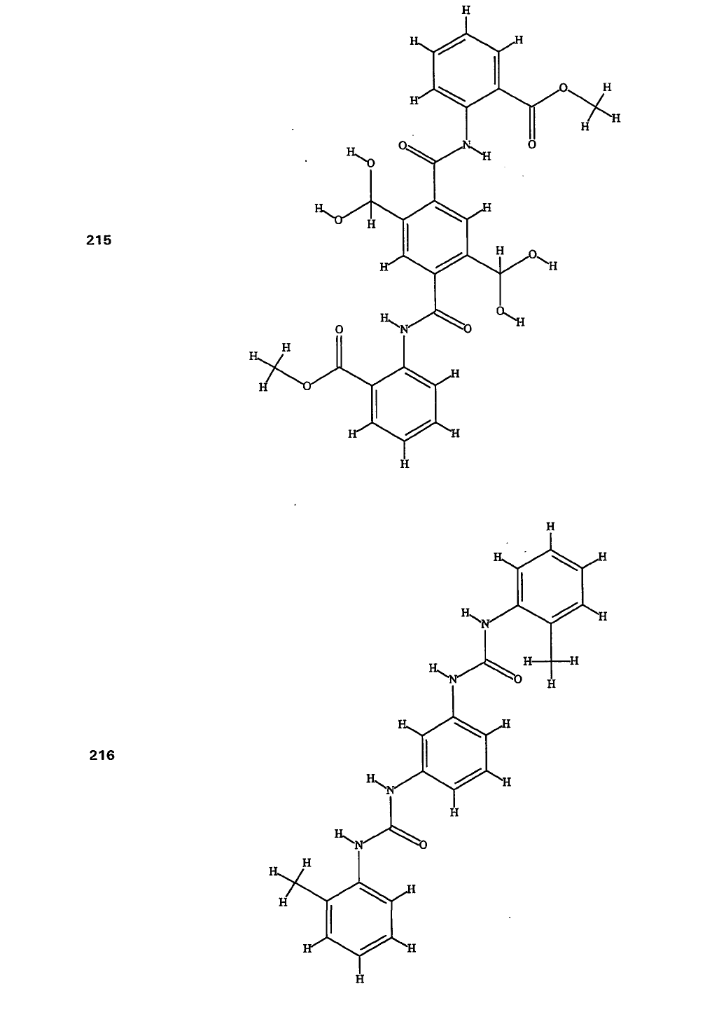

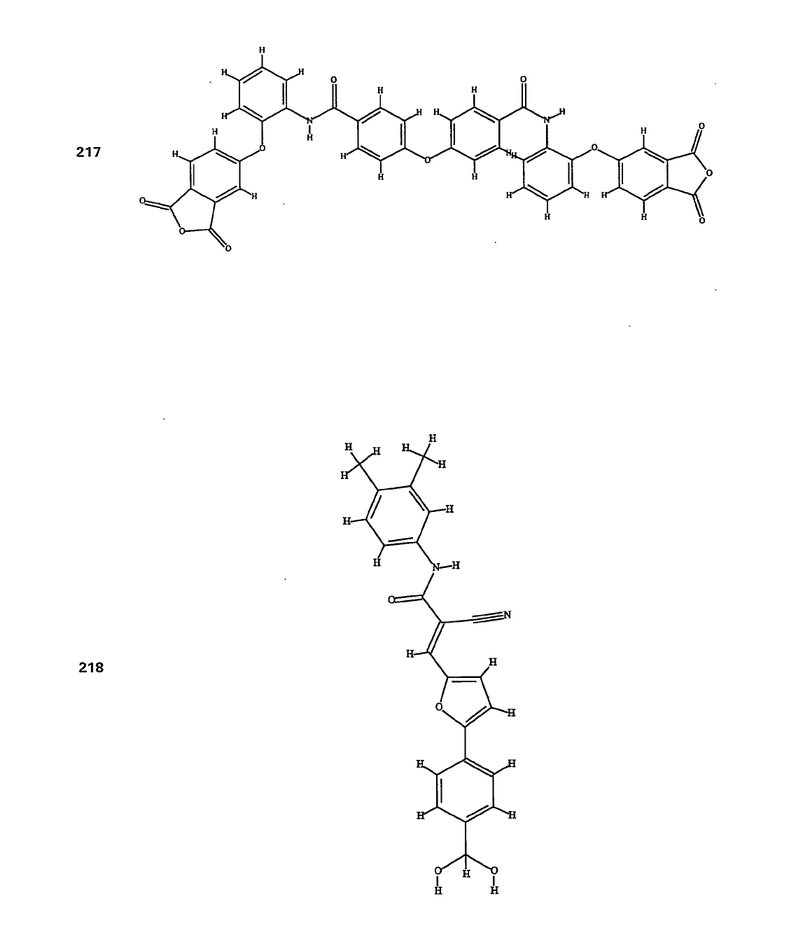

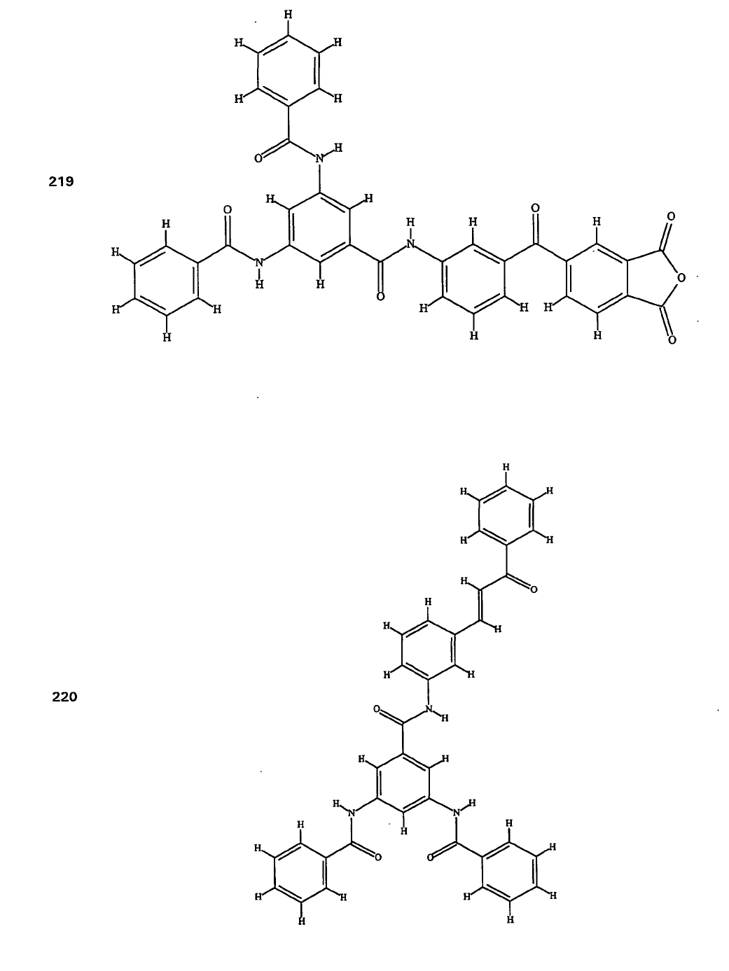

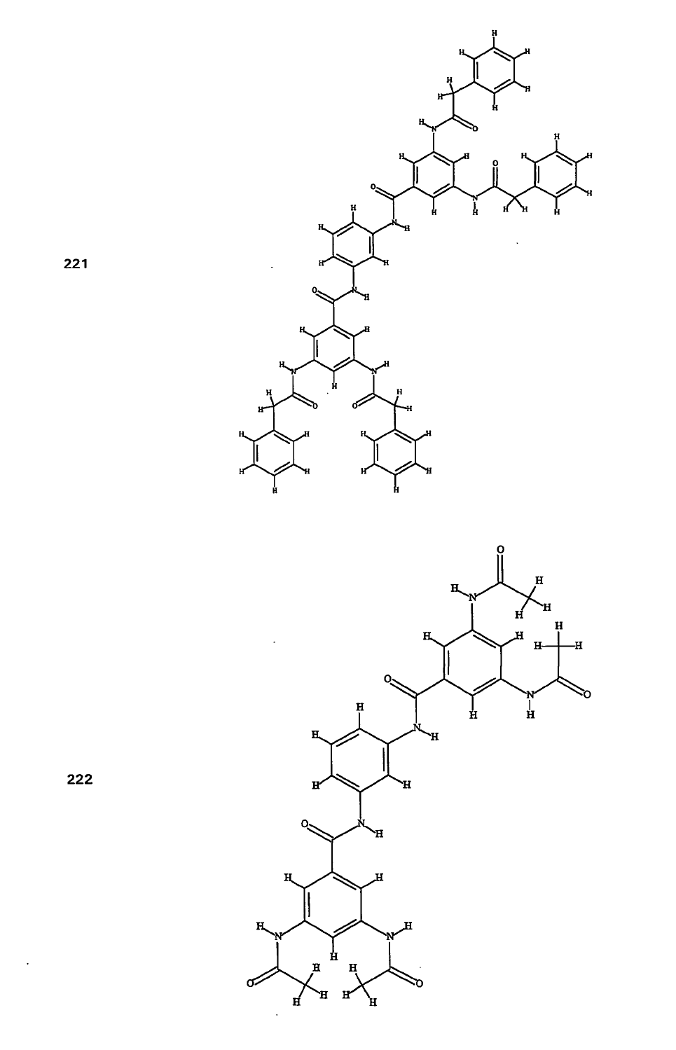

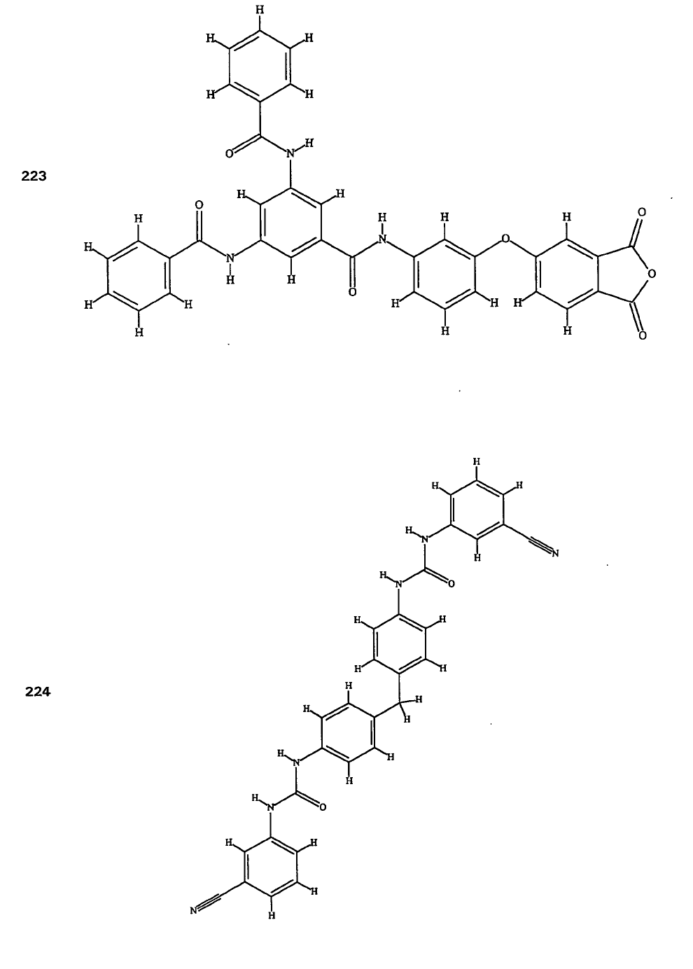

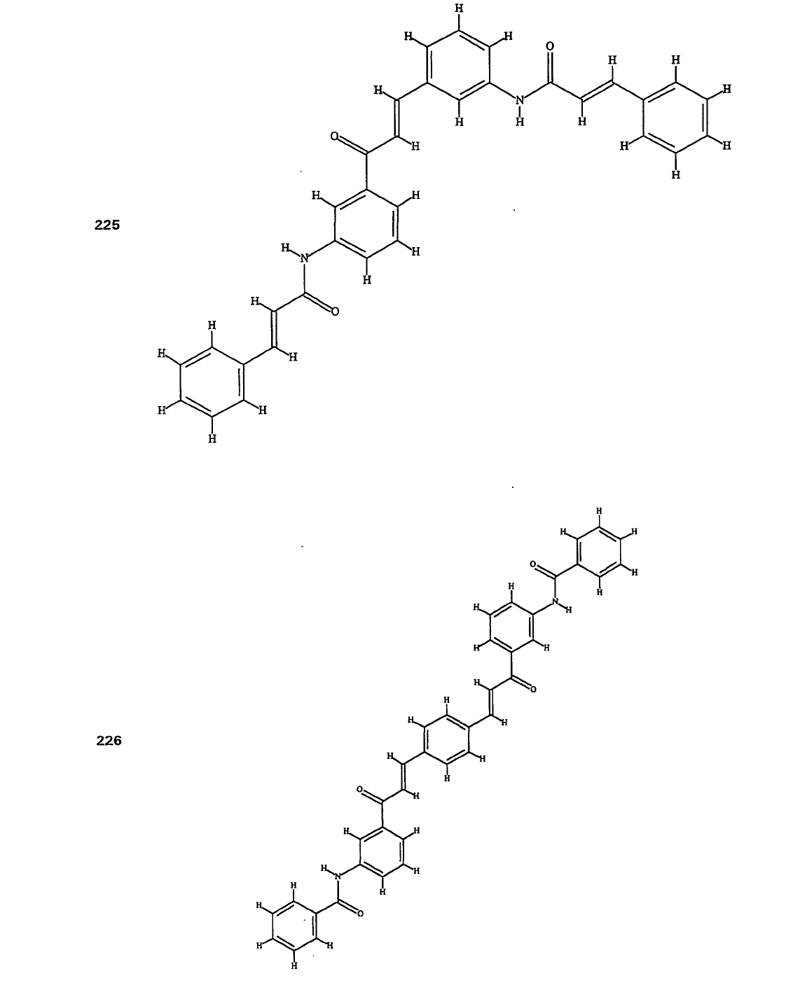

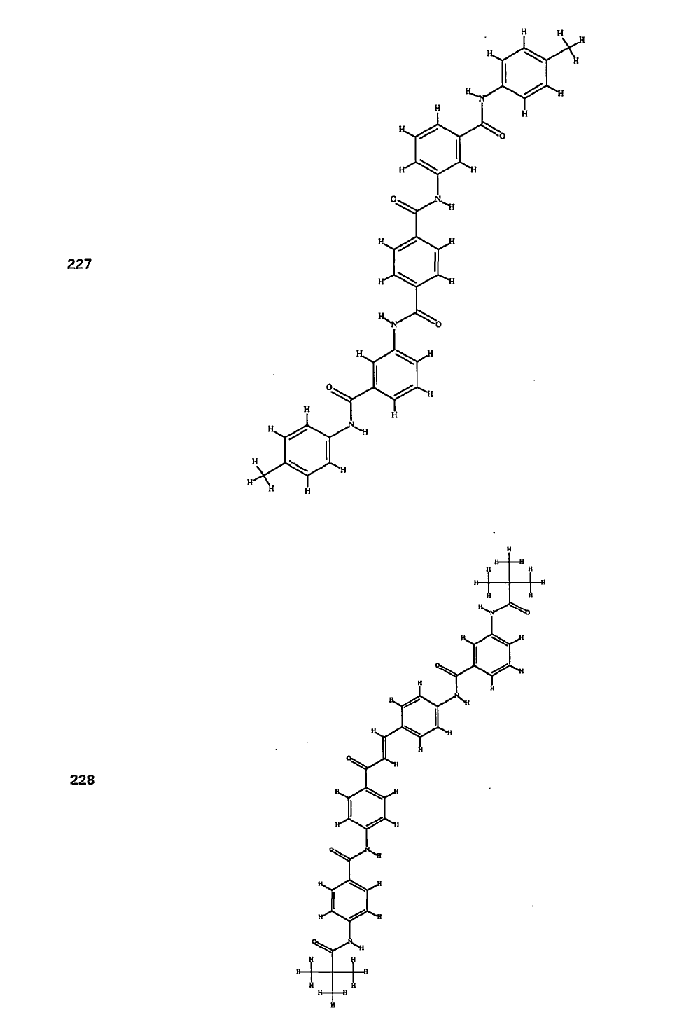

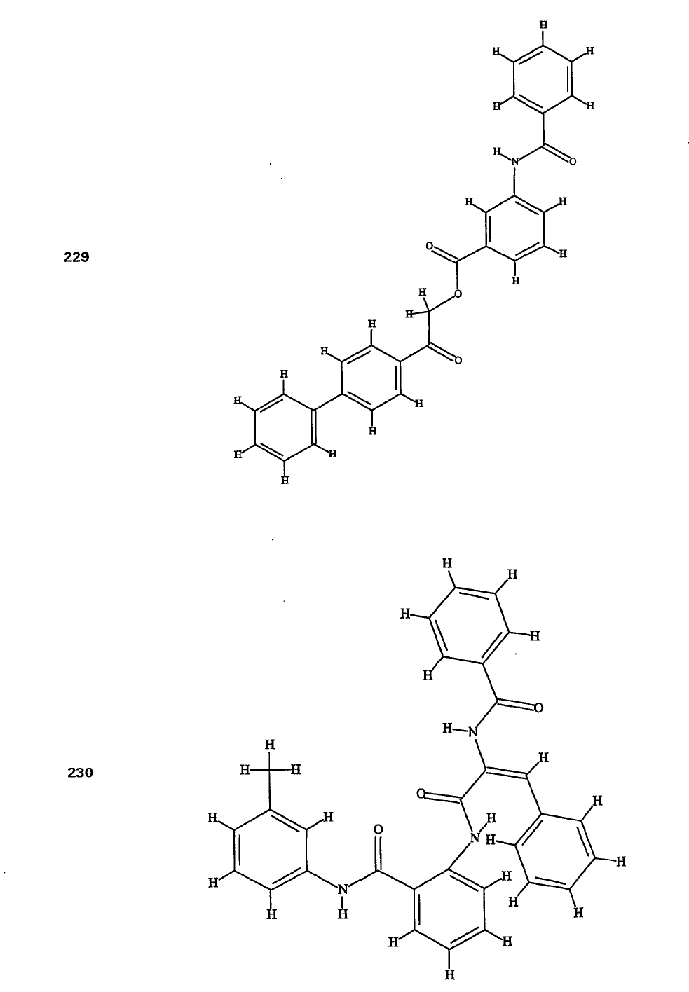

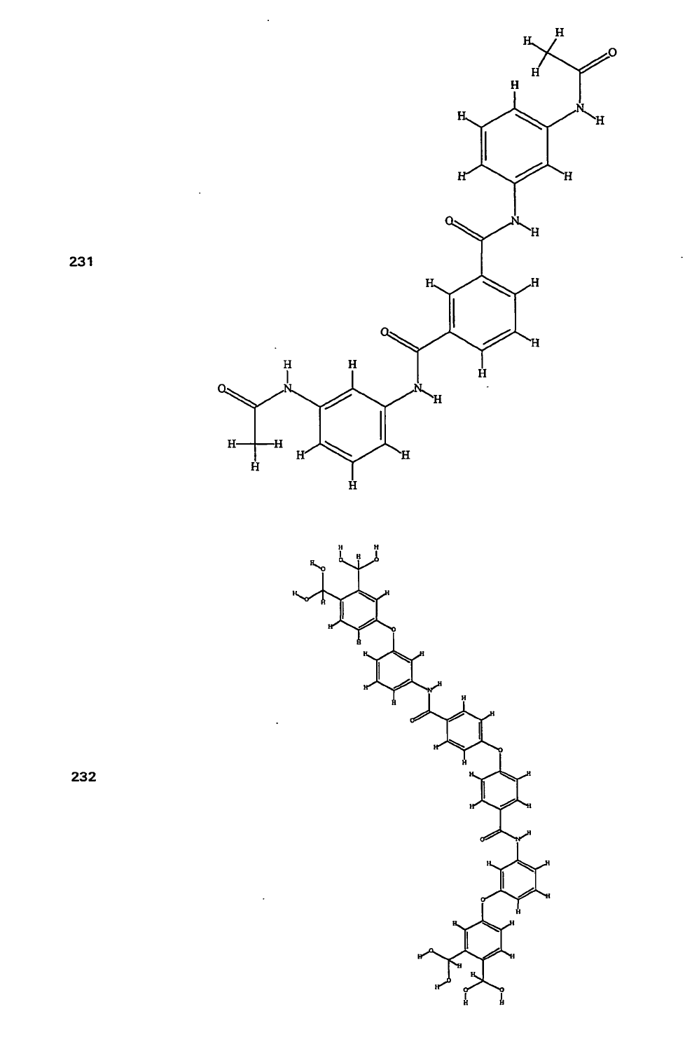

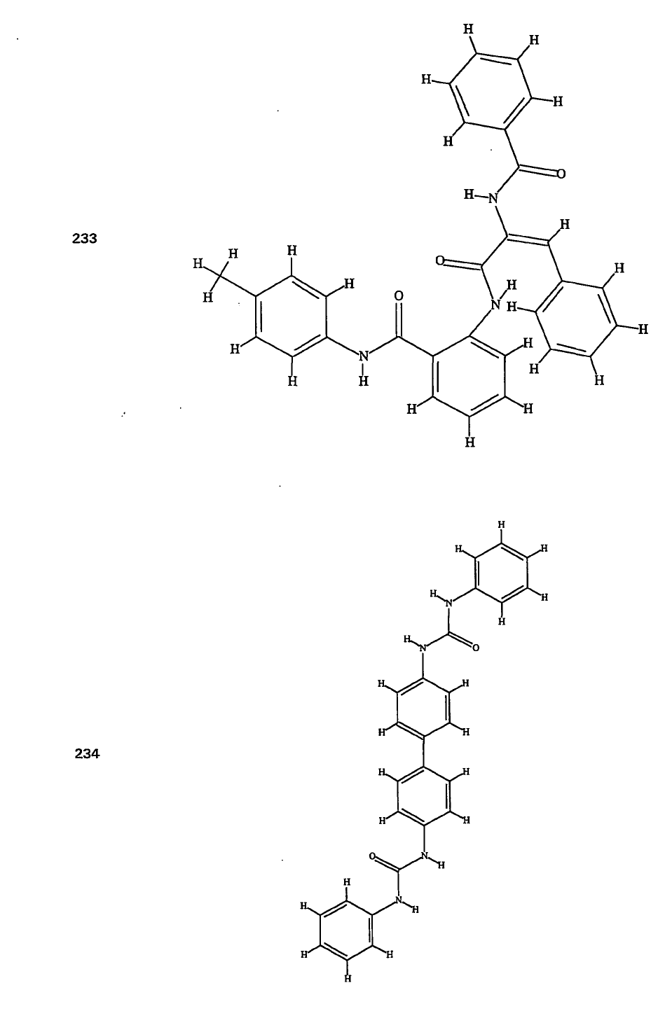

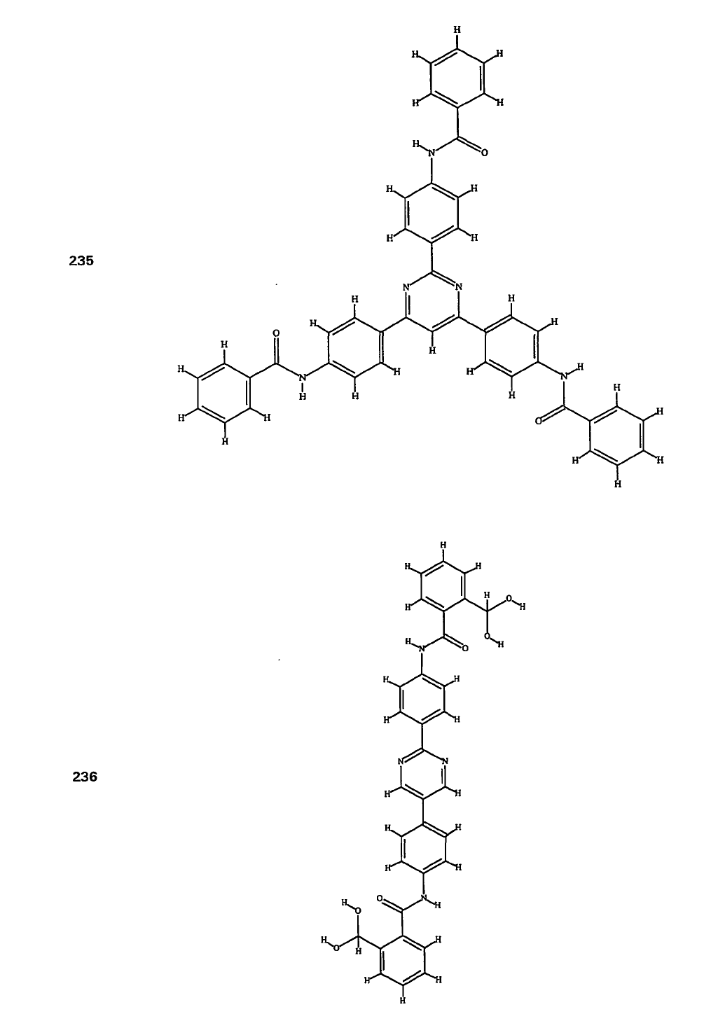

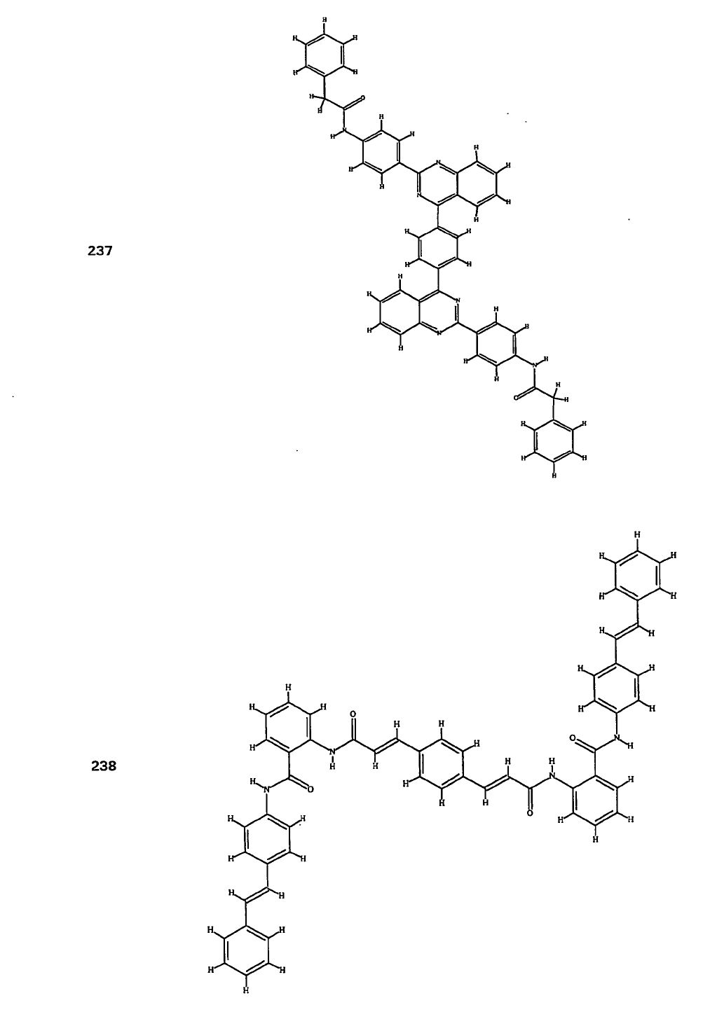

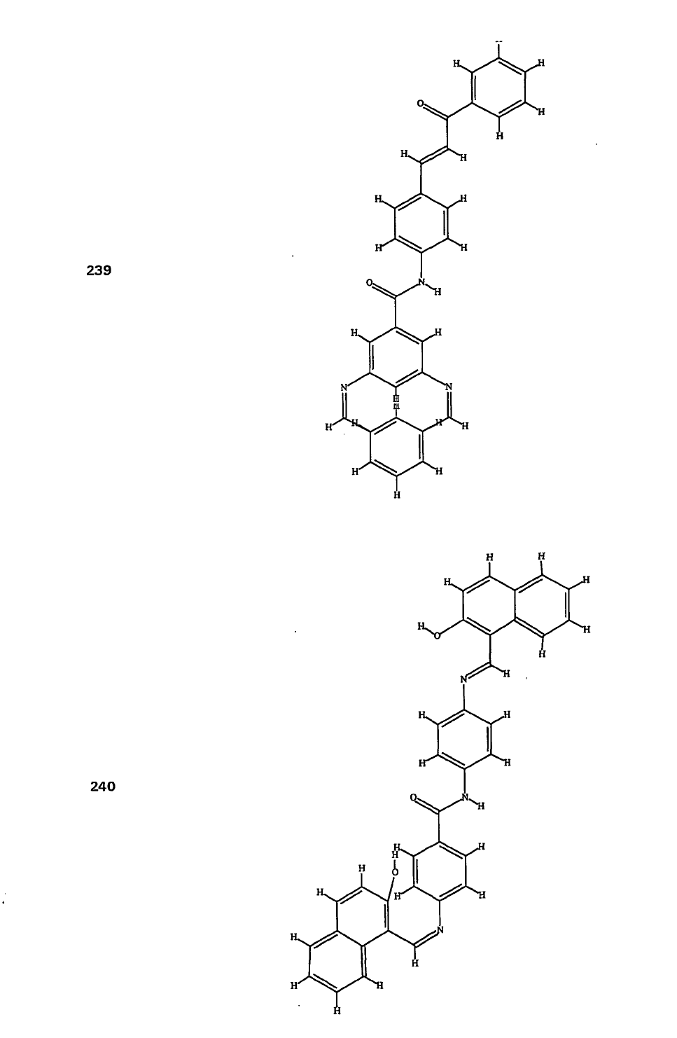

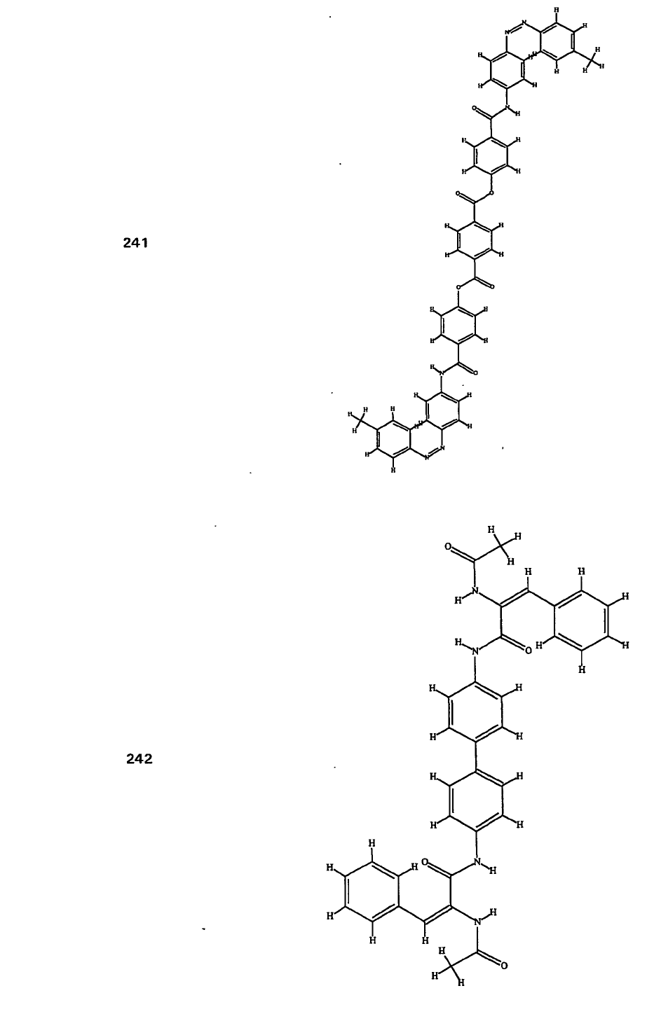

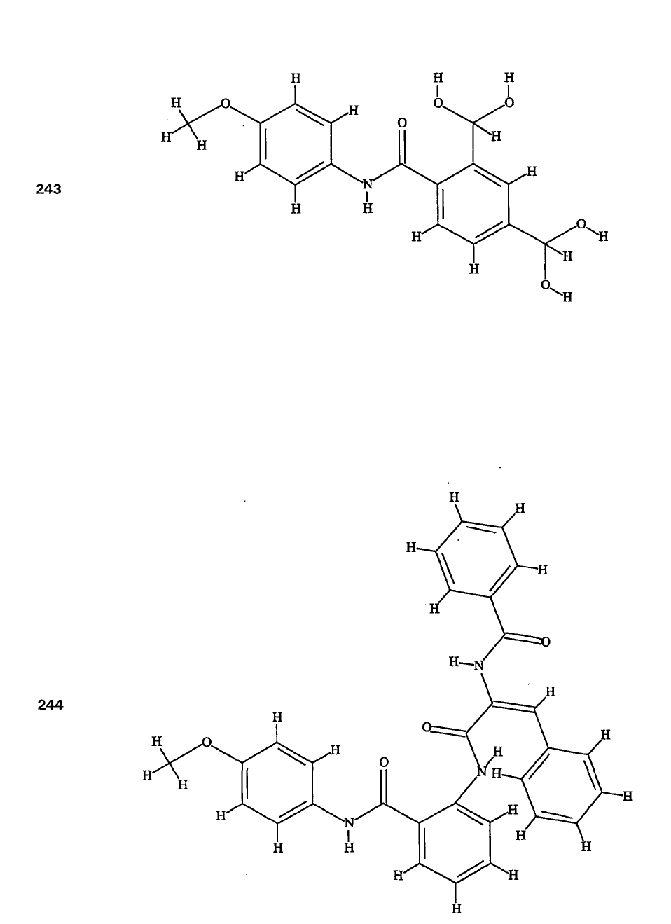

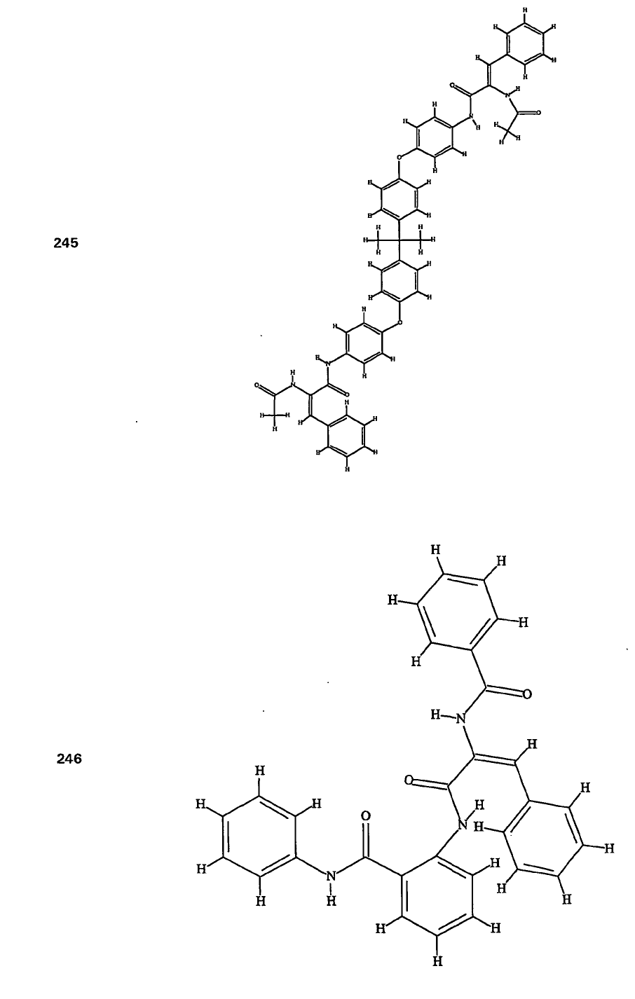

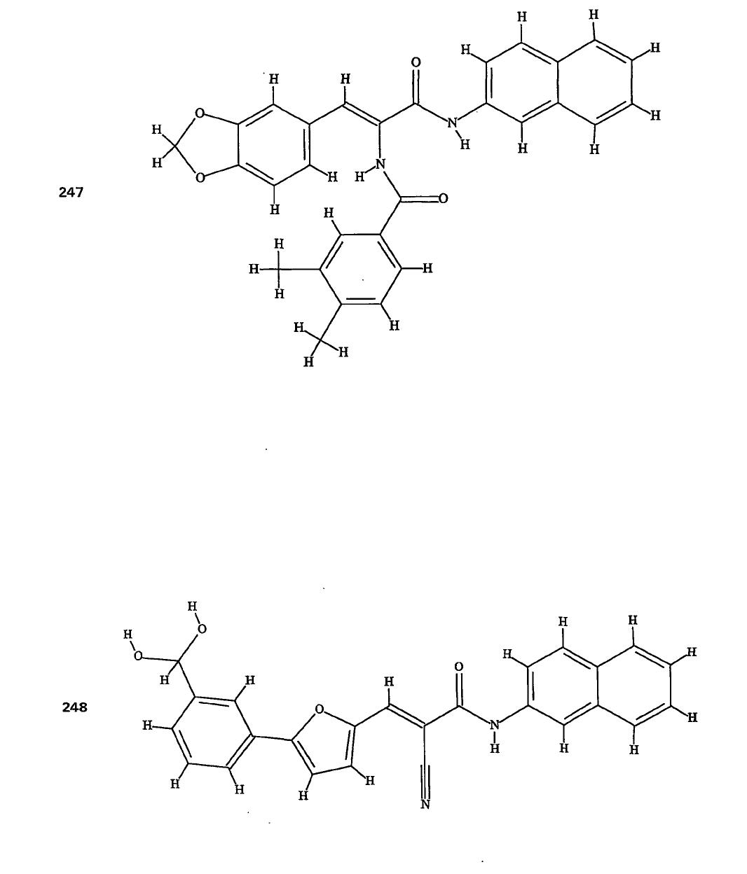

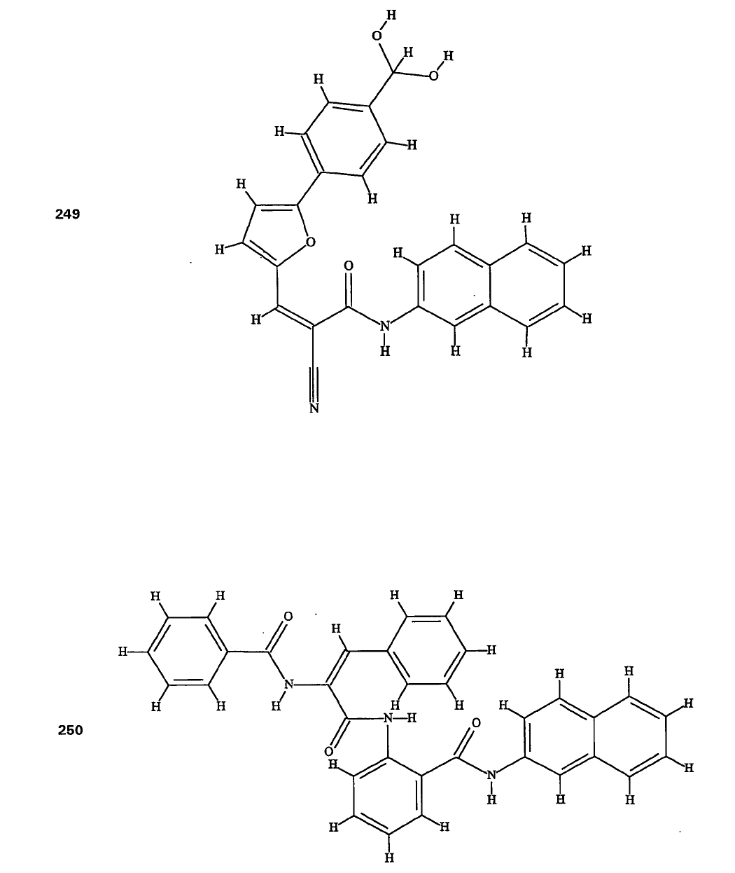

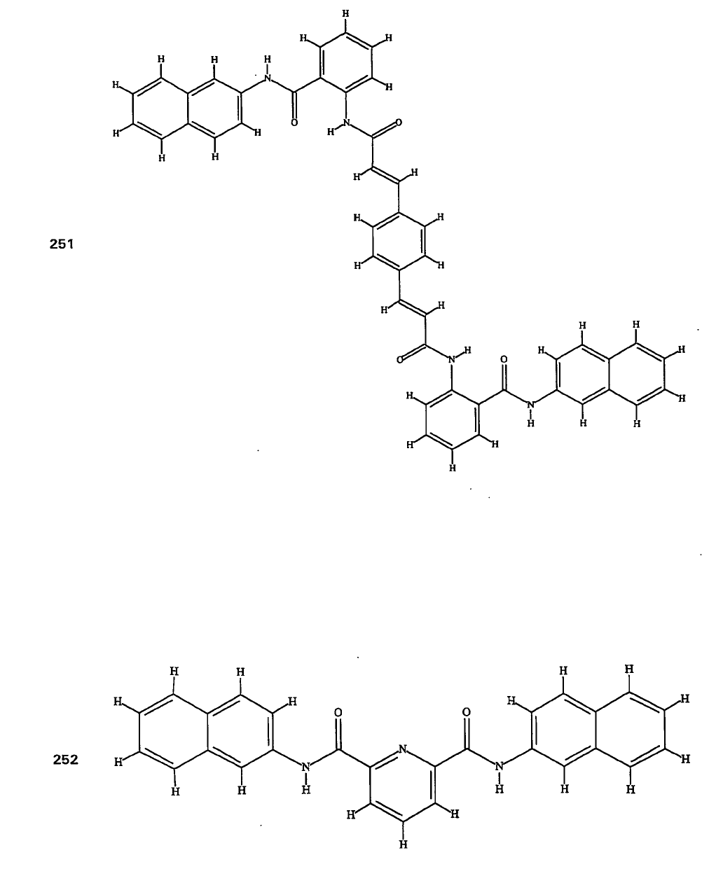

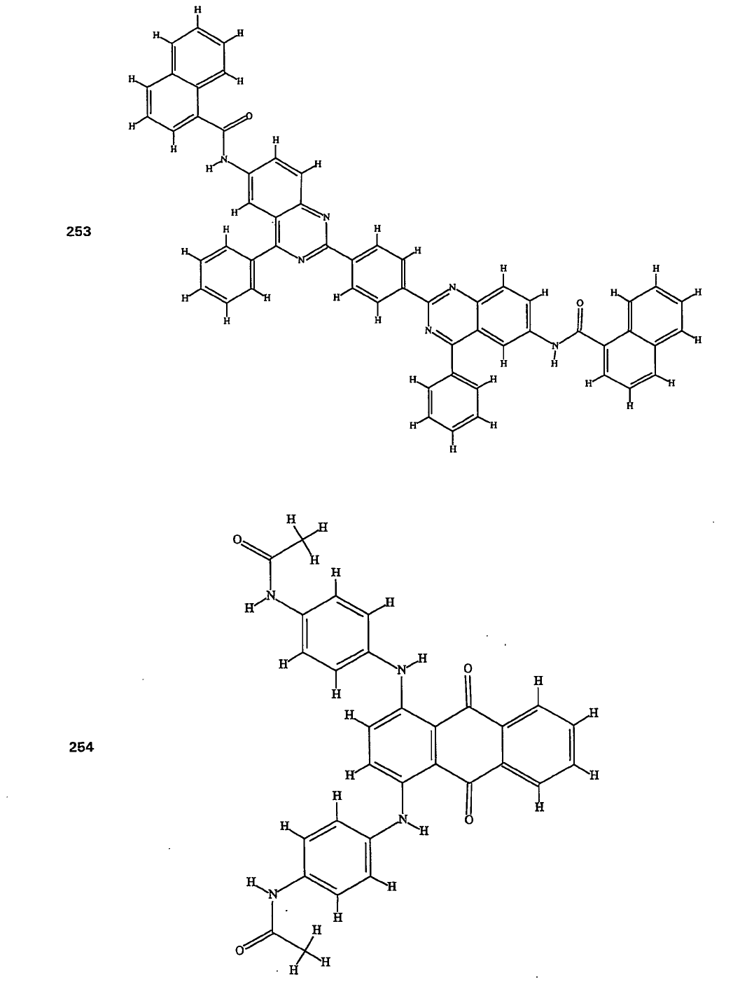

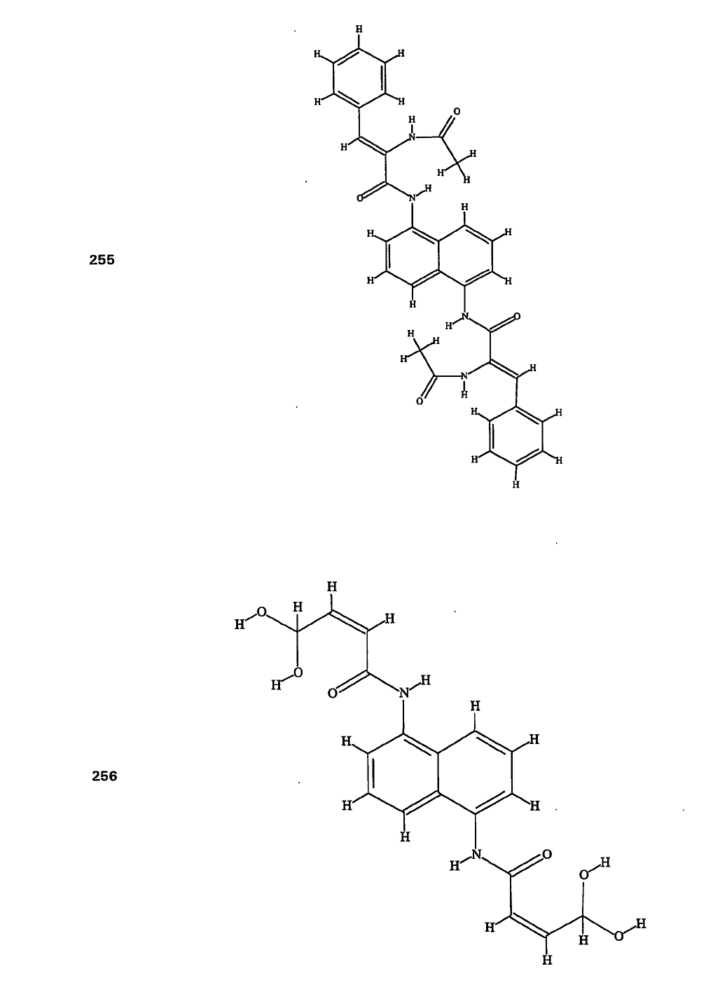

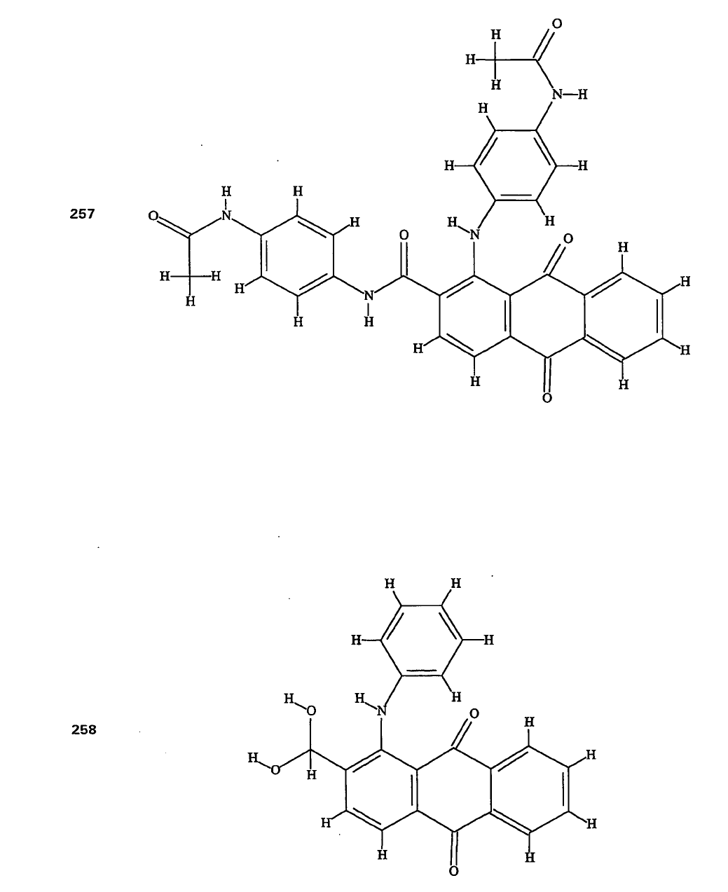

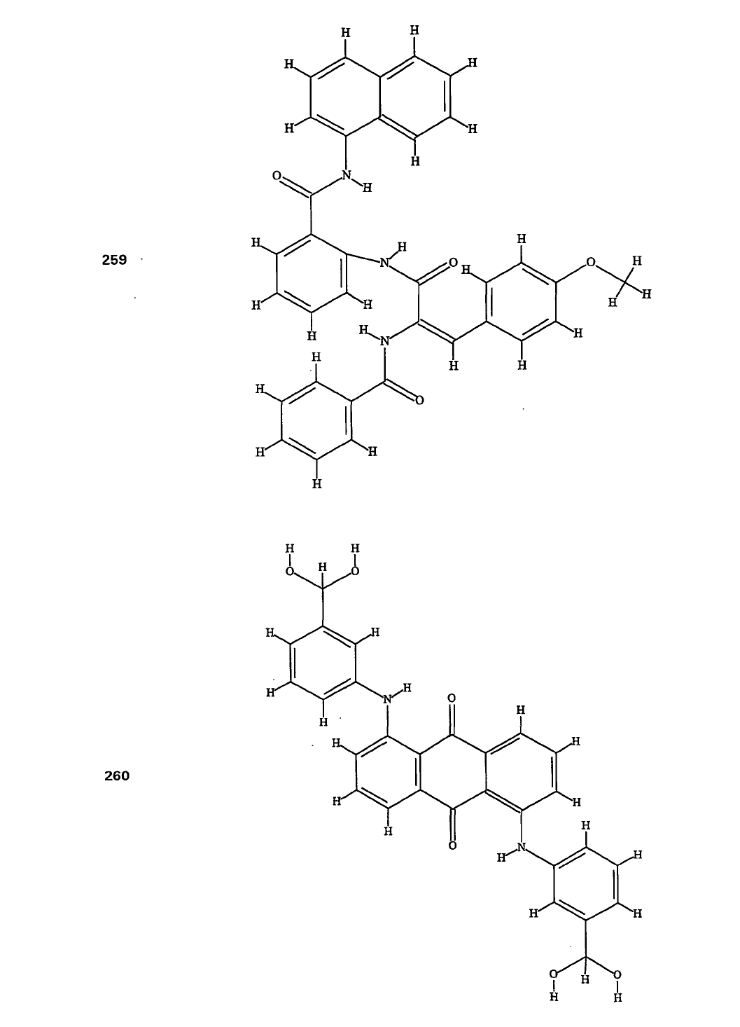

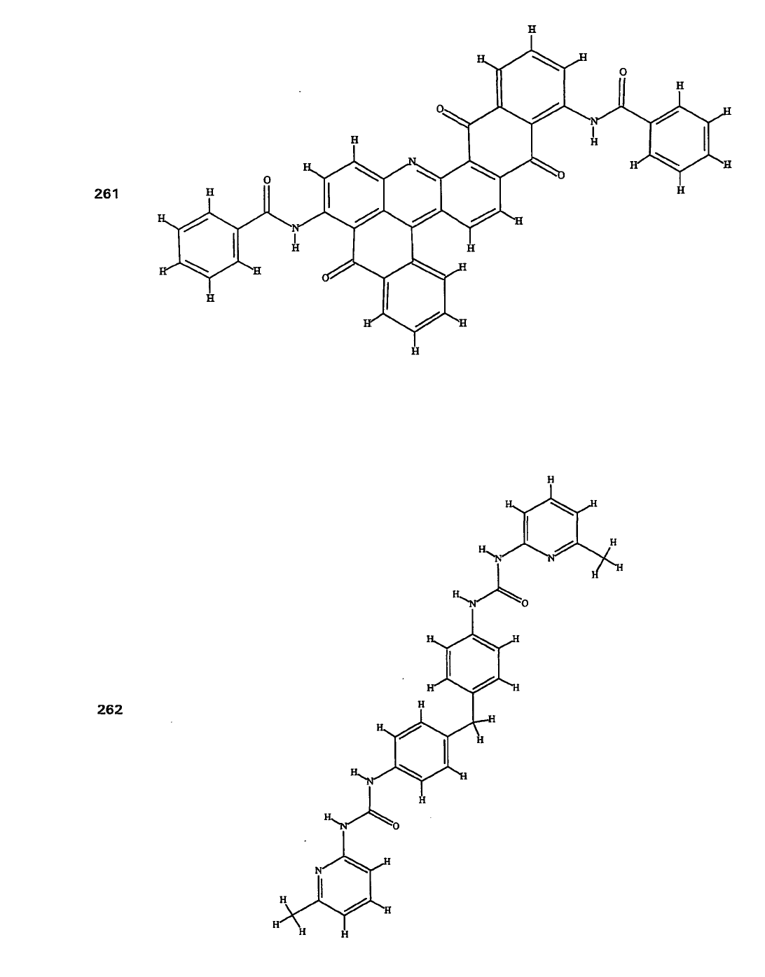

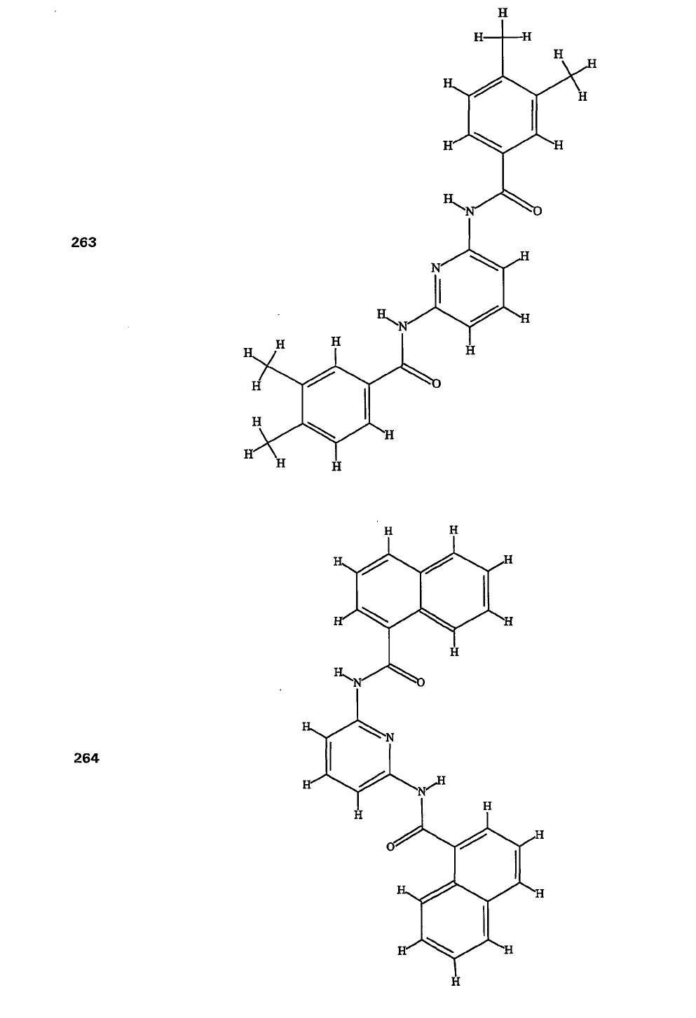

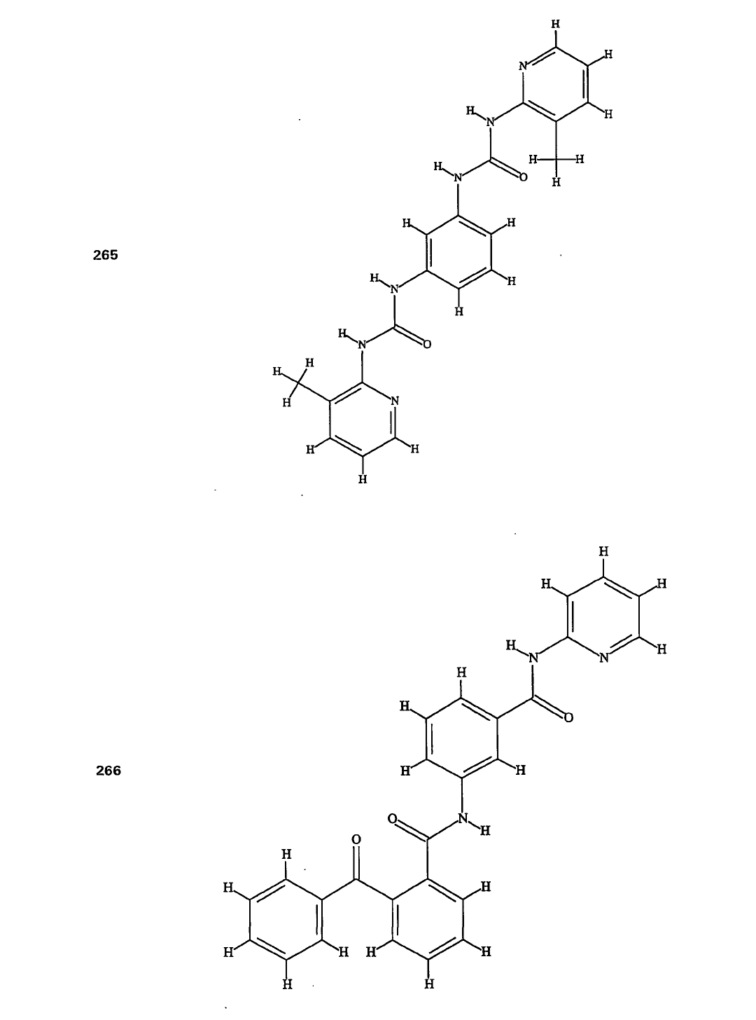

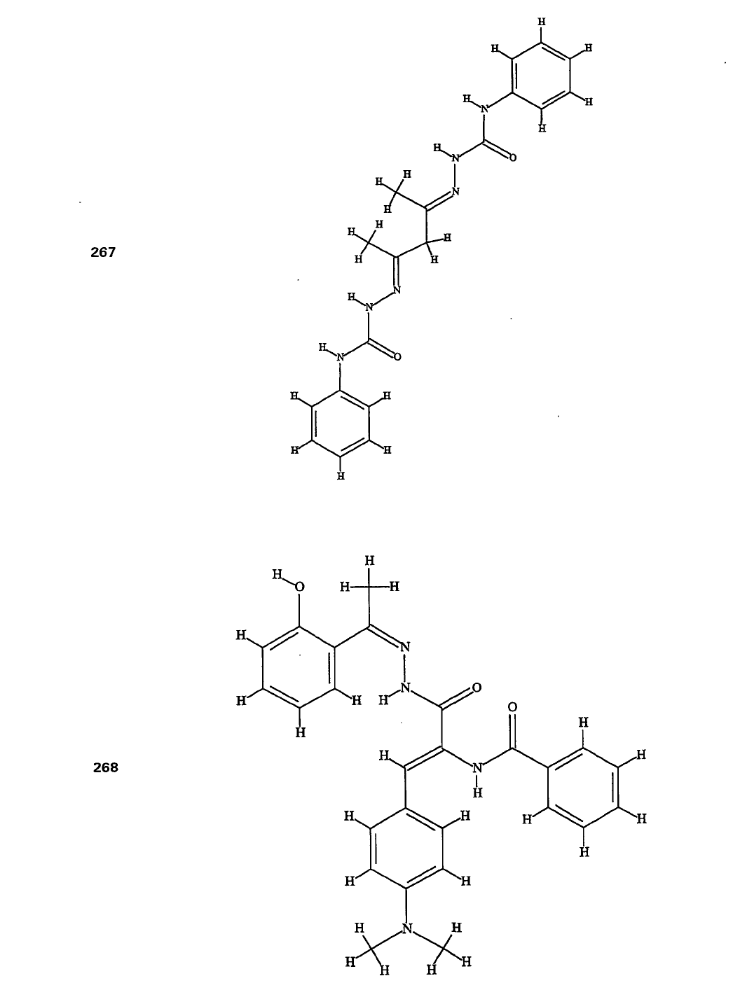

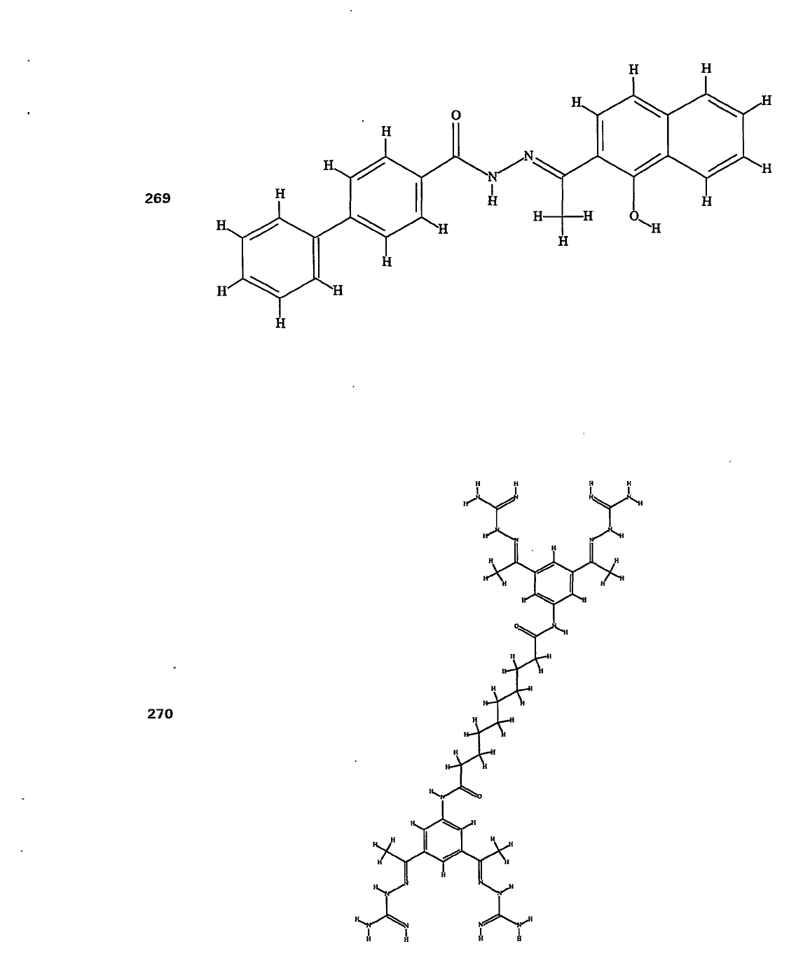

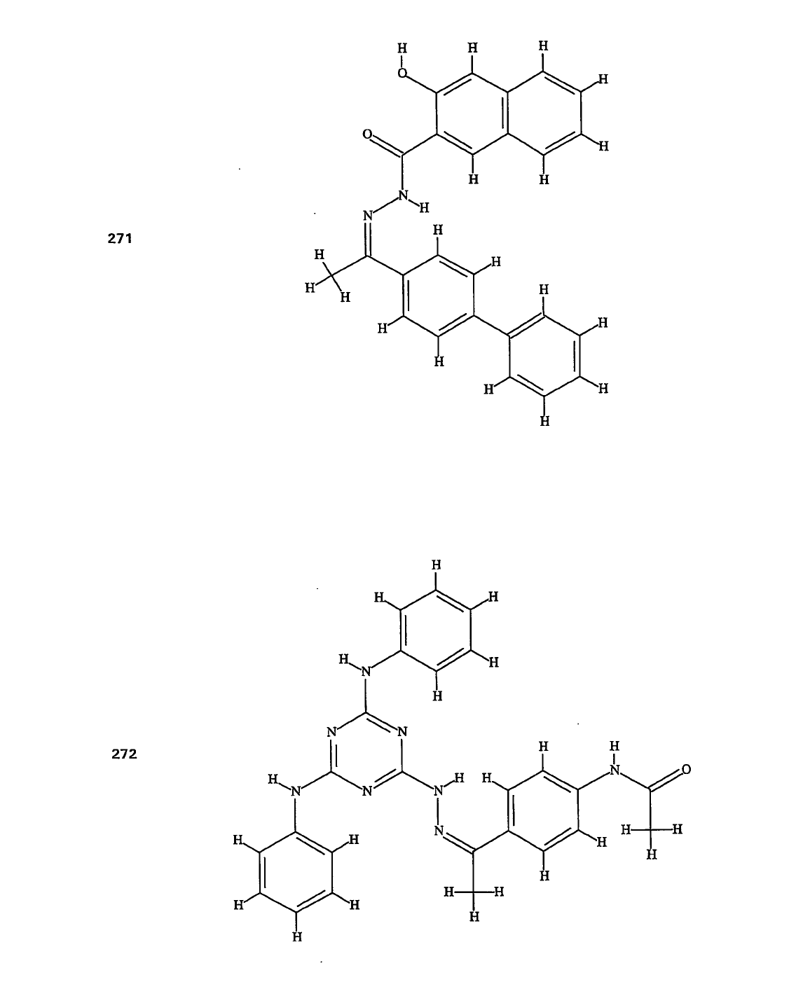

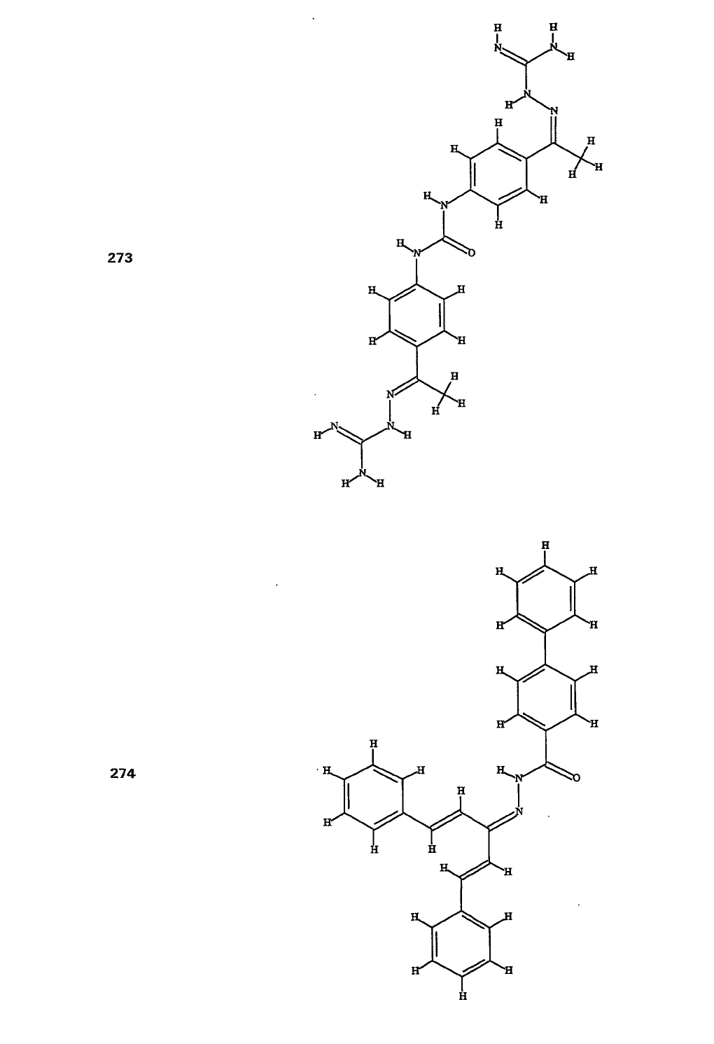

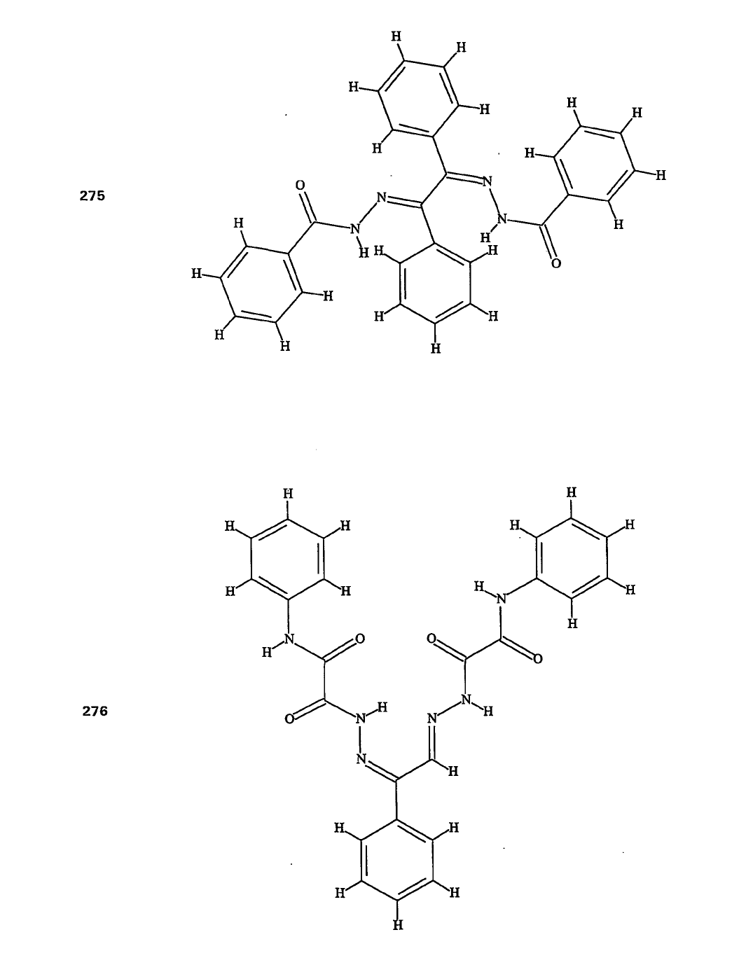

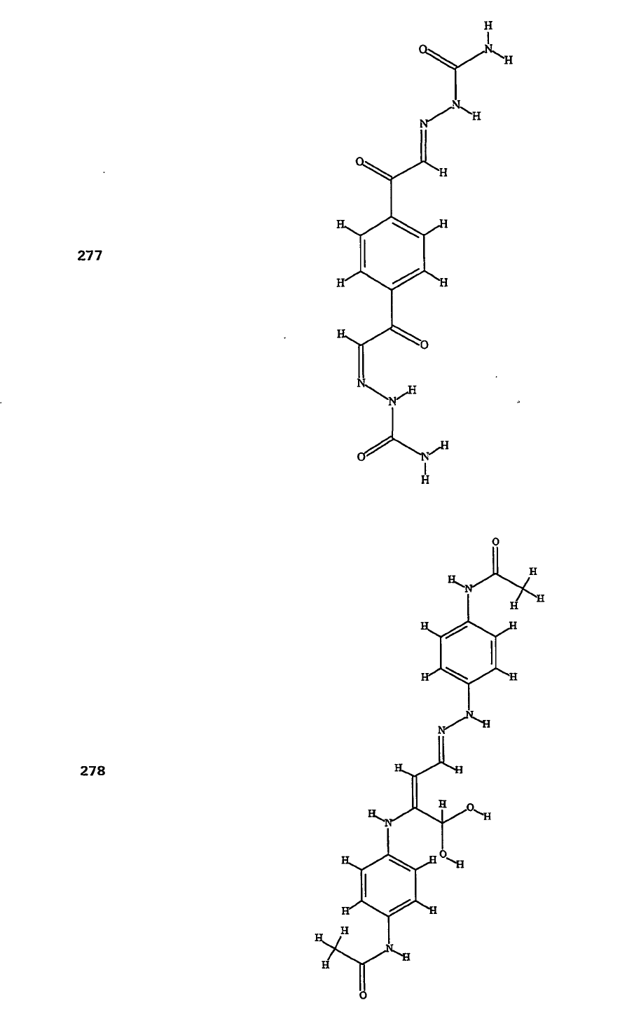

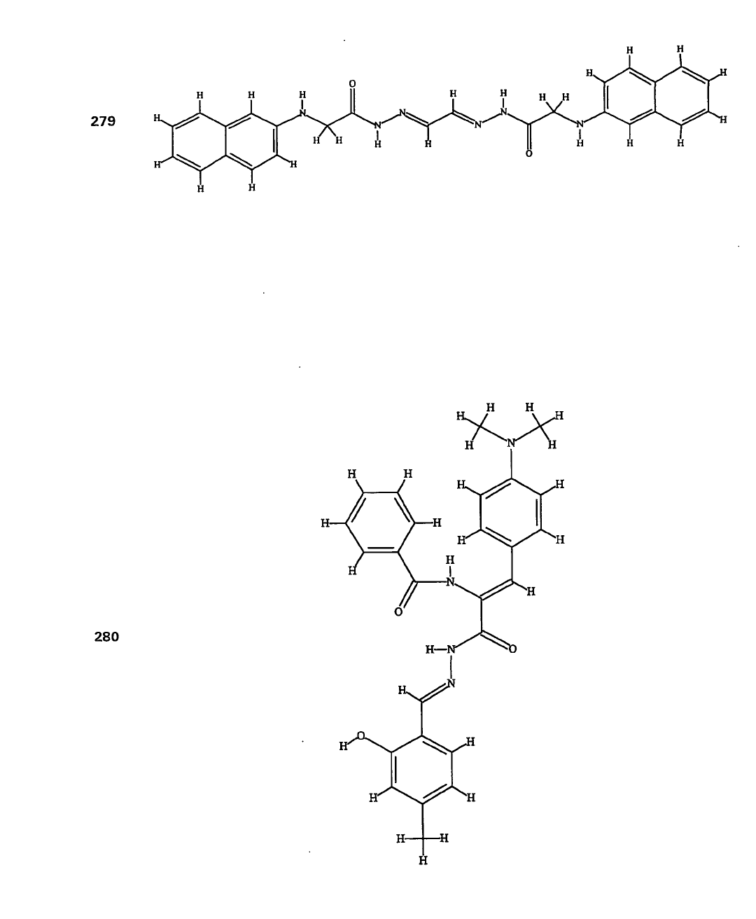

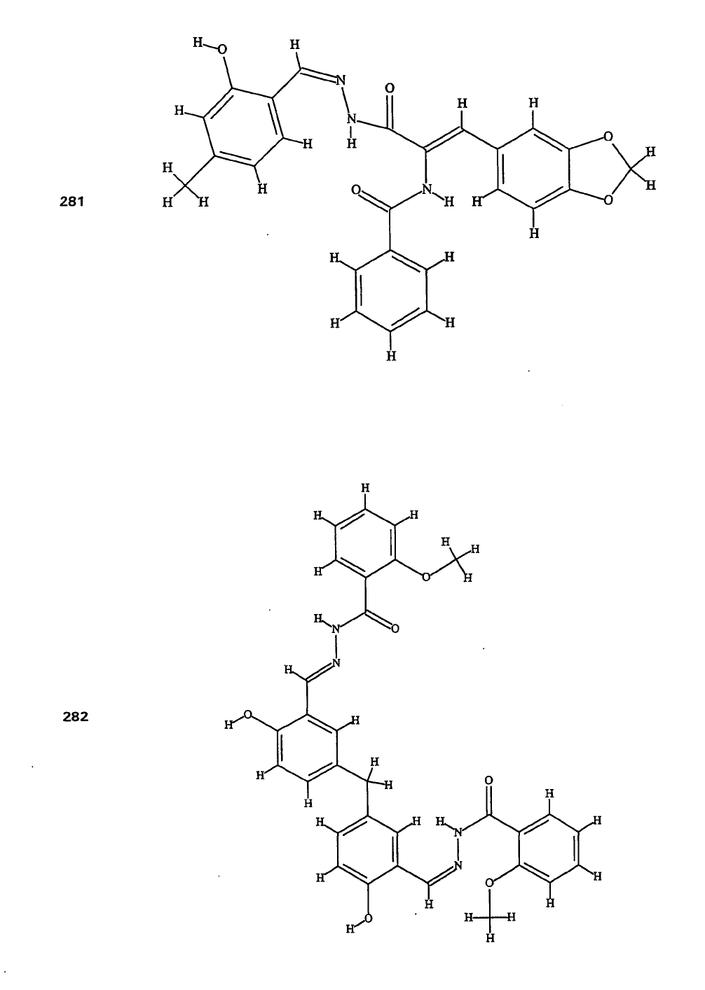

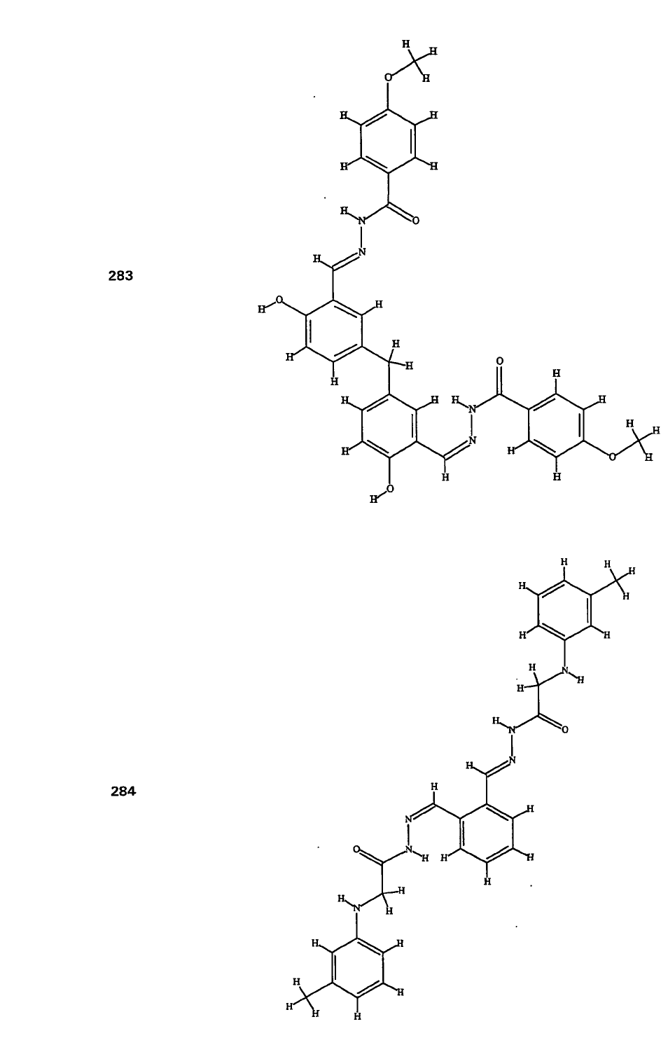

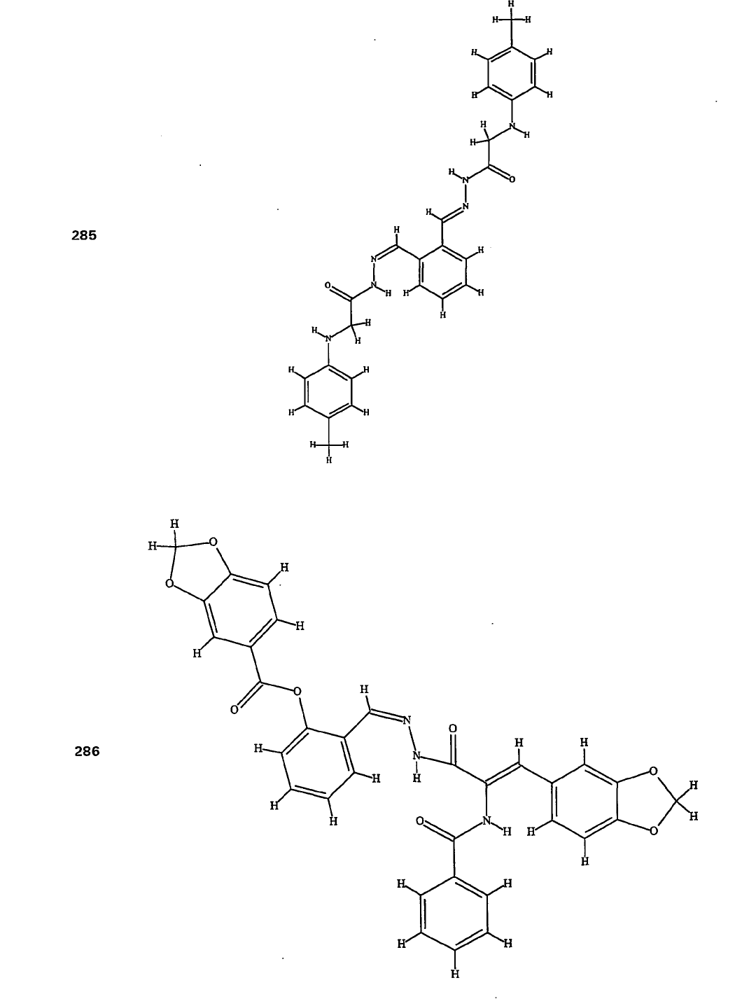

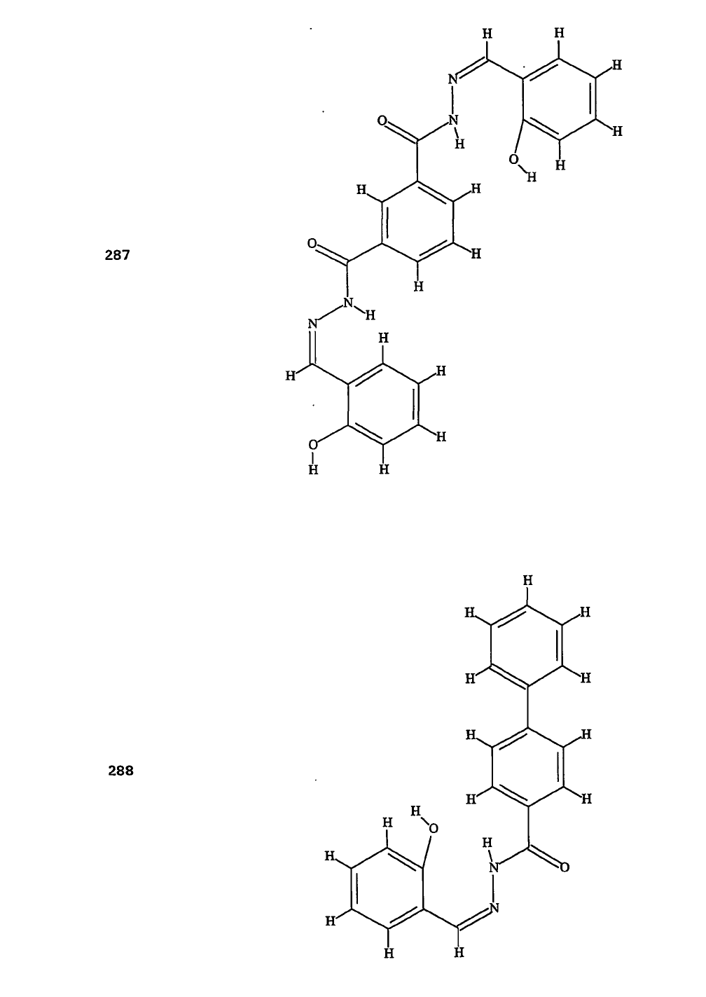

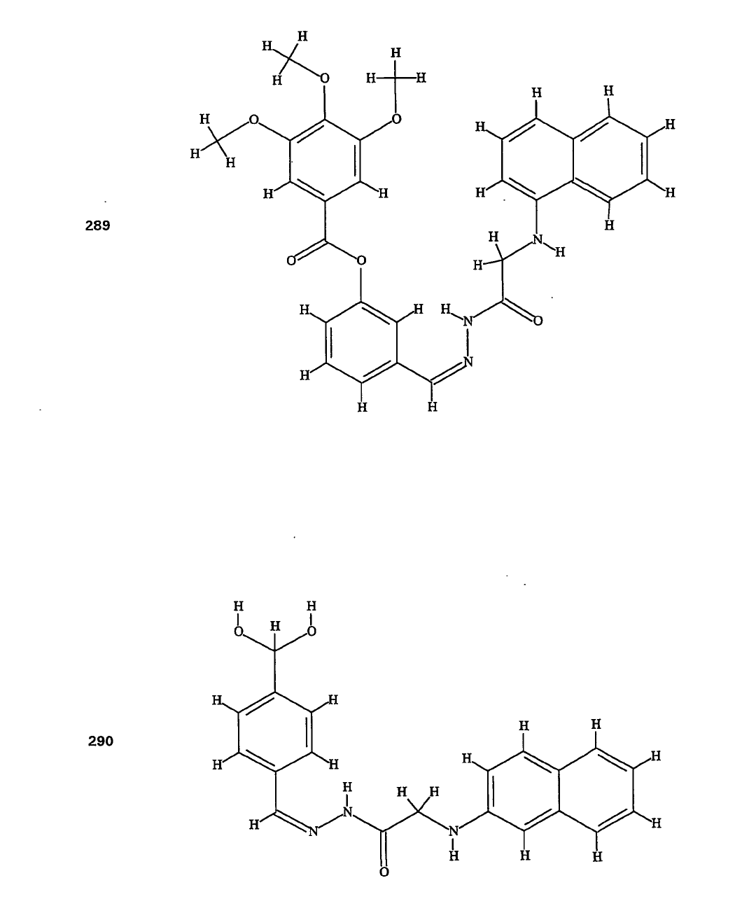

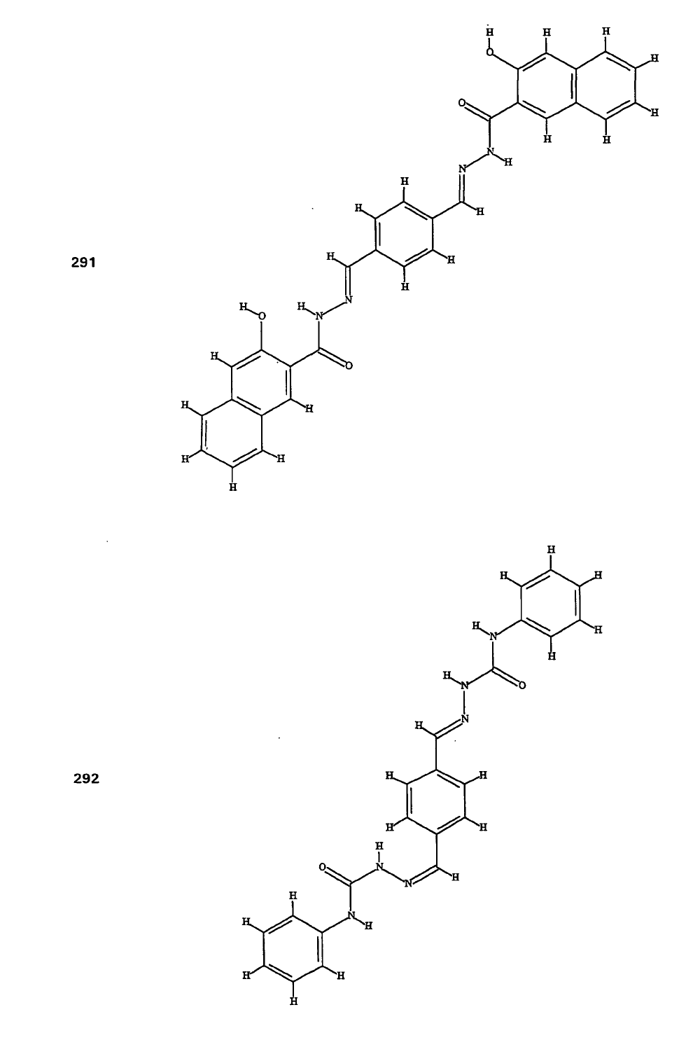

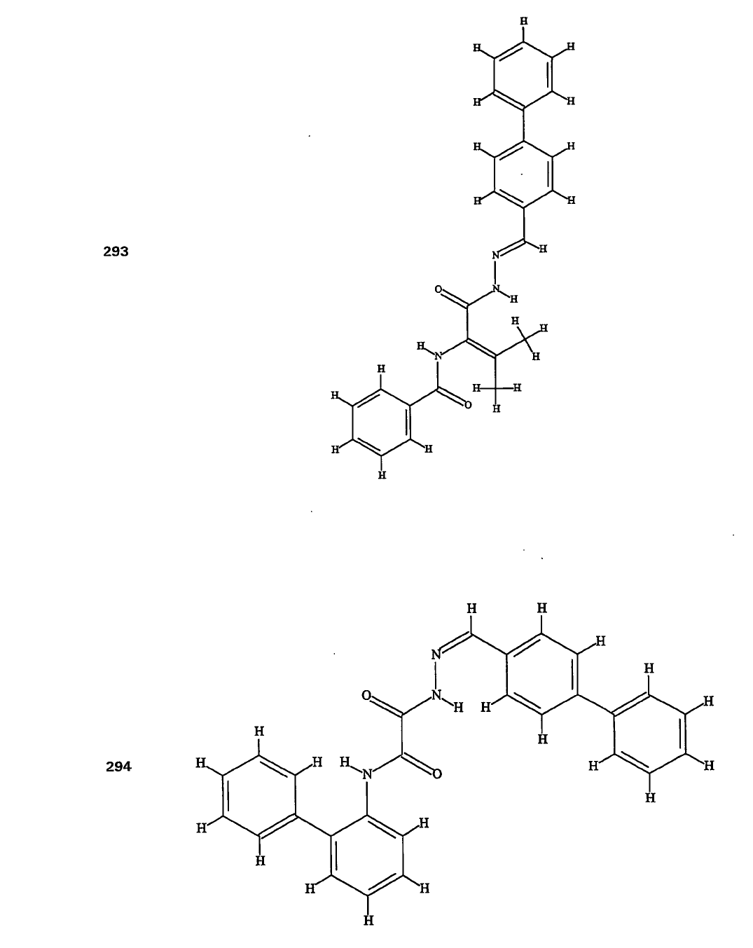

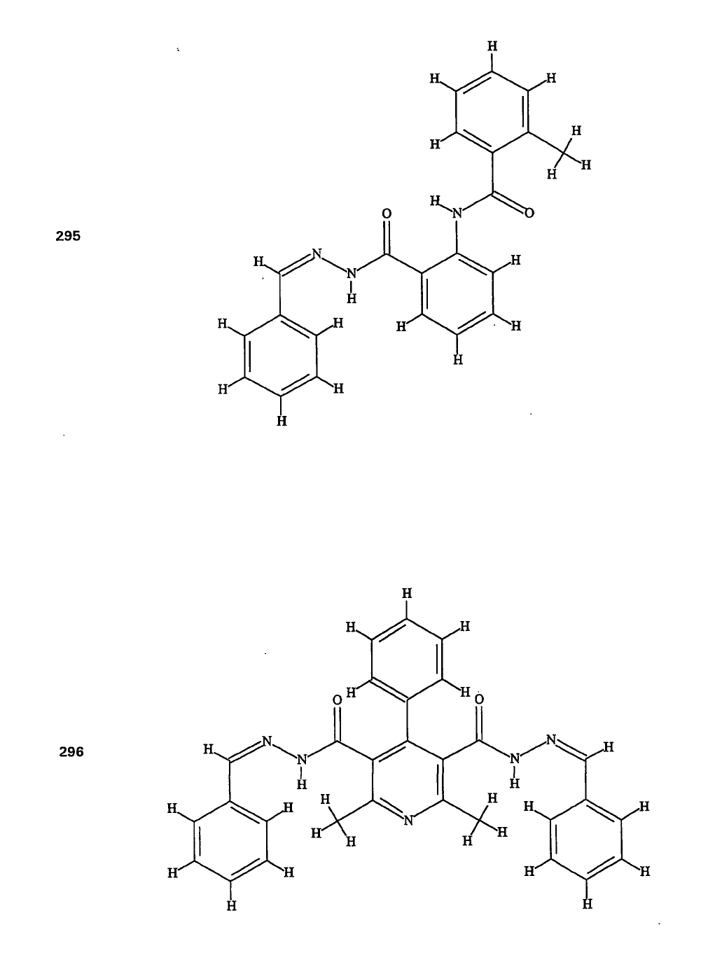

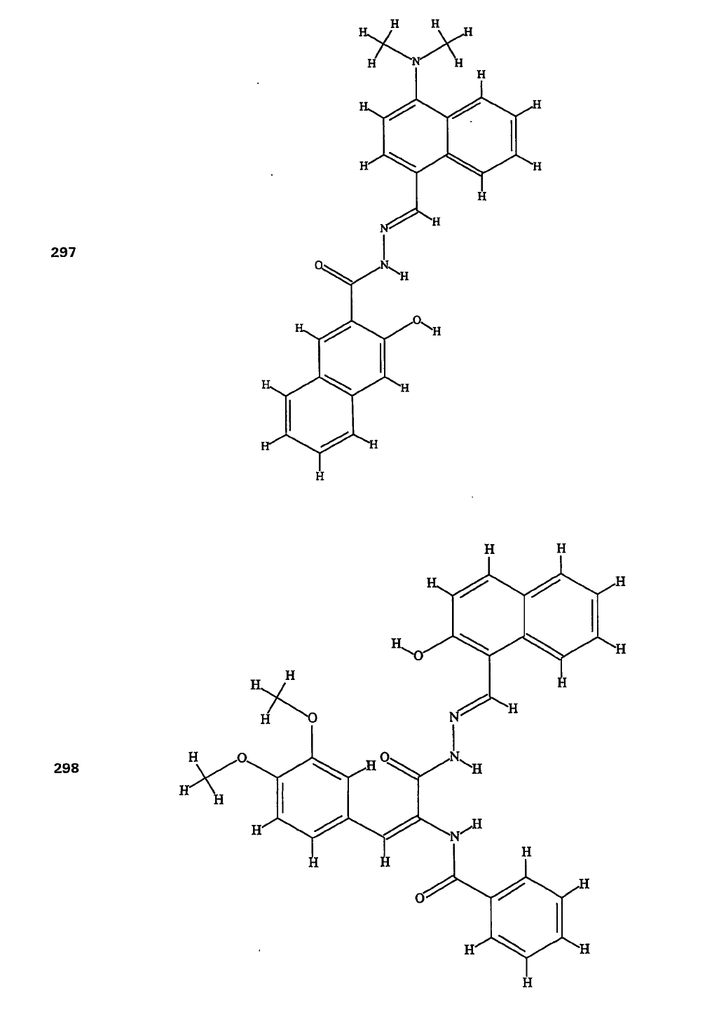

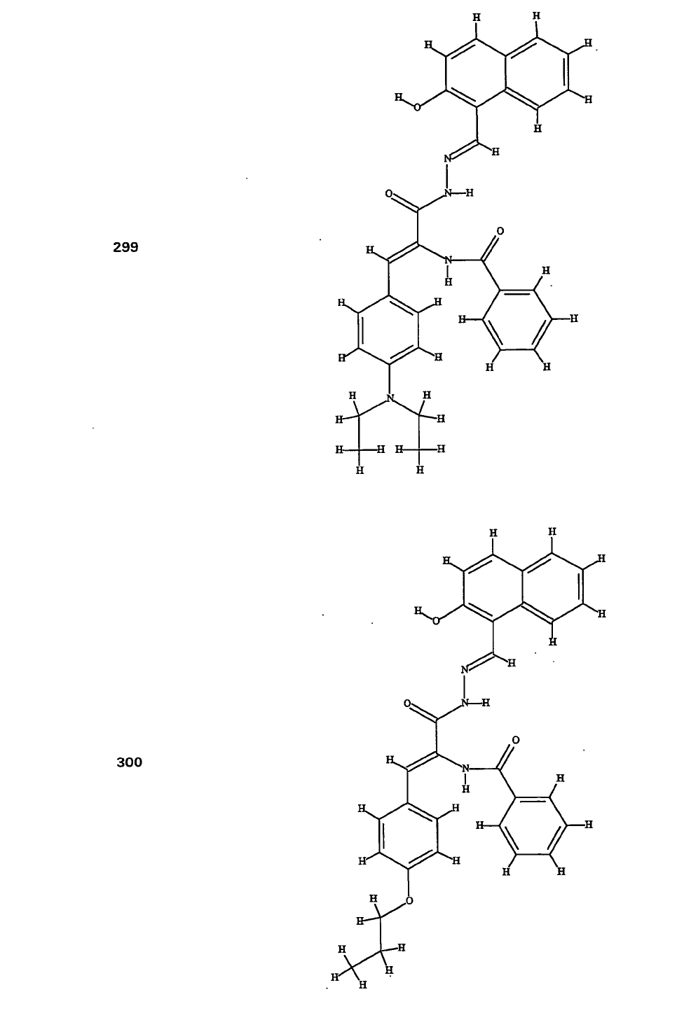

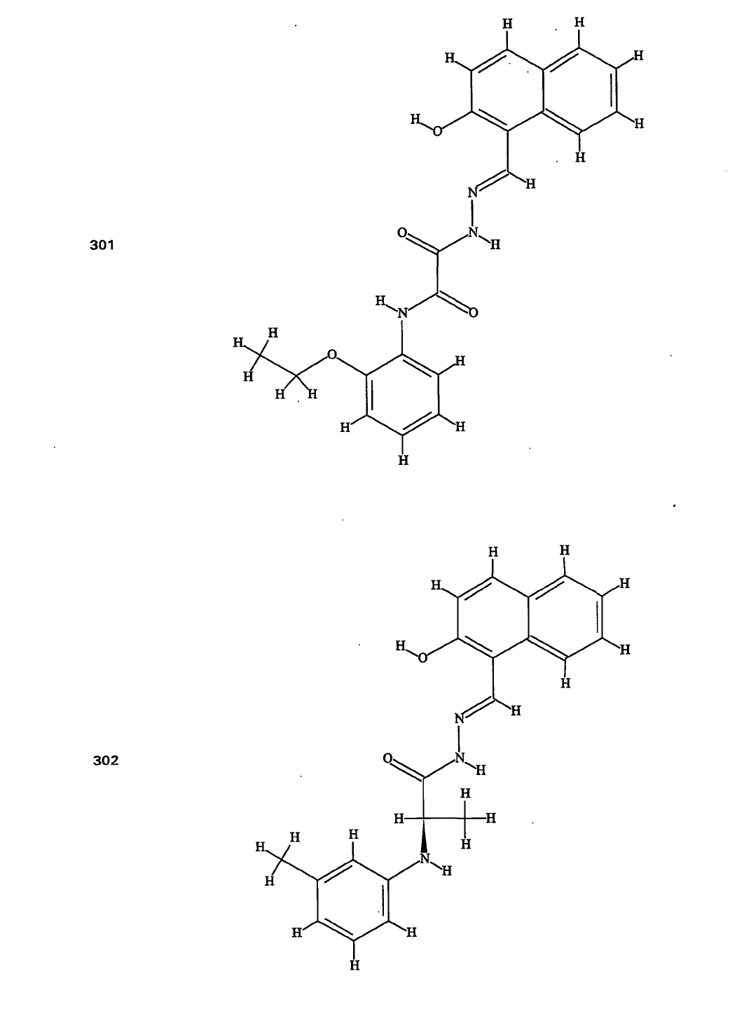

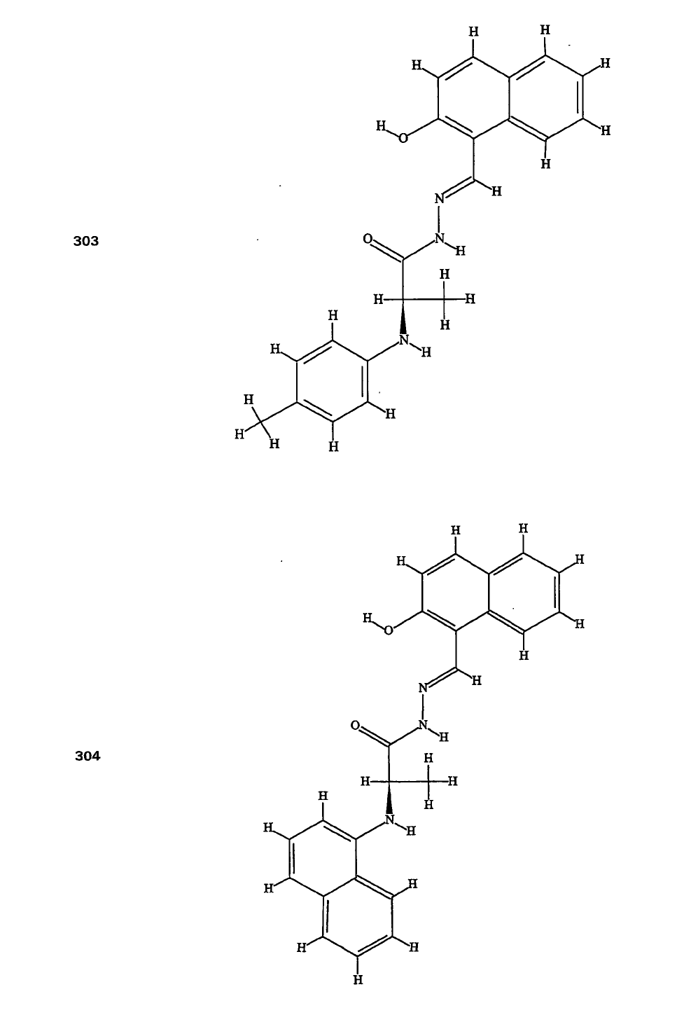

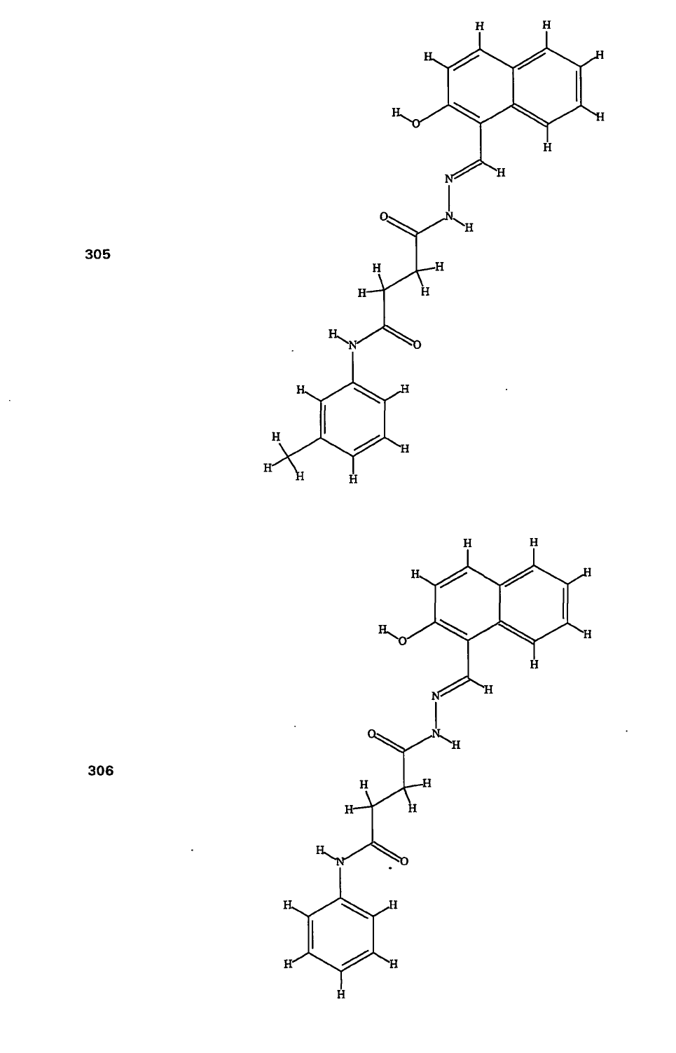

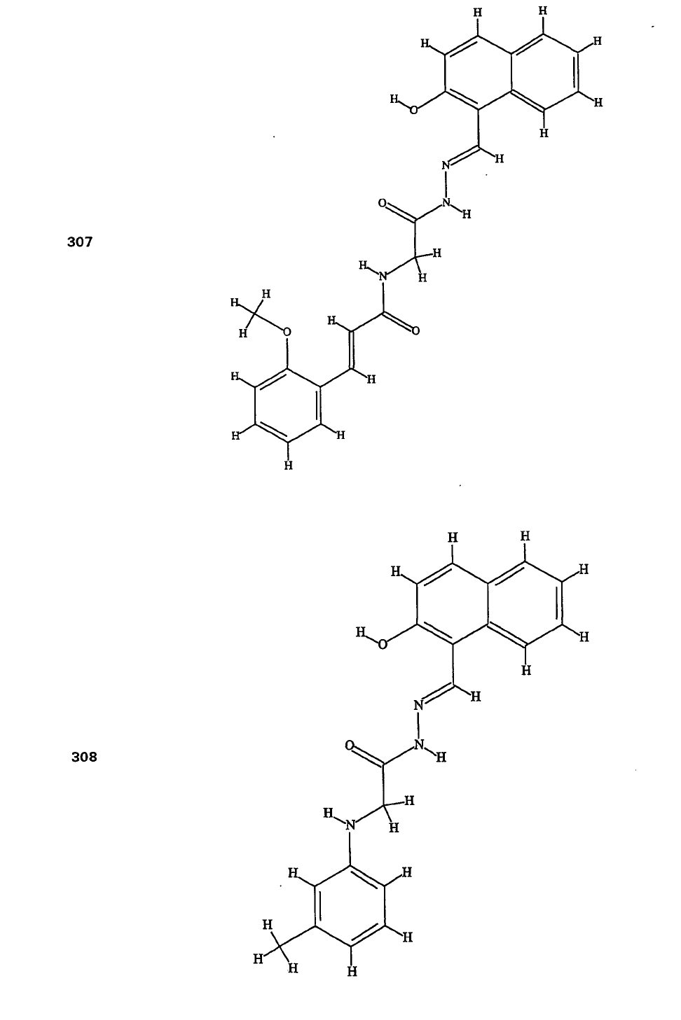

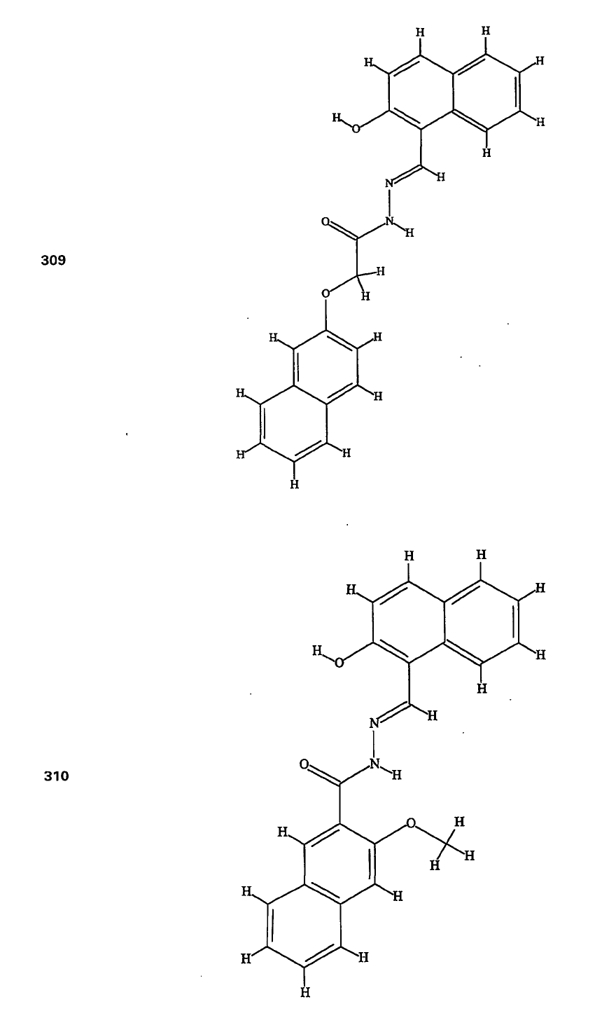

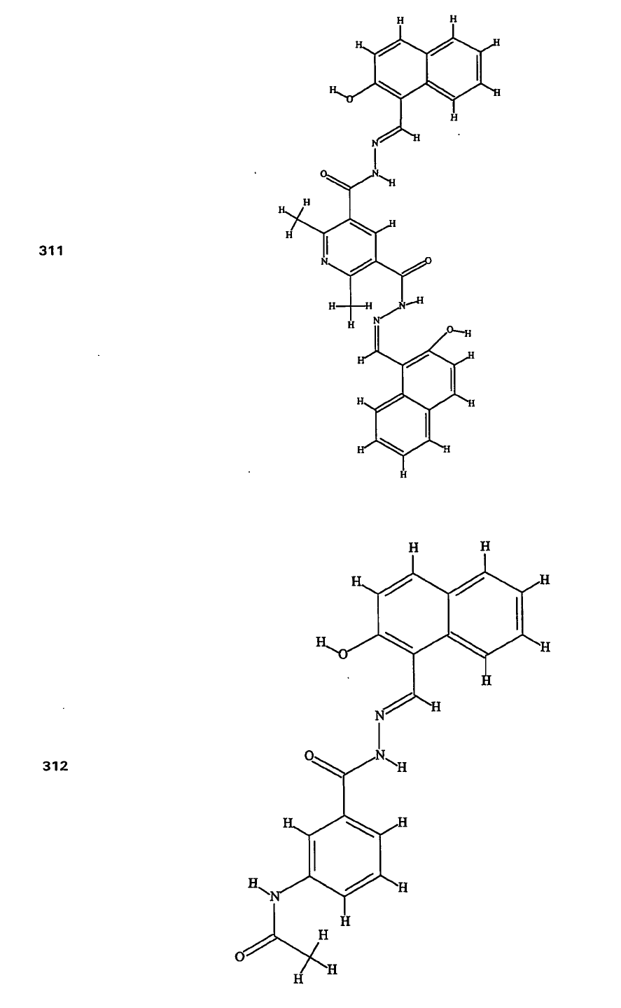

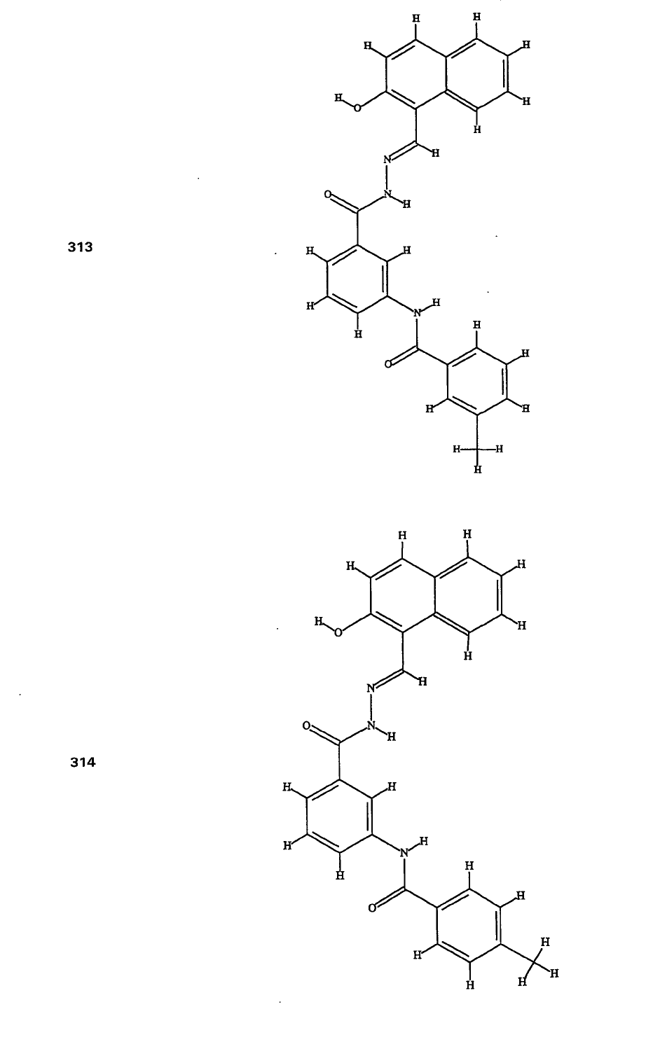

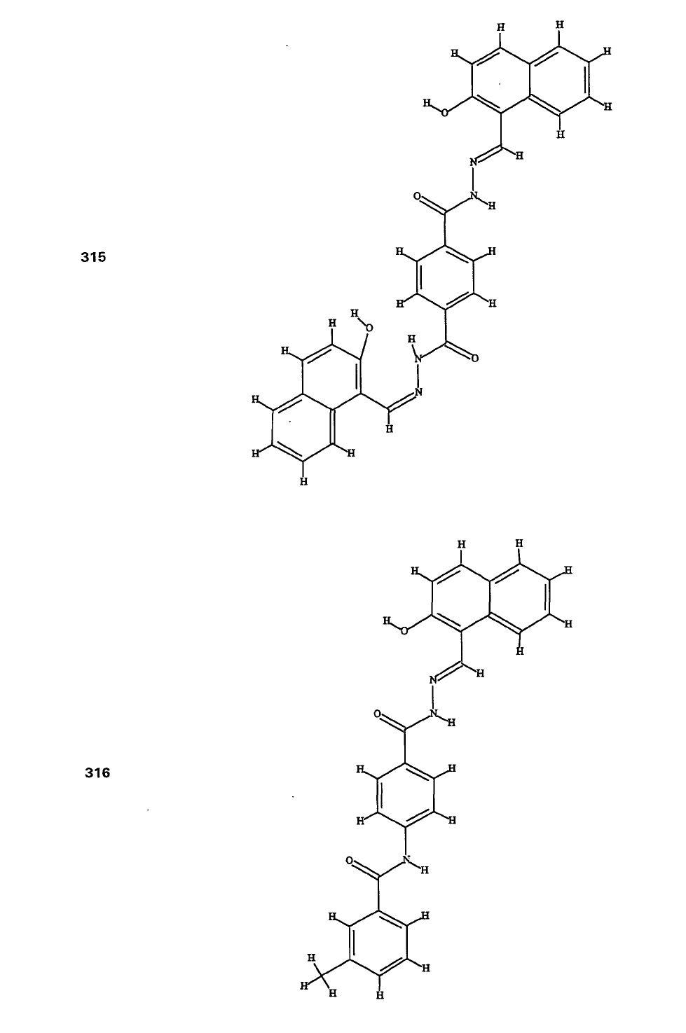

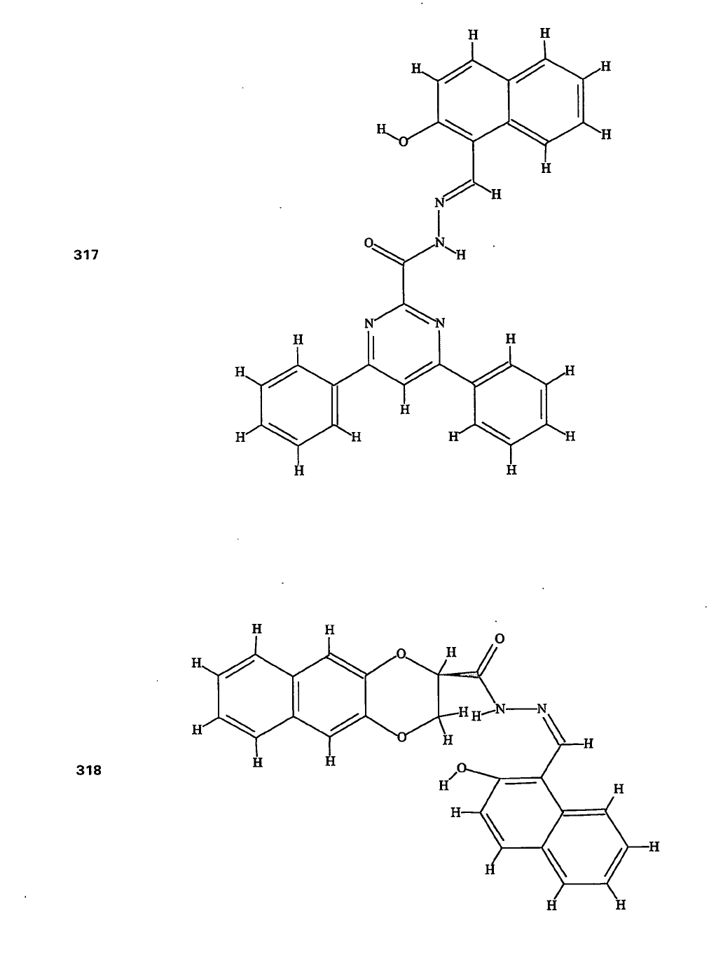

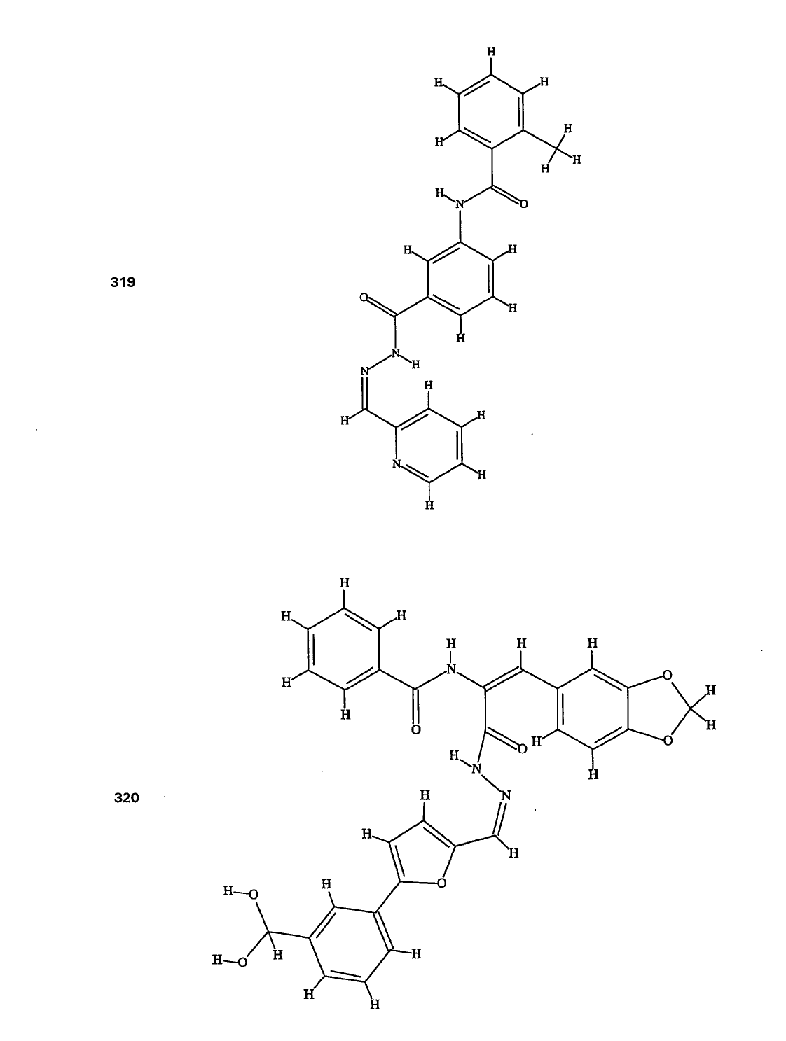

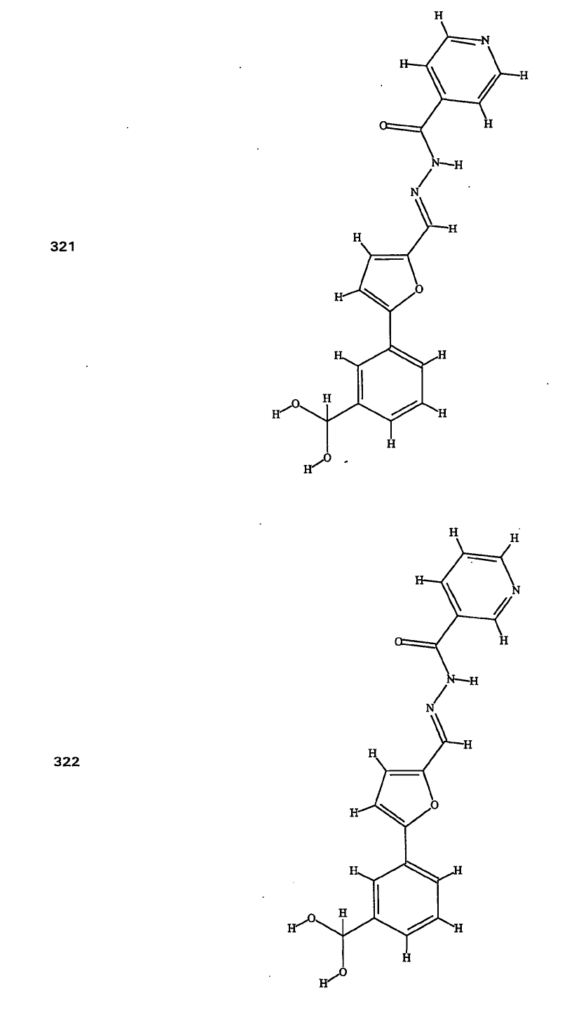

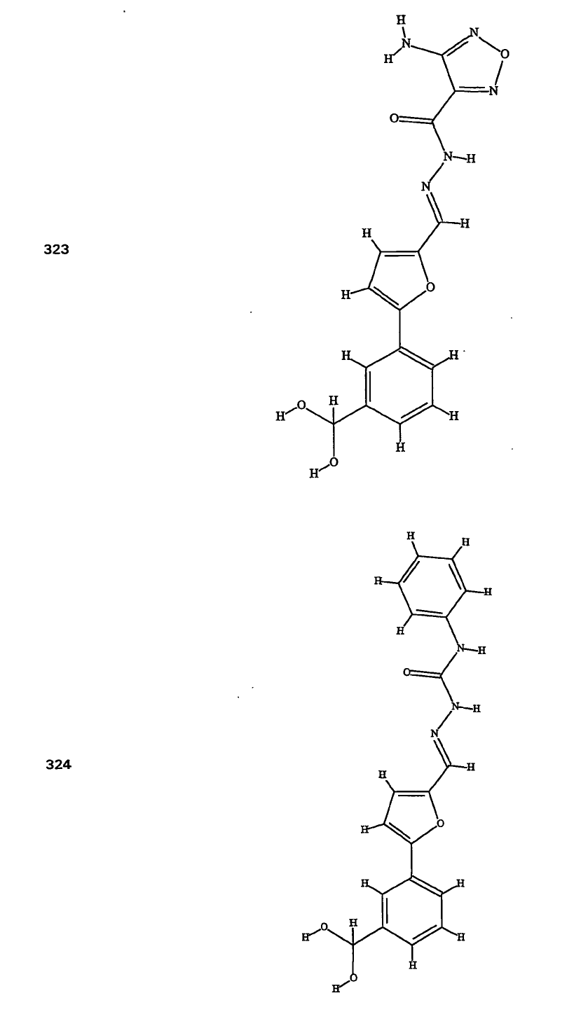

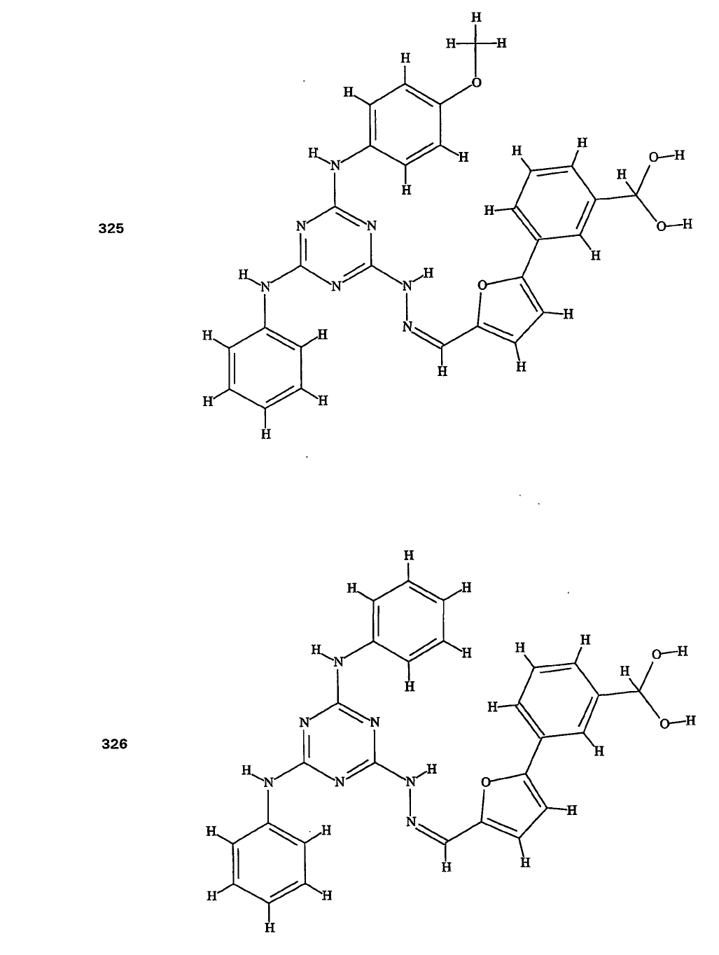

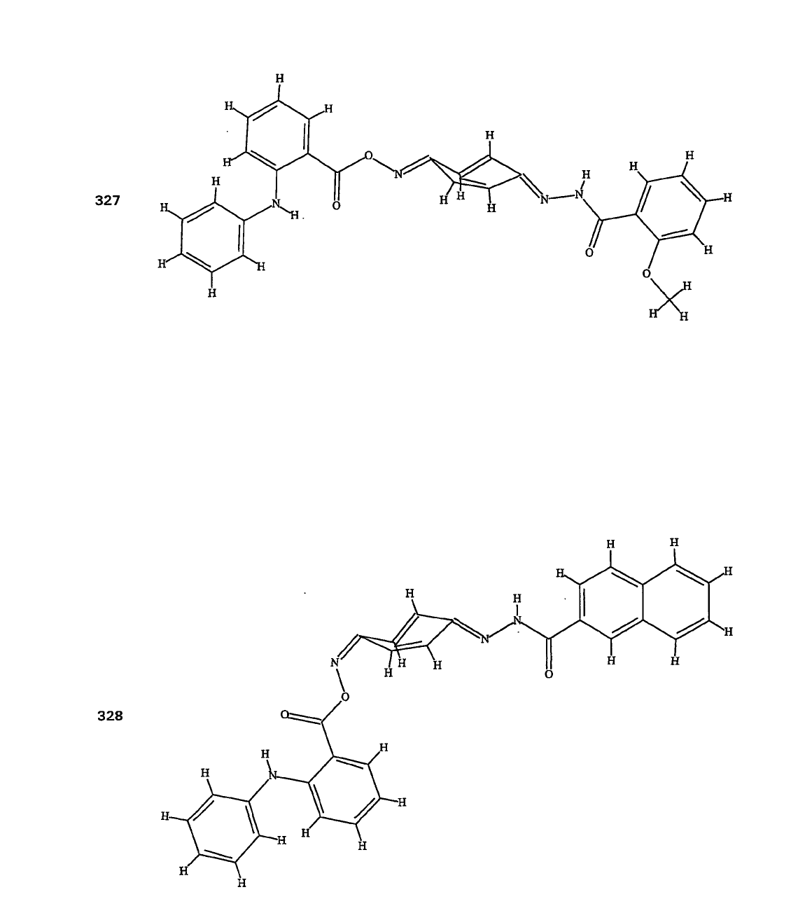

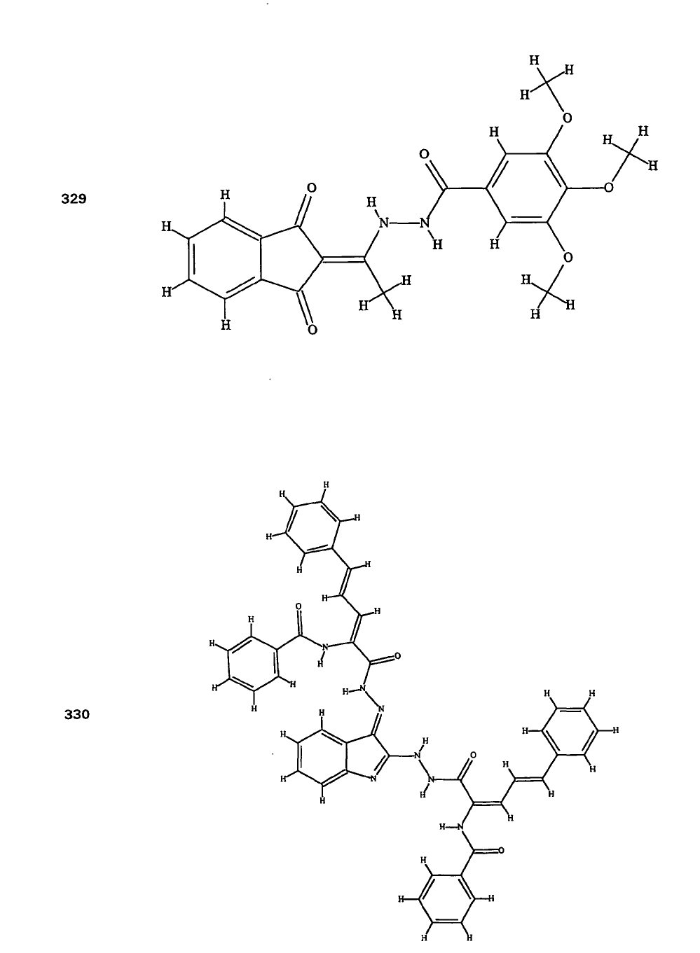

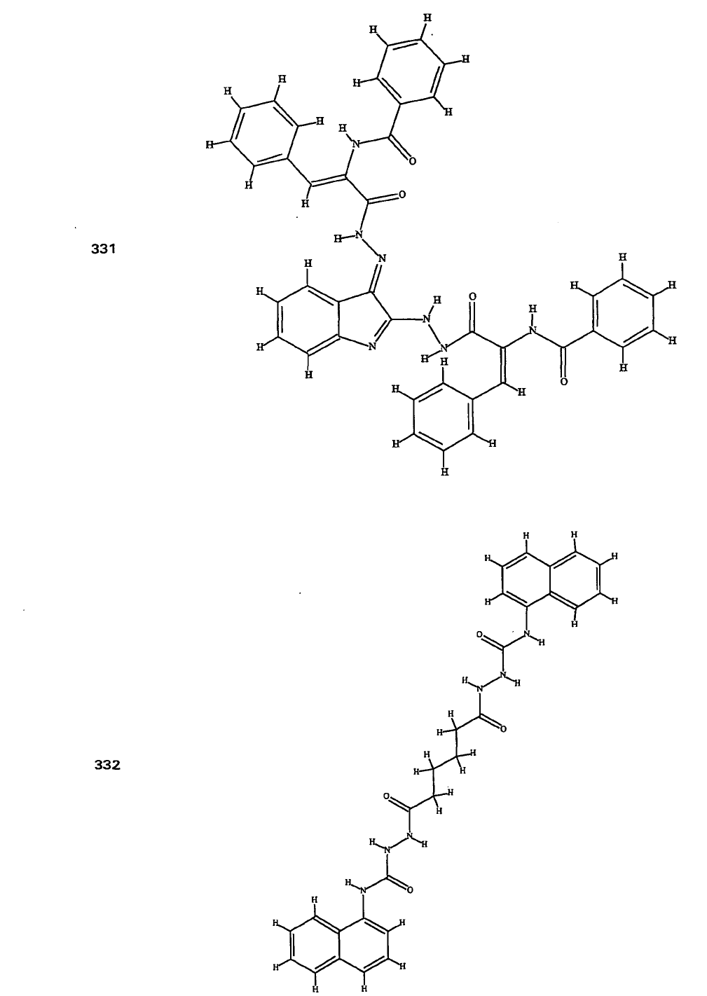

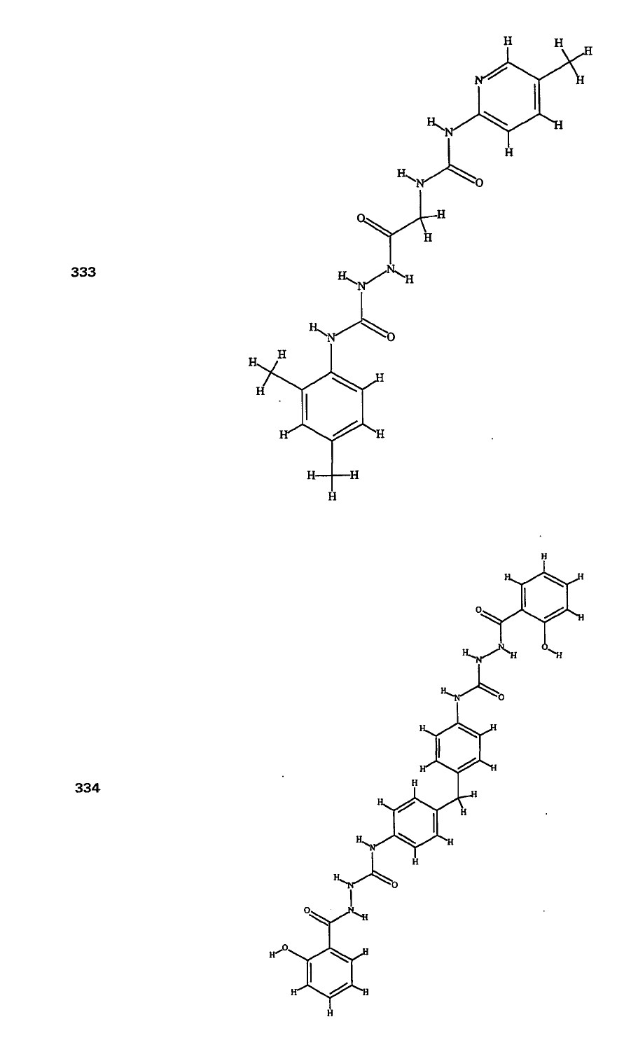

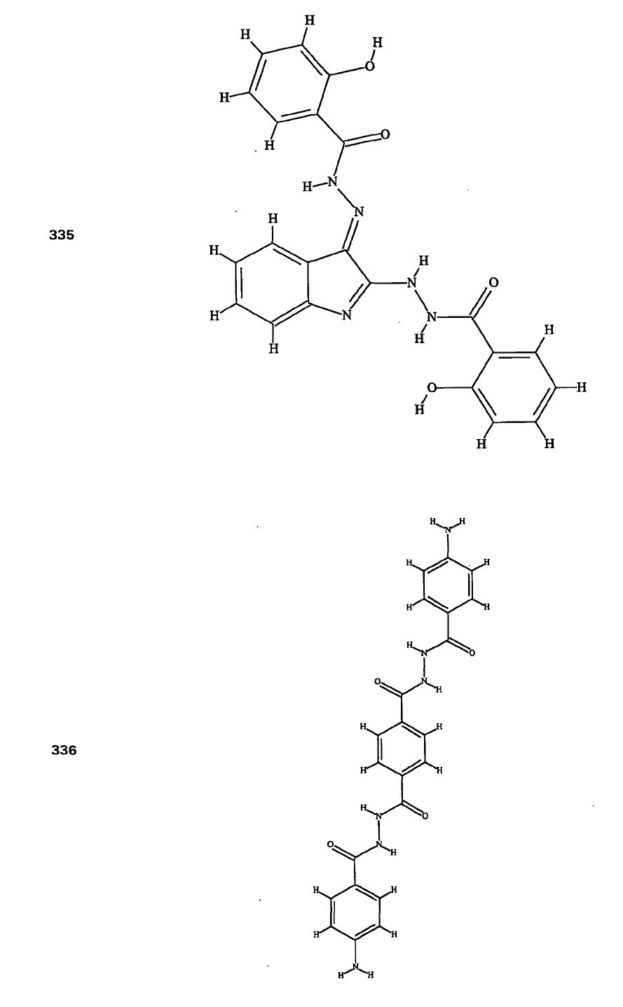

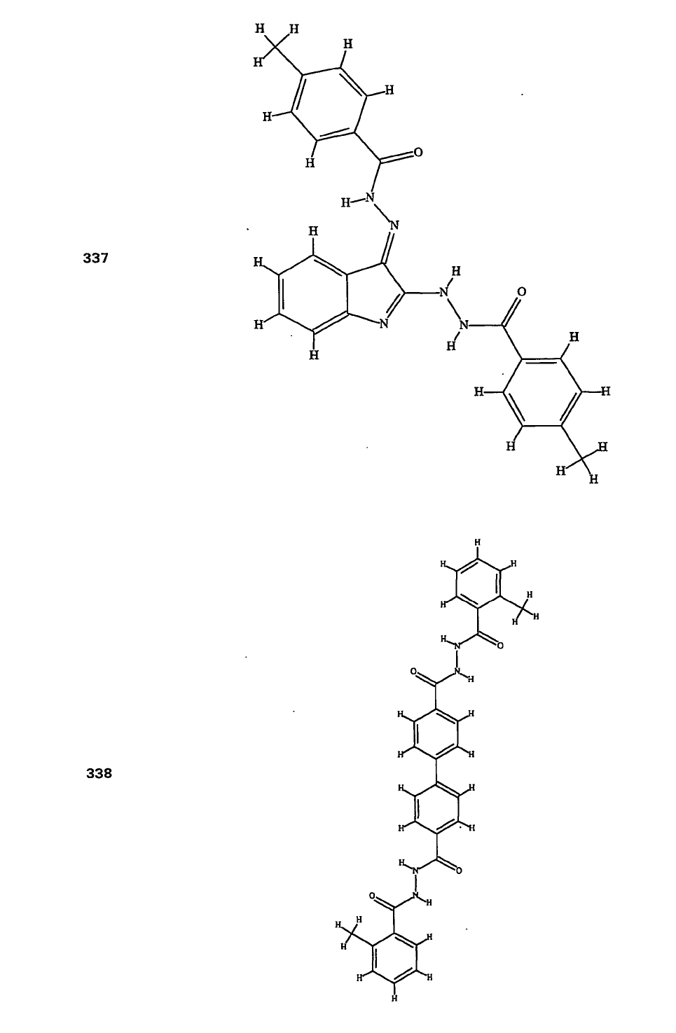

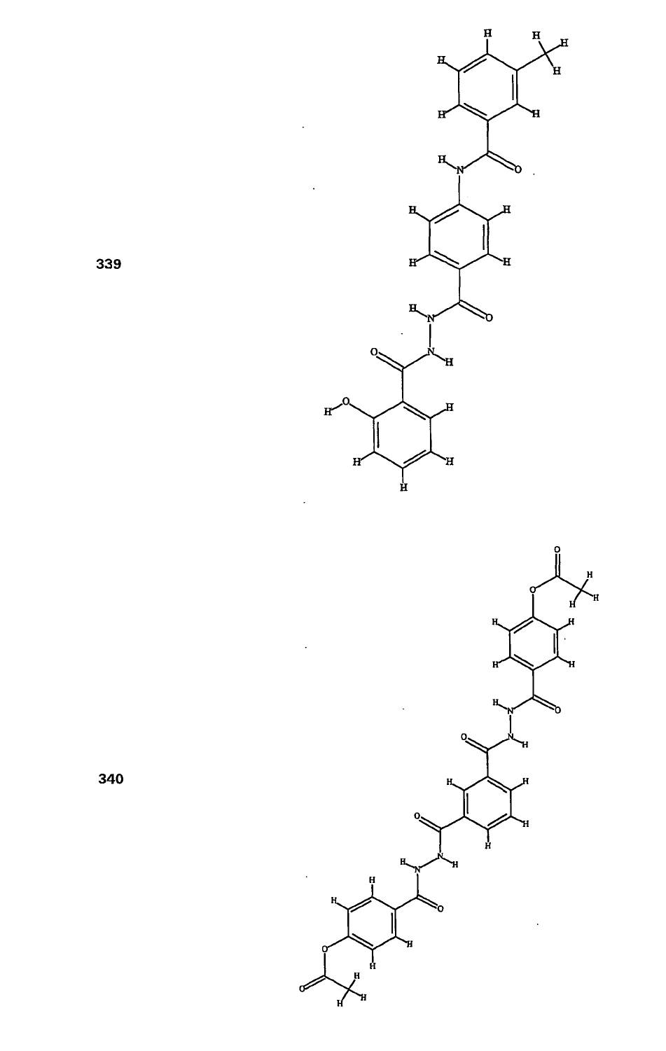

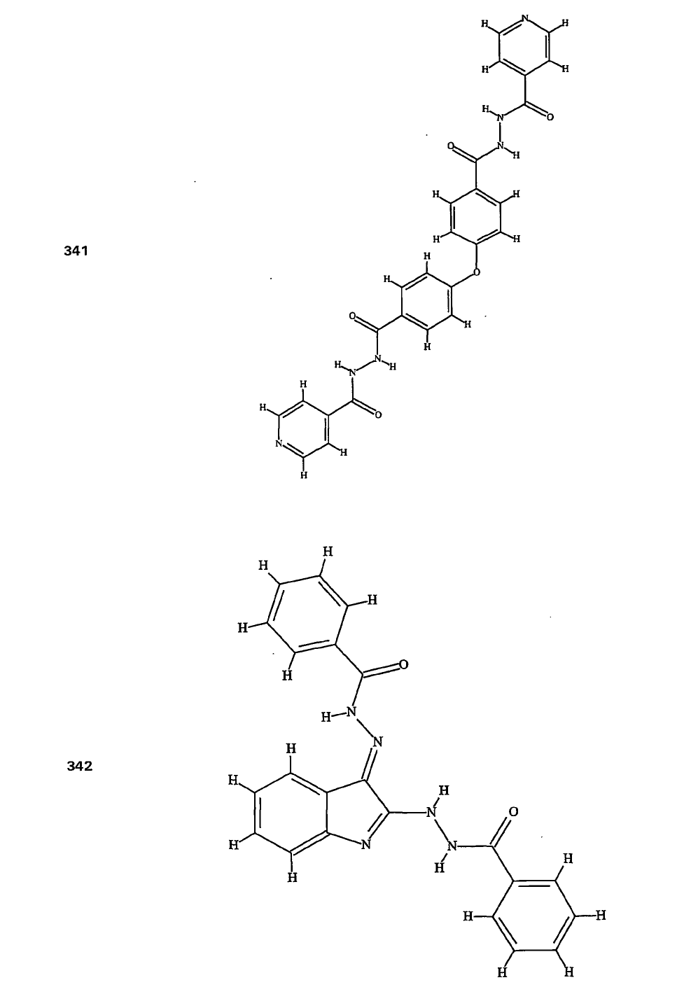

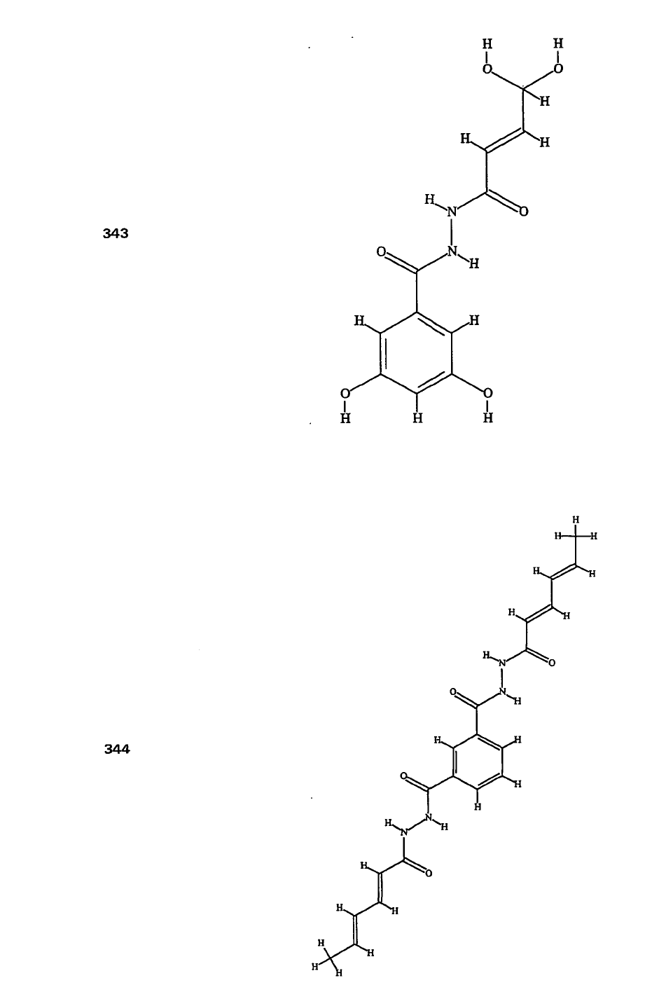

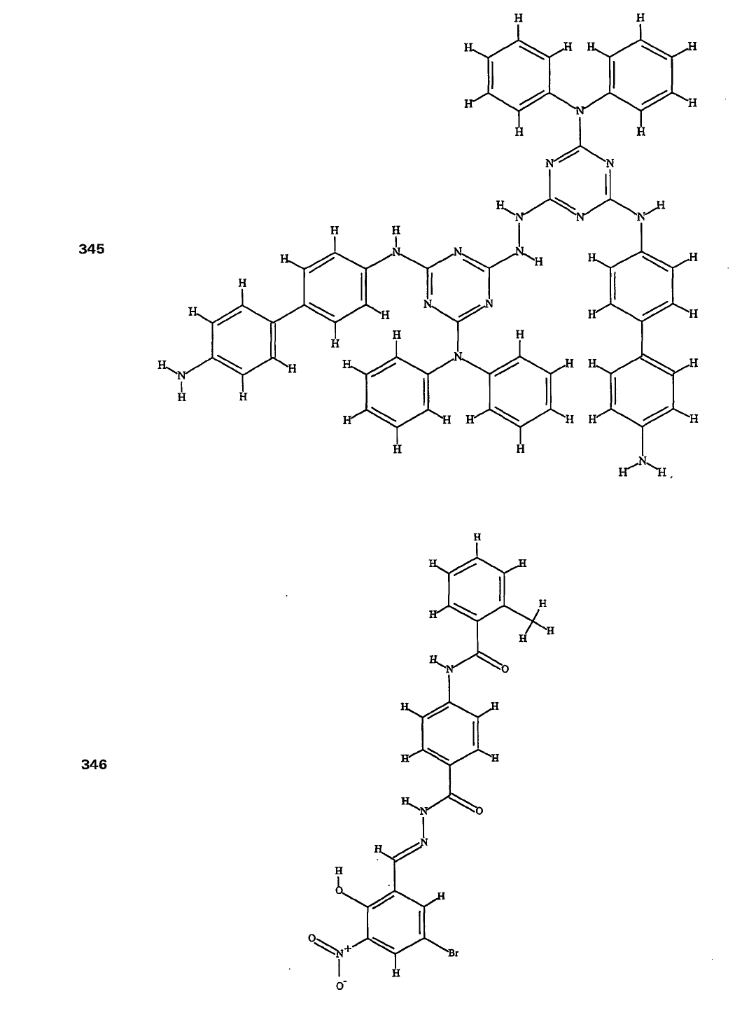

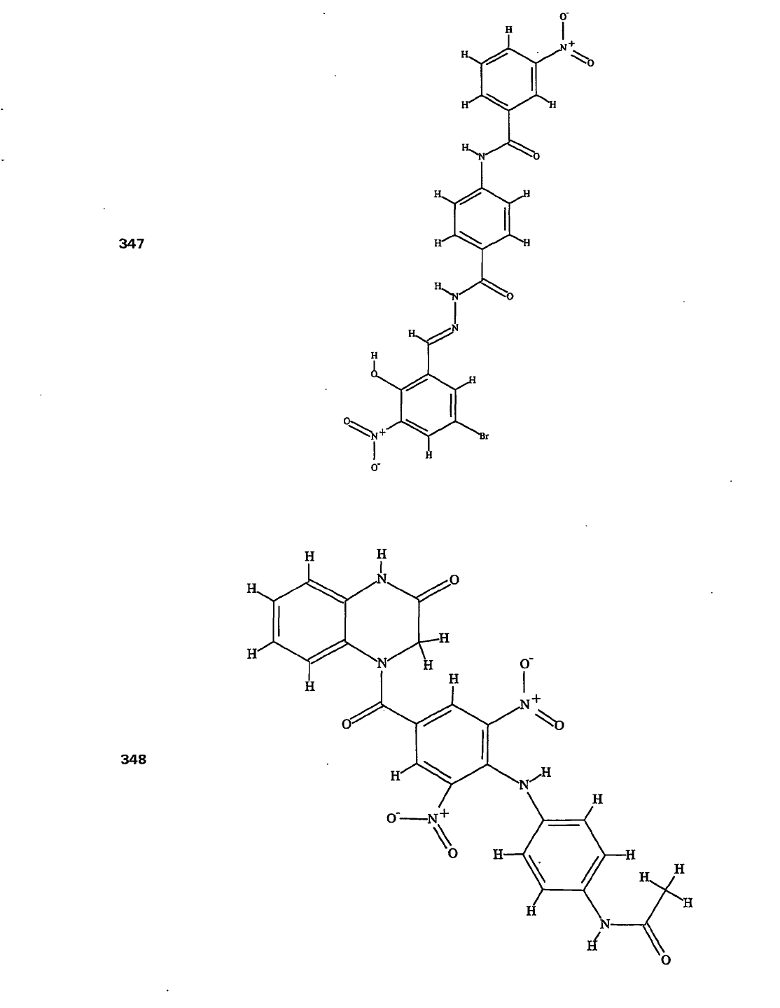

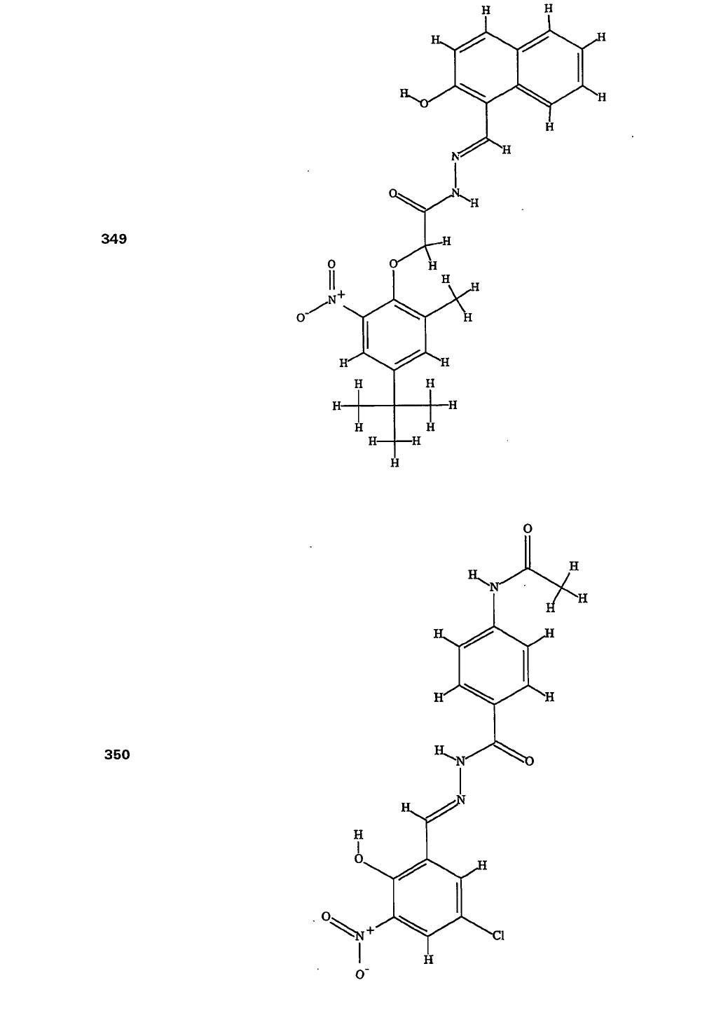

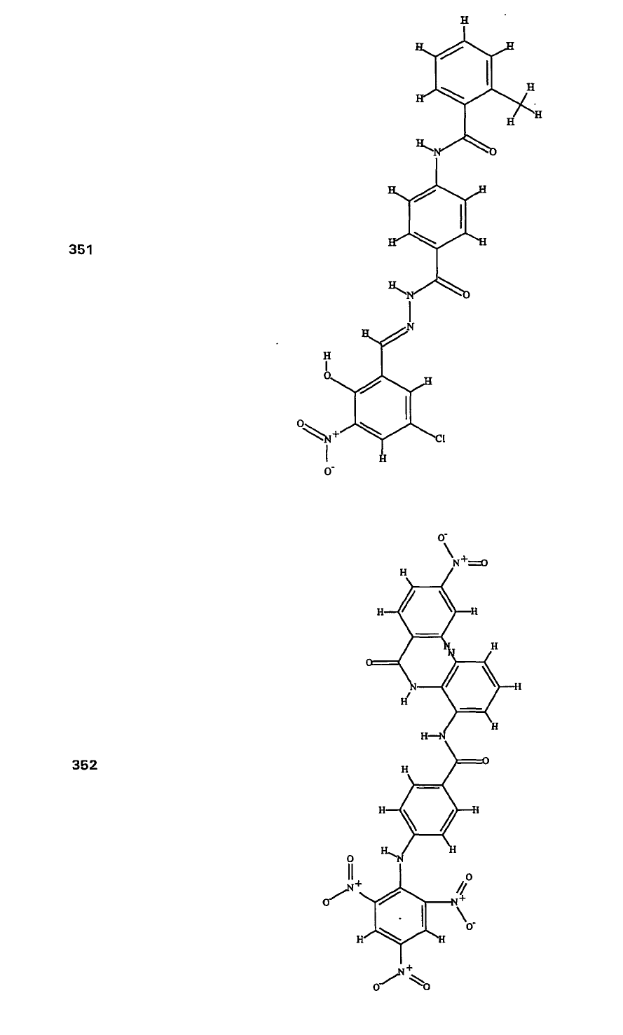

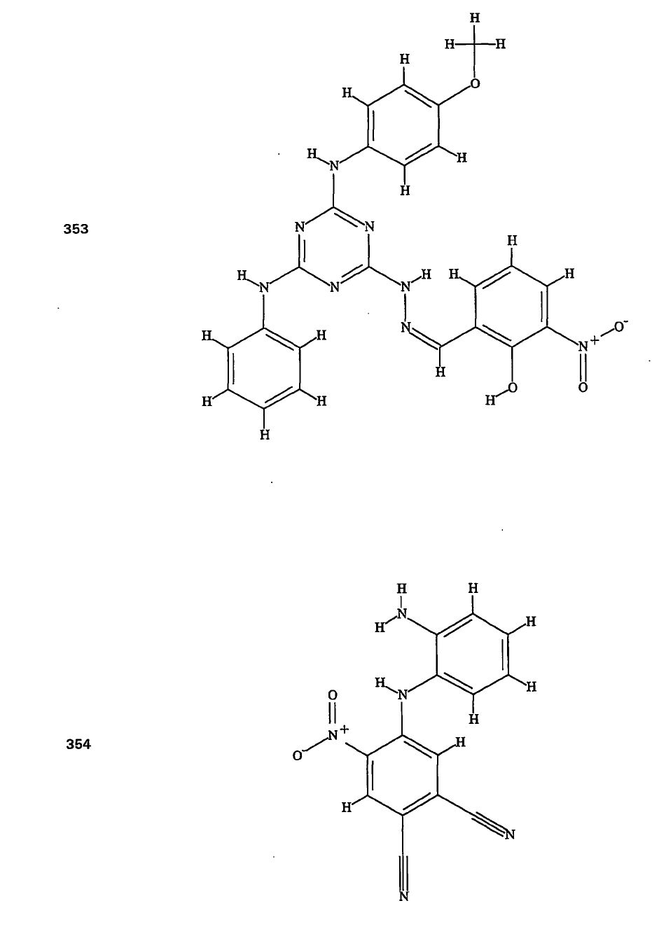

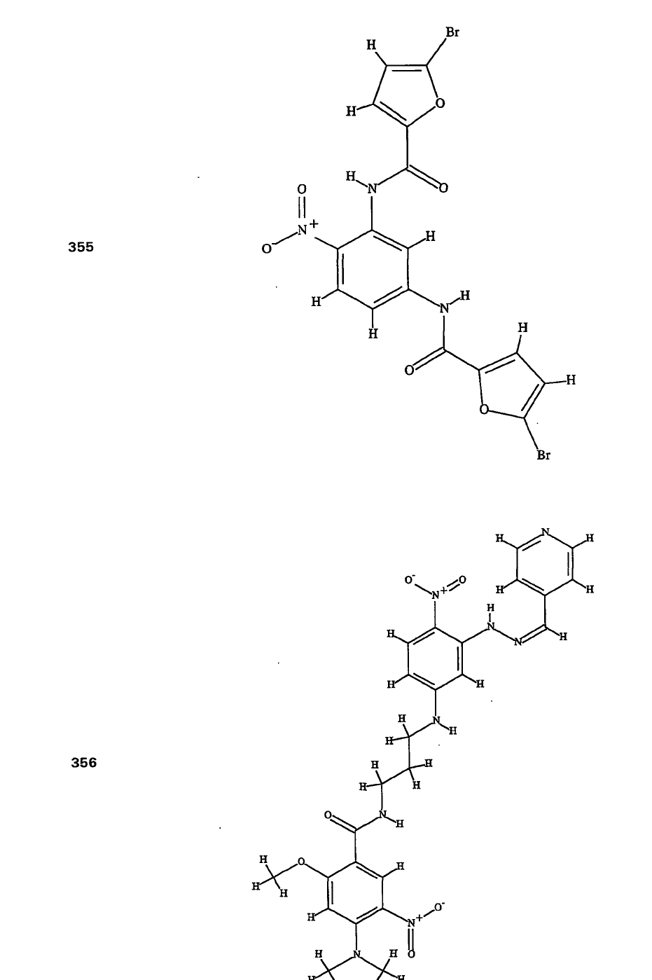

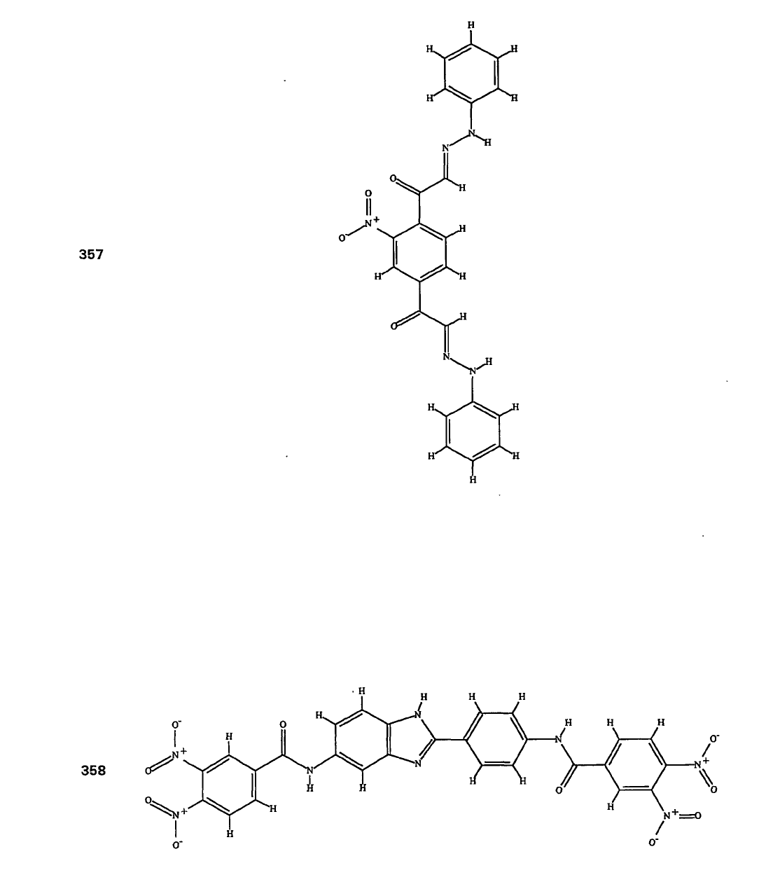

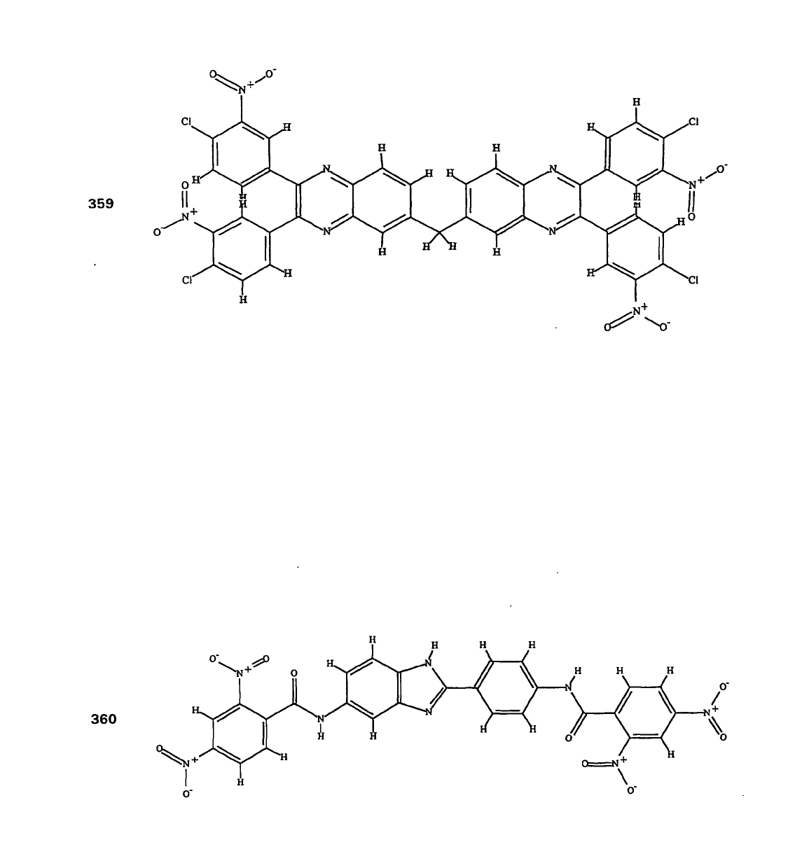

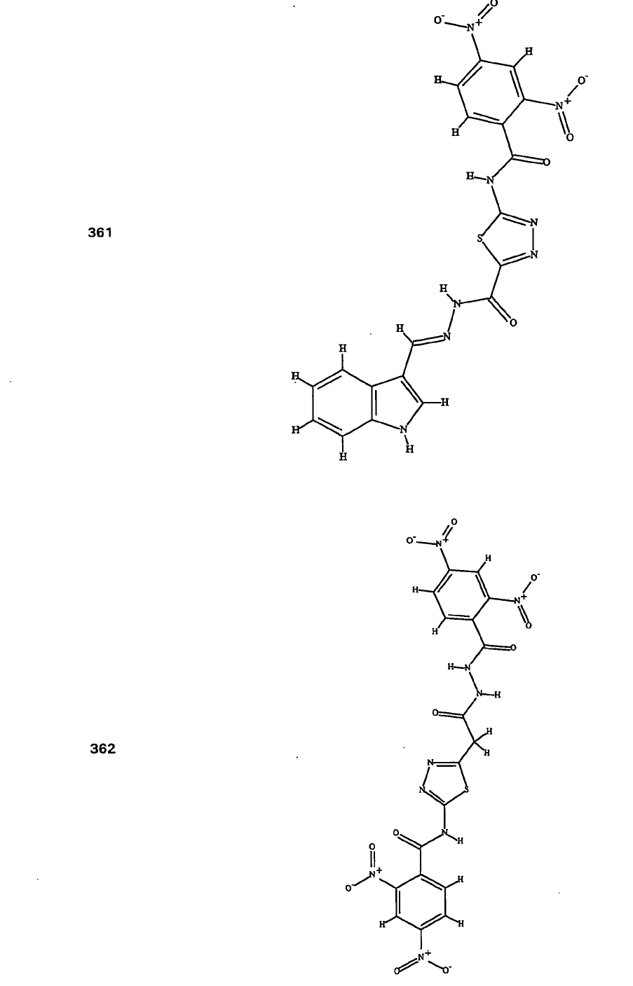

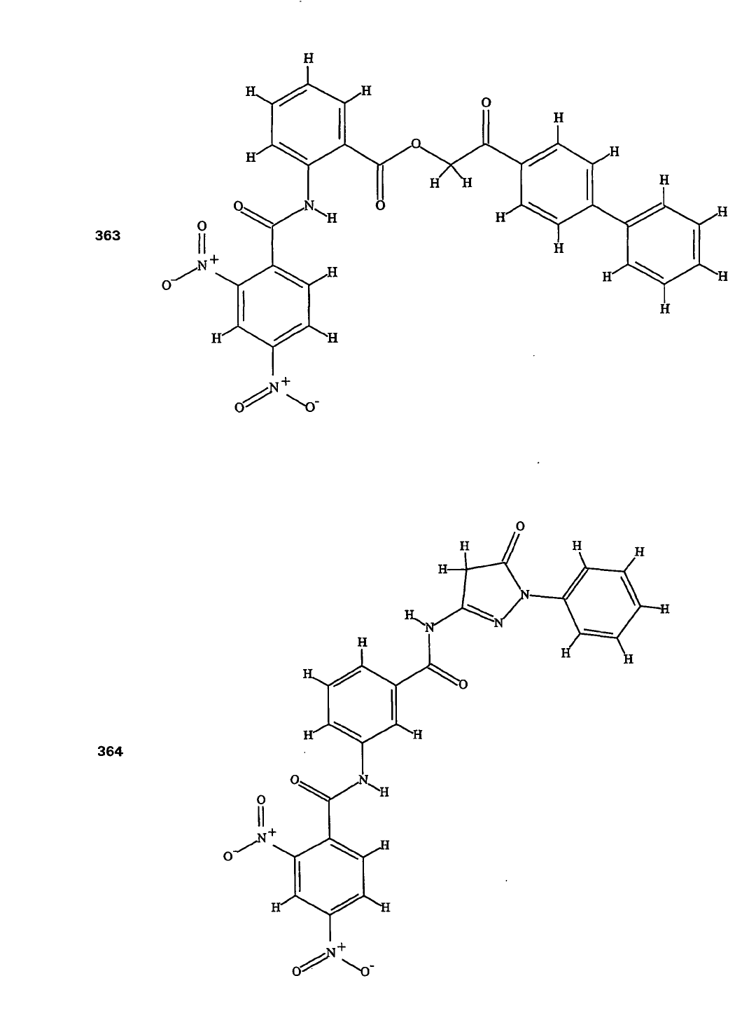

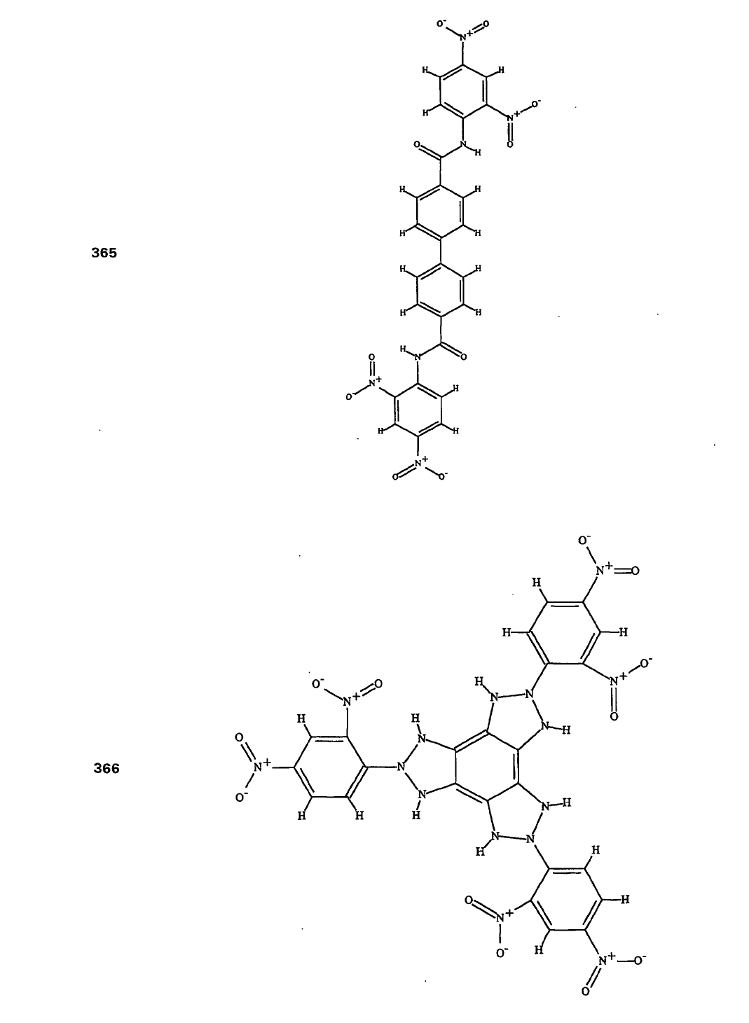

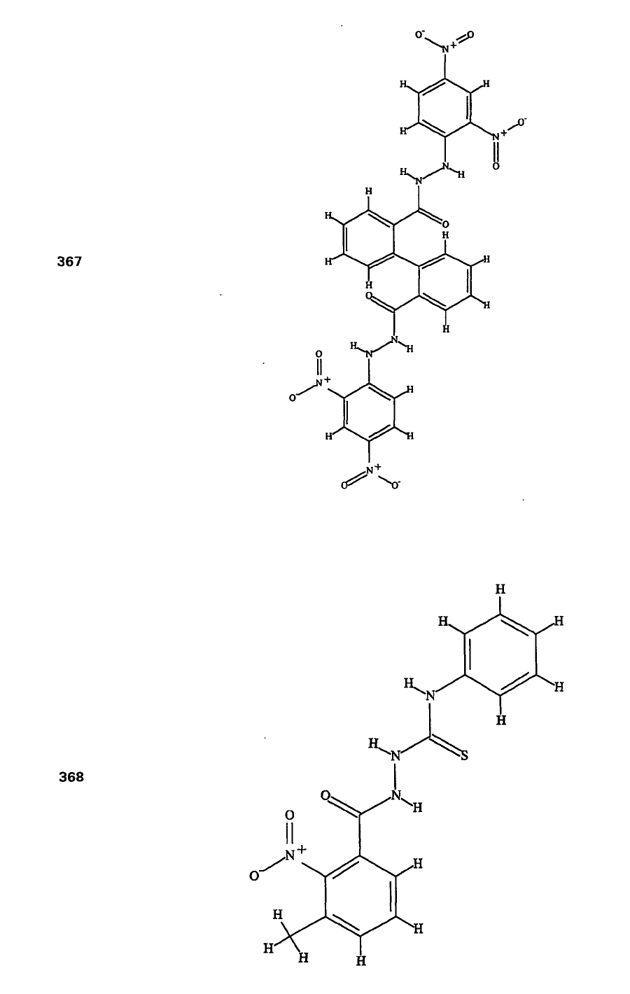

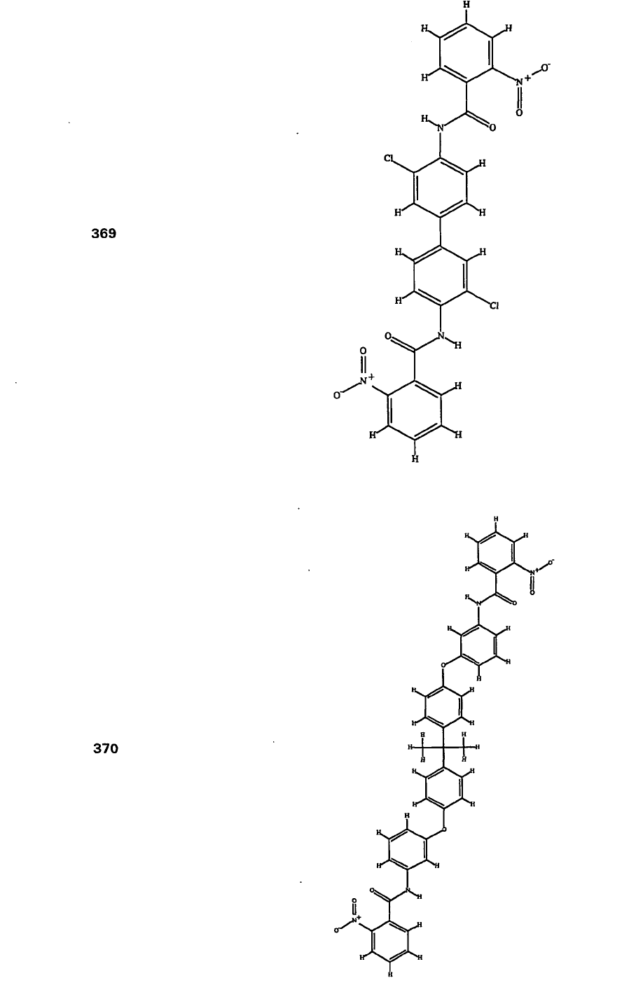

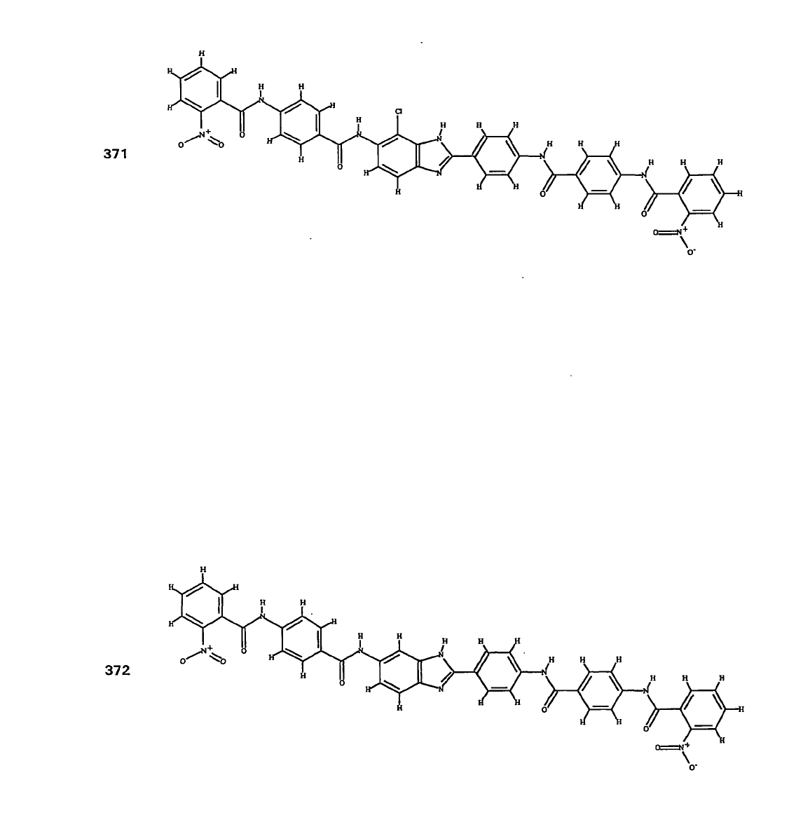

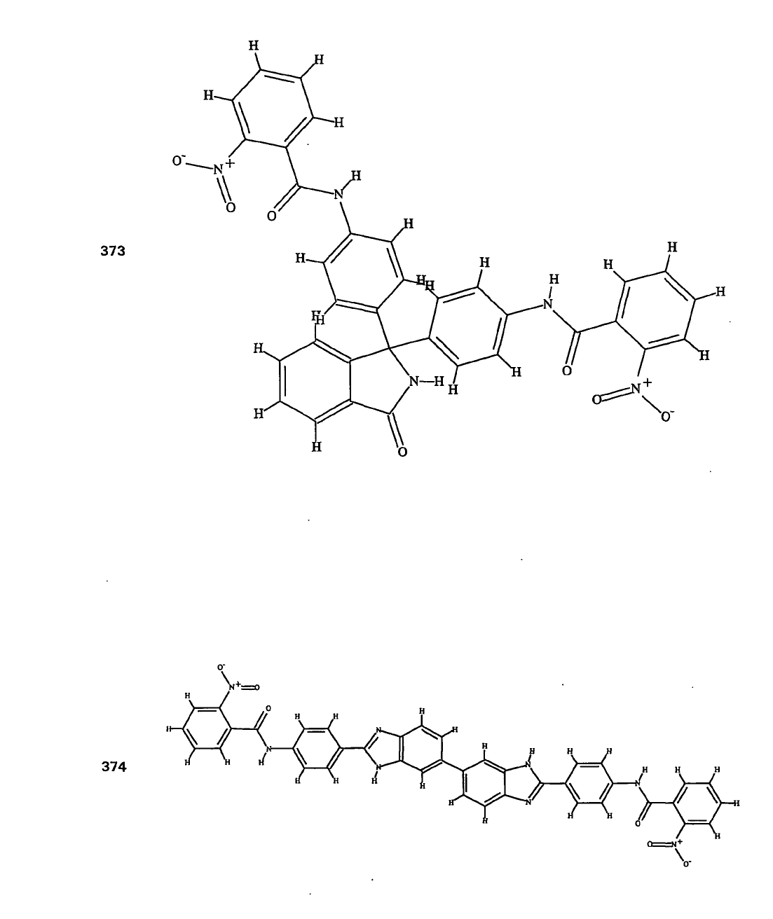

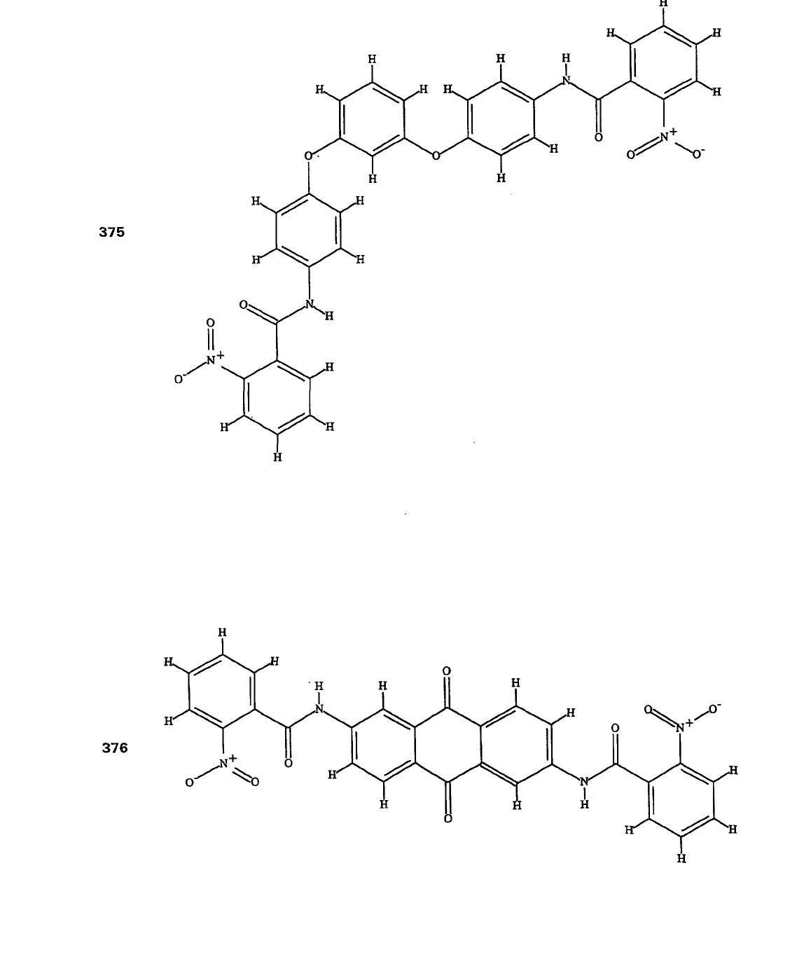

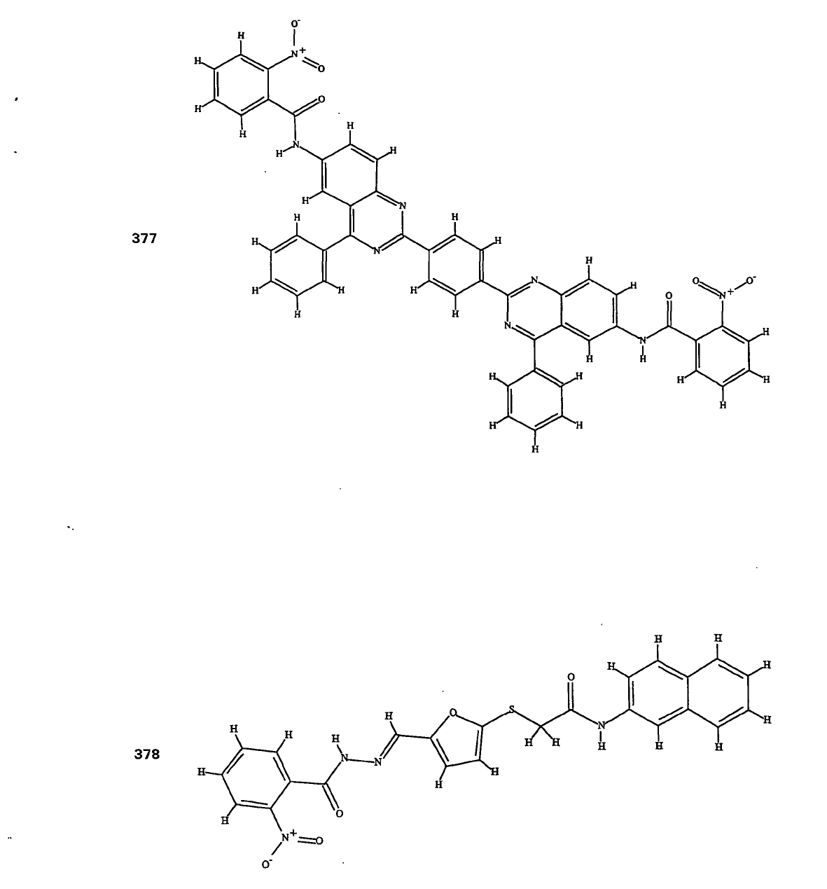

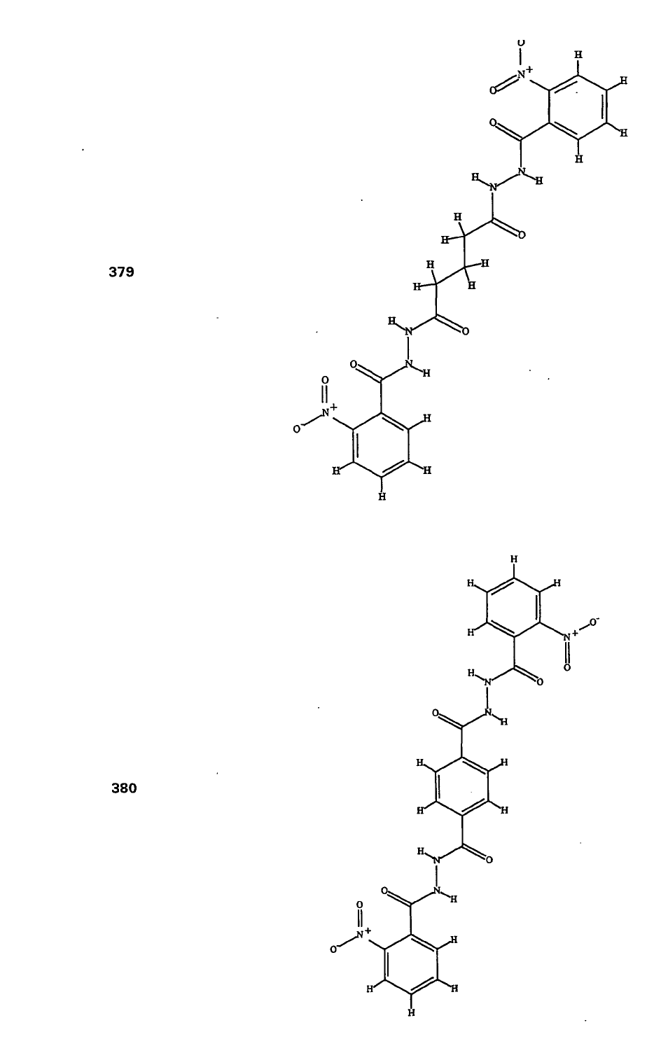

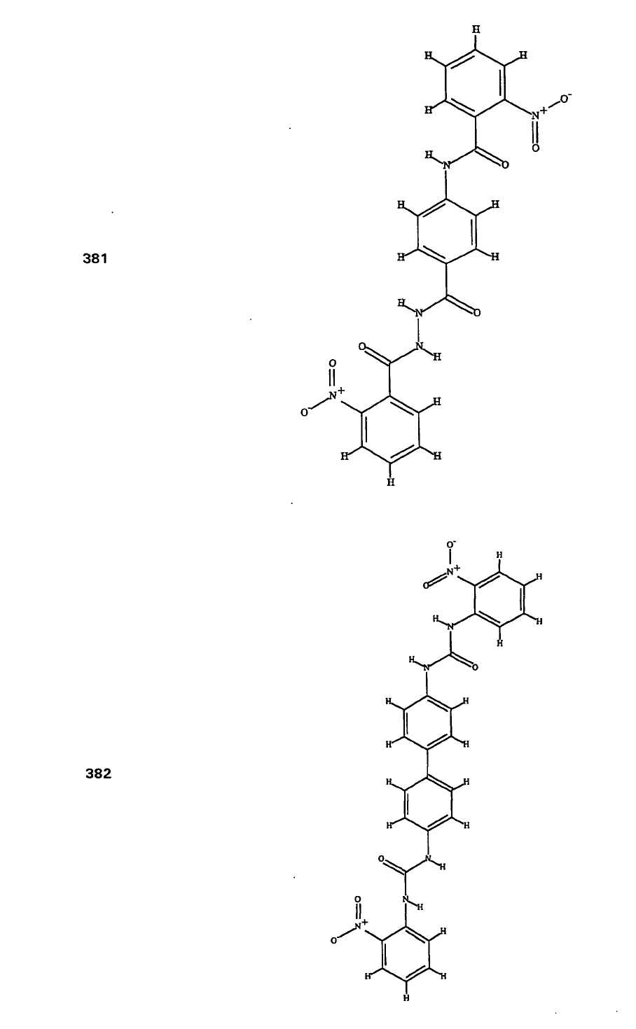

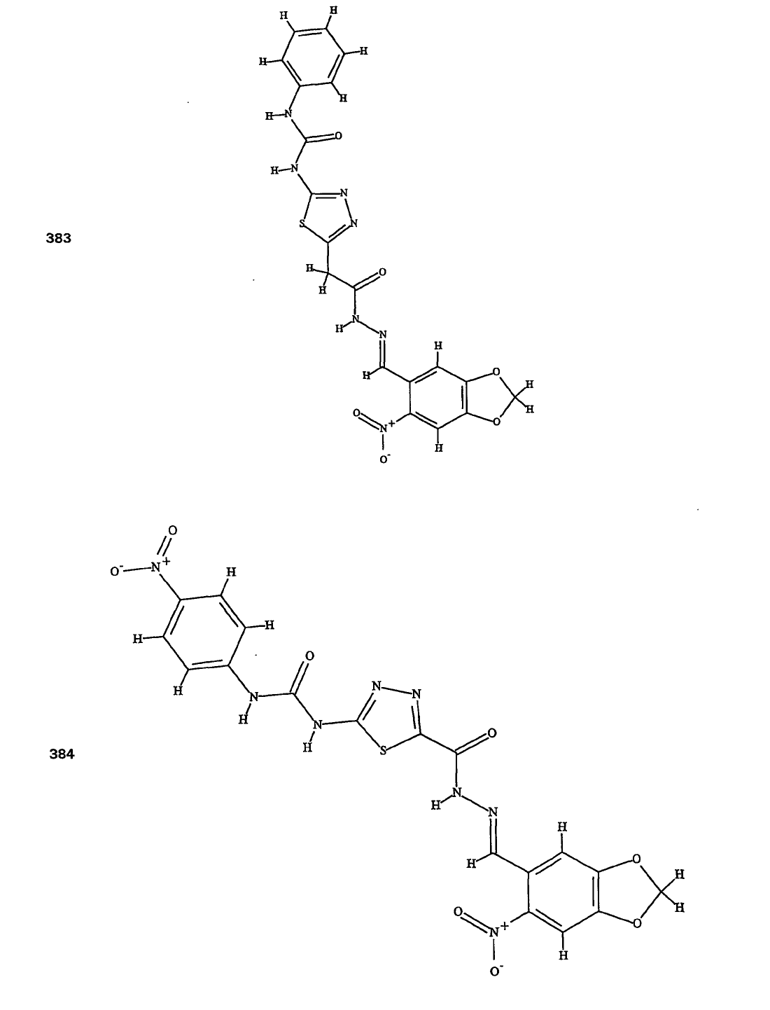

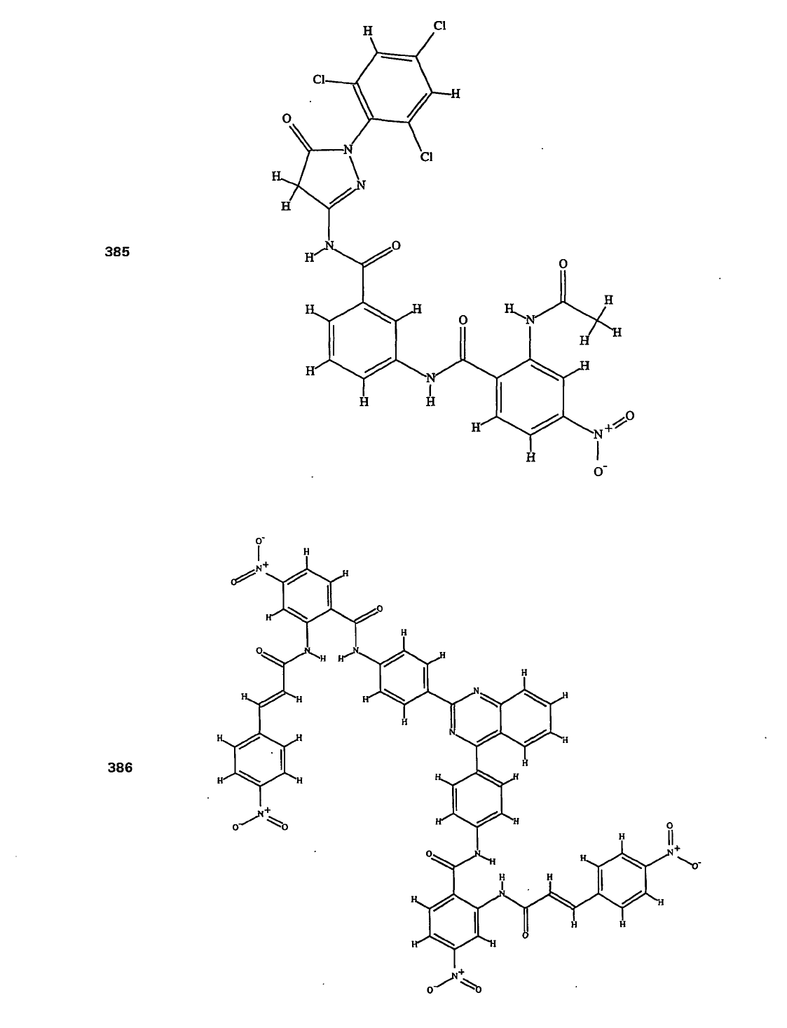

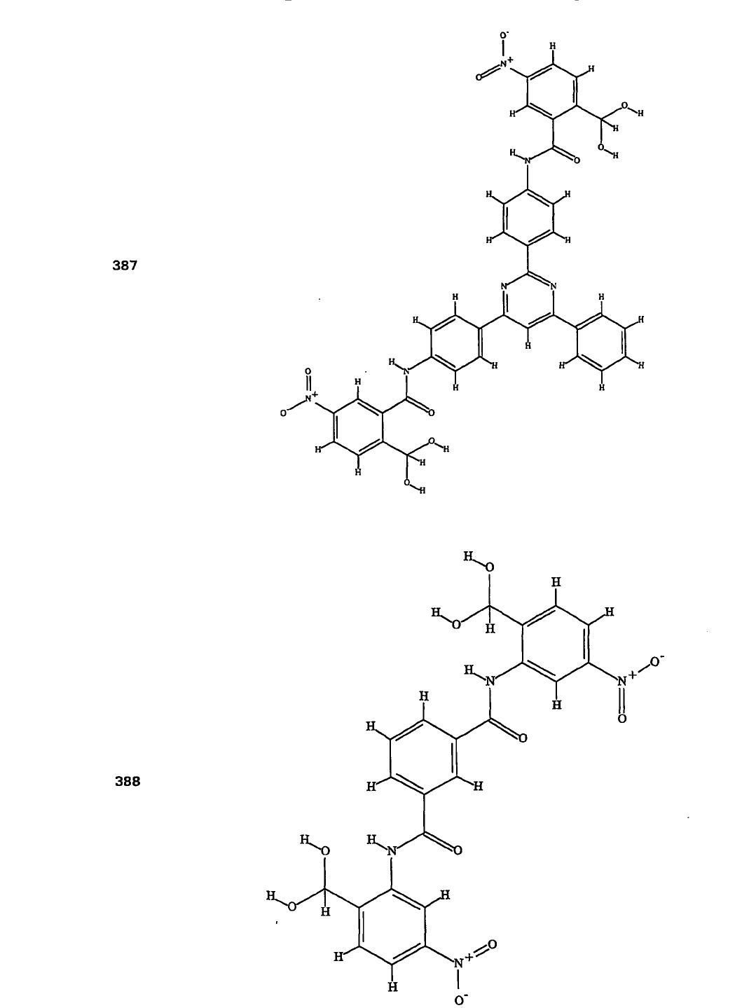

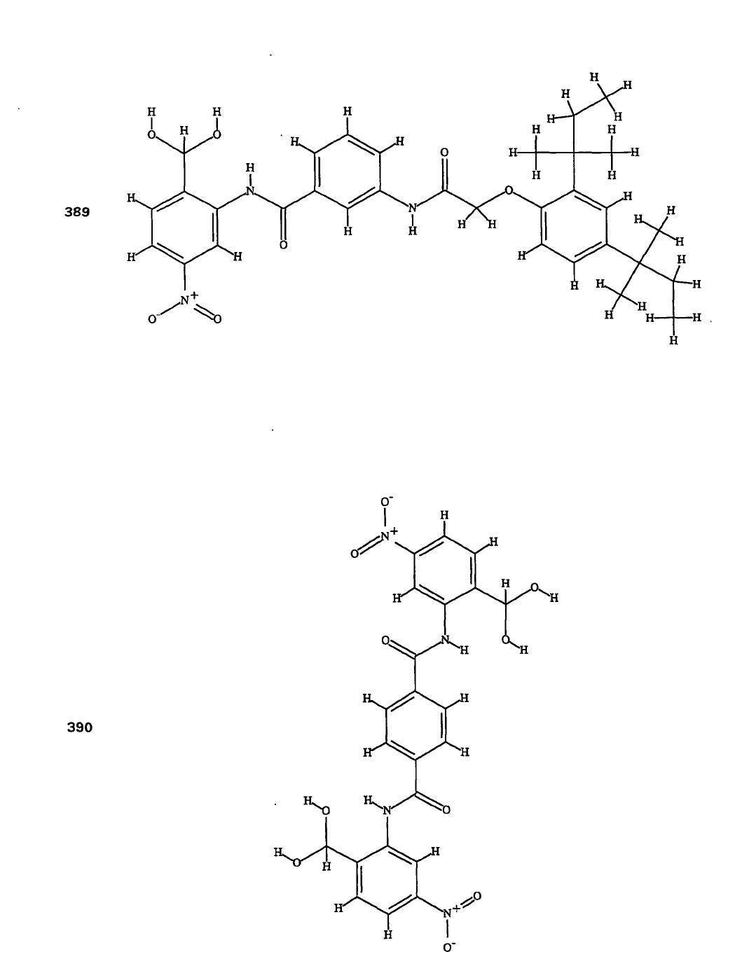

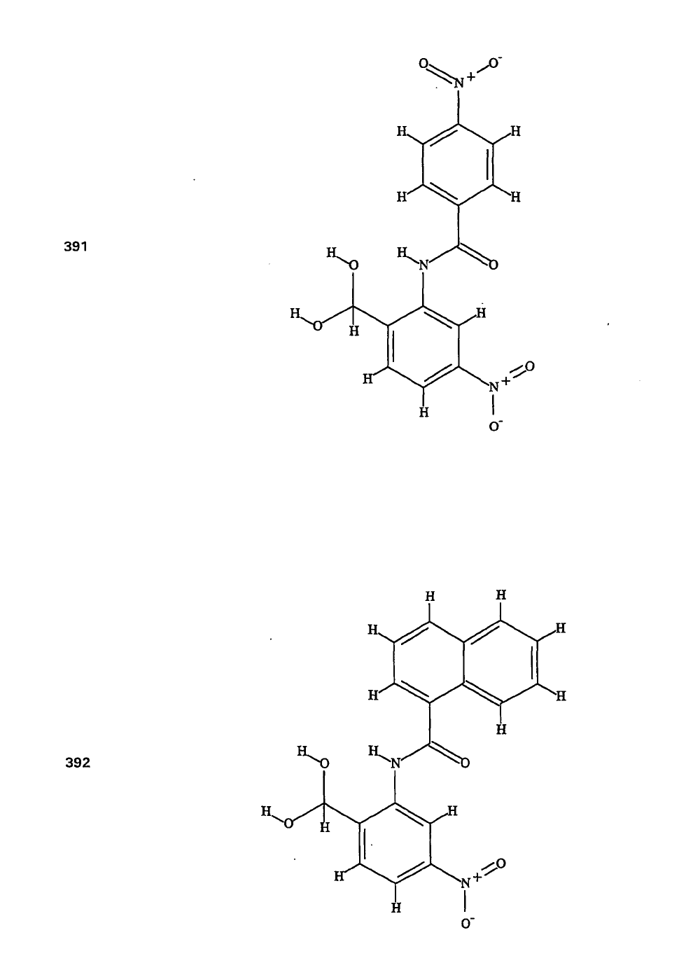

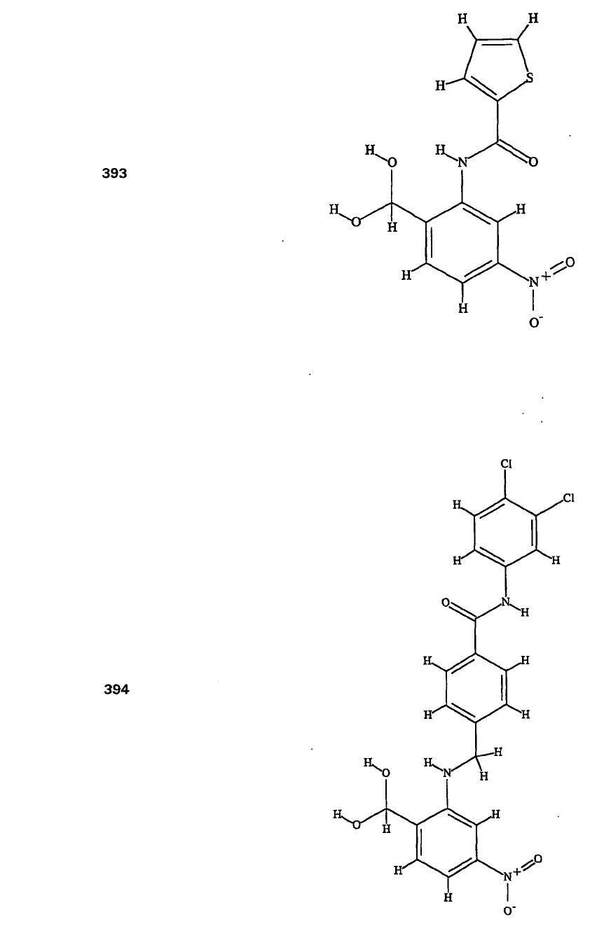

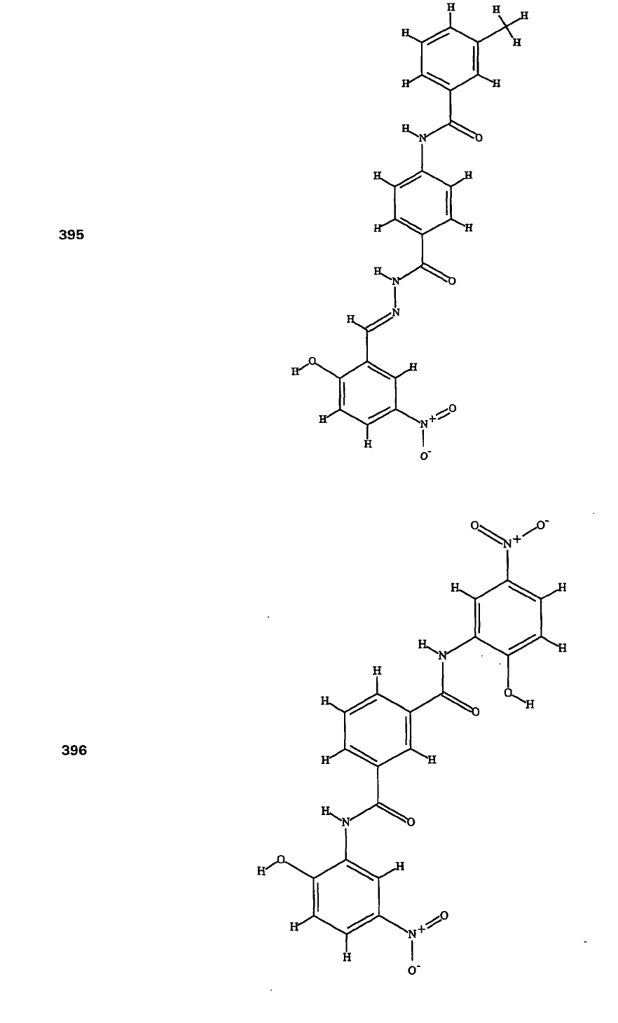

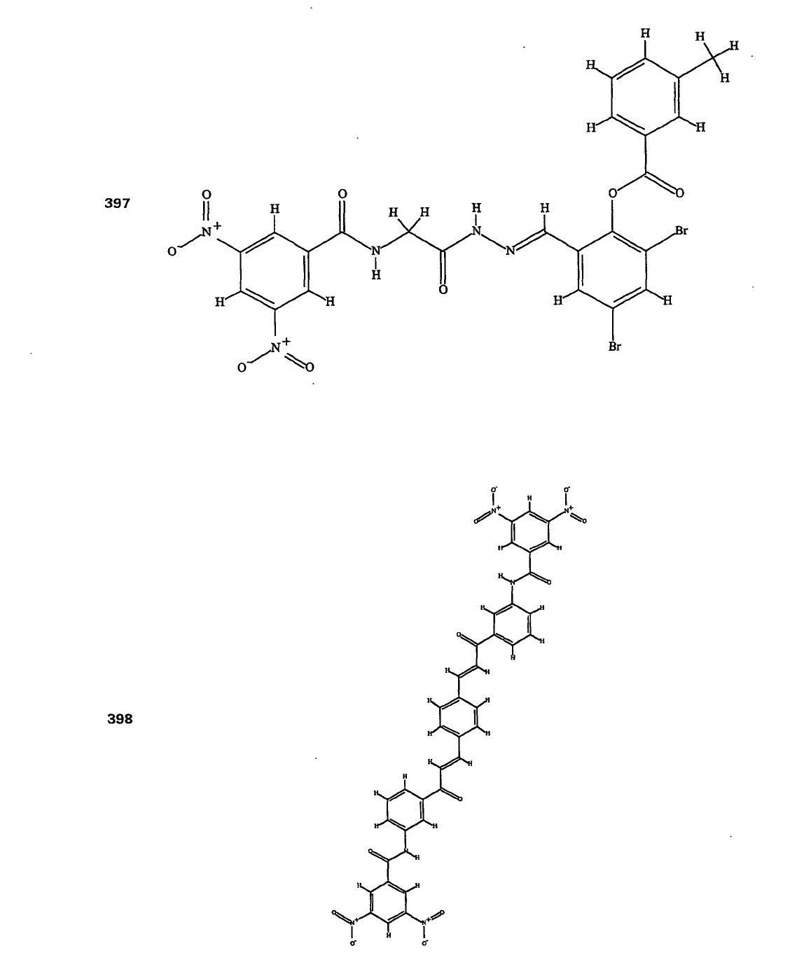

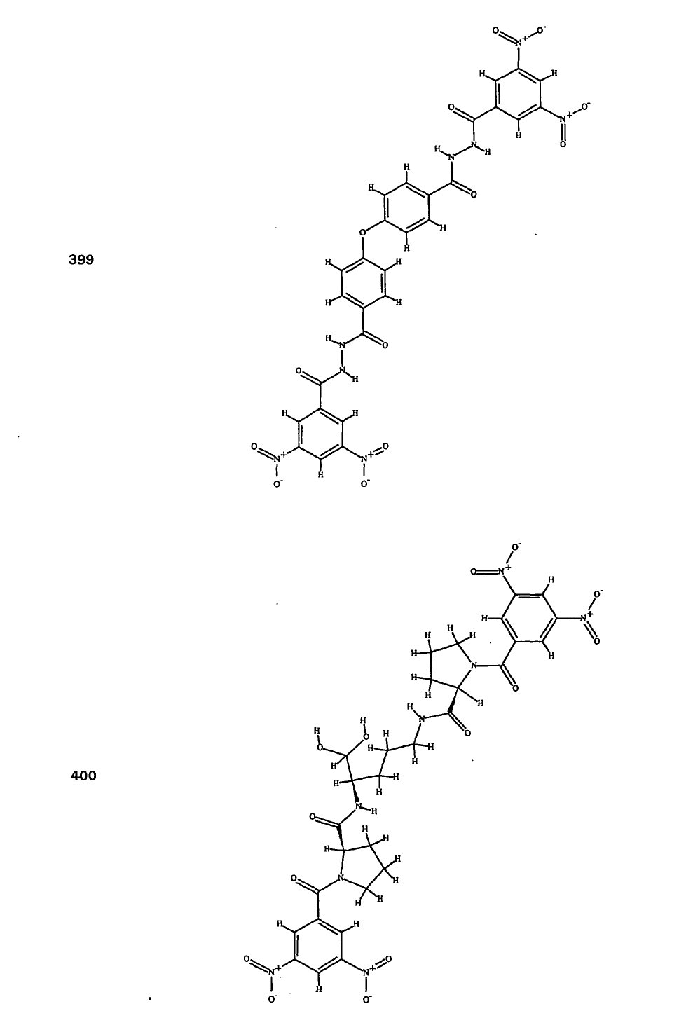

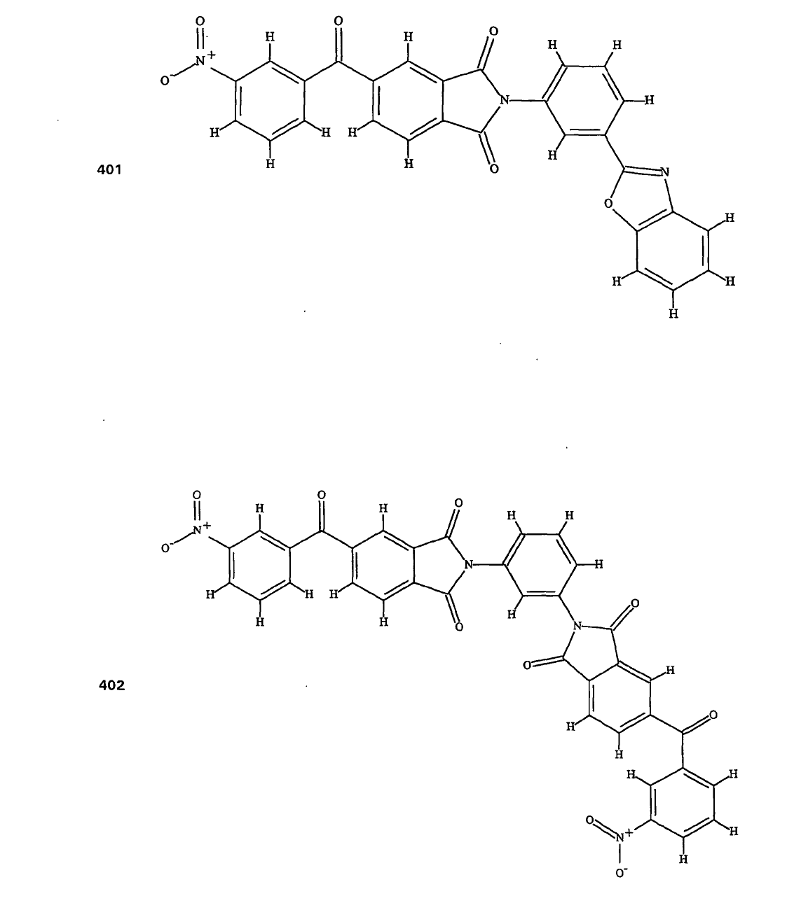

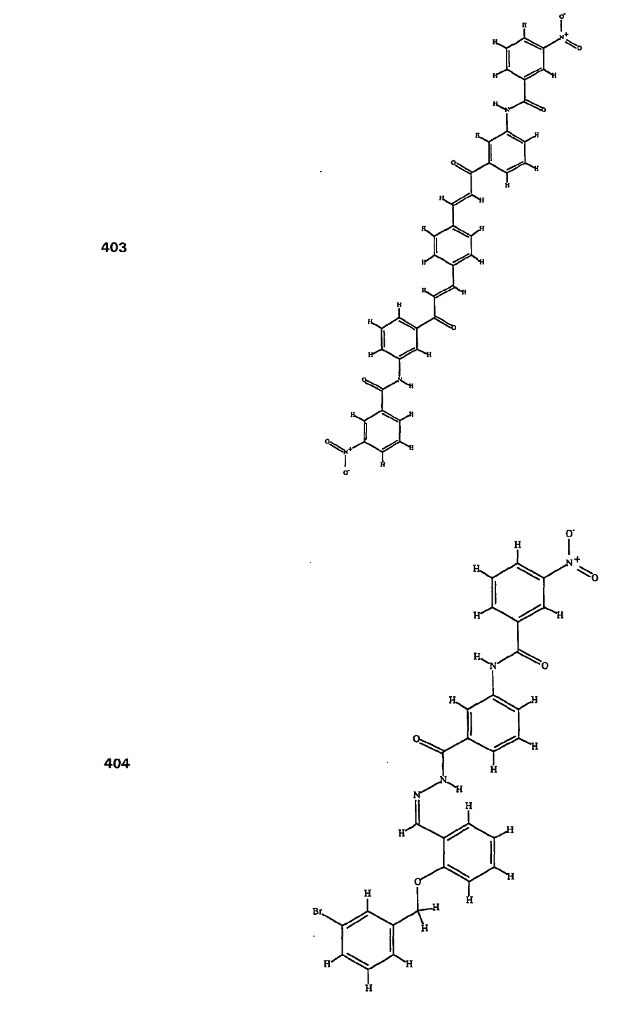

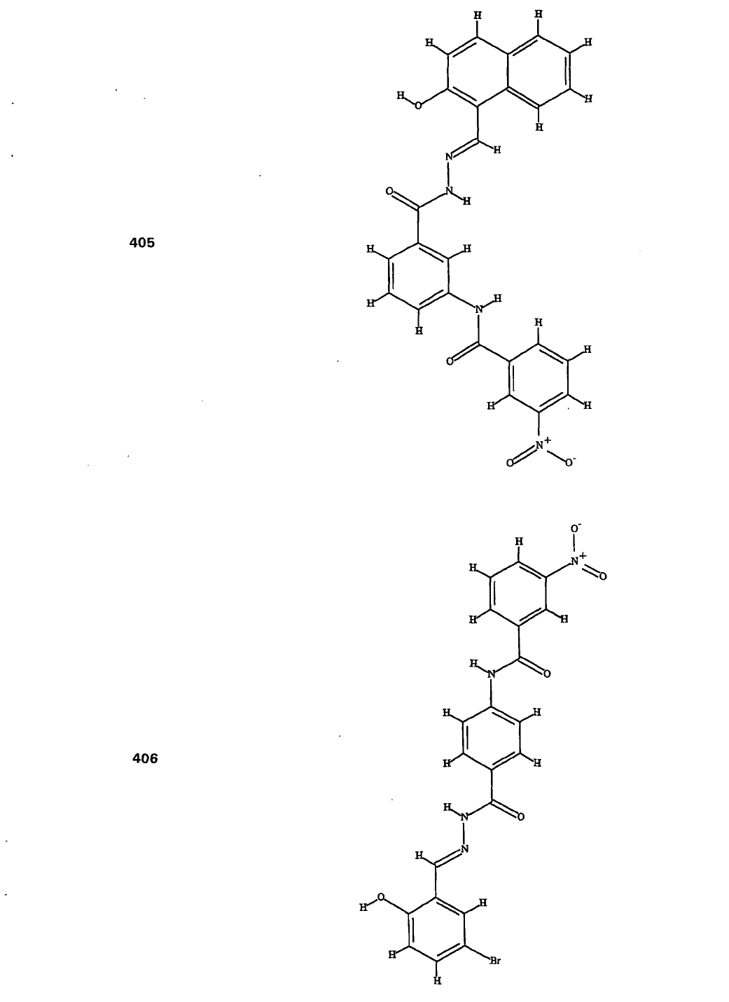

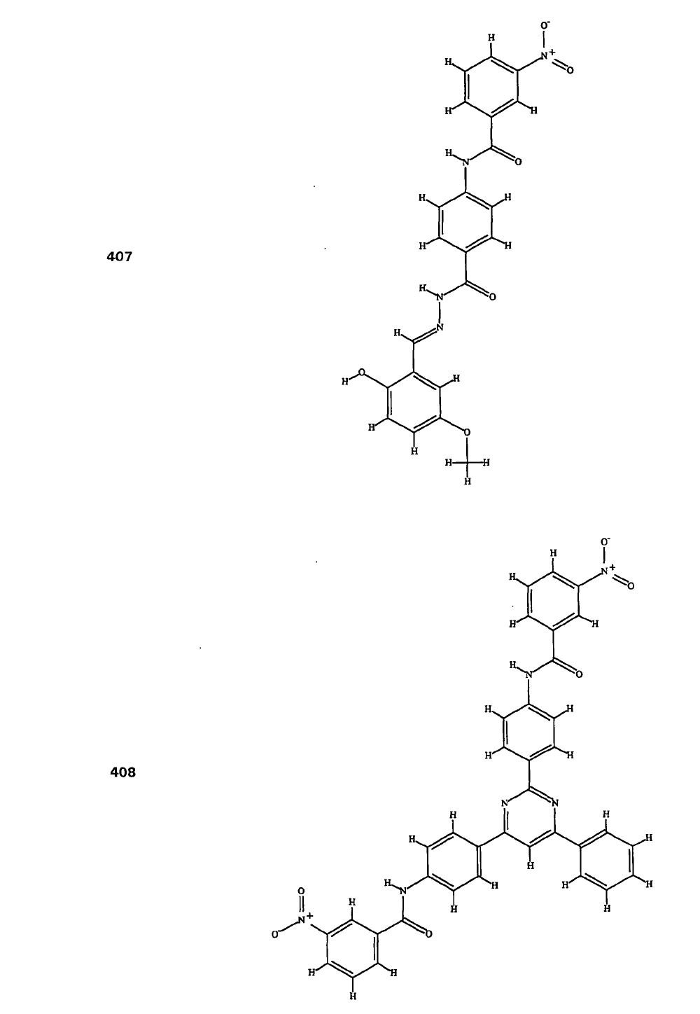

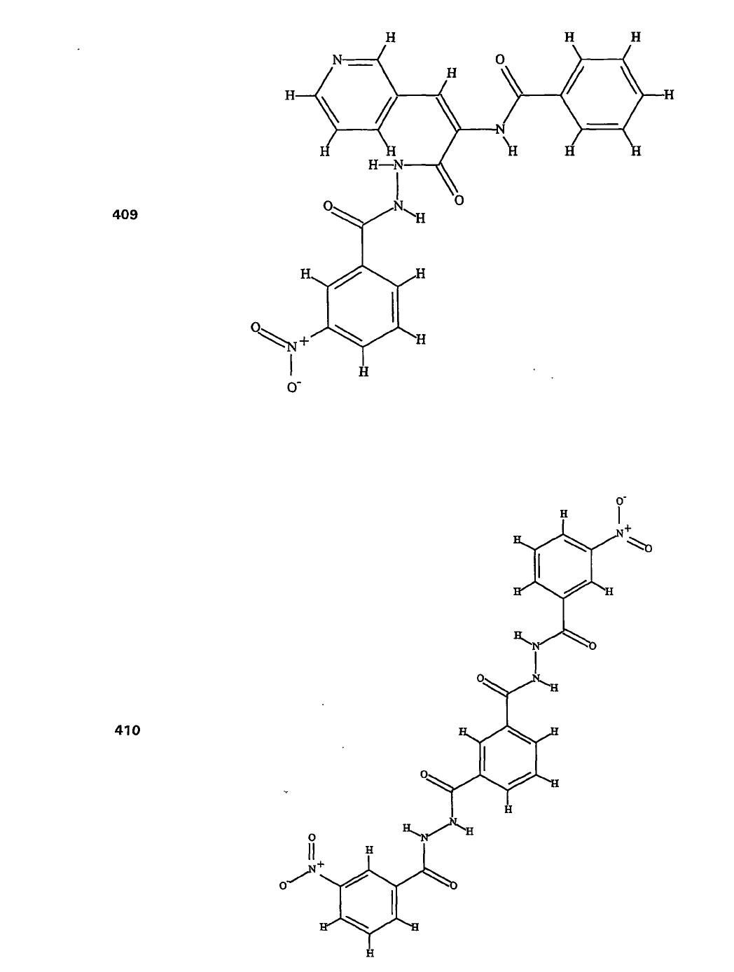

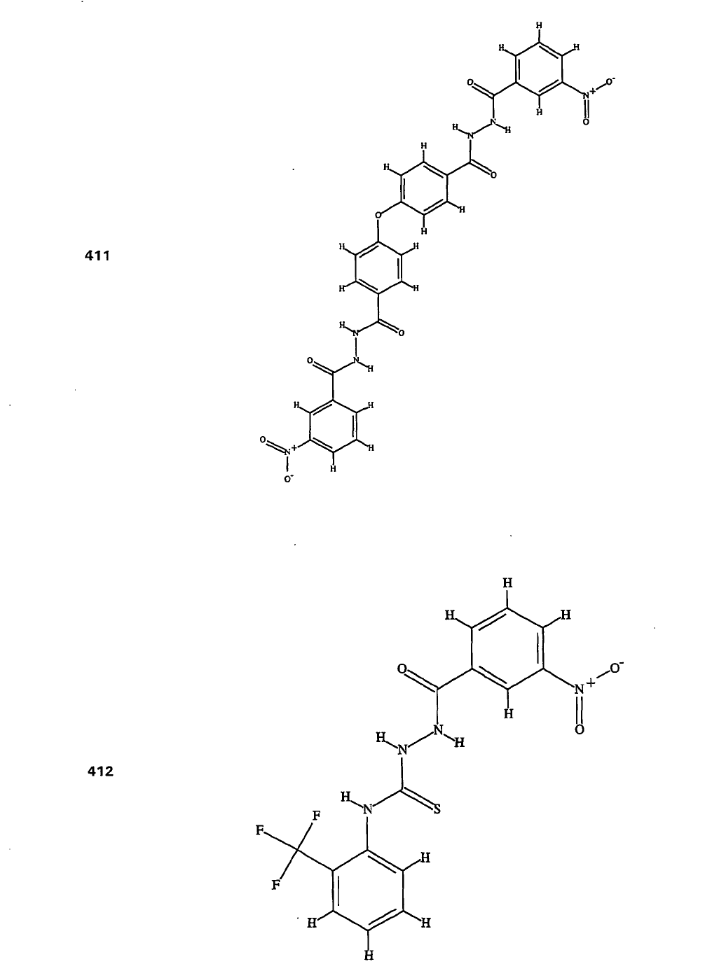

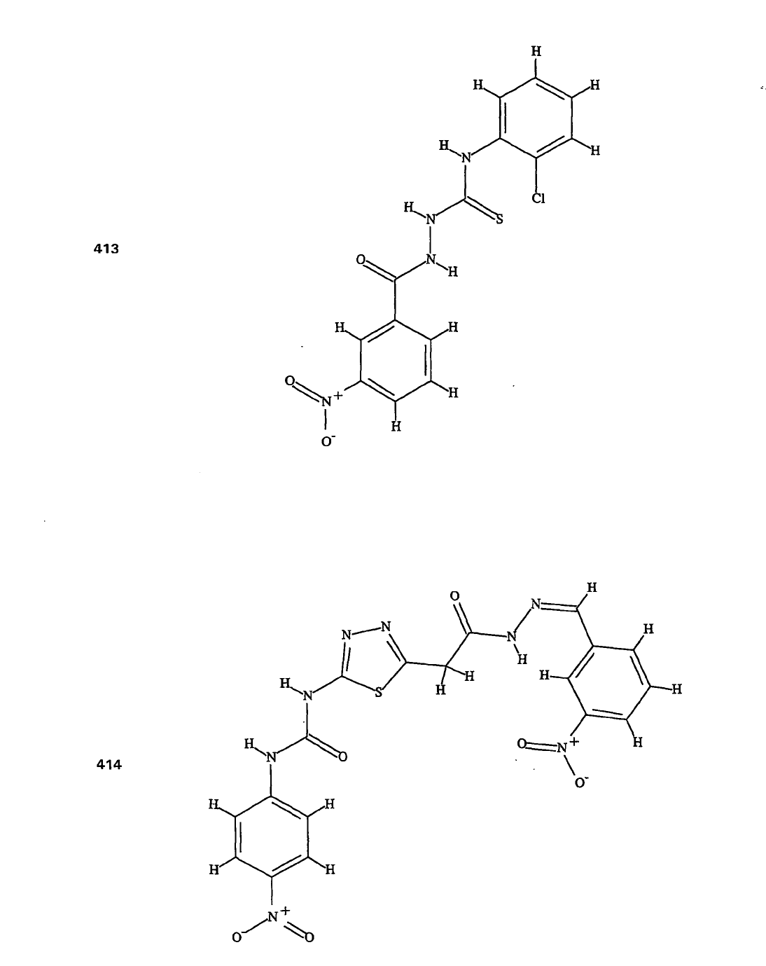

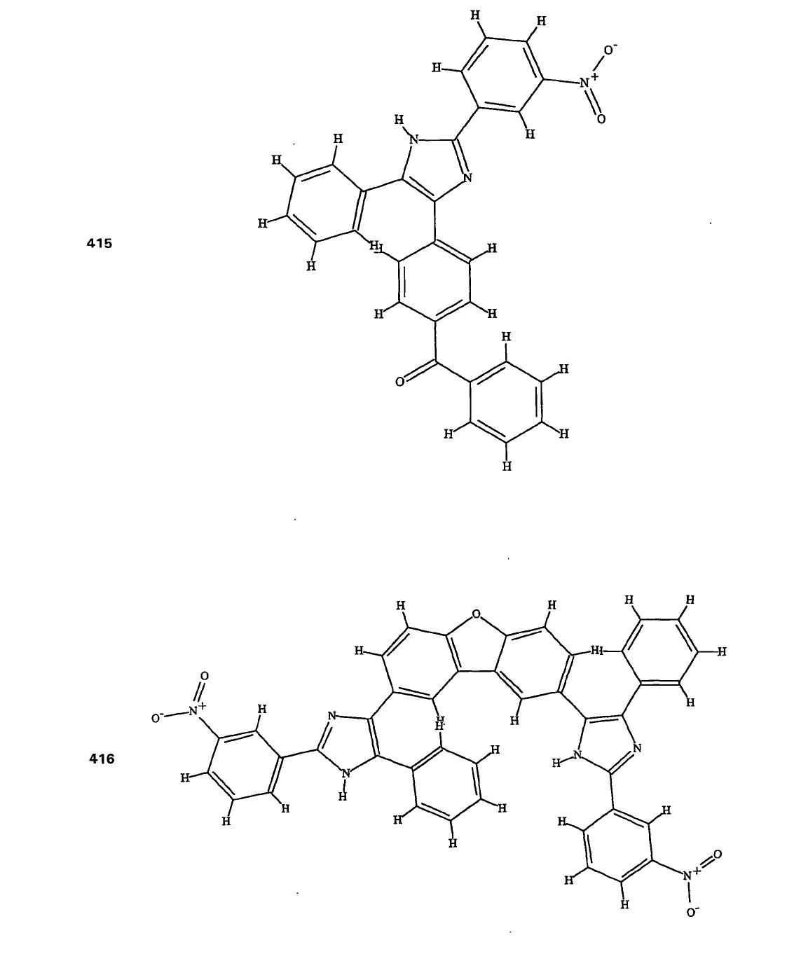

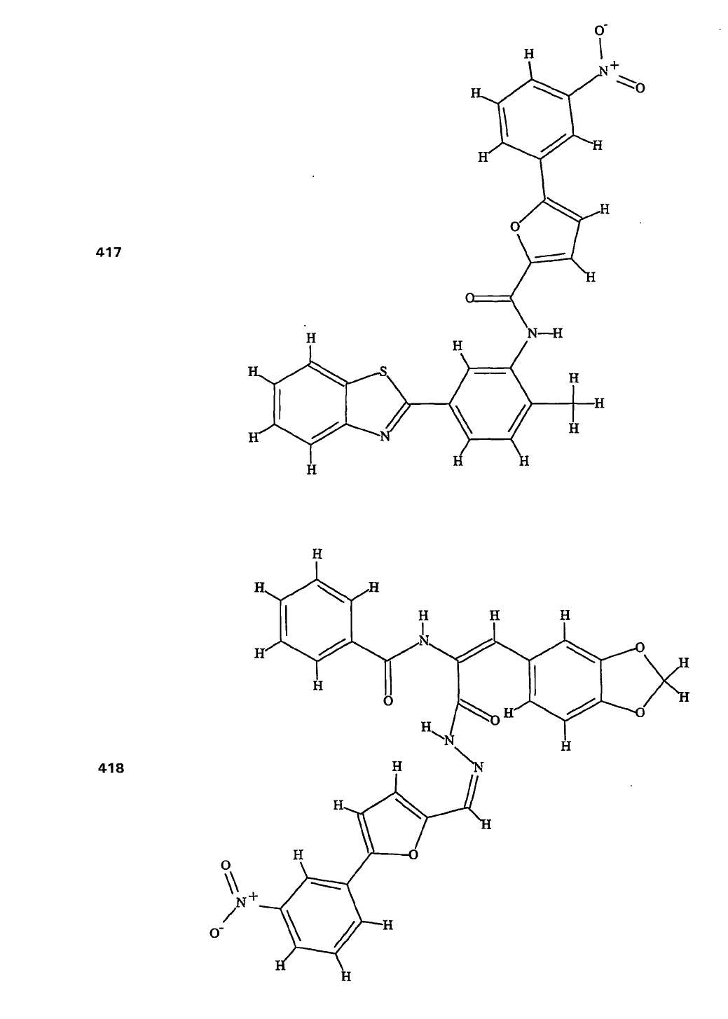

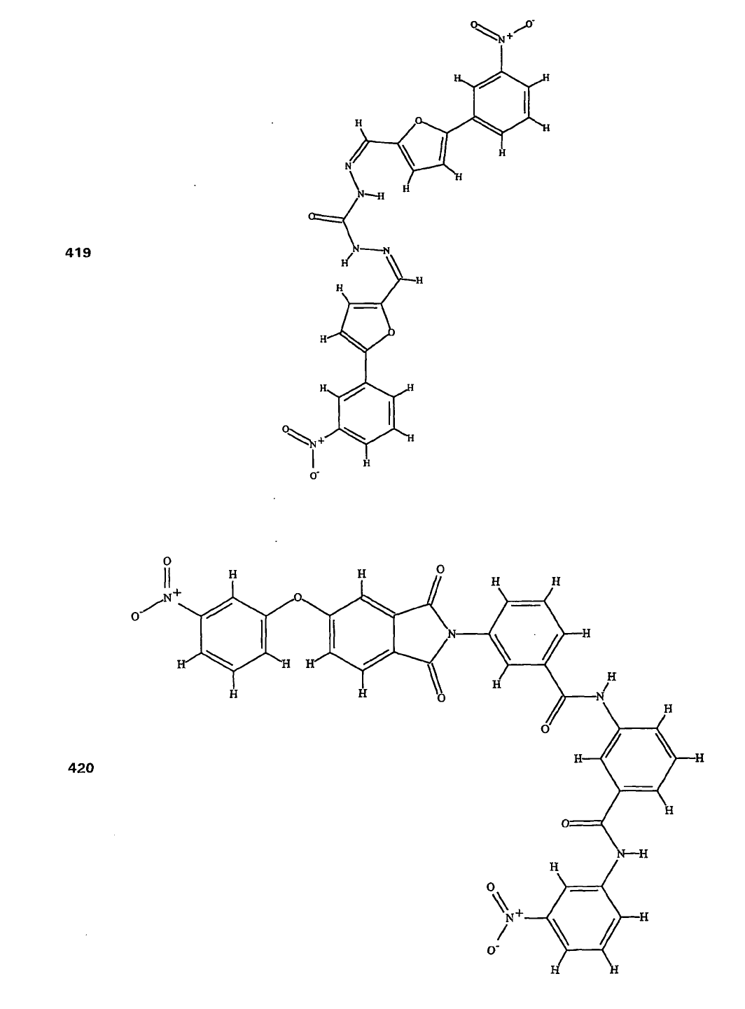

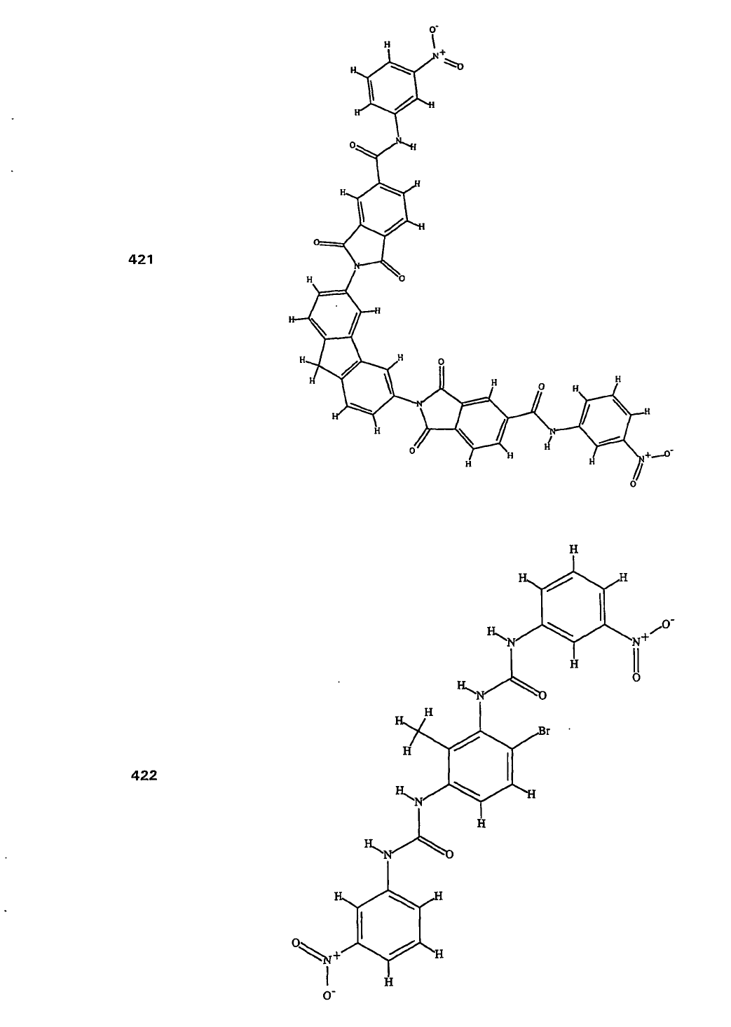

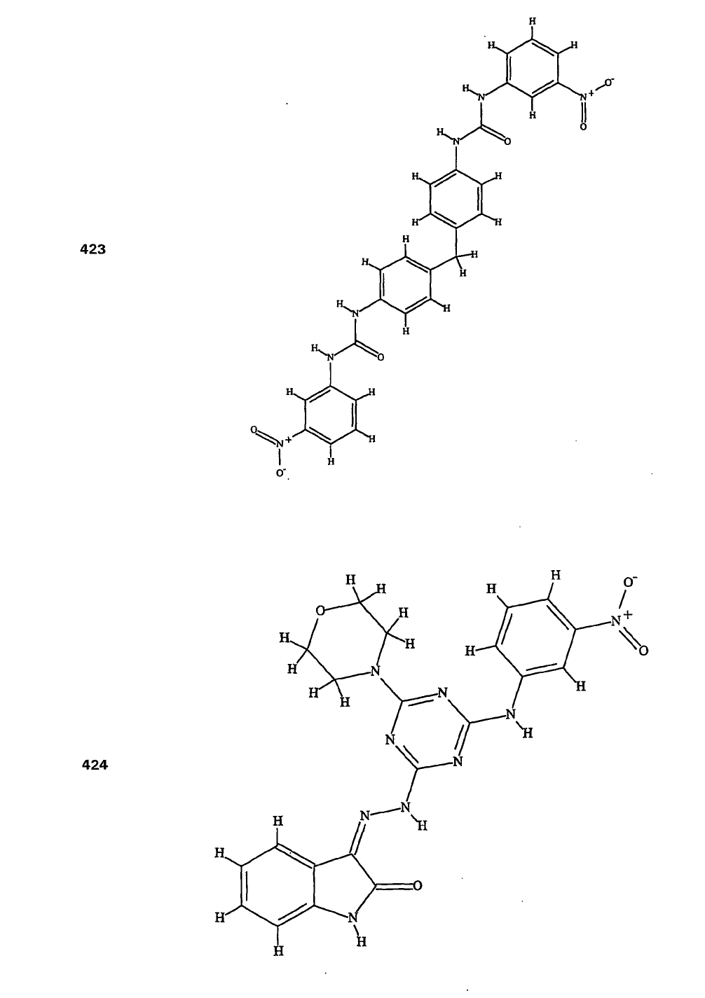

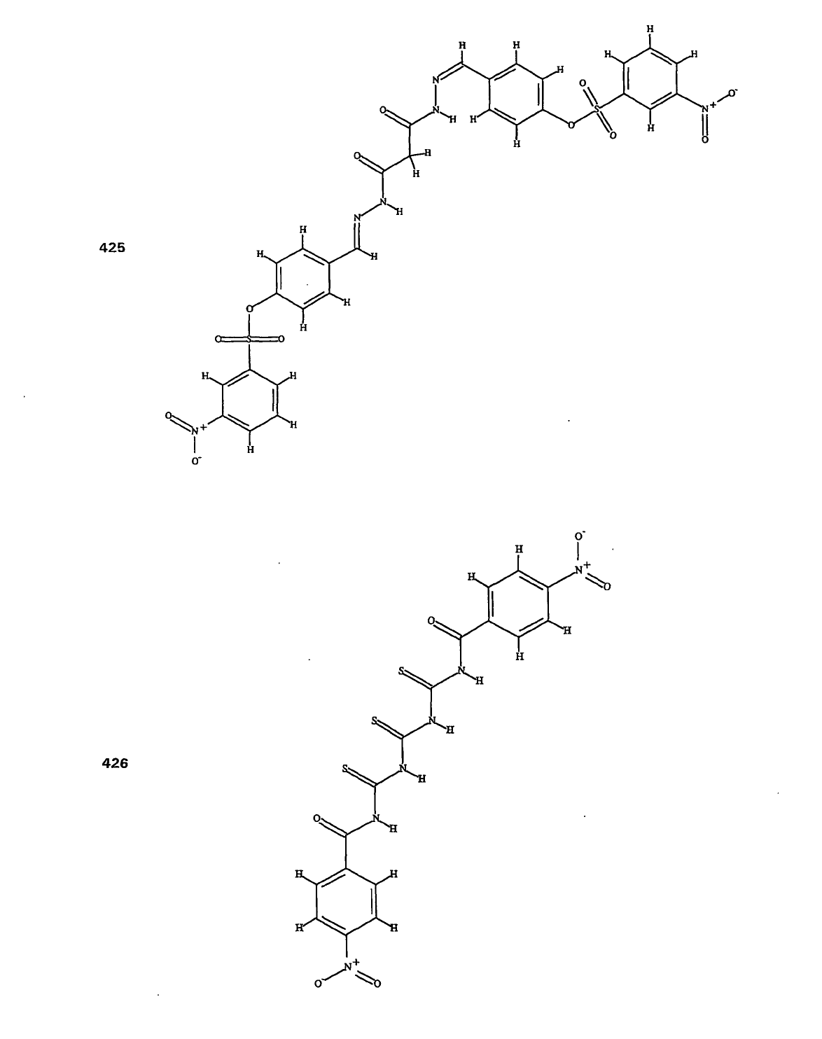

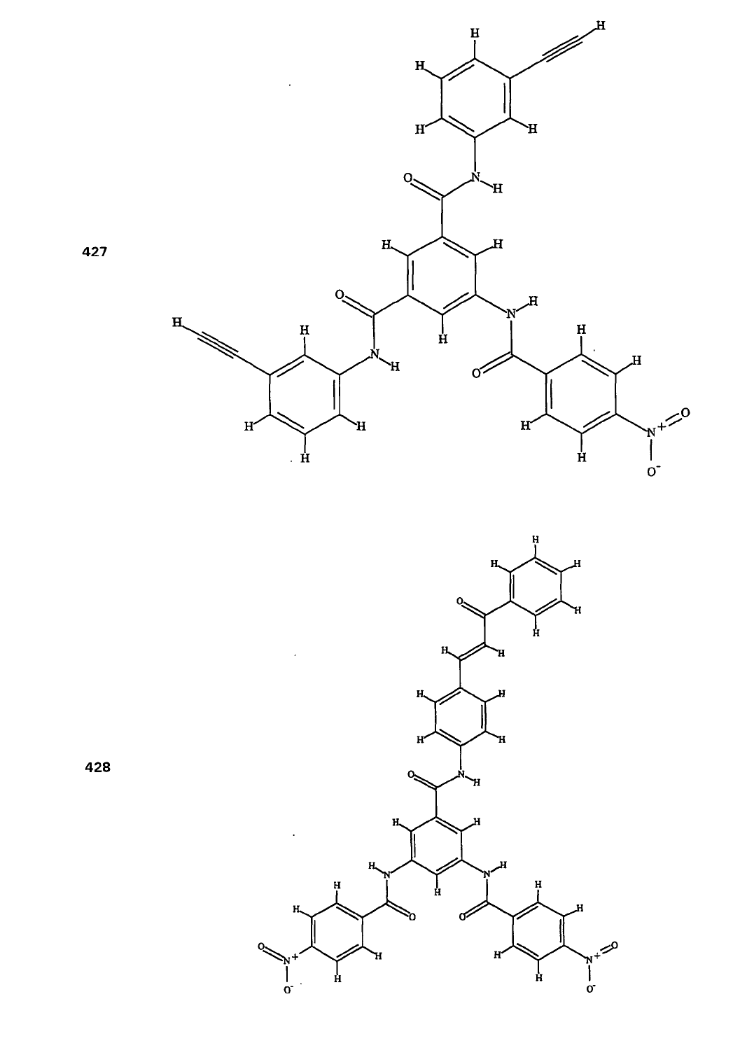

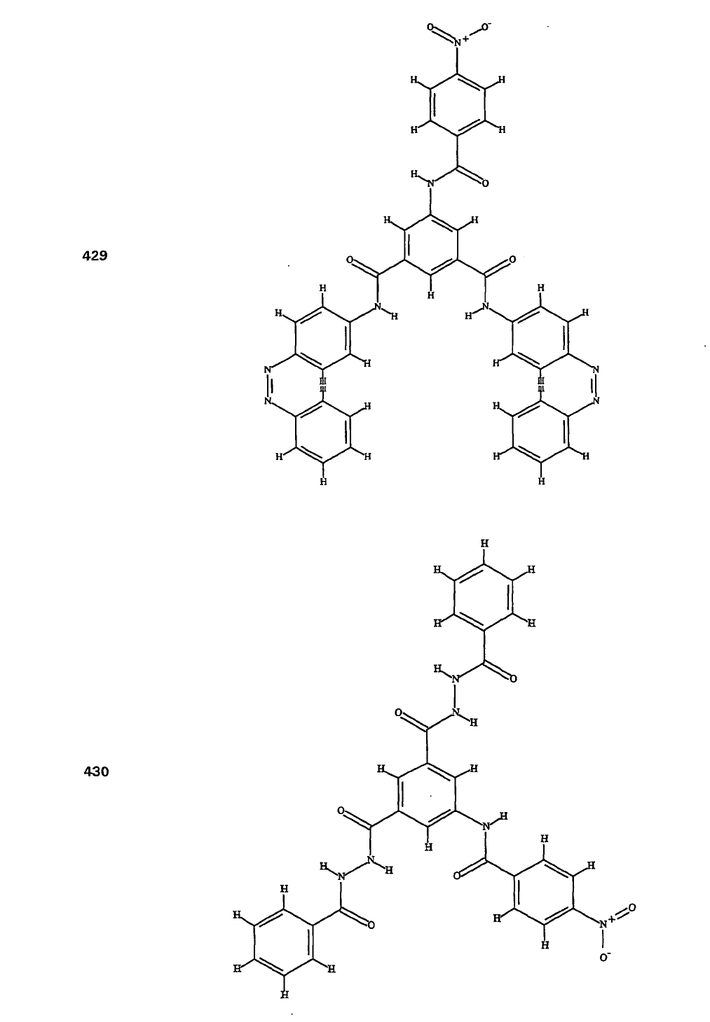

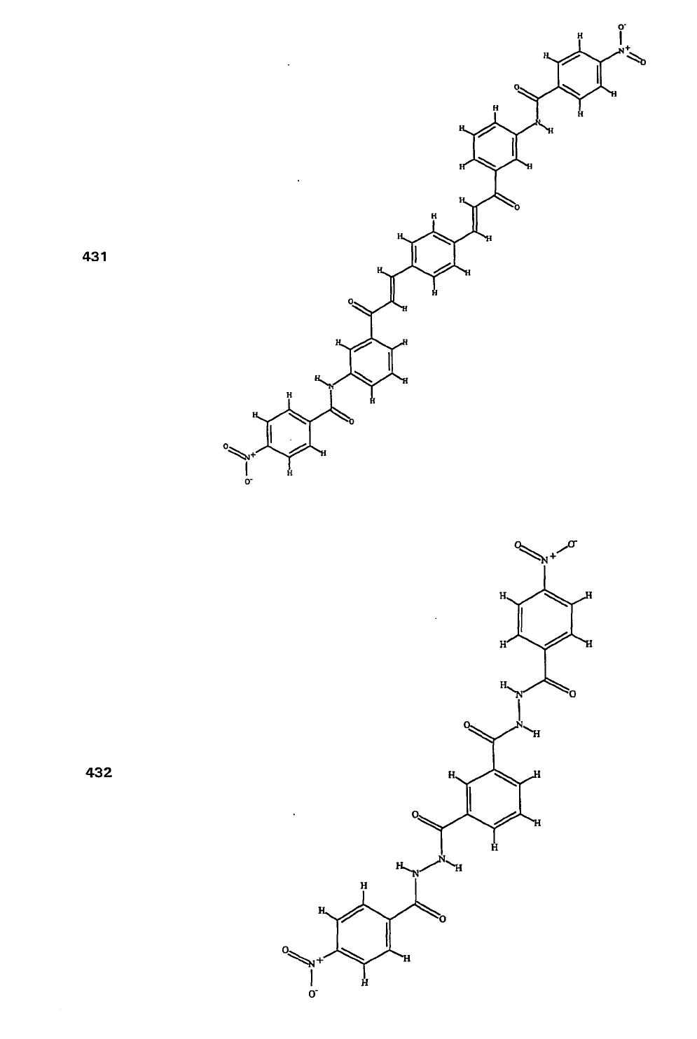

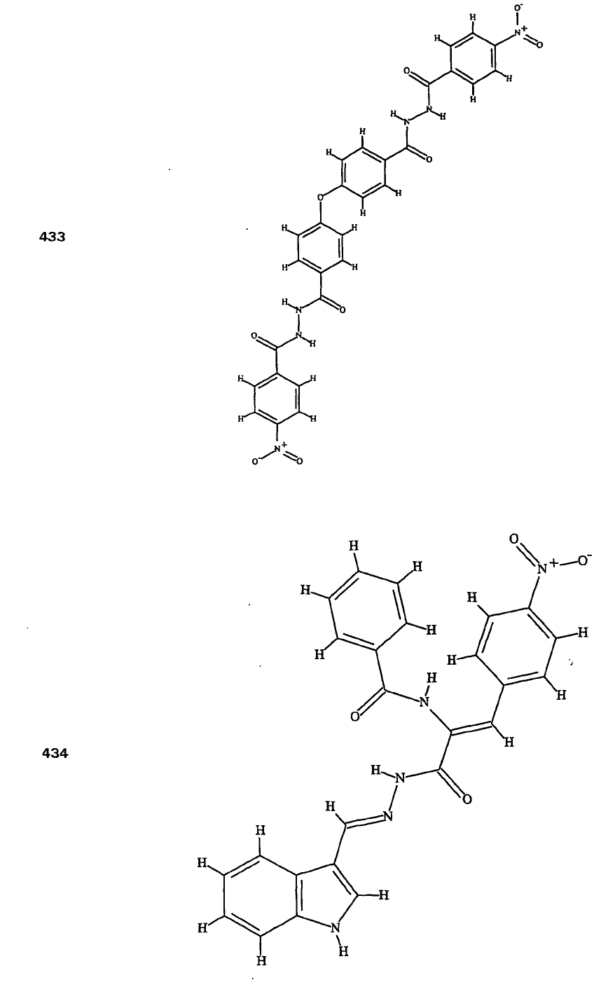

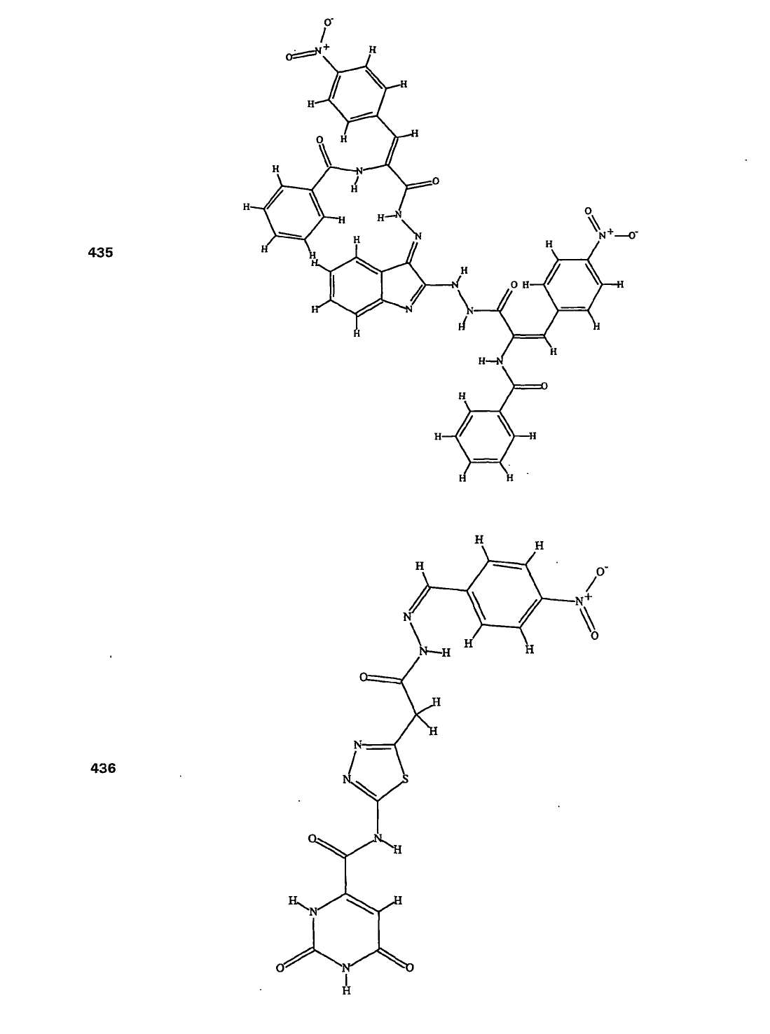

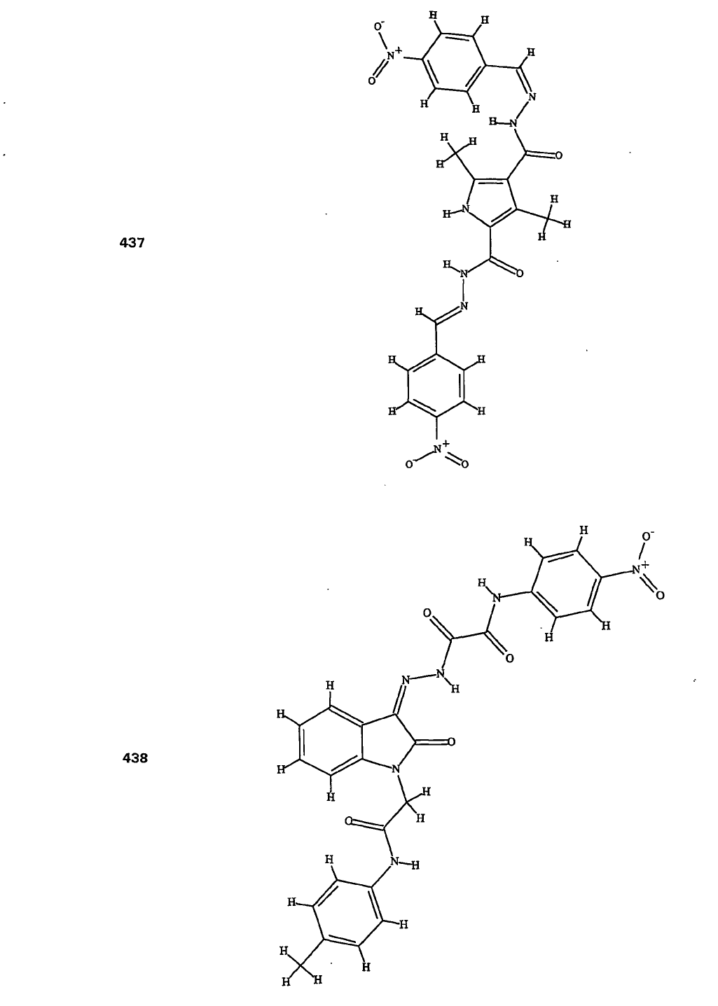

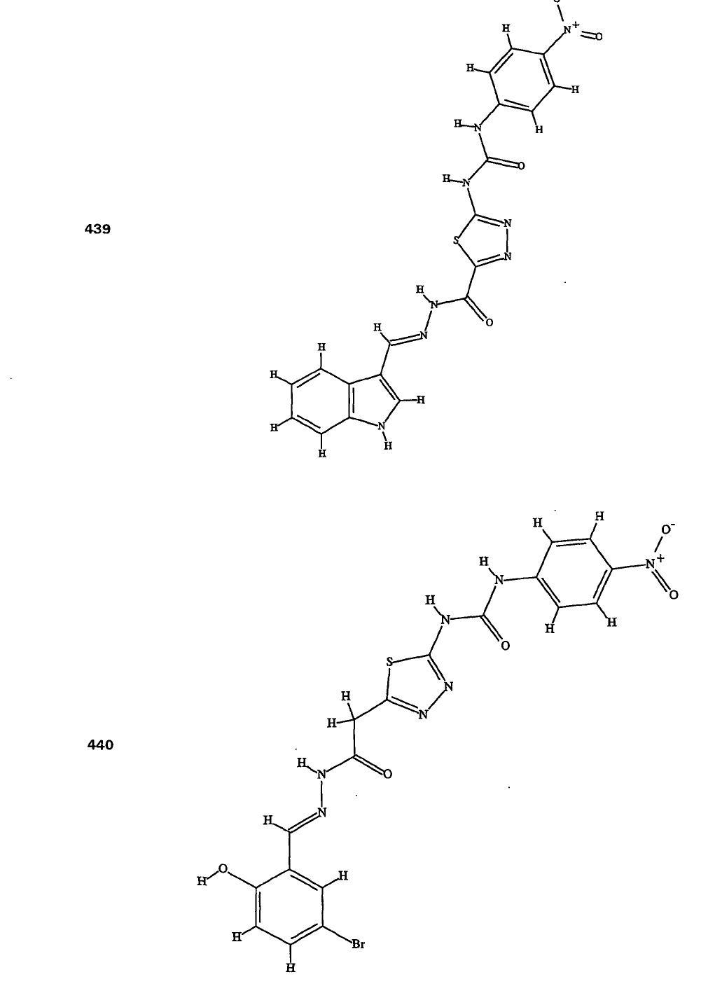

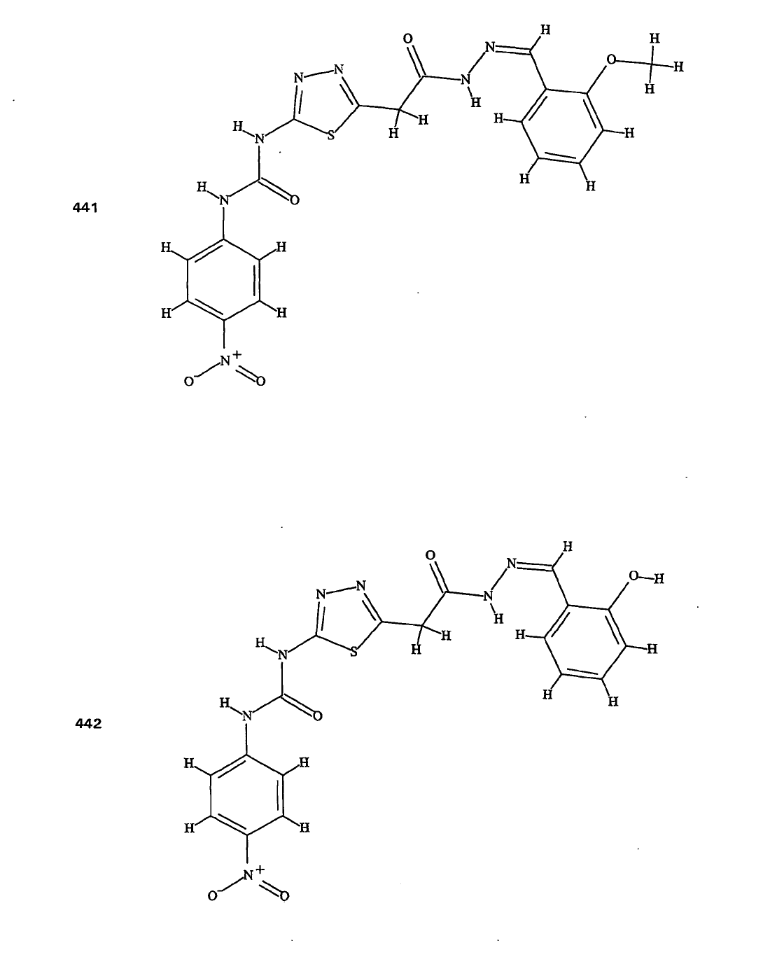

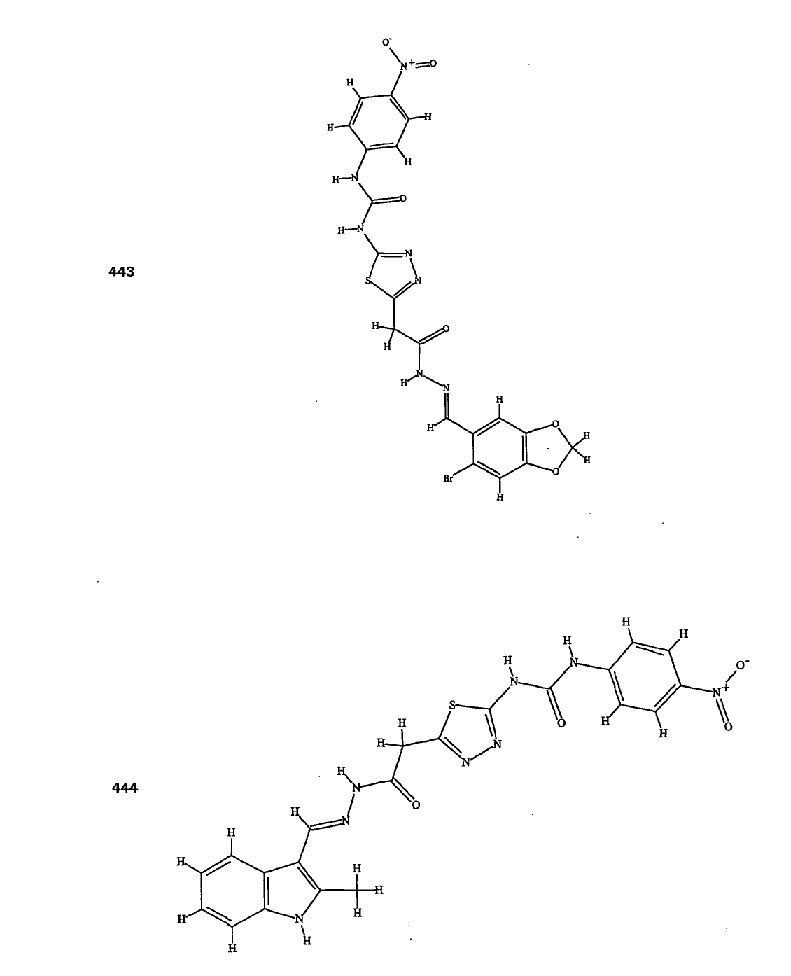

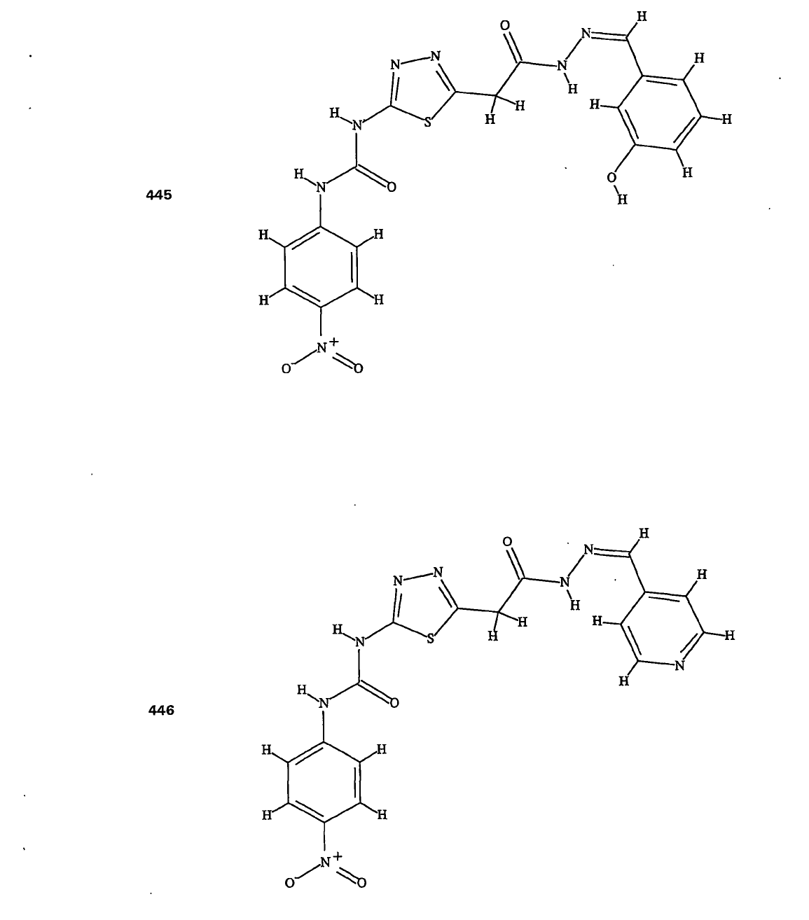

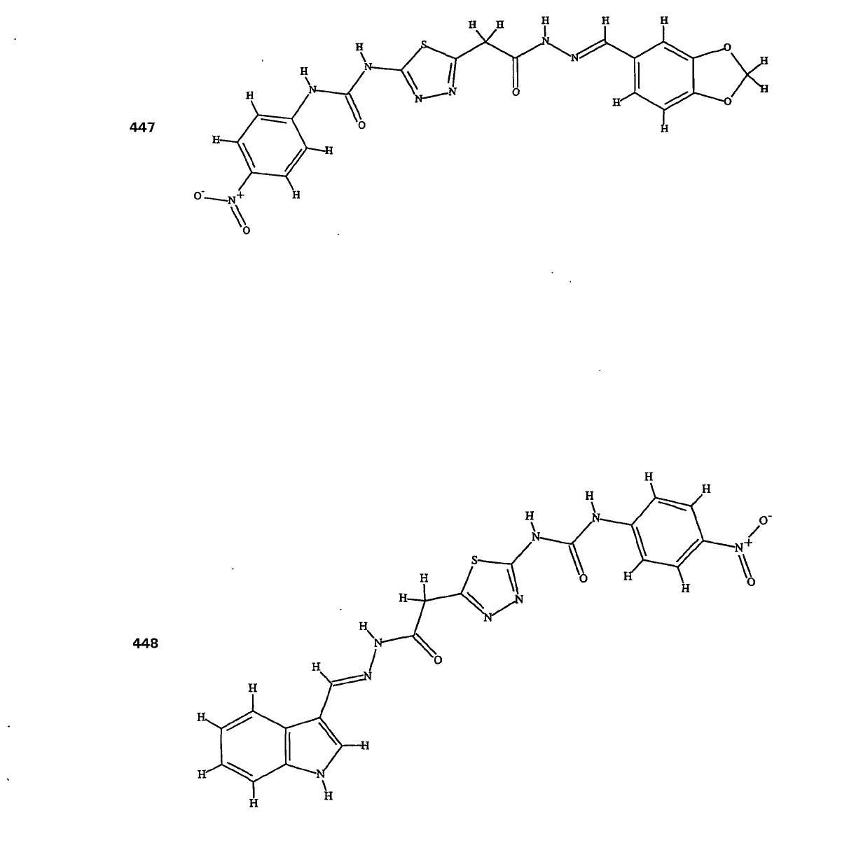

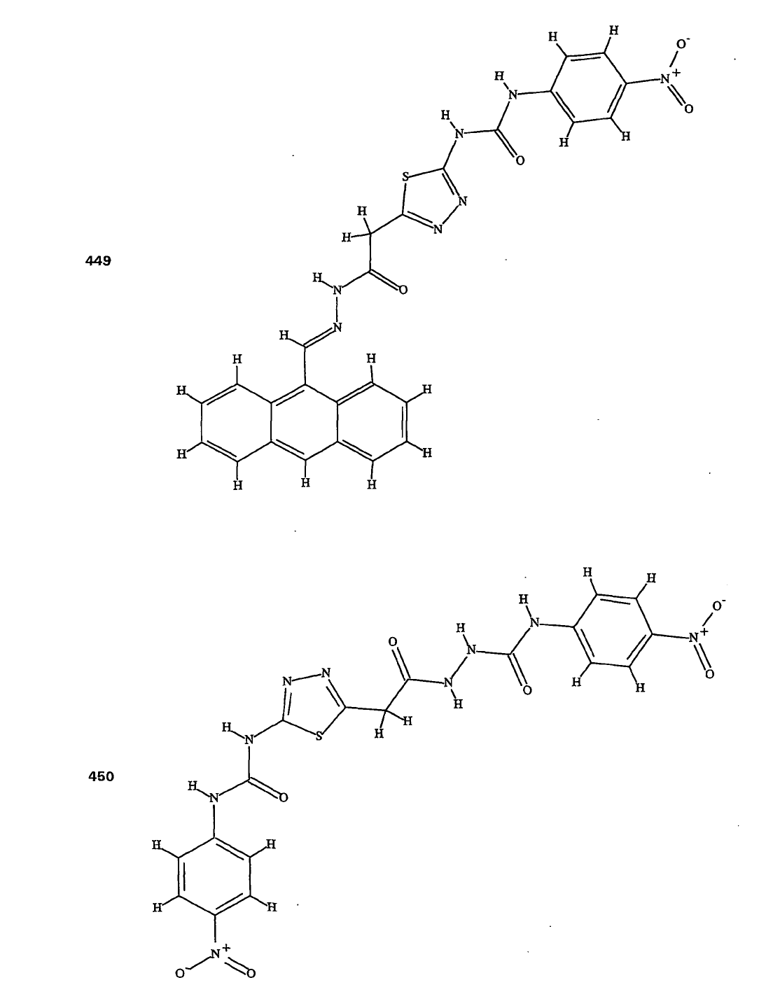

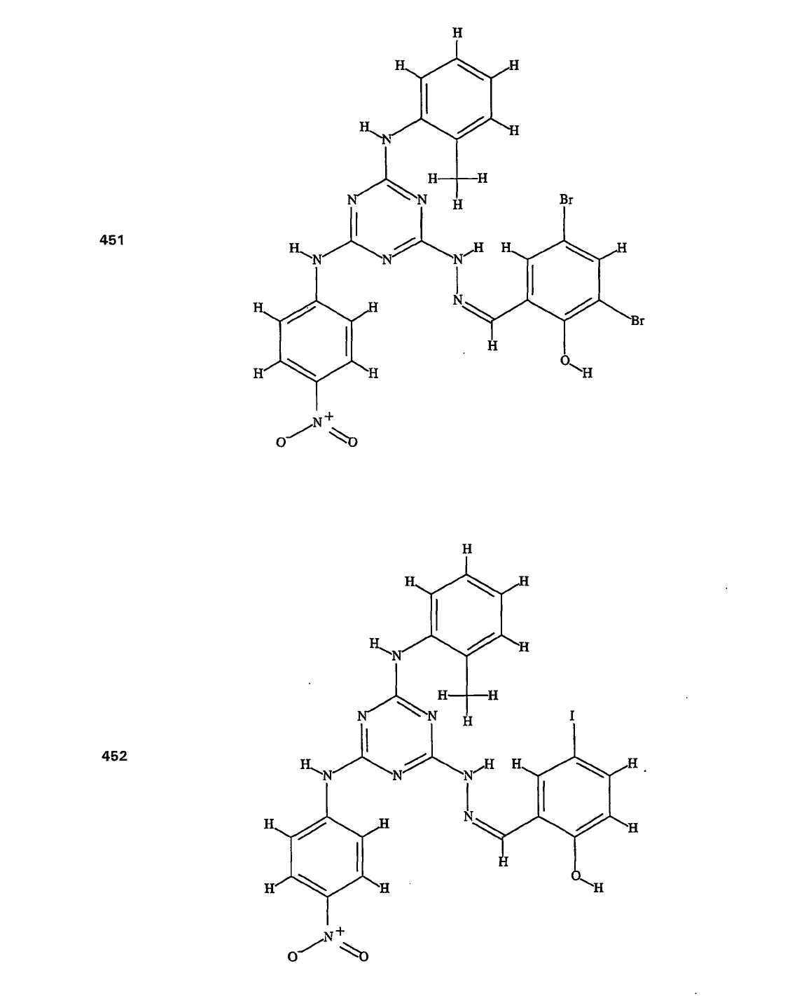

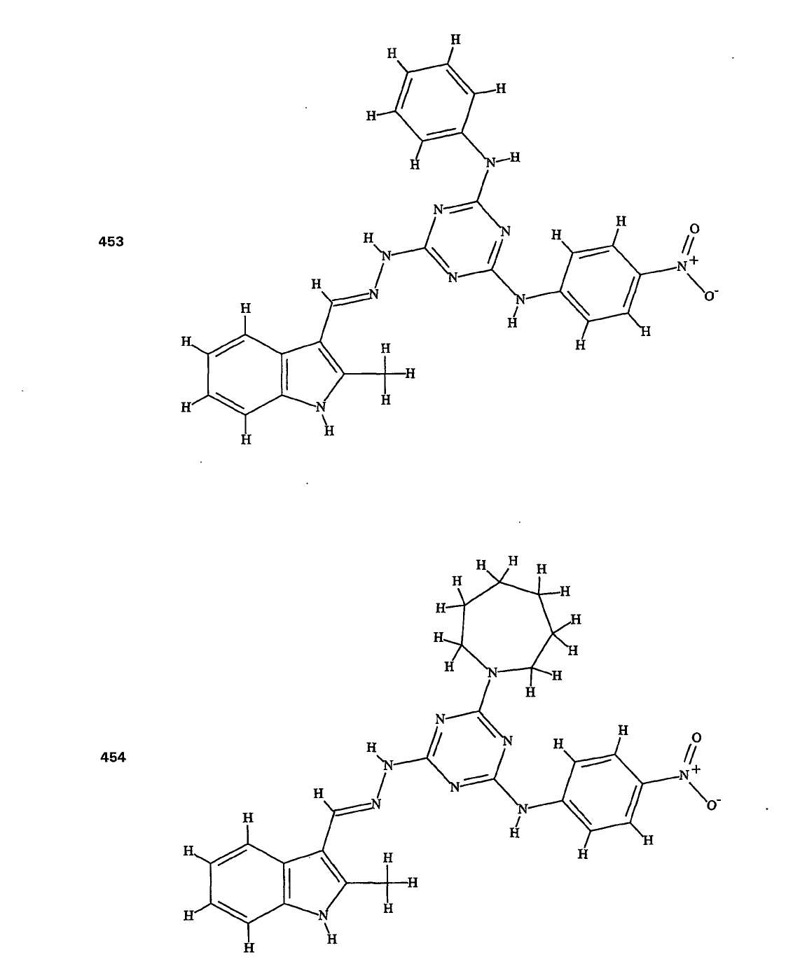

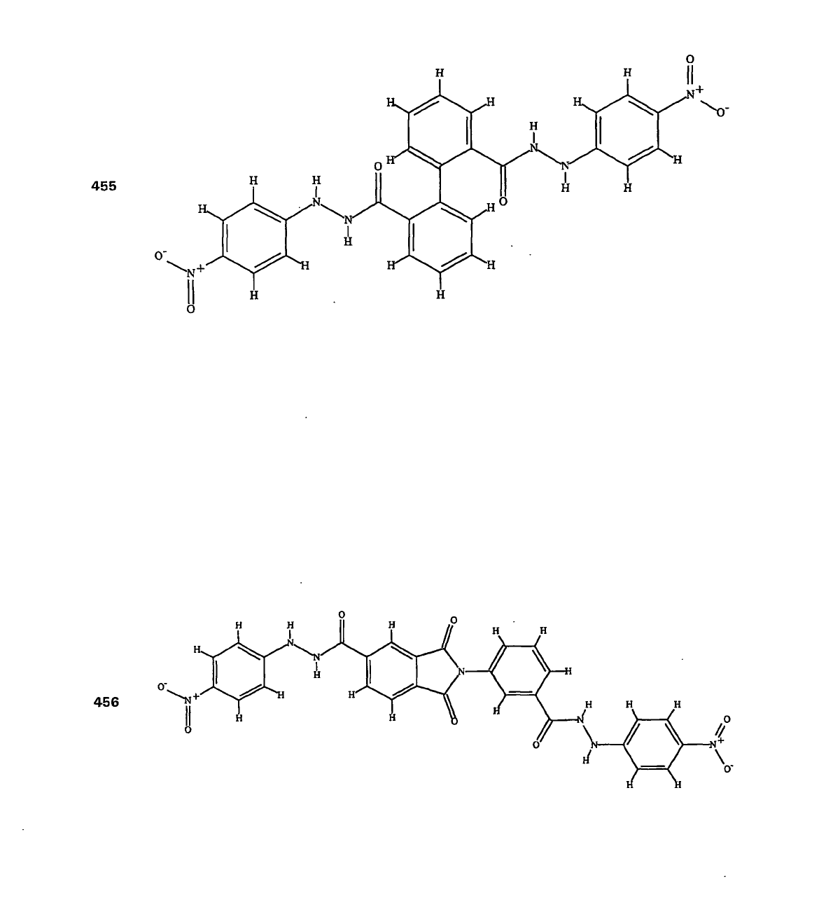

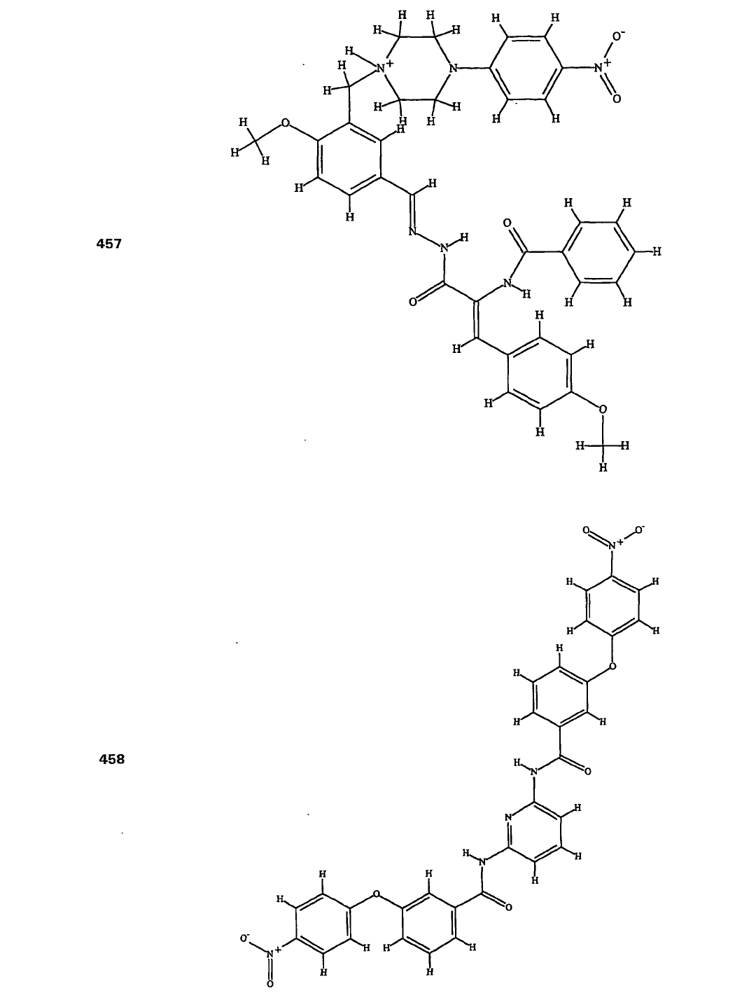

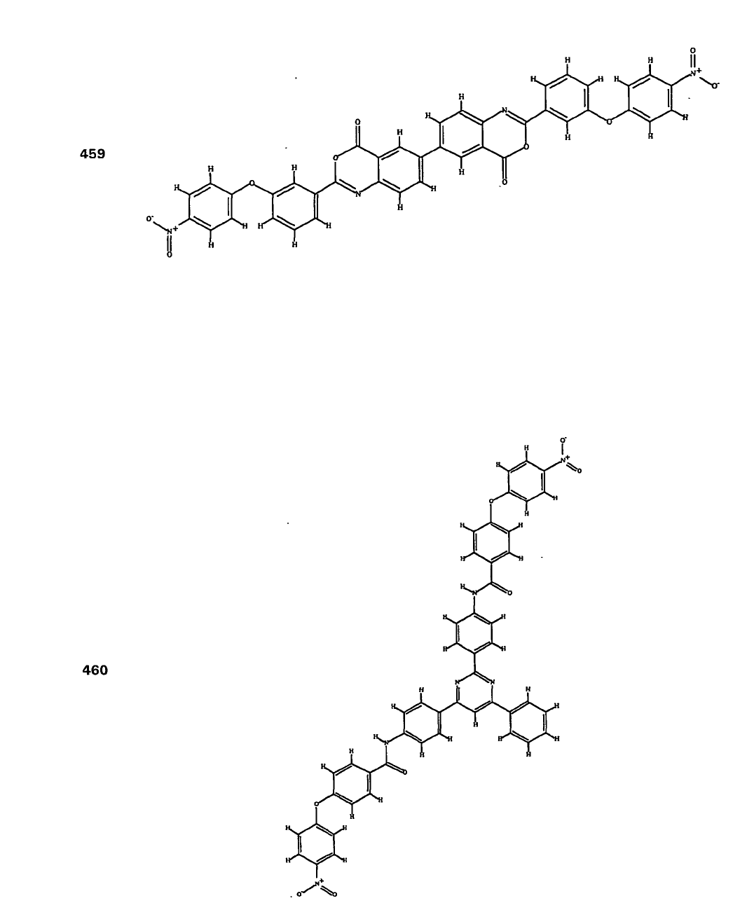

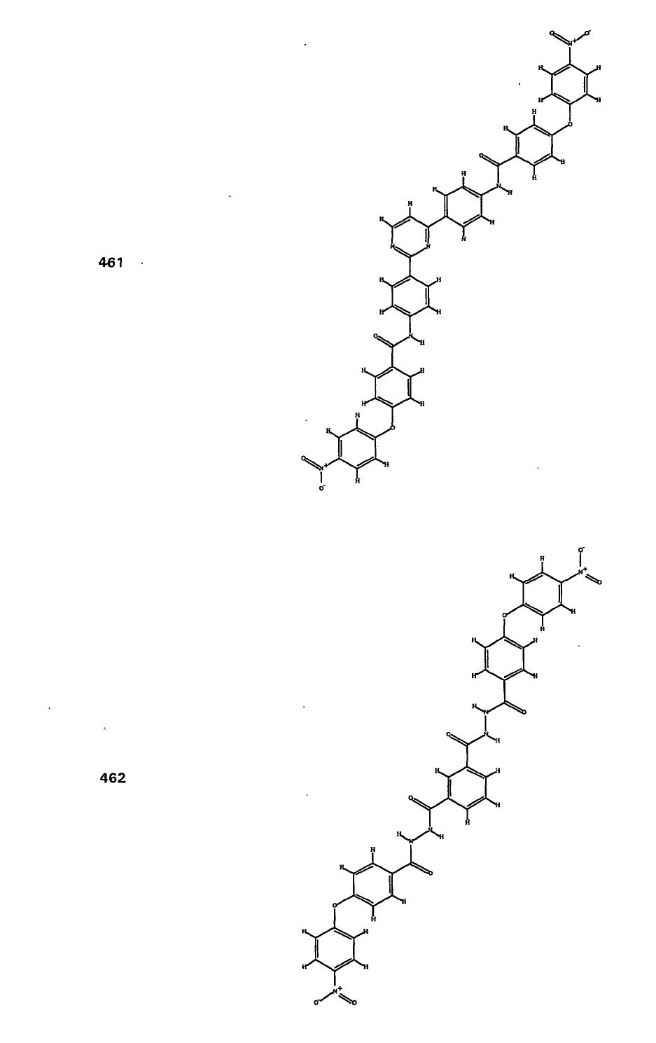

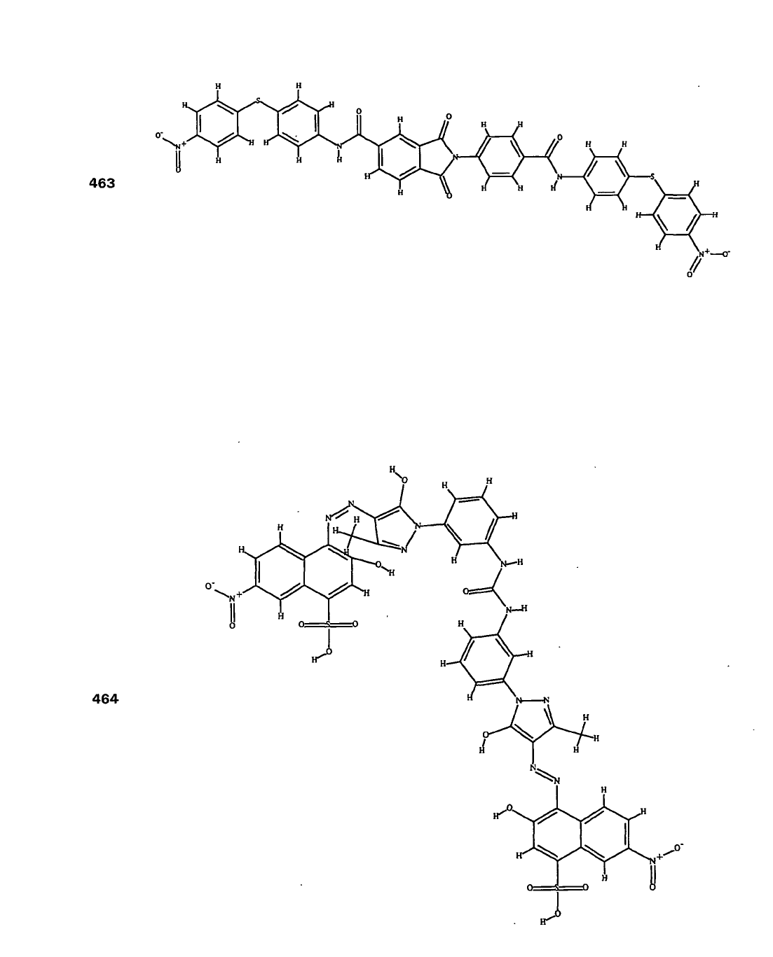

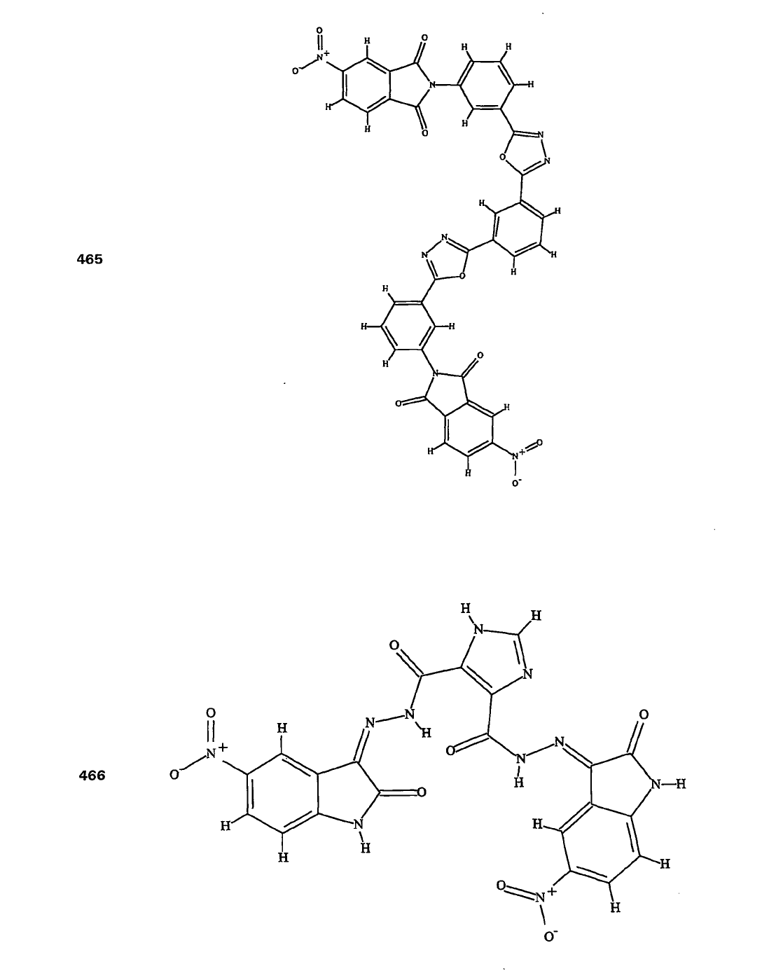

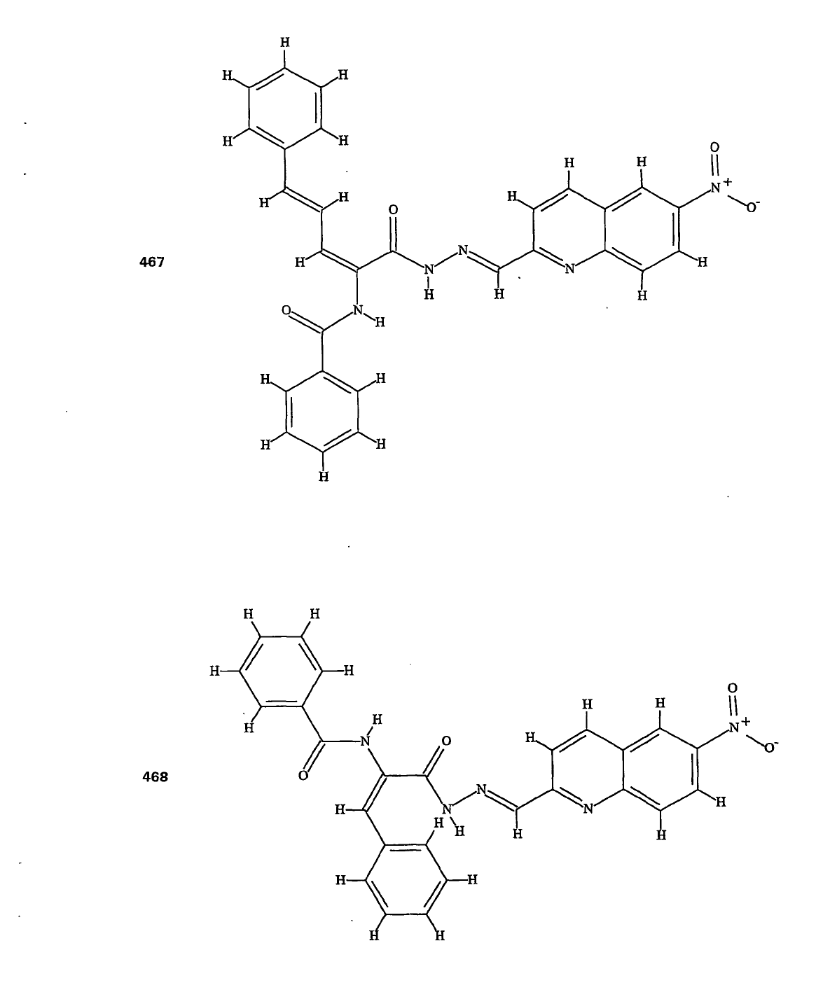

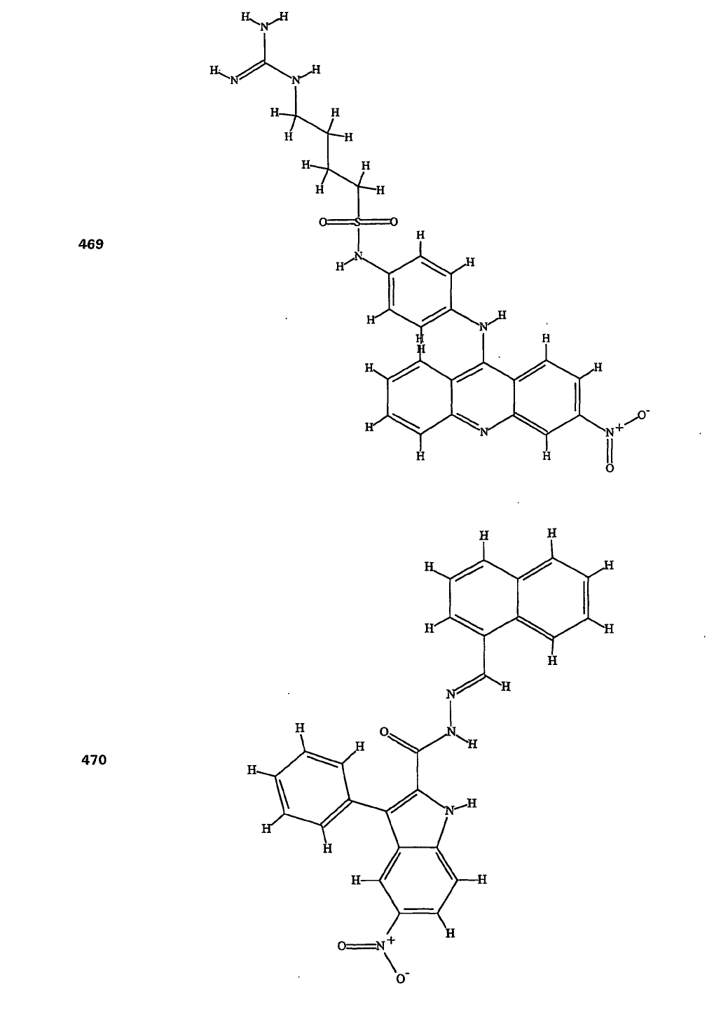

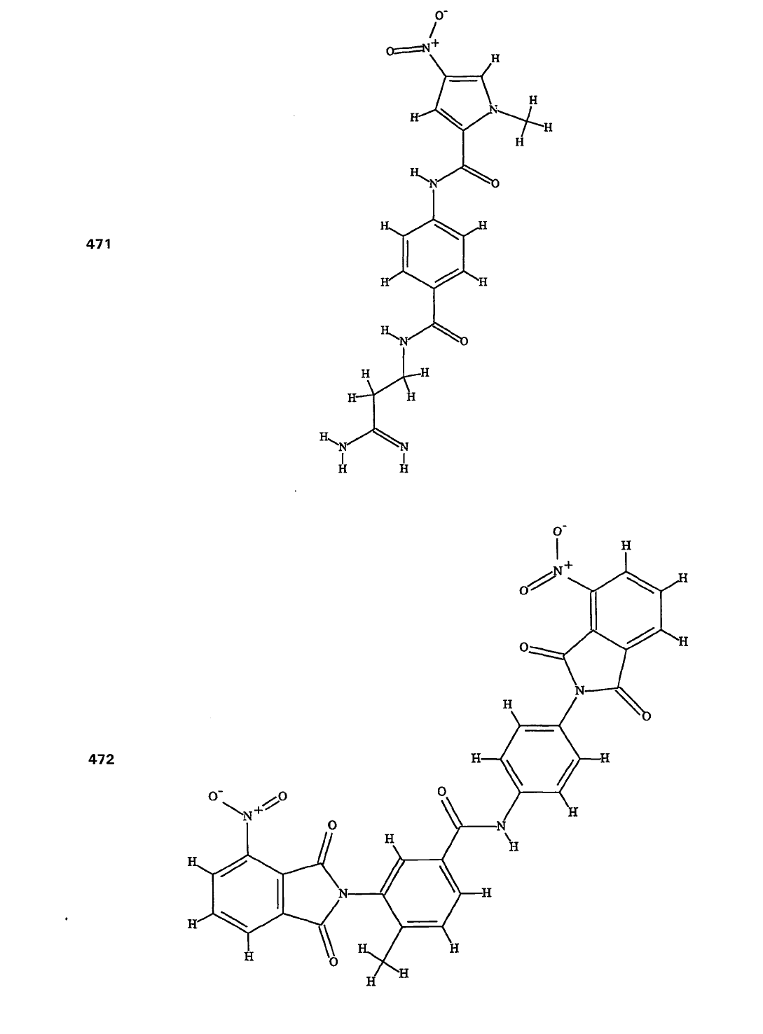

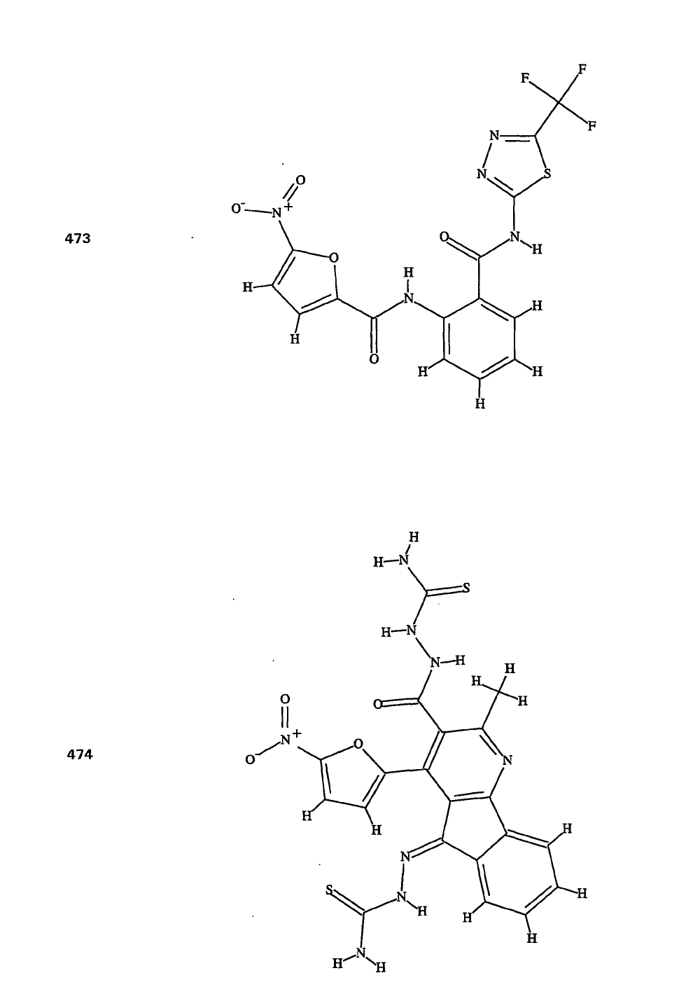

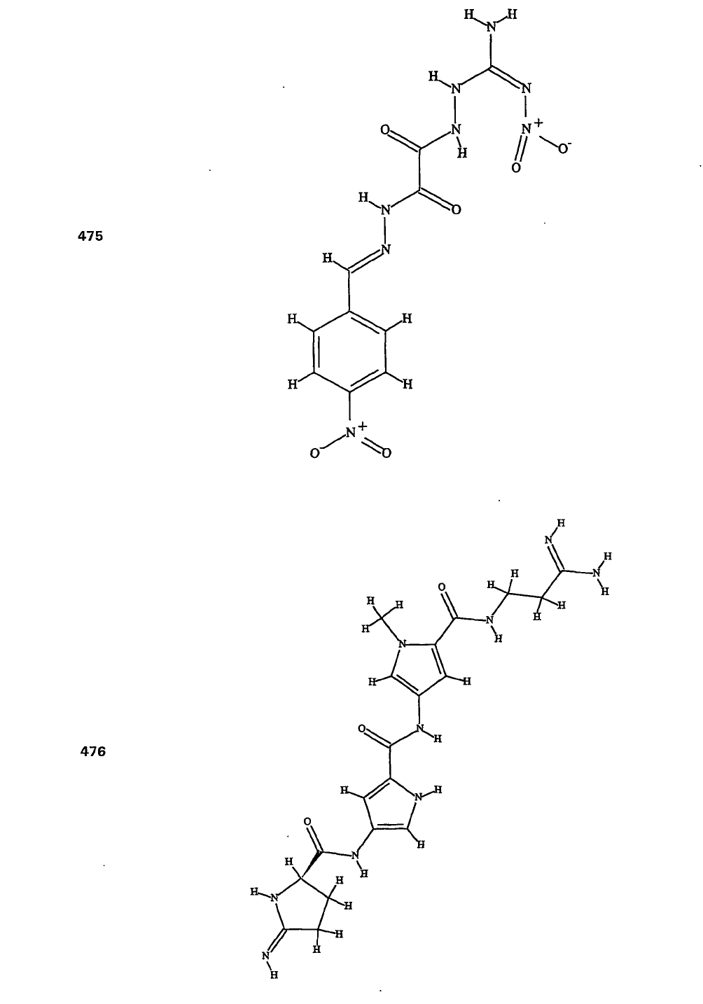

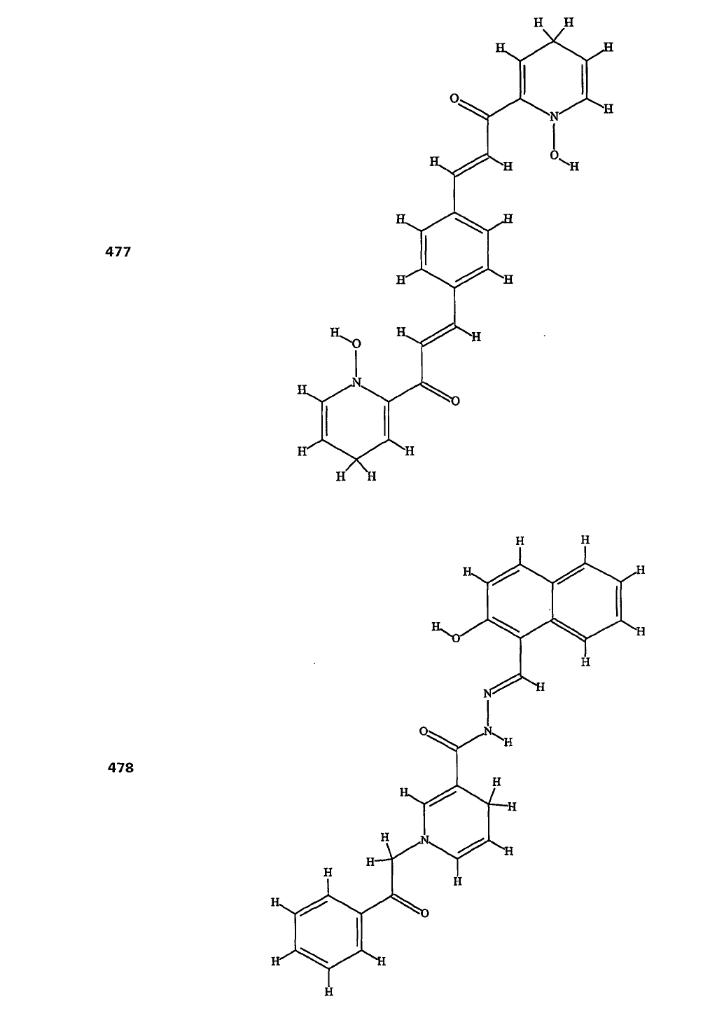

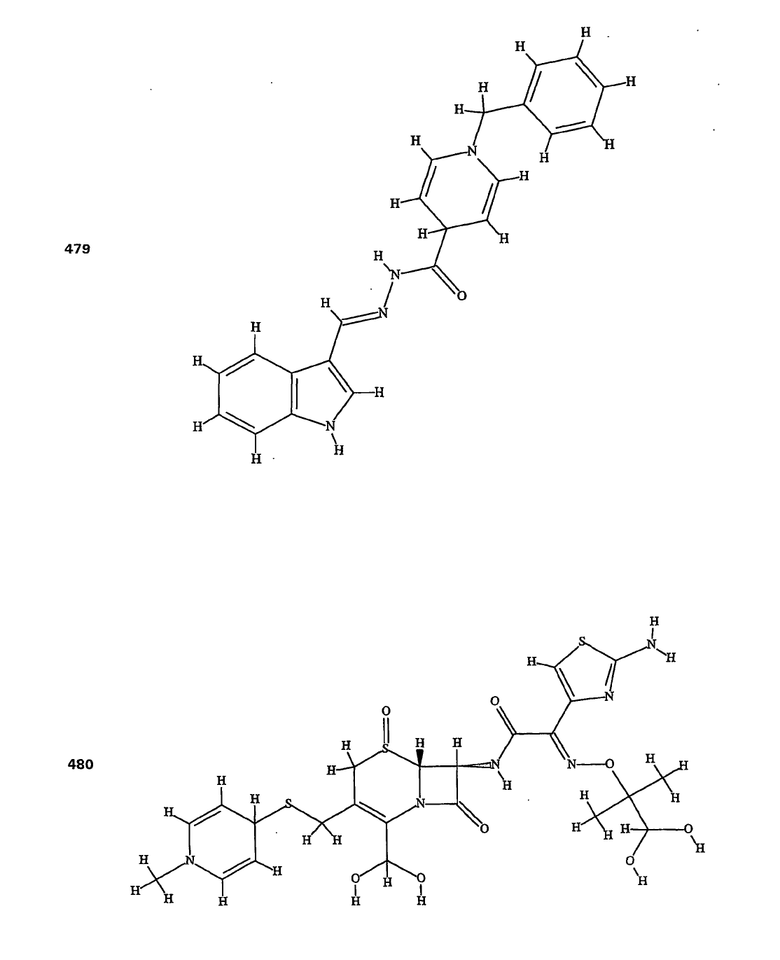

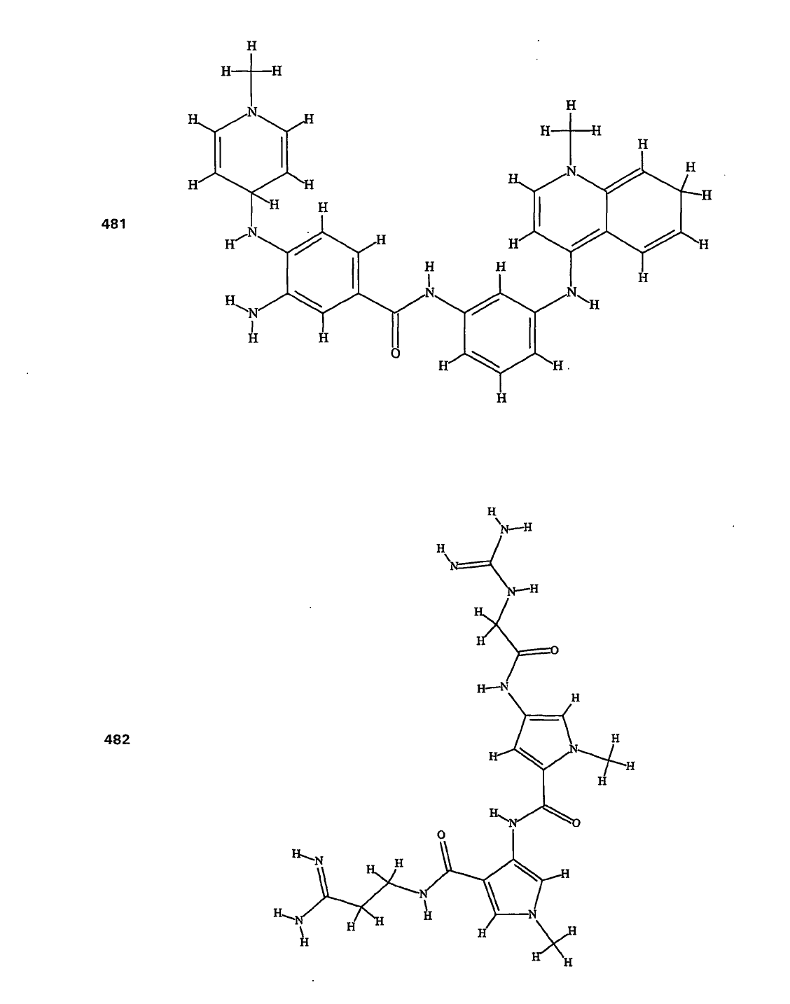

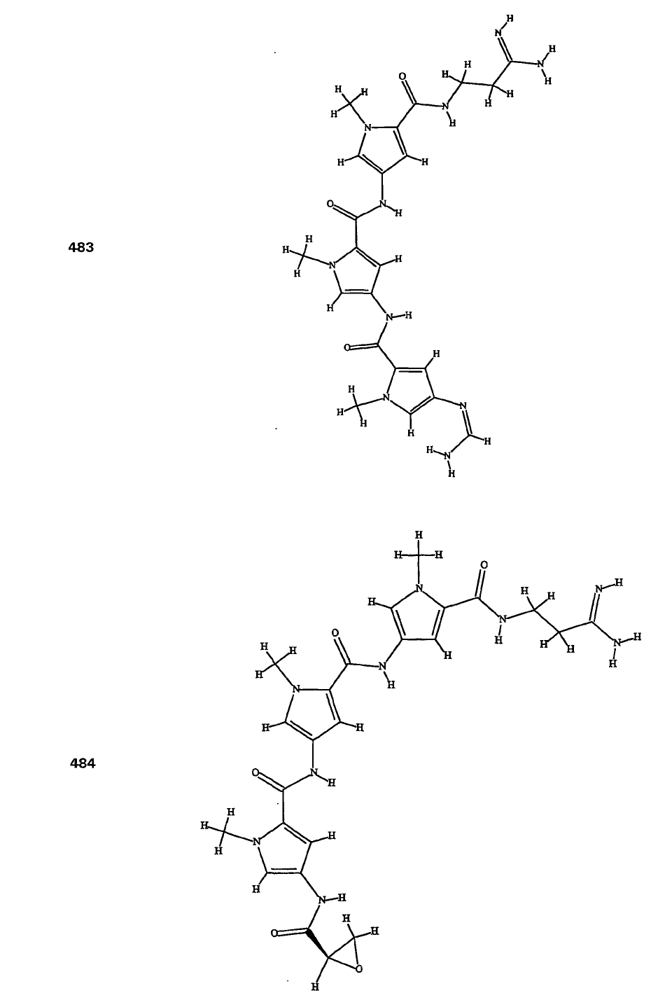

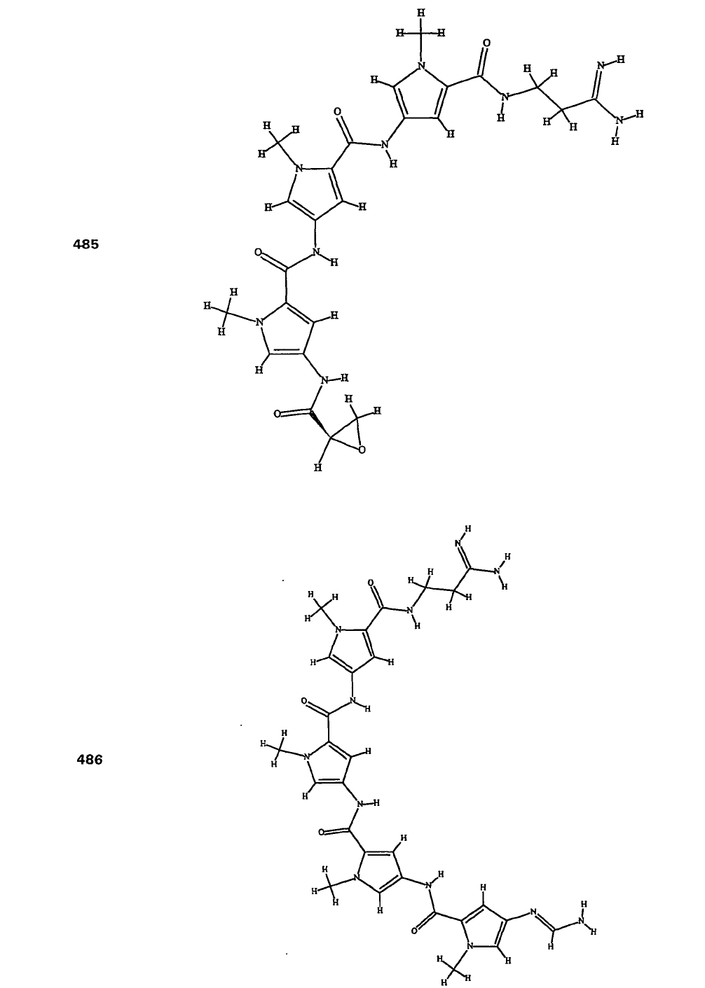

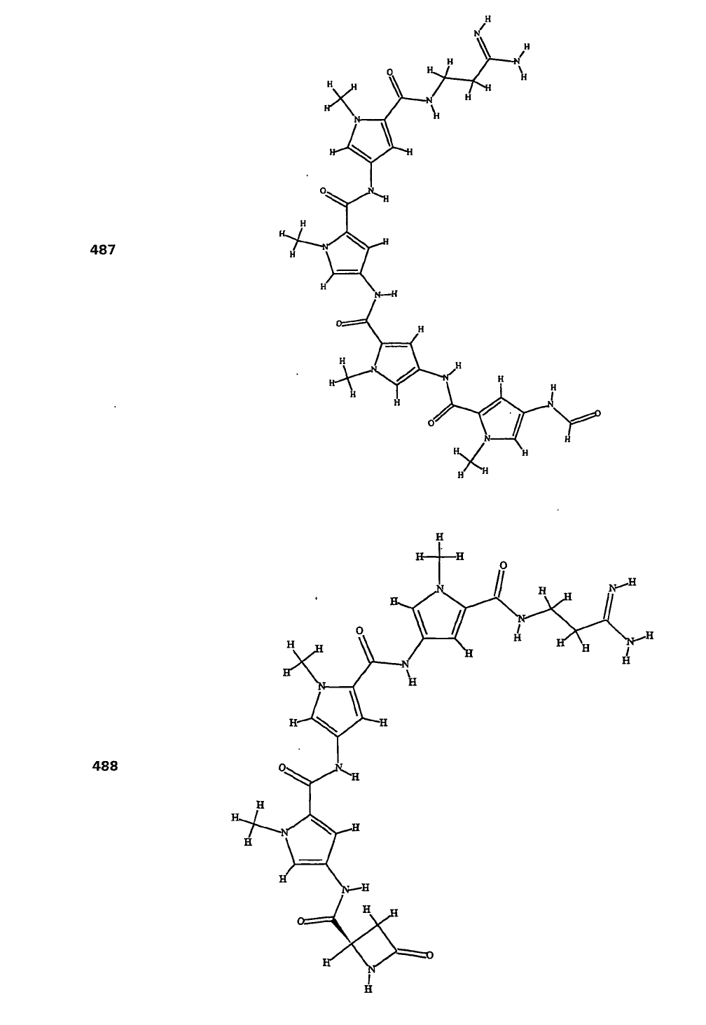

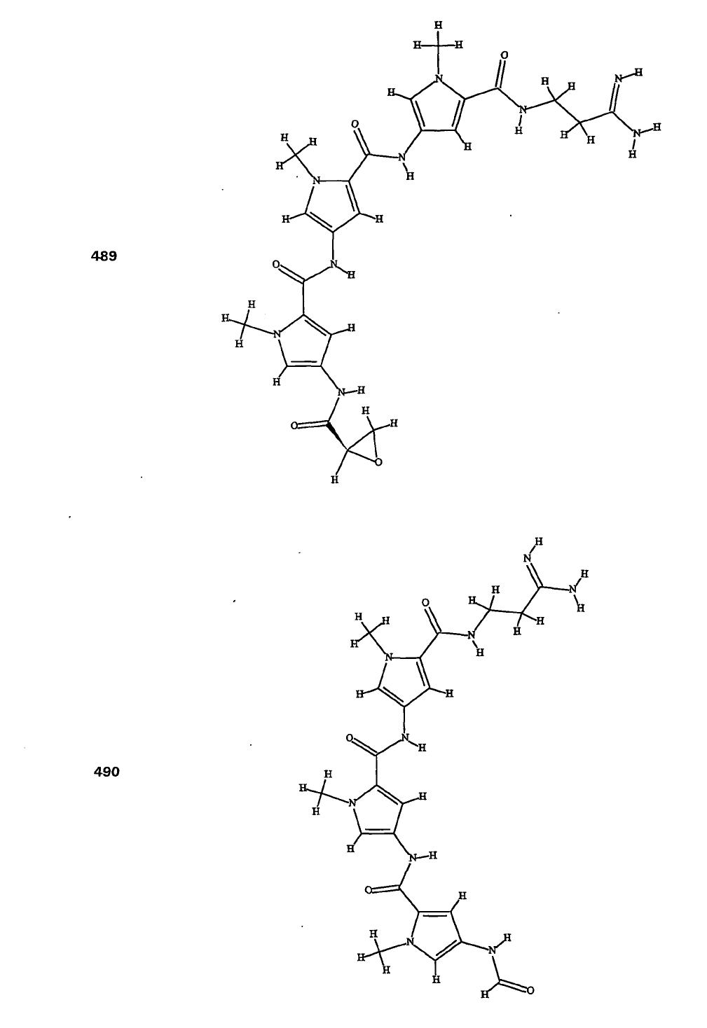

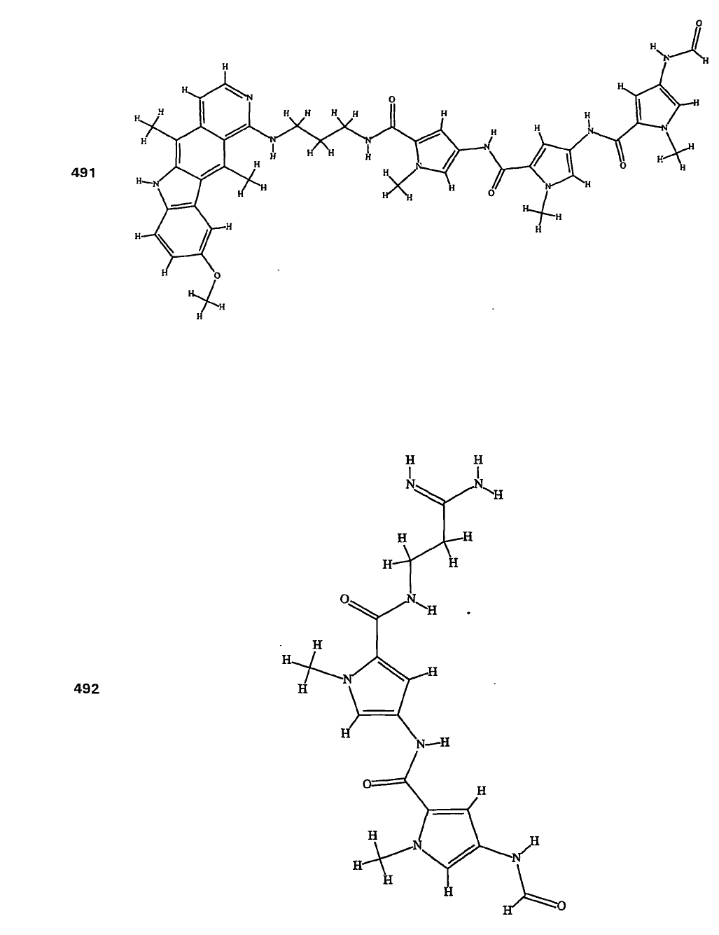

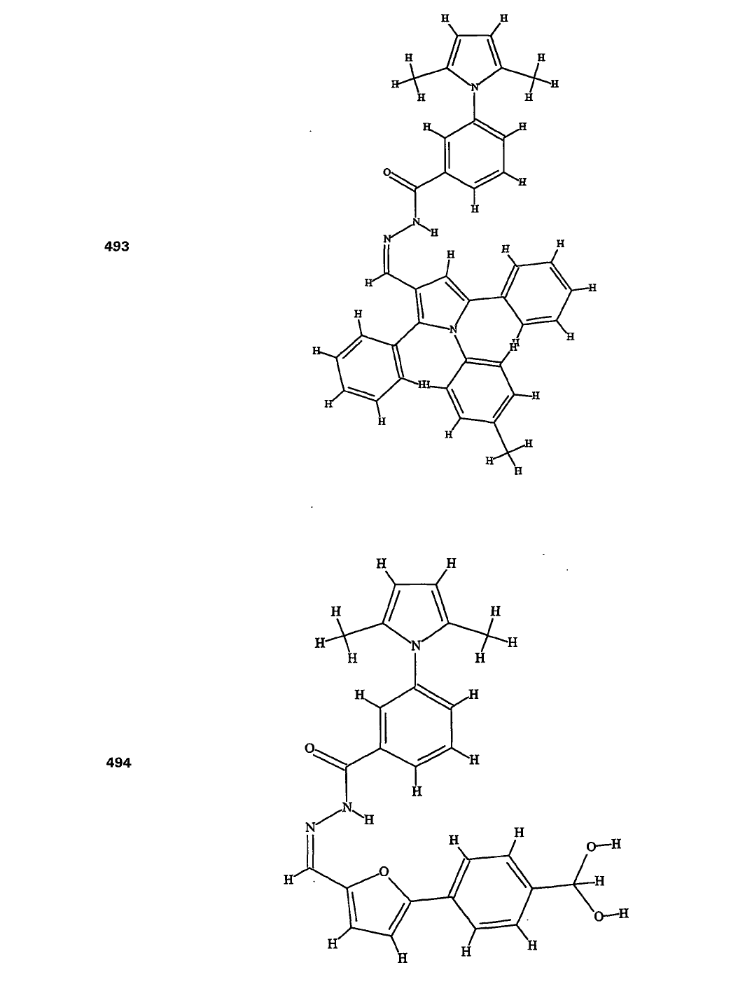

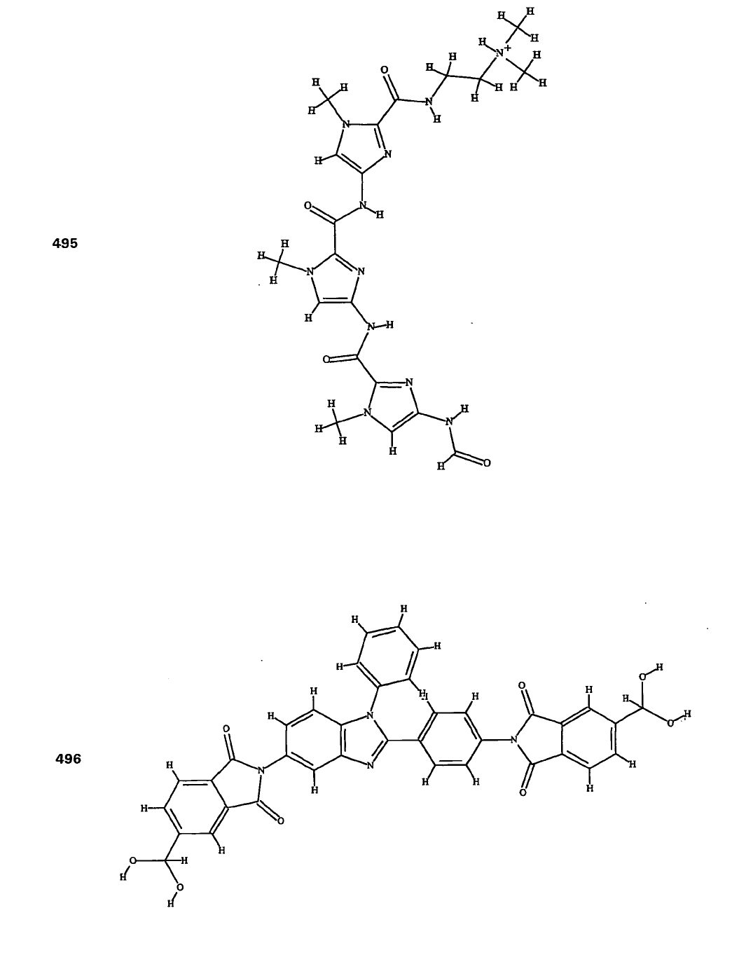

Suitable compounds are for example structures 1 to 492 identified in FIGS. 1 to 42. Also structures, which comprise structures 1 to 492 as substructures are useful in the present invention. Also parts and/or substructures of the structures 1 to 492 are useful in the present invention, as long as they comprise at least an aromatic system and an amid-bond.

In a further embodiment of the invention, the inhibition of expression and/or functional activity MIA is achieved by using DNA or RNA derivatives including aptamers and/or spiegelmers that bind to MIA.

Inhibition of expression and/or functional activity of MIA is can also be achieved by using antibodies or antibody fragments, such as F_{ab}-fragments, single chain antibodies or combinations thereof. These molecules can be identified and obtained by screening antibody libraries and testing the expression products for inhibiting expression and/or functional activity of MIA.

Any of the foregoing elements, molecules or substances can be combined with an immunostimulatory agent, for example cytokines and/or inhibitors of the expression and/or function of interleukin-10 and/or transforming growth factor beta (TGF-β) and/or Prostaglandin B2 and/or receptors for Prostaglandin E2 and/or inhibitors of VEGF.

The present invention is also concerned with a composition for the manufacturing of a medicament comprising a molecule or a combination of molecules which is able to inhibit the expression and/or functional activity of MIA.

The resulting medicament comprising an inhibitor of the expression and/or functional activity of MIA is also subject of the present invention. The medicament of the invention may be combined with an immunostimulatory agent.

Any of the foregoing elements, molecules or substances can be employed for the preparation of a medicament for the prevention or the treatment of neoplasms, infections and/or immunosuppressive disorders.

Preferably, both, the inhibitor of MIA expression and/or functional activity is applied locally to a tumor or other pathologically affected site or organ and may also is applied systemically (e.g. i.v. or s.c. or orally).

The present invention is also related with the use of a method for stimulating the immune system by inhibiting expression and/or functional activity of “Melanoma Inhibitory Activity” (MIA) in combination with the use of methods and/or molecules enhancing the immune response against diseased cells or pathogens, methods and/or molecules enhancing immunogenicity of target cells and/or target pathogens and/or

immunostimulatory molecules, comprising cytokines including interleukins, including IL-1, IL-2, IL-4, IL-12, IL-18, such cytokines being applied systemically to an organism including man or being applied locally e.g. to certain regions or organs or parts of organ or compartments of a body and/or

enhancing expression of cytokines in target cells or pathogens by stimulating their expression and/or by transfecting expression Systems into the target cell or target pathogen, capable of expressing these cytokines and/or

chemokines attracting immune cells including lymphotactin, such chemokines being applied systemically to an organism including man or being applied locally e.g. to certain regions or organs or parts of organ or compartments of a body and/or

enhancing expression of chemokines in target cells or pathogens by stimulating their expression and/or by transfecting expression systems into die target cell or target pathogen, capable of expressing these chemokines and/or

peptides and/or DNA and/or RNA molecules and or other antigens that are found in tumor cells and/or pathogens, but not in normal cells and/or

enhancing expression of peptides and/or antigens that are found in tumor cells and/or pathogens, but not in normal cells and/or

tumor cell extracts and/or tumor cell lysates and/or adjuvants.

The method of the present invention is especially useful for the treatment of
1. Solid tumors, e.g. cancer of the skin (including melanoma), head and neck cancer, sarcoma (including osteosarcoma and chondrosarcoma), retinoblastoma, breast cancer, ovarian cancer, small-cell bronchogenic/lung carcinoma, non-small-cell bronchogenic/lung carcinoma, esophageal cancer, colon carcinoma, colorectal carcinoma, gastric cancer, small intestine carcinoma, liver carcinoma, carcinoma of the kidney, pancreas carcinoma, gallbladder cancer, cervical carcinoma, endometrial cancer, mesothelioma, prostate carcinoma, testicular carcinoma, brain tumor
2. Leukemia, e.g. myeloid leukemia (acute and chronic), acute lymphoblastic leukemia (ALL), Non-Hodgkin Lymphoma, Hodgkin-Lymphoma
3. Degenerative disorders, e.g. arthritis, degeneration/injury of cartilage and bone
4. Immunosuppressive diseases e.g. HIV infection, myelosuppressive diseases, ataxia-telangiectasia, DiGeorge syndrome, Bruton disease, congenital agammaglobulinemia, combined immunodeficiency disease, Wiscott-Aldrich syndrome, complement deficiencies, leukopenia.

### EXAMPLES

### Example 1

Allogenic anti-glioma LAK immune response was strongly inhibited by exogenous addition of MIA.

To study the effects of MIA upon cytotoxic T-lymphocytes (CTL) and Lymphokine Activated Killer cells (LAK cells) a CARE-LASS assay has been employed (Lichtenfels, R., Biddison, W. E., Schulz, H., Vogt, A. B. and R. Martin. CARE-LASS (calcein-release assay), an improved fluorescence based test system to measure cytotoxic lymphocyte activity J. Immunol. Meth., 172: 227-239, 1994). Briefly, glioma cells were harvested, washed in 5% FCS/PBS solution and resuspended at 10 Mio cells/ml in 5% FCS/FBS. Calcein-AM was added to a final concentration of 25 μM (Molecular Probes, USA). The cells were labeled for 30 min at 37° C. then washed twice in 5%/FCS/PBS, adjusted to 1 Mio cells/ml and loaded into 96-well U-shaped microtiter plates at the final concentration of 0.1 Mio/100 μL/1 well (Nunc, Denmark). To measure cytotoxic activity of effector cells pretreated with MIA (f.c. 500 ng/ml), wells were loaded with 100 μL of CTL and LAK cells to produce the desired E:T ratios of 1:10 and 1:100. To measure spontaneous release and total release of calcein, wells were preloaded with 100 μL 5% FCS/PBS or 100 μL lysis buffer (50 nM sodium-borate, 0.1% Triton®×100, pH 9.0) respectively. After incubating the plate for 4 h at 37° C. the supernatants (50 μL) were transferred into new wells and measured using an automated fluorescence scanner (Titertek Fluoroskan II, Germany). The percent cytotoxicity was determined from the following equation:

$$\frac{F / CTL assay - F spontaneous release}{F total lysis - F spontanous release} \times 100 = \% cytotoxicity$$

Results:

MIA inhibited autologous and allogenic LAK, cytotoxicity against malignant glioma ceil lines (HTZ-17, -243, -374, -375) up to 40% a compared to controls.

### Example 2

Furthermore, inhibition of MIA synthesis in MIA-secreting melanoma cells enhanced autologous LAK and CTL activity.

To study the effects of MIA upon cytotoxic T-lymphocytes (CTL) and Lymphokine Activated Killer cells (LAK cells) a CARE-LASS assay has been employed as described above in Example 1.

Briefly, melanoma cells were harvested, washed in 5% FCS/PBS solution and resuspended at 10 Mio cells/ml in 5% PCS/PBS. Calcein-AM was added to a final concentration of 25 μM (Molecular Probes, USA). The cells were labeled for 30 min at 37° C., then washed twice in 5% FCS/PBS, adjusted to 1 Mio cells/ml and loaded into 96-well U-shaped microtiter plates at the final concentration of 0.1 Mio/100 μl/1 well (Nunc, Denmark). To measure cytotoxic activity of effector cells pretreated with MIA-antisense oligonucleotides (f.c. 1-5 μM), wells were loaded with 100 μl of CTL and LAK cells at E T ratios of 1:10 and 1:100. To measure spontaneous release and total release of calcein, wells were preloaded with 100 μl 5% FCS/PBS or 100 μl lysis buffer (50 nM sodium-borate, 0.1% Triton®×100, pH 9.0) respectively. After incubating the plate for 4 h at 37° C. the supernatants (50 μL) were transferred into new wells and measured using an automated fluorescence scanner (Titertek Fluoroskan II, Germany). The cytotoxicity was determined according to the equation described in Example 1.

Results

Inhibition of endogenous MIA synthesis by specific phosphorothioate antisense oligonucleotides in human, MIA-secreting melanoma cell lines (GI and HW) was enhanced by up to 20% autologous LAK cytotoxicity compared to untreated MIA-producing melanoma cell lines.

Active sequences inhibiting MIA expression were

| | | |
|---|---|---|
| MIA-2841-W | GTC AGG AAT CGG GAG | (Seq. ID No 1) |
| MIA-1278-W | CTT GGA GAA GAG ATA C | (Seq. ID No 2) |
| MIA-2842-W | TGC CTC CCC AGA AG | (Seq. ID No 3) |

Less active or inactive sequences inhibiting MIA expression were

| | | |
|---|---|---|
| MIA-2843-N | CAG TGG GAG TAG AAA TC | (Seq. ID No 4) |
| MIA-2844-N | GGT GAG TGG GAG TAG | (Seq. ID No 5) |
| MIA-0202-N | ATG GTG AGG AAT CG | (Seq. ID No 6) |
| MIA-1277-N | GAA TGG TCA GGA ATG G | (Seq. ID No 7) |
| MIA-2328-N | CAT GGT GGA GTG TG | (Seq. ID No 8) |

### Example 3

Furthermore, peptides inhibiting MIA activity in MIA-secreting melanoma cells also enhanced autologous LAK activity by up to 30%.

### Example 4

Inhibition of endogenous MIA synthesis by specific phosphorothioate antisense oligonucleotides in human, MIA-secreting melanoma as well as breast cancer cell lines strongly reduced their migration activity, as well as increasing their adhesion to matrices, both showing a strong inhibitory effect of MIA inhibitors on tumor invasion and metastasis.

### Example 5

Inhibition of endogenous MIA synthesis by specific phosphorothioate antisense oligonucleotides in human, MIA-secreting melanoma cell lines (GI and HW) in combination with application of the cytokines IL-12, IL-4, IL-18 and/or antisense oligonucleotides specific for TGF-B increased the autologous LAK cytotoxicity even further compared to inhibition of endogenous MIA synthesis by specific phosphorothioate antisense oligonucleotides alone in MIA-producing tumor cell lines.

### Example 6

Inhibition of endogenous MIA synthesis by a transfecting vector expressing the antisense sequence (SEQ. ID No 9):

GGCAGGGCCAGCGGTAGGCTGAGCTCACTGGCAGTAGAAATCCCATTTGT
CTGTCTTCACATCGACTTTGCCAGGTTTCAGGGTCTGGTCCTCTCGGACA
ATGCTACTGGGGAAATAGCCCAGGCGAGCAGCCAGATCTCCATAGTAATC
TCCCTGAACGCTGCCTCCCCAGAAGAGCCGCCCACGGCCCTTCAGCTTGG
AGAAGACATACACCACTTGGCCCCGGTGAATGGTCAGGAATCGGCAGTCG
GGGGCCATGTAGTCCTGAAGGGCCACAGCCATGGAGATAGGGTGGCTGCA
CTCCTGGTCCGCACACAGCTTCCGGTCAGCCAGCTTGGGCATAGGACCAC
CCCTGACACCAGGTCCGGAGAAGGCAGACAGCAAGATGATGACACCAAGG
CACACCAGGGACCGGGCCATCGTGGACTGTGAGCAAGAGAGTGAGCAAGG
GGGTGCTGG

or parts of this sequence in human MIA-secreting melanoma as well as breast cancer cell lines strongly reduced their tumor invasion and metastasis in scid-mice and nude mice.

### Example 7

Inhibition of tumor invasion and metastasis was increased by a combination of inhibitors of MIA with inhibitors of VEGF or TGF-β.

1. A method for stimulating immune cells and/or the immune system, and/or reducing invasion and/or metastasis of tumor cells by inhibiting expression and/or functional activity of “Melanoma Inhibitory Activity” MIA.

2. The method according to claim 1, wherein the inhibition of the expression and/or functional activity of MIA is achieved by using at least one nucleic acid molecule or derivative thereof,

3. The method according to claim 2 wherein the at least one nucleic acid molecule is an oligonucleotide, an antisense nucleic acid and/or a ribozyme.

4. The method according to claim 1, wherein the inhibition of the synthesis and/or function of MIA is achieved using a molecule comprising the antisense sequences SEQ ID No. 1 to 3 or SEQ ID No. 10 to 39 or parts of the sequences having at least 8 nucleotides.

5. The method according to claim 3 wherein the antisense and/or ribozyme molecule is derived by synthesising a sequence wholly or partially complementary to MIA mRNA and testing for inhibitory activity of MIA.

6. The method according to claim 3 wherein the antisense and/or ribozyme molecule is integrated into a DNA delivery system comprising viral and/or non-viral vectors together with lipids selected from the group of anionic lipids, cationic lipids, non-cationic lipids and mixtures thereof.

7. The method according to claim 3 wherein the antisense and/or ribozyme molecule is modified at one or more of the sugar moieties, the bases and/or the internucleotide linkages and/or by coupling the antisense and/or ribozyme molecule to an enhancer of uptake and/or inhibitory activity.

8. The method according to claim 1 wherein the inhibition of the expression and/or functional activity of MIA is achieved using peptides and/or proteins.

9. The method of claim 8 wherein the peptides and/or proteins comprise the sequences SEQ ID No. 40 to 63 and analogs or derivatives thereof.

10. The method according to claim 8 wherein the peptide and/or protein is derived by screening an expression library and testing the expression products for inhibitory activity of MIA.

11. The method according to claim 8 wherein the peptide and/or protein is derived by screening randomly synthesised peptides and/or proteins for inhibitory activity of MIA.

12. The method according to claim 1, wherein the inhibition of the expression and/or the function activity of MIA is achieved using an inhibitor of low molecular weight.

13. The method of claim 12 wherein the inhibitor of low molecular weight is selected from compounds having any on of the structures 1 to 492 of FIG. 1 to 42 or comprise any of these structures as substructures, or parts of the structures 1 to 492 comprising at least an aromatic system and an amid bond.

14. The method according to claim 12 wherein the inhibitor of low molecular weight is obtainable by combinatorial chemistry and testing the products for inhibitory activity of MIA.

15. The method according to claim 1, wherein the inhibition the expression and/or functional activity of MIA is achieved using DNA or RNA derivatives including aptamers and/or spiegelmers that bind to MIA.

16. The method according to claim 1 wherein the inhibition of MIA is achieved using antibodies or antibody fragments, such as F_{ab}-fragments, single chain antibody or combinations thereof.

17. The method according to claim 16 wherein the antibody or antibody fragments, such as F_{ab}-fragments, single chain antibody or combinations thereof are obtainable by screening antibody libraries and testing the expression products for inhibitory activity of MIA.

18. The method according to any of the claims 1 to 17 wherein additionally an immunostimulatory agent, such as cytokines and/or inhibitors of the expression and/or function of interleukin-10 and/or transforming growth factor beta (TGF-β) and/or Prostaglandin B2 and/or receptors for Prostaglandin E2 and/or inhibitors of VEGF.

19. A composition comprising a molecule or a combination of molecules for inhibiting the synthesis and/or function of MIA

20. A composition according to claim 19 wherein the molecule is selected from the oligonucleotides having any one of the SEQ. ID. No. 1 to 3 and SEQ ID No. 10 to 39 or parts of these sequences having at least 8 nucleotides.

21. A medicament comprising an inhibitor of the synthesis and/or function of MIA.

22. A medicament comprising an inhibitor of the synthesis and/or function of MIA combined with an immunostimulatory agent.

23. The use of the composition according to claim 19 for the preparation of a medicament for the prevention or the treatment of neoplasms, infections and/or immunosuppressive disorders.


SEQUENCE LISTING (63 sequences)

SEQ ID NO: 1 - DNA, 15 bp, Homo sapiens
gtcaggaatcggcag

SEQ ID NO: 2 - DNA, 16 bp, Homo sapiens
cttggagaagacatac

SEQ ID NO: 3 - DNA, 14 bp, Homo sapiens
tgcctccccagaag

SEQ ID NO: 4 - DNA, 17 bp, Homo sapiens
cactggcagtagaaatc

SEQ ID NO: 5 - DNA, 15 bp, Homo sapiens
gctcactggcagtag

SEQ ID NO: 6 - DNA, 14 bp, Homo sapiens
atggtcaggaatcg

SEQ ID NO: 7 - DNA, 16 bp, Homo sapiens
gaatggtcaggaatcg

SEQ ID NO: 8 - DNA, 14 bp, Homo sapiens
catcgtggactgtg

SEQ ID NO: 9 - DNA, 459 bp, Homo sapiens
ggcagggccagcggtaggctgagctcactggcagtagaaatcccatttgtctgtcttcacatcgactttgccaggtttcagggtctggtcctctcggacaatgctactggggaaatagcccaggcgagcagccagatctccatagtaatctccctgaacgctgcctccccagaagagccgcccacggcccttcagcttggagaagacatacaccacttggccccggtgaatggtcaggaatcggcagtcgggggccatgtagtcctgaagggccacagccatggagatagggtggctgcactcctggtccgcacacagcttccggtcagccagcttgggcataggaccacccctgacaccaggtccggagaaggcagacagcaagatgatgacaccaaggcacaccagggaccgggccatcgtggactgtgagcaagagagtgagcaagggggtgctgg

SEQ ID NO: 10 - DNA, 14 bp, Homo sapiens
agccatggagatag

SEQ ID NO: 11 - DNA, 15 bp, Homo sapiens
cagccatggagatag

SEQ ID NO: 12 - DNA, 16 bp, Homo sapiens
acagccatggagatag

SEQ ID NO: 13 - DNA, 17 bp, Homo sapiens
cacagccatggagatag

SEQ ID NO: 14 - DNA, 16 bp, Homo sapiens
ccacagccatggagat

SEQ ID NO: 15 - DNA, 14 bp, Homo sapiens
gccatggagatagg

SEQ ID NO: 16 - DNA, 15 bp, Homo sapiens
agccatggagatagg

SEQ ID NO: 17 - DNA, 16 bp, Homo sapiens
cagccatggagatagg

SEQ ID NO: 18 - DNA, 17 bp, Homo sapiens
acagccatggagatagg

SEQ ID NO: 19 - DNA, 14 bp, Homo sapiens
catggagatagggt

SEQ ID NO: 20 - DNA, 15 bp, Homo sapiens
catggagatagggtg

SEQ ID NO: 21 - DNA, 16 bp, Homo sapiens
catggagatagggtgg

SEQ ID NO: 22 - DNA, 14 bp, Homo sapiens
atggagatagggtg

SEQ ID NO: 23 - DNA, 15 bp, Homo sapiens
atggagatagggtgg

SEQ ID NO: 24 - DNA, 16 bp, Homo sapiens
atggagatagggtggc

SEQ ID NO: 25 - DNA, 17 bp, Homo sapiens
atggagatagggtggct

SEQ ID NO: 26 - DNA, 14 bp, Homo sapiens
ggagatagggtggc

SEQ ID NO: 27 - DNA, 15 bp, Homo sapiens
ggagatagggtggct

SEQ ID NO: 28 - DNA, 14 bp, Homo sapiens
gaaatagcccaggc

SEQ ID NO: 29 - DNA, 15 bp, Homo sapiens
gaaatagcccaggcg

SEQ ID NO: 30 - DNA, 17 bp, Homo sapiens
gaaatagcccaggcgag

SEQ ID NO: 31 - DNA, 14 bp, Homo sapiens
ggaaatagcccagg

SEQ ID NO: 32 - DNA, 15 bp, Homo sapiens
ggaaatagcccaggc

SEQ ID NO: 33 - DNA, 14 bp, Homo sapiens
gtcttcacatcgac

SEQ ID NO: 34 - DNA, 15 bp, Homo sapiens
gtcttcacatcgact

SEQ ID NO: 35 - DNA, 16 bp, Homo sapiens
gtcttcacatcgactt

SEQ ID NO: 36 - DNA, 17 bp, Homo sapiens
gtcttcacatcgacttt

SEQ ID NO: 37 - DNA, 18 bp, Homo sapiens
gtcttcacatcgactttg

SEQ ID NO: 38 - DNA, 18 bp, Homo sapiens
gtcttcacatcgactttg

SEQ ID NO: 39 - DNA, 18 bp, Homo sapiens
ccatttgtctgtcttcac

SEQ ID NO: 40 - protein, 7 aa, Homo sapiens
VPHIPPN

SEQ ID NO: 41 - protein, 7 aa, Homo sapiens
MPPTQVS

SEQ ID NO: 42 - protein, 7 aa, Homo sapiens
QMHPWPP

SEQ ID NO: 43 - protein, 7 aa, Homo sapiens
QPPFWQF

SEQ ID NO: 44 - protein, 7 aa, Homo sapiens
TPPQGLA

SEQ ID NO: 45 - protein, 7 aa, Homo sapiens
IPPYNTL

SEQ ID NO: 46 - protein, 7 aa, Homo sapiens
AVRPAPL

SEQ ID NO: 47 - protein, 7 aa, Homo sapiens
GAKPHPQ

SEQ ID NO: 48 - protein, 7 aa, Homo sapiens
QQLSPLP

SEQ ID NO: 49 - protein, 7 aa, Homo sapiens
GPPPSPV

SEQ ID NO: 50 - protein, 7 aa, Homo sapiens
LPLTPLP

SEQ ID NO: 51 - protein, 12 aa, Homo sapiens
QLNVNHQARADQ

SEQ ID NO: 52 - protein, 12 aa, Homo sapiens
TSASTRPELHYP

SEQ ID NO: 53 - protein, 12 aa, Homo sapiens
TFLPHQMHPWPP

SEQ ID NO: 54 - protein, 12 aa, Homo sapiens
VPHIPPNSMALT

SEQ ID NO: 55 - protein, 12 aa, Homo sapiens
RLTLLVLIMPAP

SEQ ID NO: 56 - protein, 9 aa, Homo sapiens
RKLPPRPRR

SEQ ID NO: 57 - protein, 12 aa, Homo sapiens
VLASQIATTPSP

SEQ ID NO: 58 - protein, 12 aa, Homo sapiens
TPLTKLPSVNHP

SEQ ID NO: 59 - protein, 13 aa, Homo sapiens
PPNSFSSAGGQRT

SEQ ID NO: 60 - protein, 15 aa, Homo sapiens
EQDSRQGQELTKKGL

SEQ ID NO: 61 - protein, 13 aa, Homo sapiens
ETTIVITWTPAPR

SEQ ID NO: 62 - protein, 13 aa, Homo sapiens
TSLLISWDAPAVT

SEQ ID NO: 63 - protein, 13 aa, Homo sapiens
NSLLVSWQPPRAR